SEQUENCE LISTING (27 sequences)

SEQ ID NO: 1 - protein, 1210 aa, Homo sapiens [sequence omitted]

SEQ ID NO: 2 - DNA, 197496 bp, Homo sapiens
ttctttttagcacagaataacaatccattgtccacatgtaccatggtttatttatccactcatccacatgaagacatcttagttgattctaagttagggaagttatgaataaagctgttataaatattcatgagcagatttatgtggacaacagtgttcaactcatttgggtaagtatcaaggagagaaatcattggatcatatggtaagagtatgtacacttttataggaaactgctaagctgcattcctaagtggctgtaccattgtgccttcccatcagcaatgaatgagacttcctattgttccacatcctcatcagaatttggtgttgtcactgatctgaattttttccattgtaacagatgtgtagtggtatctcactgttgttttaatttgcaatttcctaatgacatatgatgttgaacatctttttatatgcttacttgccatcagtgtatcctctgatgaggtgtttgtgtagggctttggcccatttttaaatcaggttatttatcctcttattattaacttttaagagtttagttctttgcatattttggataacaatcctttatcacatatttcttttgcaaatttttctccagtatatggcttgtcttcttctcctggcattgtccttctcagagcagaagtttttaattttaataaactccagcttataaattatttatttcatggattgtgcctttggttttgtacttaaaaagtcattgtcatacctaaggtcatctaggttttctcctacgttatctcctaggtgttttatagttttgcattttacatttatatgtatgatcagttttgagttaatttttatgaagtgtgtaaggtttgtatctacattcattttttgcatgtggatgtccatttgttccagcaatatttgttgaaaagactatacttgctctattgtattgtgtttcttttttgtcaaagatcaattgactaaatttatgtgcgtcagtttctgatctctctggtccattgatatatttgtctattctttcaccaataccacatagtctagactactgtagttgtatatgtcttgaagtcaggtagtgttgatcctccaattttgttctccaatattgagttggctattgtgggtcttttgcttgcccatagaatcaattagtcaatatttacaaaataacttgctcgaactttgactgggattaatctataaatcaagttgggaataagtgacattttgacattatggagtctttctgaccatgaacacaaactattgatccatttatttagttatttgatatctttcaccagagtttttttgtttttttcttatagatcttatacatattttcttatattcatacctcagtattccatttcagggtgttaatgtaaatggtaatgtgtttttaatttcaaattcccttagttctttgctggtatataggaaagtgattggcttttgtatgttaacgttgtatcctgctcacttgctataactgcttattagttccaggagcttttttattgtttcttttggattttctaagagacaattacattatcagtgaacaaacacgatttatttcttccctcccaatcagtattcattttatttatttattgtgtgttattgcattagctaggacttctaatacaatgttgaaaagcattggtgaaaggaaacatccttgctttgttcctgattttagctataggtttttgtagctgttctttattaagttgaggatatccttctctattcttagtttgctgagaatttttatcatgaataggtgtaggattttgcctaatgttttcttctgtatctattgatatgatcatgtaatttttctttatctattgatatgatctgttgatgtgatgaactacattaattgagttttcaaatgttgaaccagtcttgcatatctggaataaatcggagttggtcatcatgtataactttgttacactttgttgcatttgattttgtaatattttcttgagaatttttacatctatgttcataaaagatatcggtctacagttttctttcctttcttgtaatatctctgtctggttttgctattaaggtaattctggcttcacaaaattaattatggagtcttccctctacttctagtttctggaagagattgtagagaatggatgtaatttctttcttaaatgtttgacgaaaatcagcactgatctcatctgggcttggtgctttctgttttggaaggttattaattatttattcaatttctatagtggatataggcctatattgattggcaatttcttcttgtatgacttttggtacattctatttcaaggaattggttcatttcatgtaggttgttaaattcgtgggtatagctgttcataatattcatttattatcctttcaatgttcatgagatcagtagtgatgttccttctttcatttctgatattcataatttgtgtattctctctttgtttcttagcctggtgagaggcttataaattttattgattttttgaagaatcactttttggttttgctgattttcttgctgggattataggcgtgagccaccacactcttgctcattttttctatttttgttttccacactttttctgcctctgtggattttacacagcattttgtataattcgatttcctctttttagcatagcaattattttagtctttaactttttaaaatcagttgccctagatttttttctcccaacattttgggaggccaaggggagaggatcacttcaagccaggagtttgagaccagcctgagcaacatagcaaggcactatctatacaaacataattttaaaaagtccaggcattagctaggactgtgcctgtagtcccagctactcaggaggctgagatgagaagatcaccggagcctagaaatttgaggctgcagtgggctgtgatcatgtcacttcactcctgcctagaagacagttagaccctgcctctaaaataaacaagcaaataaataaaaagaaaggaagaaaagaagagcaagggcagcaaatagaaaatagtaataaatatggtagctattaatccaactatgtcaataattaccttaaatgttagtggtctaaatatactgcaatggactgaatgtttatgtctcctcaaaatgtatatgatgaaatgtaagctcccaaaatgatgttatcaggggagagtgtttttgggaggggattatgtcatgagggtggaggccttacaaatggggttagtgctataaaagagaccacagagagctgccttggtccttctgccatgtgagggcactgtgaaattatggccatctatgaagaagtgggcccttattagacatcaaatctgcaaataccttgatcttgaatttcccagcctccagaactatgggaaataaatttctgttgtttacaagtaaatcattttatgttattttgttacagaagcccaaaaagatgaagacatacaccagatcactccattctcttcttgcttgcatggtttctgaggatacgttggatgtaattctaatattctctataggtattttctttatggctcctctacaggtaaggtgttttttccctttggcttcatttaagaatttttcgttaacctttgattttctgaagtgtgaatatgttatgcctaggtatcatttgtttgtttgggttttcttggcatatatcttcctgatgttctctgaacttccagaatctgtgatttgttgtctgacattaatttggaggaatttttggtattattgctttaaatattgcttctgctcctttttctctttctttgacttacagtattcccattacatgtaattatctcacagttcttgaaagttttgttttgttctttttgttagtcgtttttctttctgtttttcagttttggcagtttctgtttacgtatcttcaatctcagaaattctttcctcaagcatgttcagcccactaatgtgtacttcaaaagtattctacatttcttttacagtgtttttgatctctagaatttttaaattctttcttaaaactttcatctctcaggaattcaagactagcctgggcaacatagtgaaactctatctctacaaaacattagccaggtatggtgatgcatgcctgtagtcagagctactcaggaggctaaggtgggaggatcacctgagcctgggaagttgaggttgcagtgagccaaggtcacgccactgcactctggattgggcaacagagccagaccctgtctcaaaaaaaagaaaaattccatggctctgcttacattatccatctgatcttacatgttgcctattttttccattaaaactcctagcctattaatcatagtttttttataattaatactccgatgtgataatgtcttagtccaattactgtggttataacagaatgccacaaactgggtgatttataaacaaaagaagctgatttaggctgatttagaggctggggagtccaagagcttggtgctagcatctgatgagtgtcttcttgcttcatcataacatgggagagggcatcacgtgtgaagagagcttactcttataacatagccactcccacaagaattaacccaccgccatgagagccatgtgaattcattcatgaggacagcgggttaagtttccaatatatggacttttcggggacacattcaaaccacagcagttagttgtaacgttcgtgtcatgtctcattctggttctgatgcttgtgcagtctcttcaaactgcgtctttgccttttagtgtgccttgcaatgtggaaatgatatactgggtaagaggagctgtagtaaagaggcttctagtgacgtagtgacaagctgtggggagagggagtgttgcacagtcctgccgcatgtcacagtcttccagtgagcctgtgtccctggactgtgaacttcatgcttgcttctcagcttccccagccccttagatggtacagaactgttggaggggggtggagttgtatatttcccttgctctgggtaggtcaccctctgataaaacaccaggttaggcctctggtgaaataatttctcctgagggcagaccttctattaataatagaatgttccaacctatttcaaaatggttcctcttctccttccactgccagaagcataatgagattttccccctaatattcgtggtaaggacctagcagagctccaggaggtaacactctcaagtgtctcatactaccctgcaccatgactgggctctgctggagttcttaatttgcagaactgcccacactgagcctcccgcaatttctcaattacagggcaaactttcccagccggcactgggtccttggaggtttctgtctgctggtttcttcctctggaggttgtgcttctgtgtttgcctgtctctccaatttggggggcagtggtttgcccaatgacctcaattctctgaaagagctaagaagaggtgttaatttttcggtttgctcagctttctacttgttgctagaatggagcgccaatagtgcctcctatagtgacatgtaaccctcaactctagagatgatgaagcatactaatgacaaaggagaaatgcttcagcagttttctgtcagcacattaccccttgaaaaagctgcttcttccacattctgcaagagatgggtctcaactcagagctcaaggcaaatgacttccttcaaggagaaggaataaacagtctcagaaaccatgaaagcctgcccccaggagtgtccctgaacctcagcaggggccacacttaccttgcagaaataggtgaggcatgctcctggtacaaaatcccaatggtacagaaggacaaattgaaaaacaagtctccctctaaacccctgaccccgagctacctagttctcctccctagaggcaaagctgttaccagattcttgtatctccttaacatatatccttagaagagctgtcaagtgaacacatgtttaagtgaaaacctattttagaagtgcattttcttaaggaactttagggttggaaggaacctgtgtcagtccttaattcacaacctccattagtacttattgttcttgcacaaaaatctttctcaaaaaagccctttccactctgacatagcttattctacttttacttagctccaataacttataaaacatatttttgaaagtctaaaatctgccactatgtttttttttcctaatcaatctttactttgacctctaagccagagaaaacaggtggtcaaatgccttttgcctaagatggaacttagaatatttgaagacctcagatcttcaccctgccaaataacgtgtttctcctcccctttcacagagcatttggttttaggaaattcagagccacattccttatagacaagactaaactcttattcaacatactcagaaacttcttctaagaggataaccactcatcagaggaaaaaagtttctcatgtacagctggcaaagggatggaaccatctgtgttattaaaattgacagacgcttatgagatttattaagggaaatactagagtcttagtacatacttgctaatatagcatacatgaaggctttatctataatttttttggccaagcagaaattttggtattactcaccctaacaaatttccaagacattatgaaatagaattttaggtcctgacatcaccatttgtctcaggttttgaagcgttgctggacaagaggggtaaaacacggctctgccttggattcaaagttggcctctcatactagcaagtataccttggtatcctggtcacttctcccggccacagcatcacattgctataaaaggcagatacaagtattaaccagctcacaggttatcagataagcttagtctgaccaatgcttaacacagcaactgggccactattgtcattcctgtggtggtggcacacacacccagcctctgtccgggccatggtctaggaccaccctccacagaggctgtgagctagagccctaactgtgcagggccctaactatgccaggctacttatctctcttaagaggacttcattagtgcctgctcggccatacagttttttacttaccaagtaacacagttatcagcacactccaggtactagccaaggactacaaaatcaacgtgaatgtcagcttttgtatcaaaagctcaaaggagaaactcaaactttacatagatgtcccatgaagatgttcagcaaacccattcttctctgttccctggaatccatcccagtattgtgctatgtgtgtgtctagtaattctttacaaaaagctctgtttcttgtgatgctatcagatcacattgaagaatatacaagccgtactatgaaggctgttgtctcatatagtcctaacgtagtgagaactgatgttcttacatgctgtctttttgggcactcaaagaaattcctgtacagtcttacaaatcagttgtagcttaaattgatttgtgttgtgacttgtacacacaggtcacattcccttgacagaaaatatagtttaaaaccaaatttgcagcccttgttaagtgaatgcacaggactttattgtattcaggtcttttattgtaagactcactcctgtcttcattttatgttccactgttgtgcttcccatttgcctttctctagttttgttttctgtgtttctacggactgctctcagcccaggtgtgcaggaagcacacacatgcctgcagagccttcatggcctctgcattcagggcatgacttcaacgcacagtggctgtactgatttgttaaaacaaaggaacagattacttctcctaattcacagggaagttccaggttgtgcgggcagtgagcagacctgtgtctgtctgcgcttgccctggtgaaaaaccccaccgttcaggctgcagggtgcgagacccaggcacaaacattttgctggatgaggaggaaagatgtaaggttgctccccttcagagacagcaaagggcaggtctgtagcttcacttacttcaggattgtgatttttgacagagccgagagatcagggttgttgaaccaggcctgaaggtcctagtgaatctcgtgaagagaggaggggtctggctgtaacatggacctagaggacatttttactgcaggagaaggaacagtggggatggggtggacttgccaaaggaatatagctcaagttcctgcagcccaaaaaagctcagtttcttttggccaaagcttccgcgagtttccctggcatttctcctgcgggagctacaggggcagtgggacacttagcctctctaaaagcacctccacggctgtttgtgtcaagcctttattccaagagcttcacttttgcgaagtaatgtgcttcacacattggcttcaaagtacccatggctggttgcaataaacattaaggaggcctgtctctgcacccggagttgggtgccctcatttcagatgatttcgagggtgcttgacaagatctgaaggaccctcggactttagagcaccacctcggacgcctggcacccctgccgcgcgggcacggcgacctcctcagctgccaggccagcctctgatccccgagagggtcccgtagtgctgcaggggaggtggggacccgaataaaggagcagtttccccgtcggtgccattatccgacgctggctctaaggctcggccagtctgtctaaagctggtacaagtttgctttgtaaaacaaaagaagggaaagggggaaggggaccctggcacagatttggctcgacctggacataggctgggcctgcaagtccgcggggaccgggtccagaggggcagtgctgggaacgcccctctcggaaattaactcctcagggcacccgctcccctcccatgcgccgccccactcccgccggagactaggtcccgcgggggccaccgctgtccaccgcctccggcggccgctggccttgggtccccgctgctggttctcctccctcctcctcgcattctcctcctcctctgctcctcccgatccctcctccgccgcctggtccctcctcctcccgccctgcctccccgcgcctcggcccgcgcgagctagacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaaggtaagggcgtgtctcgccggctcccgcgccgcccccggatcgcgccccggaccccgcagcccgcccaaccgcgcaccggcgcaccggctcggcgcccgcgcccccgcccgtcctttcctgtttccttgagatcagctgcgccgccgaccgggaccgcgggaggaacgggacgtttcgttcttcggccgggagagtctggggcgggcggaggaggagacgcgtgggacaccgggctgcaggccaggcggggaacggccgccgggacctccggcgccccgaaccgctcccaactttcttccctcactttccccgcccagctgcgcaggatcggcgtcagtgggcgaaagccgggtgctggtgggcgcctggggccggggtcccgcacgtgcgccccgcgctgtcttcccagggcgcgacggggtcctggcgcgcacccgaggggcgggcgctgcccacccgccgagactgcactgtttagggaagctgaggaaggaacccaaaaatacagcctcccctcggaccccgcgggacaggcggctttctgagaggacctccccgcctccgccctccgcgcaggtctcaaactgaagccggcgcccgccagcctggccccggcccctctccaggtccccgcgatcctcgttccccagtgtggagtcgcagcctcgacctgggagctgggagaactcgtctaccaccacctgcggctcccggggaggggtggtgctggcggcggttagtttcctcgttggcaaaaggcaggtggggtccgacccgccccttgggcgcagaccccggccgctcgcctcgcccggtgcgccctcgtcttgcctatccaagagtgccccccacctcccggggaccccagctccctcctgggcgcccgcgccgaaagccccaggctctccttcgatggccgcctcgcggagacgtccgggtctgctccacctgcagcccttcggtcgcgcctgggcttcgcggtggagcgggacgcggctgtccggccactgcagggggggatcgcgggactcttgagcggaagccccggaagcagagctcatcctggccaacaccatggtgtttcaaaatggggctcacagcaaacttctcctcaaaacccggagactttctttcttggatgtctctttttgctgtttgaagaatttgagccaaccaaaatattaaacctgtcttacacacacacacacacacacacacacacacacaccggattgctgtccctggttcaagtgtgccaagtgtgcagacagaacatgagcgagtctggcttcgtgactaccgaccataaacccacttgacaggggaaacatgccttggaaggtttaattgcacaattccaaccttgagctgcgcgggttccaagagccaggcccgtacttgctgttgatgtcattggcttggggagttggggtttggtgcccagcgcggtcgttgggggaggggcaaggcatagaacagtggttcccagaccttgctgcacattggaattacctgggattaaaaaaaaaaaaatcaaaacaaaaaccagtgtctggctcccgcccccagacattctgatttaattggcatggggcaagacctggacttgggattttttttaatgctcttcatgtgatctgttgggcagccagatttggggatcactagacggaagaaggattgttaaagtctccggagatgttacttgccaatgctaagagctctttgaggacatctggaattgttacaatattgccaaatataggaaagagggaaaaggtagagtgtgattccaataataaaggattccgcttttcattgaaggaactggtggaaaggtttcttctctgctgagcctgcaggcccgtcctgcctgcctggggtgcccgggagacgcgggcctgctccggagactgctgactgccggtcctgttagtcaggtgtcagccctgtctctgccgaagagactcttctctttattttaaattaaaccctcagagcaccaccaaagcatcacttttctccctccattggtgttctcattctttgatgttacttgtttgaacaccactattagtagttggagatttgttcctgagaaaaatataaataccacttaatttgcctgtttgtcccgcattcactcaaaacagaatgctcctgaagacaagagagagagtaggagaacagacgctattccattacagtaacataaaagactggattttcaggggcaaattattaaaataggagatgagctcttttaacagaaatttgtttaaggcctgtgtctatcaaattcagtggattttattcaagatgcactttgtttagtgggagttttgtttggttctgggacatgctaacttctagacttgctgctcttagaggtaatgactgccagacaccatttcatgagtcctaatccccacattaagcataagaggtgcacactctcctcctatgggggaaactgaggtacgaagaactaaagtgactttcccacagctggtgggaggcagacgggaaattcacaccaggggcttccaactccagatccctctctcaacttccaaactccactgccttgtccgagttctggtttcaggagatccaaatcaggtgtgtgcaaatgtctaatgtcagagctggcaaggggaaagggcccagggagccggctcatgacgatgagcctgtctgaagcttcaacgcgggctgtccggcagtctgcattcctgccgagttcctcagccctctgttgggtcaccttccatagaggcagcttagtcctcagttcagtgagcatggagtggagactgcttgaggggtgctgagcaaagccctgcctcttacaggatgaaggtgctctccagaagggacactggaaagtattccaaggcgagtcgaattcccaactgagggagctttgtggaaataagcccgcccagccccacttctggagacgttcccattcagtaggtccgagctgtcttaaagagaaaccaaagtggggatattaatggtatccaaagtgagatctaccccaccctccctcctcaaaggaggtcagatcaagaaagcccaagcccggcctggcaattgggacctttcttctcactccagcccagggtgaaggtggacaagtcactttgacccttcaggcttctgagctgttgtttctgaattcagtgaatatttactgagtgcatagaatatgctagatattctgggctaaaggttgaagggggggtgagttttaagggtttctgctcttgcttccagattgctttcaaatctggaaaggacaccagtggtttgtgtgttagacccacactgccgtagcacagaatacaagaaactggctgagagctccaataggcttttaacagtaatttctggcttcacgtatttagtttcataactcatgatttttcaaaaacttctggtttgaagacaccgattgccgaaagtccattgtgctgcataattacacttggtccacgtgacagcactaacatgttctgaaatgtttttagaagtagtctcagcaaagatgaaggattcctccctgtttgaaaagaaaatattctttgttttttctttgatctaagctctaagactagcagctagcatctgaaacttttttgacgagagtgacaaaccaactctaatattaaaggcaattgatgattatgggcactgaagggaaggtaaccccaggctggtgccccggaatagggatgggtcacaatgttgaggacatttcgcctgttgcagaacccacctgcaacacagtgtggcccttgccatgtgacttgtgtgtgtgcctgtgtgtctgtgtgtgcgtgttttaattttgacttcataagtactctagttatgagcttatttaacattgggttttactaataggggtatgtgttgagaaaatttcaaagttttagaatatggttcacccacatgttgcttccctgtaaatataatttttaaaaccagattctgggccgggcatggtggctcacctctataatcccaaaacgttgggaggccgaggcaggcgaatcatgaagccaggagtttgagaccaggctgaccaacacggtgaaacccagtctctactaaaaatacaaaaaaaattagctgggcgtggtggcaggtgcctgtaatcccagctactcaggaggctgaggcaggagaattgcttgaacccaggaggcagaggttgcagtgagccaagatcgcaccattgcactccagcccgcgcgacagtgtgagactccatctcaaaaaaaaaaaaaaaaaaacagattctgttcctcagatccattccatttttgttttcctttatcacttatggacatttgaaattatggtaataaacattgttagtctcagttaattattactggtttattcttgaaccactaatccatagagaatagagtgtaaatcttaacttgttcctgtaggccatccccattaaacatcatagtgttttctcattcgttctttttcgttttcctcctacaggaatgaattttctaagaaaattccagcagttggctctttggacgacatctctagattgtcctccattgggcccataggcacaagctggccagtttgaatttgggcaagaatccaggcattggaacttattcaaataactagtttgcctgtaattttcactttttcagagtcatctgataaagctttcttgctacacatttagatagatacactcaatccagttgtctagaaagttccctgagccagctgggagcaggaggggtagttggggccaggaatattgggggtgtgtttactgagcccctagaaagtaagtgctagatttgacatttcaatccctgaaggccctgaagttcagtatcaaatgactggtcctgtggactgagcatctgtgaattgcatatgcttagagtaaattttactcctaccagtttcagcagcttgctttagcaagcagtatggaaacactaacatgggggagtagaatttctctctctgatccaagttttatctcattctggtgggttttcaaggagagactcggagtccaagtgtcctttctgaatatatctggaacttctcattaacaaaagactcaagttataatttaggggacaaggcacccaatgagaatgccttgcaggcagccctaagtacacctgcaattacaccattactagcgcggcagcacacatggccctgacttagtttaaataattacgtaagtcaaccatgattgtttgccctttgcatagaagggcaagtattggtacctgttacaacttaggcttttttttctttatgtttgagccatgatgagtgatttacactgttgcatccatatgttgagatgtaagaataaattagacttggtaattgcccttaagtgtctggaagtcaactggggaaagagagctagagataataagtgtgaaacaatgtcacagaatcaatgacggaactcttcccaggacaaaggatgacttttgagttcagtctttgcctttaattctacatggggaggagagcacgtttagccacaaatggaagggattactcatttgagctatttggttatatgattatttccccagagaataggatgtgcagggcattacacaagcagtgccaatagcagcaaagttcttgagagtgctagtaattcaaatggcaggaagagaaggaataaatggtaaggctacctacagttcacagagagctccatcctcactgtggctttggattttgtcctgtgtgaaagagaagtgactgtgaactgacatgctgtgtttggtgttttagaaagatggctgcagcagcggtttggggaatggactgcaggagtggcattggaaacaggaaggttcatgactattgccagagacagaggatgaagcaggagcaaggaagattcaggacaggggactccggggctgatcaggaggcagaactggttgataagtatatgtagcagcataagaaagaaagaatcccagattgacacccaggcttctcacttggaagcctggatagatactgaatgcaatcacaaaggctgggaagtcaatgggactgcagggaagggaagggaagggaggagaagaggaagggcaggagggtccaatatcaatattcagcttttagatgtgttgagcttgaagtgctcagatggagaagtccaggaggcagtagaatacggtggtccagagcacaggagagcaatgtggcttgagttgtcatttgctcacatatttccgtgtcagttacttgtcttagatcacagaacaagttctcctctcacagtttcctggctccacctgtctcatgctcaccgtcagcatcgaaattgagccacaccaggggttctggataccagcttctctctaggtgaggctgctatagtcagcagctgattagttgcagttatcagcaactggtaatataatatattgtgcatataagtgtaccagaagtcatgtttatatattgctgcaaatactcggaatggggatctcttgttccctgcttaagaccacatcacattacttggttttgtacgctagtggctgaaccaaaaaaagtaggagatgattttttttcttttttcttaaagcagtagcttttgaaccttgaccatgctttctaaccagctgaggggcttttgaaaaagagggtgccttactgtgccccagaccaggacaatcagtatttctggggaatggagcctggcacacacacatttcttaaagctcccttggcaattctgaggagtggattacatgttgtatgtagctcgtaacgaaagaaatcttgtctttgctctcagacccccatttcttactcatctcatgagctccttcgagatccagaaacagttgcatatttcattagtaaatcagttccagagtcacattttatttcacaagttagtccattaaaagtttcctgcagtgaggaaatagccagaaagaacactccacccctcctcctttttataactatagggtctggctcgacagagcaggagcatcgccatcttggacaagcccctcattctaaagttcaccttaataaaaaactgcctaaattcaaactgcatcagcctaatggctaaggtcagcatgaccataaaccacaaataacatctccaaccggaaacattcgaaactcctcctcgaccagagacatgctagtcccgagataacccccctccagcagggaagatgccagtctcgggataacctctctctggccggaaagatgcctgccccaagataaacttgcctcctcccagagatattccaaccctgccataaaacttctccctcaaacaggaacattccaaaattctgataatctccctcaccctaaaaccaatatatactcctagtctgtaagagaaagcgctcttgaccaaaattcaccaggagtgcctcccaggttttaactaaagaaaacctctctttaactgccaaaaaaaaaaagggaaaaaaaaaagctttctgcagtggctttcagcgggcccagcatggcagcagcacctgagaacctgttggagatgcacactcttggaccccaccctggcctctgagtaagacactggaagggcaggccccggtctgtgcacacaagtcctcagggagattctgactgatgcatgccagattttgagaactgctgatatactccaggcacatcgcatgctgggatctagatacaccaagggaacaaaataactgcacttgtcctctgaggaccgacttaccttttggaagggctgagaaagagagacacatacaagatcactccctgtaatgcaatgttttataacagatgtgatttgggatttcagtgggagcccaaaagagggactgactaattcagcctctgtgacaaggggagtttctcagaaacagaatgcttagctgggcctccaggcacagggacaggaatgaggaaatacttgtaggccctgtgctccttcagcaaaaccctcagtttcttgttatttttataaatgcaaacatcttattaaagtagatgctaaggcattagaatttcctgctttatttttctaaatgaccatgaggaaacctggaatgtcaaagataaagtgcaacacattctgcatttaaaaattaaaatgatcctttttaaaagtagcaaccagatgtgaaaaattggactggagtccaggttatagttgatagctttaactttctccccaacagcaacagcacaattttccctaaaatgtgttatgaataagtaaaatgactacttcacatcctttaactcttcctacagaaatctaagagagaaatgaaacaaaagtttgcacagttctagacacgataaatacatgtgaaatcacacaactcagaaaatgtcccttaaattaattgagccattggtacttgtgaattagaagagacatctatgttctgatccactgttgaaagctgtacaatgttacctatttatttgcagacatcctttggaaacaaataggtagatttgcaacaaataaagagtggagtacagctgctgacattaccttgtatattcatgcctttatgtaaaaaaaaaaaaaaaaatatatatatatatatatatatatatatatatatacacacacacacacatatggaggtaaagaccactgcttgctttgcagttgttttaagagcattcatgaaggattttattttataagcagaaatgtgatatctgacgattttaccactacatgcttgcaggccagtgcacagcagatgacgtcatgattgttttagcagtcctatcgttttacttatgatgtcattacaaccctttgctaaaatttctttcctttactccaggttttggataaaattgatgcattgcacatagtctctctgataagacaaactggcatttgtatgtgaaaaactgtgcatgttttagtgtctctgctgatactcaaattatccattattttagtgctggaataaaaacaaaccacttagtgaatttgtgcaggtccttaaggacaggcaaaggtgtcctgagattttctgatcattgtataccaaattttagaaactttttcaaaaacatttttttaatttcaaaaacctggttttgtttatttaccagcaatcattgaatacctgaaagctttcaggagattttattacaatggtttctattcacttacaaaattatctcctagttcattctcatacactgtaagccattgtaaatgcttcaaattgtgccgaacaagataaactagacaaactattttaagtttgttctagtgctaacttgcaagatctaatggctccaactagatttttaaaataaagtatattttaatatattattagaaagttaagcaattatctgtttataggtaacaaaaaccctggaaccccaatgtcagatgtcatccacttttgattaagtccaaacatatgacagataaacaaaagatggttggctgggctcagtggctcatgcctgtaatctcagcactttcagaggccgaggcgggcggatcacaaggtcaggagtttgagacttgcctgaccaacatggtgaaacccgcctctactaaaaatacaaaaaaaacagctgggtgcggtggcacgtgcctgtagtcccagctactcaggaggctgaggcaggagaatcacttaaacctggaaggcaggggttgcagtgagctgagatcacaccactacactccagcctaggcgacagagcaagactcagtcaaaaaacaaaaaaaaagtggtcattggagaattattgtgtcacctgttgttttttaatgtactaattttgagaggcttttaaatagagtgcactatagaactttttcttggcttcaatttgctacaatgttaatagagaatcagaaaccttatccttatagatgtttcttgatttttttaatttctggtgacatttatgagtgagaatagtgtattgccctgttttctttcttactcccctttcttcttccttccttgctttctttcttcttcccttccttctttctcttcctcgctccttcttttttacaagctgttatgaattagccttcacagagaaagaaaaatttttataaataactggaaatgaaactttgcaaaggactgcagatgaaaaactttgtcaaatgactgtaaaaatatactatataattttcaaaagttagaaagtaccaaacacactcagtattcatggttatacaagtatgcatacacatgtattgctccctgaaaagtggtgttgttaagggagtttttcttagtacgcggcttaacatatttttttctgtaatttgttgttagttataatggggagagaaaacaggttagagtctcccctctcagtttcaccttccataaaacagctaaactagacgatcgtcagactccttccagctgaaaacatctgtaaaattaaaaacaaatctaaatgtatgcaagatatgtatttaaacatgctggtaataagtgtgctgtccctataatttagatgctaaaacattgatgtcataataataacaacacctcgcatttgtacagcacctcatagtttacacaatgccttaacattcttctctctcagcctcctacaaccccacaggattgggatagctttccagattgggaggtgagggacccaggctcagagcgattctgctgttgtccgtaatcaccaggctggtgatcagtgggcactgggtgctctcctgctacacagcactgtctctcaacatgcaggtcaaggttacttattcctccttcaagacgtcattgggttttttagctatggatgccccatcacttttagttctatttgtgaatcaaaggctaaataaagtattcctcaaaatttgttatacttctgttactaatgcttaatgtccctcacaatttctgtatatttctgtgtatttctgctctgttttggttcctttcccaggtttcttttttgttatgaagtagtttttagactcaagtctcttctgtatgtgttataactgcccattccataagatacagggcagtgaatttgtgagccttgaaaatatttactttagaaatgagaagtatgacttttcaacgttgtgtcatcaacttctgtaaattttccagacctataaatacttgcagaaaaaaaatgaaaggagaaggcaacttgatttagcagttgggtcagttagcaatgcctatggcaagctgtagtaattcccttacatagatttgtaagactcatttctatgatttaaatgaaggcatacacttaacctctttagggtgtgaaacagcttttacaaaaagagacaaacttaagaaacagtgtggccctccaagagtgttcattttccatatcataccatttgtaataagctattctggctgggatttacttgcaagcattggcttttaagaagagatggtttcacacatcaaattattcacttggaggcactttctgggttgaaggaatggaatggagagtgcggcagtgagtagatctctcagtgacggtgatgtgcctctcccagaagaaatttcaaaatgcagtgttcattttcctccacaagaaaggaagaaactgttttgttattgtttattcctaacatagtggaaacttttcagtactctggcagaaatttcccaaaagcaattttctatttcatgattataaagtagcaaaggaaaaagtcctgcactccagctgagcaatggatctccagttgttatctaggtgctgcaggtttagagaggattgccaggagaacacatcgatttttcaggcctgtgatgacgtatctcttgttgaataagtaaacccttccagtaaacagacagttagtatattgatttcagggtggctttagccactgaacctgtaagtcttgcaaaggttacttgggcaaaagcatcattattttaccttcagtcaacaaaaatctacctggccaaggcagaacagaaagttcagcaatttgatgaagtgggacaacatgaagaatcaggtgagttgcctactttttcacttcactttccacctttagagattcttgtttagatgcagagtagtgacgtgcctggtgtcagggagagagttgaatgagaaaagtcccagaagggcagaagacttgggtgattatctgagtccatctttccttatcacatgacagagttcttgaagtcttggctaggaattctaggcttttagattctttgggcaatggctactaaatgttcataatgttgctcagttgcaaaaacaagacattcaaactatagccagggagataagtagtcacgaactcaaggcctaaattctgctgatggagccgatgagaattgggtgctaaggcaaagagagttgccaatattatattcttcggggttttttgtttttattcgcattttggaaaaggaaaatattagcattcctctgacttaatattgagaagacattgggcactctttttcctcccacacttgtcttctttcactaggtgacaagggaagaggtagcatgaggtggtggtcacaggtgagaggggctgttgtgagcacaggcatgttgactgcacattggtcacctagtagaagttttgcaggcttggtgacttctgaacactgttttcaaggttgatttttagttgagagaacctctaggtaccacgtaatgttattaacagtagtactgatctcacaatcgccctatgtcccattcacaagatgttctgccaagccataaaaggcccagttaagtttaagagaagtctcaaaagtaacagatgataactaattaatacccagtgattttgaaatgtagacatcaaacataccaattcagtggtatcatccttagaggcagacagaggatgattaaatcattcagcccatctctgtctgaggacgcagcttagcacagcatggtggaggctaaatgggccttaagggaaaaaatgatatctgaagatgcaatttatttcaaaaagagtttgctcccgtgaattttcactctctatgtagaacggcaccagcacacacttttcctgagcctttgcatgtgtggcaggcagcggcctggcatcctggggaactgaatgaggacgcagatgacccggacgtgttcacagtttgacacatctgactcccagatcagggacagctagctttgctggctggttaagttgatgattccatctttgcctggttctctgactgtctcatgctttctgttattactattttgcagcagatatttctgctcatttttcaatcatatatgcatcctggatggcatagagttgattctcctaacaaatcagtgtccctttgtatttttttctggccataagatagaatatatatgtcatttattaaaaatggagaaaatgttcaggagtttcttgactcagagagggaaaagggatactcagggcactttttcagccaggaatttactacctttgcagggtaaaggggactcaccacgctggaagtcaaaataagccaccagtgccaagtgttcaaagcccttagaatcacaatgctcttaaagcaaagtcttcaacaatgcttgaaaacttccactggttctcagtatgtccaaaattgtcatgtctatgaatgattttctcaatctgaaaatttttatagcaggctaaagaatgagataggtcagtgtgattctagaactaatcattaacattcaatagatgactattttattctagaaaaagcagcaactttctatttactctctattttgagggtaaattctctgtaagtagaaaaagcaaaatgtggacatgggactaacatatgaatatacaaagcaaatgtaccgaaaaaatcttaagacctgccttgtggtgttttttgttttgttttgttttcattaaagtgacttgttagcctcttgctccctgtgaagcacagggaggtgacgtgatgtgcacagggcagactctgccatatgccctggccttgaactcagggccccctggggactgcaggggatgctggccatgctgagcaatgcctgtgggtgtcagtttcctcatctgcagaatgagggtaggcctggtgcttatttcatagggtcgcagaggggattcagtgacagggtggtgtagaggctggagcgtgccccatgtgtgcacgacagccttccaactaggggaggcgggcctgggctctcaccagagagcctgtgttctccatggctacatgactttgccccagacgtccttcccgtggtctggaccctgggaagtcgccaagagccagacaggagaaaggctccacttggctctcctctttggtgaccatcccttgcctccatggcgggactctcaggtgacatcccaccaaccctcactttgcttccctggtgggtctcactttccctcaagagtgttgcttttttgtttcctgcatagtcctgggccagttttgataaccctcttcatttcacttcagaaaccctgatgatttcttcctgtgctctttttaccttaggacttttactatgacgactgtgactggcccatttcttgttttttttctcttgctctgctttctcccccatcatcactaaagcagacatggcaatgatggccatgcacactttccaagggtccagctgtagatcttcatggttccccaggtgcctggaccatcttgtgaggagggaggcaaacacaccctgcctggagcacttggccctttcggcaatgttttggcttcctcaagtgagaaaagaatggatttgtattccccctctgcattattgtttttgttttgtttgtttgttttgttttgtattgagacagagtctcacttttttccccaggctggagtgcagtggcccgacctcggctcactgcaacctccaccttccgggttcaagtgattctcctgtctcagccccctgagtagctgggactacaggtgcccgccaccacacctgactaatttttgtatgttttgtagagacagggtttcaccatgttggccaggtgcccattattatttgatctggaattaactgagctactgcaggaattgcttgattcactgatgactggtgttgagccagtacacacccacacccaaggactgtgactgtcttctgaggtccatcctcagaaattcctgtctcttcacctagtgtgtaataaggcctgcgcgtgttatatggaactgtaaaaaatgcgccaaccatctgtccttcctctttatctgattacttatcattgttctctaagttgcaagttaatagactgatcataaattaatgcatgctggagacttgctgtttcctactagcagcatataaaagttatttttaaagttgttttaaatctgtgagtaaaaataaattgctttgctgcaagaaacaccaaacatggaaaagctaacggttcaaagttaataatttatcttatggacatcactagtggcatagttgctttaaacagtgagaggatttaatagatatttgatttgcaagtgggatgaagggtggtctaacctttgtcctgtgtttaccttccatgagatcctagaggttgtacagcacagtagtggcatgtgacacacttgagagtgcctgttctgtttggaaacctggaaactatgaagggaagtggccttcgagcttaacacataagacttgggaggcaaaaccttttattctctttaaatattcactttaggataagcatttttttaggtgttaggaacagggaaaactgtgtggttaggaaggaagaaagaagaaagttaactgttgtacattccctaggtaatgtttttaagcattgttattcactttcaaaacacattttatttatttggacttaatattttgatcttattttttcaatttcttttaatttaacagacaggatgagtttttttatagttgtattacttagaaattatactaaaaatggccgagtgtggtggctcacacctgtaatcccagcactttgggaggccaaggcaggtggatcacttggatcacttgaggttgggagttcaagaccagcctggccaacatagcaaaaccccgtcttcactaaaaaaaaaaaacaaaaaaaaaactagccacgcatggtggcaggtgtgcctgtaaccctatctactagggagactgagacataagaatcacttgaatccaggaagcagaggttgcagtgagcagagattgcaccactgcacttcagcctgggtgacagagcaagactctgtctcggaaaaaaaaaaaaaaaaggataaagaaatcatactaaaaacaaaacagaatgctgaccaccttatagaaatagaaatagtggtttgctgtgatagcaaattttcttgttaactttttatttttaaagaattgcacattcacaggaagttgcaaaaaatctactgggaggtcctatcccccttcccccaacctcctccagtagtaacatcttagtagcaaagttttgtatatttattttgatatcattatctaagtttgacatcattatctaatattaacctaagccaaaagcccactattttaattatctagtgatgcagtgttatagaactcatagcctttcacagcattatttggaagttaattttcttaagtgaaatgtttttggtctttaaggtttggaggccatggaggcatgaggagaaatgggatgagggagagagagctaagatagataaagacagagatggggagatccactgattcgttgaacaaaccagatacttccttatagtttttggattaacttacatgagctaagtttatattctgttcagatcacaagtggtcaagtttgtgtgtgtgtgggggggggggggtgggtgtgtgtgtgtaccactctacccatcctatatttattgtcctgtatttggtctgttctgccttctttattttcaggataggtgtcctaaatgagggtctttggaaagctggtgaggccatgttgcccgtttcaggtgttccgtgctcaaatgtattcatttcttgaaaaattcagggagtgcacacttttgtacattttcctatgtgtatatgataccattatataaatcttaaaaatatatatggttcacctgaatccccagccatttggtagagaagatagaaaacctacagaggaggctaagattttattagaaaattcagcttctcgacggaggtattggctttaaagtcaaggcaatgcatctattctttcttttgatataactagctaaaagatctcttaaattcaaagtggccctcatcttactgttactgcaatttactcttaattacaaattatataaaaataggttttgaaatactgtagcgacaaagtaacatacctctgctccattacacagataaaacctctaaggaacacctcctctcttaacaggcattaaccaactgcagaaactgcagaaggacagggctatttgggaataacacagctcccttccttgtctgttccctcccattgtcaggcttctgtggagccatattcagagcaacatagggagggggaagagaaaatcaaccccttggtgaaggaaagctcccaattcacagagcaaacatgggtactcttgtttgtgggagctcccagggcctcccagctcaccgagcattctgagccctgatccttacactaattgtattatgcaaccataaatgatgtctgctgtaccagcggggacagtttattttaatagattggtataacttggcagaatcttatctgcatgtttcatcttggatttttagctcaattcaactcaataggcatgtgtcaaatgtctactgcagactgagcactgaaaagctgctgggtacagggttacatggatagaaaacgtagcctctgacccctaaggagcctgtaatccagatccccattctttccatcccattctcccaagcaagaatttacctaatgtggtttgcgagaatttaagagctggaaaggtggtcacgagaagccggaatgggttcgctaaaatgtgtctatatgattaagcataacgtagctttgcagcactcttcacagcttcctcagagccttccgcacgcggtgtctcatttgaatacttgtgtgaggatagcctcatacccctcagtgagctcttcatggagtgatgcagtagacagcaagcctcacacttctatgctcacggaagaccaaatttgccttgaaaaatctttatagtctcttcacatttctaagttgacatcaaaaatcggttaccataaaatcctaatagttgaagagatgtaatttcaattatttggtaaacctgaccttcattgtcaaagcaattagtcaactcagatttactttctcccagataatagattctgacttctttttttctgattaaaaaacttaacaccttcctcaggagatctatctcagttctgaatgctgattctaactaagaaggatatttggctacatgctgggaagaggggtactgaggcacgccgcgattccactccagcatttccagttagtcgggtgcctctgcactcccggtgttccggcgcccagttagttgtgtactctgggctgtccctatactggagtcctaaaacacttacgactgcagatagggggaggtttttcaaaaccttggtctgaaaagccatagaagggagataggaaagcgggggggtggagccacagtacattcaggtggatccgtttttggaaatagtacaaactggaggtgaaaccctggaaattgatctgtcgttcacatgcttcatgccgagtccttgtggacccacagagacacactcgccccagtttgaaggctgctaacttgattctgaggacaccagtgaggtggtagtgtgcaaatgatgtgtgaggaaactttggaggagtctcaccctgcctggagcacgtggcccctaaaacagcgcagcctcccaaagacagaagatgtggactagtgagaagccaggtatggtgactgctgctggatgaagcttgtcccaccagaggctcgcttgtttcattgagcacctactgtgtgcttgtgggatgcaaacacacgtgtggtccctgccctcaggttaataggcaggggtggaacagttatgaaactgctctaaagtcattttctcaaactgggagtgacaaatgtatccacttggaaaagattgagaattttataagatttttaaatttttgtttattcacattgaggagaatctaaattcttttgaacttatgtatagatttcaccattttatagtaataaatcagtcctcctgtgtgtgtgtgtgtatgtgtgtgtgtgtgtatgtaaacctcaccttgcaatattattattttaaatagccacttgcatcttaaggaaattaagaggacaaaagaaaagctgctgttttgtatgtatccacatatttaccagctgcttccctgccggcaggtgctctggttctgcactgcctgttgtcccttgcctgaaaatggttgcctccaatattttgctcagttttctgattgtttacagtggcagaggagggtagatctggtaccagttagtaattgccagaggtggaagtctgtggatgaaatttgtataacatggaacgttagttccacagttaatgctactcaattggaacccatggaaattattttttggtgaaaagggcccatgcgttatgaaatttgagatccatcactttaagtgaatgtaggccctggatacagtgggagctcagaagagcaaatcagttggtcaccttgctcaacgtattttactaagggcatcagtaaggctttctatgacctgctccttcaatgcttggttgacatttggggagcaaagataaactaaggattctaagttctgtcctgtgatgctgtaaggggaatctcaaacctctaggtggaggagtgcagagatgaccaggatggtggaagcctgcaggagagctgaacacctgaagacacccagtgggaagaccaggacctttaacgcccatatctgctgctcaagactggcagagagaagagggtttgtgatgagaaaaggtggtgaaaggcacaaggaggcacagagcatgtcaggtcccatatcccaaaaggaatgtgcttgggtgagggagagctcctccatggctggaggcattcagagaccaggcagtcgcttgtgggtttgtgattagagtgaggttcttttataaagggagtgagaagagaaggtctgtggatacttgagtgtatcggtaattaagaaataaattgtgtacatcccatttctttccacattttcctgggctgtcacagtggctgcaaagaaagcagtccgtgaactgaactgtgatcccagacaggcaagcacaccaggaatctcttctcagctgttgataatgagggagcgctggggagagaaatggggtcctctttgagtttcctctgtgccgatacctttctctttgttaaaacagctaattaaacactgaagcagtatagctctcttactatacactggtagtcatagttctcttactgttctcttcactgacagttctcttactatacactgatggtgacgcagaaattcagaattccccgcatgtgtcccggtttgaaagccactgtgctttgctgtggattaggatcagacagttgagtcttgttccaacaaggaaagttgcttattggaaagttttgctgcagggagccttgagttctgcatcaggcttggaagtgggctctgtggaggtcagaaggaggatcccccacccgcagcctcaagaaaaatatgaaaagtggattatgcctctgtagctatattgcctataaactttctgcagaatgacagtattcatatcctacattttttcaaagcgatattaatcctgagacctgcagctaaagtcaagtagaatttagggataattaataggaggaaggtggggttggaagatctgcatgattatagtcctctgatataactggaaaattctttccattagcaaggagctttggttaatataaaatggacagattaaacctaggcaatttattttactcattgctgtatttttatttcagagctggttgaaaatattacaaagtaatattttaaagtgcttatctaaactcttactctgcattttatcattgggttatgaaatgactggggaaagacttttcttgcttttatttctcagtgtctacttataaacatgttttttgaactactgtttttgtgacaacatgcctttttcccagaaaatctcaggttaacattaaataggcactggatgtttatctgatcttgtttatagaaacacaagaaaattttaaccttgtatatactttactcaattaactaggtaagaggtcattgaaacatttagaattccactctacatttcaataattatcaggtgaaagctactgcatctacatcagaagatgtttgtaatttatttaagaataaaattagctatgcaagaaatagtatgtggagtcctatgtggaaatcacagaaaccctgacaacttgatgatctttccgcaagctaaaaatatcactctggatcacagcagtagaggactctgtaaatttaatctgtgtgtctcctgtaaataagtgcattagcagtacacaggtggtgtcagagtcagtgatgatggatagaaattctacataaaatccaggctcagtggctcatgcctttaatcccagcactttgggagtctgaggcgggtggatcacctgaggtcaggagttcgagaccagcctggccaacatggcaaaacctcgtctctactaaaaatacaaaaattagctggatgatggcacatgcctgtaatcccagctattcgggaggcggaggcaggagaatctcttgaacctgggaggtagaggttgcagtgagccgagatcacgccattgcactccagcctgggcaaaagagcgacactccatcgcaaaaaaaaaagaagtaagaagttttacataaaaacgtggagtgagcccaaggtgccatttatccagcccatacacatcgtaccatgtacagagtggacaccagataaatacattgactgcatgccacaaacatatatatgtaggcaccgttgcattcaaatacacatctgcagccctaacacatctttatttgctaacgagcatcaatgtatttaaaaacaaacatgtttaaactagtgaatgattagattataatgatcttaattcataagttttctcattggccttttgtatacttcaattgtaatacctagaaaaacagttatgtccaaaggagtgaataggccttatctgaaacaggtgagcgtgacaagtgttttcttacttattttacttttcagataattcatccttaaagtacattagtttaaaagtactgtttaaggaaacagtacttggattaaaacttgaatcattgttaaggaaaactataccttaacttcatgtaatcacaattaaacctcttcatatagaaggatctaagaattttctgcagcattcaccagcaccaaaaagctcagagacatatatttctttctctgtatatgtattttaaattcaagttagtataaattgacaggcaggtcagagtaatatatgatcttctgagtccccttagtaattaaaagaaatgattatttttgcatgaaatatgataaagtgattttaagtgcctgataaaaagtcttaaccatgacaaccattaaagattacatcaaagaaaaataagtttgactttcatttaccttggaaacagctattaactggtaacctcaagaaacaccatgaagagtcagtttgctccacacatgtcttgtaaaagtcaaataactggtggttatccagtaatgacaagaggtagaagttacatccttgctgtctgattgaaccttcccagagctggcacaaggctgggaagaccataggtgctaaatgaggaactacttaaagaaagaaaatggaatttcacggacaagaaaatccatgtccatttggttctgtgacccacatcctttgtatcctatgcttttttacacttggtacatggttgcaagattgcccctgttttctacttatagttccatgcagcatggatgtgggaaaaagtctcctctgcaaagggggttaatgcaggtcactctacgtatgtgcacgaggtcgttataaagctcgaaaatatgggctcaccaaccaggtgatttttttaattatccaaccagaagacataacatataggggaatcaaaagaaatctctgagtaaaataatgataacaggtcaaactttgcggtcccacgtgaggctggagatgcgtattgtcttgactttgcatctacaagtttaacaaatgatgctttctcagtttacctctggaaatggaaattagcattgcaaatgacttcatgaggaggtagaagctatctgtgaatttcctttcgctgtgtttacgatagactctcacgtctagatgtgtcatgtattatgttaaattggtatgtcttgaagttataaagcacagccctctataagtatatatattccacctctttcaaatcggatggtacctatccttcaaactgctatttaatgactgtctgctatgttcaaggcactgctctcaatgttaatacttgatgagatcgggcgcgttcaaggtggcatggccgtagactcaatgttagtatctgaaatatggcctacgagctgagttgtgaatcaagttaatagattttcggaatgttaaggtctaaaccagtagctcttaactgagacaatcctgtcctcatctcacctgggagacatctggcaatgtttggagaaccttttggttgtcacactggggcatctagtgagtagaggtcagggatggtggtaaacaagtttttttgtttgtttgttttgtttttgagacagagtctcactttgtcacccaggctggagtgcagtggtgtgatctcagctcactgcaacctctgcctcctaggttcaagcaattcttatgcctcagcctcccaagtagtagctgggattacaggtgtgcaccactacactcagctaatttttgcatttttagtagagacggggttttgccatgttggctaggttggtctcgaactcctggcctcaagagaaccgcccccttcttggcctcccaatatgccgggattacaggtgtgagccaccgtgcccaggctaacattctttaatgcataggacagcccccaccatacagaggaatccccagcccagaatgttaatagttctaaggttgagaaacccaaggttaagccaagtcaacttatctatcttctttaaaattgcataagaatgcagtcctgttcttcattcctcttgctttgcagttaatgatcctttgcctggactttctaagtgcccagaagagcaacagccagcatgcaggatggcattcctgaccagttgcacttggcctagcattccaacctcacctgcctcagcttgttcaacctgaaaacctaccaagtgaaagcaagagccacgtgaagacgccttagttatatgcacccacccagacacttgctcagaaaggaatcagtggggccctggccttagaaactggctccttcactgctgtagaaacaacataaatttaacataaaacacgtgcttttcttttttcttcttactttttcctgtcttggcaatgcaaggatgccattaggtaaagaaatccttcaccacactaatcctgcagagccagaagagaaaccagcttgttctaacccagctttgtcatggagagaaggcagctgctccagtctgaactattctttcttttggtagcagcctgcccaagggtgaaagtgtgtttaatagtttgaattacacaagtgaacagtaaatgtatgcctgtttctgctttatgggactttgaaataatgttgtttgtgccaaggttttagattactatacctaacaacctagaaaaagaaatgaaaaggaagccttctgccaggcagaggtcactacgggcctggagctgggcacctgactcagcagctgcccagatccccagagctgagaagtcaccatgcatttgtggtgcttcgagcgagttaccagagtcctggaacagagcagcacacctgcggggtgtccccttggcatttgggcagggcaggtgaccaagggtcttgttggaactgaagtccagcttgaaaagcaaatctggttgtgagctagagtccagtaacacttgtttcccgccgccccccgcataactcgtgtgtcctaaaatacaataatttcttgaacttcagtcacttatgcctataagcgggcatacaacaggggcacaataaatgtttgttaagtgaatgaattctttcagaactagatgggatcttagtccaactctcttatttaacgaggtccacagaggttctgcgattgtctaagaaagaaggctgtgttcatggcctttgttgtttacgtggccctgtgattctcttggctccgtgaaagtcctgatgcagacattccggccatctagaaaggcatgcagacaagccatccagctggcatgatcctgagtccagctttctttaaaagagcttccaaaactgcttaagctttgactgcacaaaacctgcatcacctccagttgagaaactcaagagaataagtaagttatggagttggagaccccagcttaactactagttttaaaatagtgaaatcaacattttcaaatctttgacttcactaagatttaataaagtttattaatcatatattatgagttattgctctctctttatgtctgtaatgcagttgctcctctctgtataaattaataagttttagagatccaaaatgagaattttaaaataaattacgtatattttaatcaagtttaatttgactatatccagctaaacaattgattgaacttcacttgcttttctatgacaggttttttgttcttagtaaaagaccccagttttctcacttgtgaacagaaggggttagacttcatgacagctaaggttccttccgtctctaacaaaagtggcctgaagagaggcttctagactatactcacggtgggttcttgggacctcagagtcagctccatcacttaagtggctgtgtgattgagtggagacacctcaatctctttgtgcctcagtttcctcacctgtcgagtgtcaacatgatggcacctaaagctgttgagacttcagaaaggtaatgtgtgaaaagtgaaaagtgcctggcatccaggaagtactcaataaataccaactattttattgctgcagctgttcttatagatgtgatttctagaacattgccttctaatagggtagccatgggccacaattgttggctgttcggtgtttcacatatggttagtccaaactaagatgtgttgtgagtctcaaatacacactggattgtgaagacttaggacaaggaaaacaatgttaataaaatctcattgataacttttaaattaattacatgttgaaatgaaaatatttgggacatattgagttaaataaaacaggagattaatttcttctgtttctttctactttttttattagtgtggctactcaaaaatgtgacattatgtatgcatctcgtattacatttctattggacagcagcgctctagacagtactatgggtagtatctgtggggaggttctcagaaacatgtcgcatgctcttttagaaccttaaagtattcctagtctcctctacttccagcccttggctcttgggcctcagtctttttacttttgcggctgtgtttctctgaaggcttggcattagtagattgaaaagaataaccatctagggaaatgtgaattcagtttctttctgacattctgctctctacaaggggatattatgtacacataaacctacttccaaaataatgaagtgaggcctaattccttactcttcagagagcccactgtggaagtgtcactgaccttgtgtatgggctgcccttcatggctctgggagtcattataaagggcagcatttggcgtggtgcgtcctaagccagtgtttctcggctctgttccttagacatgtgttagtgttaatagatgttcttggaaaaaaaaaaaaaaaacagcattctgaggtcaaacatgctcagaaagcttggaatctgcactacgcttctcgtacacatttcatattaaagattttggaaagtcctgcaatacagagccctgtctaatattgccacaacccacaattgctcaaatgtaaatagatttgagtttattcacattcagatcacctcttaaggccccacctcccaatgctgtcacaatggcaattagatttccacatgagttttggaagggacattcagaccacagcaggggaaagcagggtacttgctgctttgcaagtgtgtccacatctaattaatagtacagttcttactcttggtgtgtccggtgatattaaaaattaatgtgccttatttagataagtaacataaaaatcacaaaatgtatgccttagatttatatgtatttataactagtctatttcctgaaaacagttgagacaccttgtaaaagttaccggtacgatagggccattccaacaaagctgtaaagtggtgataacacagtcataaagaagaggagatagctctgggagaaaaggtggcccagaaaccagctctgagcctcatggctgcaggcaaggtctgcaggttcctggtcctgattgcaggccatttgctgccttgagtggtggttacacaaggccagccctgggggtatcacccagaacacctagtacacgaatttcagtttagaggacgaagcattactggagtattgttatgcaggaaaactttttcctaaaaatgccctgaaaagagagtagcctaatgcattcaatcaaaatgtttttaagtggaaaacatattgtgtgtacttgatctggcctgctgcttttaaaagattaaaactgggactgggcatggtggctcacacctgtaatcccagcactttgggaggcagaggcaggtggatcacctgaggtcaagagttggagaccagcctgaccaagatggtgaaaccccatgcctactaaaaatgcaaaaagttagccaggcttggtggcgcatgccggtaatcccagctagttgaggggctgaggcaggggaatcacttgaacctgggagccggaggttgcagtgagctgagatcgcatcattgtactccagcctgggcaacaagagtgaaactccatctcgaaaacaaacaaacaaacaaaaaaacactggggccaaagaactctgtgtgctgtatcacctaaccacatttcatgacacggctagagaagaatcatgcaaataaaaatttccaacatgttcgtaaactgggaaagtatttcactggggagtgagcagaaaagtaatactataacctctatatctagacaaatgtgaattcagtttcacatataaatatataagtgaaaaaatatataaatataaataatatgaaataatggttatctcaccactttctacatcttttgtgaatattttatagtgctcaaatatattagtgcactagtatatgtacattacattaaataactaatcatttattaggaggatgtgcttgttttttgctaataaagatgataataaaaaaatccttagaccccccctcggtttgttttcagttaggaattagggatatttataagaatatctttaaatgacacatgccttgctctgggacgaggcatctgcatgggtgacacatatgtgttgtgtgtacaggctcccagcatttccagggccctgctcagaatgtaggccttactgattcttacagagttacaagcgctggtgaggttggcgaagtttaggtaaacacagctgggaatgccccatggcctctgggtgactttggacatcactgaactttacccttagagatgcatacctgcatcttttttaccctgatagggccttccatgatgctttcaaagtgtttttgtctgcttttcggttaatagactttcacagtagccaattgaatatattggttaaatgcatctctttatacacagactggattcaaactgaggttgtgtctctccctggctgtgtgacgttgggtatgatccaagtgtcagattactcaacttcaaaatgaggacagagcctttcccttctagggctgccaggaacattgaatgagagagtgctggcagcttagtacaggtgttcattgctcttgtatggtactgtctgtggcacggctagataaaatacagtagccactgattcaaatttcaactgaggagtaaaataaactgaataacttagaaaagttttcttcttttgaatgactctaagaatttaaggagcatgtgagtgttgatggctctaaaagggtaacagagcccaactagctcagttctcagcatgaaaatagtcatatggcacagactcagtggagtgggtgcacttcaataactggaagcacagatgccctacagcagcatcaaagatggcactctaaactactttcaatcctttaaaataaatggaaacgcacatttagtatgcatatgacaacacgaaggacttcgattttgctgatgcaatacagttttacaggattttttatactcaaattagtaaaattctgtattgcatccaaattataaattataatatcatctagattggacataggaataacgaccactggtatctgcccagaaagctctaccgcctgtttataagctcctgcaggagacacaaaaagaagagaatttgaatataacttgaaatgaccgtaatctcctgccccaactcatttcattaccaaaccgcctctttcttcattatttctcctgaagcacaaatctatagagaactcagctgccagtctctcccactgcactcagcagtgaaagggttaggcctaggcttttcaaacagaccagtgcttgtatcagcccttaaacatctctggagaaggaaatgggatccttctttggtaattcatttttgacagttggggattaggtgttctgtatctggggggccttgctgtcttctctcctcctcctcccactgcagaccctctcctcccctcccctctccagctctctgatgactgcttcatgctccttccacctgaggactgccagcacagcctattgcaggaacagccaatgaggggctggctgtgctcttttatttataaaattataaactcaagcaaaatctagactatgtgtccccaagatcagaggagcacaaatcccttgcttacagattgcatggggggcacattctttaaaattggtccctgatctagactctagcctgagaatcatctttaagttcagaatttccactcatgacctcacatctgtgggctcccacattgtcttccaaaacacacatggcatctggcatcaccttcacccccaccctcagagcctcatctccctgcaggtagatagtcaaggcaacctcttcactcttctgccaagcctcctctcctcagctcttcccttcctctctctttttgaaaatatttttaattgtggcaaaatatacacaacataaaatttaccatcttaatcatgtataaaagtggagttcagtggcattaaatacattcacgttgttctatagccataaacaccattcatctccagagctcctttcatcttgcaaagctgaaactctgtccccattaagcaatggctctgttttcctccgttcccccagcccctggccaccatcctcagttttctgtctctgtgagtttgattactctaagcacctcttataagtggatcatacaatgtatctgtctttttgtgactggcttgtttcactttccataatgtcttcaaggttcatccacgttgcagcatatggcagaacatctgtccatttccaggctgaatggtactcttttgtacgtgtggaccacatttcatttatccattcatccacgggagggcacttgggttgcttctgctttttagctattgtgaataacgctgctatgaacatagctgtatgcctttgtcttttaaagcccaaatctgatcaagtcactccccagcttaaaaccttccactgctccccagcagtgggataaaggccagtctcccctgtaggtctctcccgccagccctgctcagtcttcttgcttgtcatccttggctaggccttgcattgccatagccctctgcctctgttcacgctctctcatcttggagcatgagccttccatcatctctaccagatgaactctcatttcttctttcaaaaaataaaaaacccaaaaaacccagagatcccaactgtcctggtgtctgcatagtctgcagcacacgccccctccatggcccttcctccataagcagaatcactcctcactgttcctgcagcacctcctgtgtgcccacacagctgtcctgcggtgggctgtgtgtgtgagtgtgccccctctaggacctgagctccttctggagggtgggcacagcatccattcattctgggaatcctggtcggcaccatgctagaacttctgcaagtgagtgcctttggtgctggcccatgggagagctgttggtaaggcatacttttgcagattccagttgctgctgaggttgttgctctttgcacaagtttcttctagtcaccagtgaagtgacatgtgtggcaggcatggcccagggaggctttttcataaagaagaggttgaatctttggggctgtggtttgaatatgtccctcaagcttatgtgttggaaacttaatcccaaatgcaatagtgttaggaggtggggcctaatcacaggtgattaggtcataaggctctgccctcatggatggcttaacatgtttagtgaggcagtgggttagctattgtgagagtgggcttgttagaaaattgagtgcagccccctcttgcttgctggctaccatgctctcttgcttttctgccttctgccgtggggtgacacagcaagaagaccctccccagatgctggcaccatgccctgggactttccagccttcagaaccacgagccagacaaatttcttttctttataaattacccagtctgtggtattctgttatagaaacacaaaatggactaagacaatcttctttcatcaagttagggtaccaacctttaaagactgccagtccaaggttaaaggaaacttttcaagagcagtccaaacatgatctggccctcagctactctccagggtcatgccaccctatcacccactggctcacacagacgctgaccactgcttagtttctcaaactgaagttttcctcctcagagcttttgcaaaaccttttctttgcctggaaaactccccccacaaatctttagttgtaggttccttctcatcttgcagaattattagtttgctcttcaaatagtctctccagctagactatcaactccaggagggcagagttcttcttcgcttccttcacccatgtgcccactgagtccagaactgtatagcagtttgattgaaaaaatccacagggtggaggatgagaggaccctggatcccagcctcacagcctcttacttcacctgtgtgattttggtcaagtcctttattcttcctgggctttagttttcccttatctaaaatatgagaaaagttcccctctcctgggtattctgggagactcatgtaaaaggcactgagccagtgcagcacatctatgaccaggaagggtcagcttcctgccttgcatgagacacacattcccttcttcatgcacagttattcatgagttaaatatgtattgagaagtgggttctcaggagatgatgcatccacagcattgtttgtatgcctctgtctttgatgtccctgcctgagtcgcccactttagagcccttctgttcttcagaaaccagacttttctttcaatagtttcagtaatcaatcgatcaatcaatcaaccaatcaacagtgataataatcatgagtgagcccctgcccgtgctggctgtgtcctgctgaaggcacactaagtgctgcccttcccagaagcctcaggaagcttgcgaagctcaggtgcatggatgcctggtggaatgaggaagggatgcagccaggtagagaaatgccctgccatcacttgcatcagcatctgtgaagagctggccaggcttttgctcacagtggttgacacagtcaaggagcaagggccccgtaggagaggggagtcaagggctccgggtgggaatggagctgggggctgatgctggcttctggagcactgtaatgtgactgagaaaggtgaaggagccgttctgaaaaagaagaaggcaggagctcgcacagctcttgactcatcttgacttctttttcctgcttcatccaagcaggtcgactctctcgtgatctcagagacagagtgaagtcatgagtgggaggggagcacagaaaataagaccttgattcccagcattgggagactccctgctcccctgagtctcggaaaatagcacccttcaaatgttttagggatccagatttgatgaagagatgttattttggcttttagattcttaggagagatttgtctttctcaggtcaggaagaaaatgctgcccgctgcacattcttcgggacagactcttttaattattactagtttaatgtatgttttgcttagttaaggaaaacccctgtggtttcttgacgtgcttcagtattctaactcacagctgattcagttcagggggctggggagatgtcctcgacctctggaaaggagggtgcatctctagaaataaggctaagtatgccactgacactgtctgcataaacgtgtgtgatctcaggtccaaaggatggggcctggtctaagccagggacgtgggaaatcattttcctgtggcaacttgtgaagaccattctgtgaccttggtgtctctgggccttctcttagattttctaagttggctagtcagtggagctgccatccctcctttgcccatgttctactcccagagttcctccaagaaattgcggagcaatgcctgtttcatgagagctgagtttgctgtgtcttccacttagaaacaacactgtggaccaggaggacacacagctcccagggccatcaccacacaaagtgaaggctggtgaatccgaggcttctagcccttgccgggccaggcccgcagcactccgctccccaacccagccgctgctttgtcgcaggaacctcagcagggcagggtgtttcctaggaggacatccgattcccagccattcctttcagtgaatcacctgagctcacattcttttttcttttatttttgaagctcttagccaatctgcttcgcgatgaaccagttttgcttgaagcagacaaacccgattgtcaggagacagtgatgatttcttcagtctctgaggaagagttttcattttccccaattcgcaaaaaaagtcaggtccctccctccctccctctccgtagaatattttccatgtgtgttaacaatggctgagcgtggtagatgccaggaatttctgtcaaccctcaaagaggaaagccctgcctaatggtctgcccgttcttgttcactccctgccccaggctccccacccgccttctttctggaaggtataaaggctcctgcttatacctggcactgcacgcttcgctccctctgatctcctgactgtcatgcccagtgtctcagcctatcattctacctctaactcgaccttgagtgaccttgagcaagtttctcaggattccacctccaagtcactctccctttgggatatgcagcactaagttaagcttgcctggaaaacatcacttgaagctggaaaaccacttttaacacagcgggaaaagctatttgttcagacaggagtggggtgggtctgggcagagcactgctctaacttggccatgccgtggcagcagctcctttaatgccactttttcctggcgcgcccgcggggcctggagctcagaaagaggggaacgctccctcgtctctcaacagttgctccagacaggtcagcaaacatggaattcagaatgttcattaaacactggctgtgtcttttgtgttcaaaagcaagacactctctctgaaccatggccccacagagagtgcagaatgtgtgaaacctgccgggaaggtctggaccccttgcggggcagtgggcagcaccgtgcctccgttcacaccactcacatggctgtgcctctgcttccttctggcatggctgcttcttcctcaggtctcaaccatctccctcagatgctctttcccatgtttgtggctacaggtccccgtgacctgcagaggcagagcactcaccagcagcccagcctcgttgcgcacccatgtttgcatttgcaggccctagaaccactccaagctccgtgtggcgagatgcaccctcctgcccttcactggggagctgccctcctgttcacagcggcacctgagtcacacatctggagccatcctggactgcctcatttccccgatggggggtttccctgacttcatccatcctgtcttttgggtccccataataactgacatgggtcggcccgtaccagcccctgtgagaagggctttaactgccttcccaccccctgctcatcttagagtctctctatagtgctgctgaaagaatctctaaatcagtggttctcaacctcagccgcacattgagaatcacctgggacccttaaaaaaatcttaactcttggtccaagaattctattacaatcggtctgggatggggccctacaggtatttttttaaagctctccagttggtaatgcatagctagagttgagtatcgctgttctaacgtgcagatctggtcatgttaccagccttttaggtggtcttctttggctttctctatctaaagttcaaaaccgaacatgtgcgcattcagtgcacccattttcaactgtgcattaacacattcagcccaccagcaagatttatgaaccattttctgctgttgtatataacatatcatatgcataatggcataggttattgttttcttcaaaatatatgagatgtgagtccttctacgaactgactcacactgattgcccaacttcctctctcgaggtctcatcctctttccctgcagccgtctccctcttgcacgcacacacacacacacacaccacacacacacacacaccacacacaccagggtcgatgccatctaccctggacttcatcttgaactccttcgagtgtgagtcattactcctttgtgcacctctgctttctcttctcaagatgttcacctgcttgaggtcagttccttgagcgtcttccacttgccatgttcaccacagtgctcaacatgcctgaatgcatggatggcgacttctcagatcctcagtctcctcatctgggtaataaggcattgggttggcgggtccatctggtttcttccagctctgagagtgcatttgctctgtgattcattcgttccacaacacttcaccaattaaagagagggtacaaaaggtgaacatccttggctcccagcagatgctcctcaaaacctgaaaaatcagataggtgagggaagattgaatgaaaggcctcttatgattctgcagcaattttggtggtttaagaactctatggaaaaatcatcagtatttctggaattgaagtaaaatggatagtgagcctctgtgtatgtgaaggcccgcatctggaacatgaaagaacctgtctgatgtgttctagtcaggaaagcaggtagccaatactatttatagaatttacagaaactgaagattttgtttctactgattttcaaaatagtattatgtctgatttttttcctcagaaatatacttcctgctcttctcaacaaactcatttgaaaatatgattagaacatgatagaattttactcatttgccaactgcggttcccatttcacatattgttagaattctgcatggtggctttgccctttaaccactaactgataaatgatgtagttagcttttaaatgtgtggaaaaatataatttcaggttcaaccataggtcagaagtacacgtgttttgttagtctatttgtctctcagtcatctcatggaaaattctcagcttttggtatggaaataattttcttgaaggcaatatttgttgagtgactgacggaatgaaaaacgccagttgcgtaagtgtgaaaaagatctgggtgttttcattggatccaaattccacatgagccaacaacagcgtggtgtggaggctggagcacattaataagaacagtgtcctaaattcaggaggtaatgctctgcccatgccctgtgcagctcagacggtgtgtgcagtgcagtatgtaacccagggcacatttcaggggcccacagggagctgcagcttgtaaggtggagtgcagccaacagagcagagagtcagaatccccgcagagtggttgaaggcacaaggatgcgcagcaaggaagacagacttataggtggtgcgactgccatcctctggtactgaaggtgctatcatggagggagggaagtagattgaccctcctggctccagagtacggaactcagacaaacggtcagaagcttacagggaggccaattttggatcaactttaagaagaattttttaaaagctagagcaatcctaaaatggaatttgctctttataaagttgcgaatgcctcaccctggaattgcttaagcaaagttgggacgggcagttgtgagtaatctcctttccaatccatacccgcaatcaccagaaacgtggacttccctgacactgagcacctcttaattaagcatctcataagtgaacaaaacccagcccttcaaagaagtcactttatttatgtgtgggtctgcagcttggatttcttgataatgttaaataaaactccatctactcttccacaaacacttcaagaaacctaagacttttggccagagtaacaccgaggtttgagagaaaggatatgtgtgtgagaggtgtggtttcattagaacatattatttgacttcatgttgaatcaacacttttgtgcaaaatgcagttttaccagcctctttccttgttttggtcacataatttaacttaacattctcggtacttgattttctaacataaaatgggattgagaggggaattttgaagttcccatggtctgtcctctacattctgacagctcattatctctgcggtattgttctcacatttaagtgaggttagcggaggcagaggcctctcaggcctgaagatagcctctgttttcagggaaatactagactgtgagatctgtgacactgaagcactaagttcatctcacaaaagcaacgtgctctttttaaatggttgatcaaagttactttcaaaaggaagtgttagtttttgttattagccgaaacaagagctgctttaatgtagtatatttaaaatcatatctcaattaagatgttattcaaatactatttgacccaccaatctcattactggatatatacccaaaggaatagaaatcattctattataaaaacacatggctgggcacagtggctcacgcctgtaatcccagcattttgggaggccgaggcgggtggatcacgaggtcaggagttcaagaccagcctggccaagatggtgaaacctcatctctactaaaaatacaaaaattagccaggcgcggtggcaggcacctgtaatcccagctactcggaaggctgaggcaggaaaattgcttgaacgcgggaggcggagtttgcagtgaacagagatgaagccactgcactttagcctaggtgacagagcgagactctgtctcaaaaaaaaaaaaagaaccacttgcatatacactattcacaatagcaaagacgtggaatcaacctaaatgcccatcggtgatagactgcataaagaaaatgtggtacatatataccacgaaatactatgcagccataaaaaagaacaagatcatgtcctttgcggggacatggatggaactgcaggtcattatccttagcaaacgaataagaaaagaaaacaaaataccgcatgttatcacttataagtgggaggtaaatgatgagaacacaaggatacactggggcctacttgagggtagagggttgaagggagagaagcagaaaaaataactattggggtactaggcttagtaccagggtgacaaaataatctgtacaacaaactactatgacacaagtttacctgtttaacatacctgcacatgtacccctgaacttaaaaaaatttttaaaaagatgctatgcaataaaattctcaattaagaatttaacttggtaaatgttcatttaatgatctaaaaatatgtgtctggatggctctagcaaaaaaataaataataagtttctcagagatggtaaggctgaaataaatggggaaaaatctgaattgtaatcctttttctgttggacctggtgttggggtttcacacttgtgggtgaatgtgggcctcctgtgagcaccagcacaaaagactaaactgaacaaaagattaaatgtcacctctaaaattctgtgcaacaagacttccagccacagaatgtgcaactcagatttccaagtaaaaacacaccaggaagcagatcttagatctctgttatctccttggcaccagctggtattcatcctcaatgctagctagagttgaaataaagagtgaaagaactttctcttttattacttaataaacttccttttttgagctgttttaggcttacagaaaaattgagtggcagtttcagggagttccagcacggcccctgtttctttctcatggtccctgcaggtttcccctattattaacgtctgtcattagcatggcacatttgttacaattaatgagccaatattgatacattattcactaaagcccacaggttgcgttaggggtcattcttggtggtgtacgttcttcaggtctggacaaatctataatgacatgcattcaccattactatatcacgcagagtcgtctcctggccctacaagtcccctccttccccacctgctcactcctccttcccaccctccccaaactgtggcaaccatttaacttttgactgaatggatttattcttattctgccttattgtatgtacaccatattttaataagataaaataatagtctatagtagacttctgtaaatactcaatgaataaatacttgcatgaatgcaggaaaaatcaatcagtcttgcaggatttcttatgcgttacatcgtccttataagaaagcagtcattctcaccgagatgtgctgagcagatactggacatgttctgacccagataagggctgggtggaagtagggctggagacacagagacccagtgccaacttccaggacctcggaagaactgaaggcagagaggtcctctcagtgtggactgggcctctgctggcagccaccagcgggcacagagctgatgtgtgttatgccacgtggggaaaacctacagacgattctgagaaaggctcacagggacaccctctgcccctaaaagaacaatttaactctaatttatttctgtcactctgcattttctgacctttcccaagtgtacagttttatatgcatttaactgccaaattgtcatgtgagattatatggttatatttcattaatatattctagtttgttcagctgttcttactgggtgaatttgtgtggtttcctgacatttttgtttttagtagtgcctcagtagttttatacataattacgtttcccttctggattatttccttagtatctagttcaagaagtgaaatcgctggattcttgtggtaaatttttgaatttcacagtataatgctgattttctcaaagtctcacattctaagaaagtataatgaggcaaaacaaacaacaaacatcttaagttgattttttcctagcatcttttccttccatctttgcttgtagaatctagactatttcatgaacccaagatataatcagtatccttcttcagtatggccaaagtgagtttctcattattttacctccccttcaggaaatgacttttcatcttgtgttttgggagccatagatggttctgggcaggaaactggctttggatagacccagcatgtagatggctatttggccttgctcccagtataacgatgcagttccctgtgaaagggtatgagtaggttttggggctctggataccgtgtggcctgaagagacaagggctcaatgccaactctgcctgtttccaactgtgtaaccatgtgagcgtcaaaaatcatggacgtgctctggttaacactgagtgggagctcaacaaattattatttttaattgttacttggacatggccaagttgactacactttatgttctgctacctgccagtctgaaagtgacgccacagaaggtgaaccgcatgttgggagatgctcctcatctgcttaaatgaggtgcaaacacagcccatgcgcctgctcttcatgactgtatctgtaccagcaatatttgtattggcaaatcacatgccccagtgggaactacttaaggggaattcaatggatttcattccttttatgtaattggccacttagtaatagacgtgtaggtctcttgtgtggataaggattctgccttttatgtaagatatgtgttgcaattcagctttcaggtcccagccccgggaaggctccaggccttcacaaactggcccacccacgagaaggaaagcaattgtccaaatgtgggtagcttttcttcccactgttgtcagctgcttccaattagcccccatatacataatcccagtttgtgtctgtatcagtacaattctcccatgtcaatgtgaattttaagccacagagggaaaggggacagagaatatgctttcattcagctctcctcgtctcacacctcttgccctgcatgcatttctttgctctgattaaacgagcattttataagccacatttgctgtgtgaaaggcaaagtcttccctcccacggatgacggtctccagggatgtgtgtgtgtgtgtgtgtgtgtgtgtgtgtgtgtgtgagagagagagagagagagagagactgtaaacatatatctctgtgaaacttcattttccatatgtgaatttttggaaccgagacaaatggaacttagctaaaagatgggaaaggtagactgactctgacttaatctacttaacctaccaggcaatttataacttgatggcctaatttttgcagcacccagaagcaagcctgtttcagcacggcaaaggctcagctgctaagtgggcagcattgttggaggtgagcagcttaggctgactgttcatcaaaggaccaagcgcttgaggttcgctcatcgctggaggccagagtggggagggccatttaactgctcaaggccatggaactctactgtcagtttcagggaaatttgggaccctggagcacaaaccaaaactccaattaaccaggagaggaactcgatccccaggagataagtgaagagtaagaagtctatctttagaaacaagagatgtccaaggctagaaagatggggaaggagggtggaactgttctggaagtgggtctcaatctcagcaccagcagctctcaagactttctagagaaggaaacttcatttctgaattaaaattagtcttcaatgacatggcagggatttcggcacactctcttgcgtcataggccactgtgttggaggcaggagtgttggctttggaggcatagagattaaaattagagtaacacgtgagcactgaaaaggttaaacagtagagacatggaggactcccgacccccatgtacccctttcttaaccctttaattaagatcacagccctagaaatagcttgcaaaataattaactactgatcatttataccttagtgcttctgtgagcatgttttctctttcattgctgctcatctgcatggaaaaatgtgcatgggtttctgaatataactccatggtgcttgcttccattatatttgtgccatttggatcataactgataagcaaccaaagagtcccatattactgcacgttcccatcgctattttatgtgaaggtggtcctgggggctgttctgaattctcagtttccttttttcccctccccagttctttgaaaatatcagaaacggacttgtggcatctttgaaaagctacttaaaatgtgctgctgtgctctgaacttgaaaatgtgcttttaatacaaagtttgtgcagcccttgctgctcatacgagatgaatcttaccatgtggtggatgcccgtctcatgccaggcactgtgctctaagcccattggtttatttcagtgcttgaaattggctttcgagagaggcaccacggttccctttttacaggagaggaaacaccagaggatcagagatggagagtctttctccacaaactcacagaccccaaaggcaagctcagggttgtcagcttccaaagtctgcctgctccaggacctcatgttgcatctccattctcttcactgagggtcaaatggaaagaacacatgggggtcaagtttcagaaaataagagaaatgaagaaatatgtgcccggaagcaagaacgaccgacctcattaaactggctcccttcacctcctctcacatctttttctgccttttggccaagttttctctcccccgcatttcctccttgatctcgtttgaatcctcttccctggtgaagtcatttaggttcaggctcttattttactttggtccataatttagatcgaaccacatgtgctgatgtgattgaaacgatgtggaattctctggacagagatagaattatggaggggttagtgtgtgtgtttaagattaaaagaccaggtgtatgggaggaaatataatgaacaaaaaatagtattttaaatgaatactaaacttgcactcatggaaaaagttctcttcccatgaggttctcgcaaagcattttaccatcagcacacgcagtttttctcagttttctgagatggggccatcttgaatccaacagacaacacacagcatcagccagactaacacaaaggacgtcatgggcatggacgtaaatactggtgtcaacactaggtctgcacctcgagaggagtggagcaaaaggatggagtggcagatgaaggtatgctgttcagaaaggaggcagaaatgaaaggaagaccatcagtgcgctccacagcttgaggaccgtcctggagggcaaatgccagctgctcacttctgaaaagaaaaattccagtgaaatgagtacagtcattcttaggattactcacttgatactgtgtatgtctcttcttggcttctcatctccacacaaaaccctcaggtggtaaaaatctaattaaaaaaattatataaagtcttgtagatttattagcctgaacataatagattttttttaagcacgttaagtcttccatggactaaaagaaaacttgtaaacctaagagaacctctatttttgatatacaaaataatacatttccttaaactatgatcttgatactagaattttaattaaaaaatacctgcagtttatatgcaaagttatagattaatgcttaaaaataggttgtatgtagtatccacaggtcatgtttgactgtcaaatagatgtaattttaattcataataattgtgtcgtgttcttccccactagaagccaattatgcaagcttcaccattcacacatggaaaataatttaatggagtactcattgcaatttcacttatccagaattggctgttgttctcagagcagcttgtgttgccttgttaaggagaatatgttagtatccagacatccagaaaggatcctttactgtttcagagtccattttccccacttttgaaatacacacacaaacacccattcatgcaaaccaaacagagattgtaaagtgattccactgacatttatgcacttcttttttctctttggttcttcaaactctcagtcagtgcgcatttactcttaatttagatacggtttaaacctaattagaaaccagaagctcttgtatttccacaaaggattatgacagccccaagaaaagatagtgaaaccattatataacaagataaaggcttcttaacaatacaaggatggattttctcattgatcttagccttctgaattttagaaattgccatttcaaagtctaaaacaaaggaaaatcagggaataaaagaatggtaagtagacacaaacctactggctccatcatttctgttttagcaaataacctgccacatataccaatagcccaagagatgggcatgtccctgcatttcctggtcaaggtgacaacactgcgtcctcctggaagaggtctgccactcaccataccacaaaccaaatataataaaatcagaaggcacactatagtgaattttttagaggcatgtattgaaaagcatctcaaaaagcattctcgaagcttccagaagtcaactcaagttatctgaaaagtgacacttttgatgattgctcgcttaatactgggagagccagatgaagattcctccccacttcctcagatgtgcaactctggaatttcttagtgttactggagattcctgctgcattctgggcctttaatgcataaacactgagatgttctaaggaaattactccctagggaggagaggggtggacgaggagtaagctttgctggtgactcatgcgctgtgtggaaactccctgcacaagtgagctgcgcagggtgagtctaaagggttaatgcactttcaaaagcctctaatttgttattccagaagagtaatttactcactagaagtatctgggtggctactaacacatttgtgtctttaaaaagatcagttttattttaagattaaaaatataaagcaagagctggaaagtcactaaaaactgacagccagtttcccattttcaagagtatttattaaaaggttctggttgcagaaggaataagaaatggcttgagatcatgacacagtgaatcatgttgtaaacatgttagctatggctgtgaattcaaccagcgatgagttcaagcgtccccagaaggtgttgggggaattagggacatggctgtgtttccccagagaaaagtggccattttactttccctcttcactaacatgcttttgacatgcatggcagagctgaaggcaaggggaaggggacaacatagtaagtgactaagtggcttttttttttttttttttgccaagtgaagctgagtcatatggcctctgtcattccaaaactattctctacggctgcattcctttcgctcttgccttcctttagaaccctggagaaggcctcctgaagcctggccctattatgtatcctgacaaagataaacttttccaaaaagctgcatgttgtttctagcacagtttttcctcgcagtgactacgtgatgaaagtaccatgcagaggaggtgtctgactgaggcgttcgtggtgtgtgacagagtcccctgcacaggacagccgcactcccctcttgcgtcctttcctcccatgtttgcaaagcctctttccctgtcagcagggggtgttctggcagttgacatttctgaaaactacagcctacatttttaaaaaatccagtaagtgaaaactaaaaaattaataccgtggtcataatagtgtggcatttgataactaatgaggcactgtcgtgccagctattattttcagacatttacagtccttttttaaatacaaagaaatatttggtgtgaaatgttccccgggagctggtgcaagcagaggcgacagggcaagggagcttgggttgtagcctcgaattcctccggccagggctaccgtcagcctgcggcacacaagtaaatcaaatataaaaccaaaatttctgtaagcaaatcagtttctaactcactgtaacgaattatctttcgcacatcacagaggcatctcttttcactgtcgagtttggtttgcttggttacaaaaagggcagttcaaaagctttggttgctattgtgaaagtcagctgaattccttccaccgtgctggggtggggtggggttcacgcaggttctcttttgtcaccaggggtgctgtggattcacaagtaagcaagaggctcctcaggtcaagcctctggctgctccctgaggtcagctgcctagcttctcctcctctgagatagacgggaacaaagtctttgatgtgtgcatttctcaagcttgacaatgatacagctacataaaaacccatgatttcatatagatattccaaaacgtaaaagtaaaccatgcatccacagagacatggaattacagaactggatgctgagctggtcacttgggaggcaggcgtccttgccattggtttatgcctcagccccaccatgcagtggctggccaggtgacctaggccagtcctgcatcctcggctcctcacctgcctggtgggacagtgacatctctcctgcagcactgctgtcagggtgagggaggtagggcgcagtttcagaaaaccattgggctgcacctgcgtgagcacagctgcaggagcaaaagtcagaaaggtcagcaaaggatttcaggagcaaaggtcagaagaaaccctcaaggtggttgtgtctgcaggaaagtgctgtcgtctcctgcaatgctttcaagactattcagaagcacagtgtgaagggagagccggagcccatggggaaatgactccagagtgttccacgtgttggaaggcatctgttggaaaacggacattcaagcaaatagttgcctgcatagacaacgcagaatgactgggaaagccccaacaagttacctactggtaaatgaggtgagaagcttaaagtgagaaccccattgctgcctctttttcactttaaaaacatttaagttttgaattatggtaaaatacacgtaagatttactactgtaaccatttttaagtgtacggttcagtagtgttaagtatattcacattgctaaggaaccaatctgctacttttgtttattaattttttcctgaggggaaatatttttaaattttaaaatatttaattgacaaataaaaattgtgtatattcaaggtgtagaacatgatttcatatgcacgtacattgtatactcattaccacaatcaaagaaattaacacatccaacccacccatagttgccattgtgtgtgcgcggatgtgcgtgtatgtgtgtgtatgtgtgcacgtgtgcgcctgtgtgtgtctgtgtgtctctgtgtatacgtgtgtgtacatgtgtgtacgtgtgtgttcctgtgtatgtgtgtctgcgcacgtgtgtatgcatgtatatgggtatgtgtgtacgtgtgtacgtgtgtgtgcatgtgtgtatatgtgtgtctgtgggcacaggtgtgcctgtgtgtatgtgtatatgtgtatgtgtgtacatgtatgtacgcgtgtgcatacgtgtgtgtgtgtgcacaggtgtgtatgtgtgtgcctgtgtgtgtgtgtgcatgtgtggtggggacactaaaaatctctcatcacctttttagtcaaaagaacagttgttttggtttggctcttctgttttaaaatatcagaacaataataatttcccacagacaaaatcctcaatcctcaccatccttctatttcctatattcatcataaacttcatgcttgatgttgaaattgttttctgaaaatagagaatacaaagaggagattttaaaatgtcagtggcagccccacactcctttttaatcttatttcctgatatcttgagtttacttggacgtagagttttccttgactatggttatttctggtagtagcagctccagattaggcaatggttttcttcagagatagcttagagtgagccccagaacaaggtcaatgcgaagattgcttgtgtctgcgtgtccagggcacagtgatcctcatcactagccggggggctccgtgaggatctgctcctggtcgtttctgttctgtatcttctctgcagcccttactgaagccgttaccaactggcacaattcaattcctactgtacccatcatgcacagatggctgaagtattgagaacgctccagtgaccgggaggcaatagtctgtccacatctaagaacacacttggaataaccttagagaagagagagagagagagaatgcatggttagtaggttatcaaactcctatgacttttcacaggaaaagccctcatccacaccaactttaggaatgtgtagaaagaagggtcagggacaggggtgagtggtgggcagagcagttggagggcacagggaaaaggcatctggtcatgtatttggagtaggaggtcttgctttactattgaattgcagggacactttgggaacagtgttcacttctttttgcaaccatttcttcagagaaaagtcatgatactcaagtcttcttacaaagcagtttgaggctttgagtaccagactgattacagagatgagtatgaagcattattgtagtatttttaagtgaaattcactaaatgcaaataaacctagcaaatgctctatggttaatttttttctaaaattcagataattaagacaattcattctcctgaaactgctgttcatgtaaaaaggaattttatcgaggtggcccttgagtgccaaacagcctgtcctcagctgcaaaatgagtcgttgatgatcctccagcaagggatactttttagctcgtgtggtgattgctgcacacgggatatgtgcagcaagtatctgctgagctaataataaacagcctcagacagaaagacagtgggcacaaggtcatgcttaaaaagaccccttgttctactgcatcccagctccccaccatggggcctcacaggccctggtgaccaagcacatcagacctggttcttgctcagtcctgggagccacagaacccagcacgtactttacccccaagaccagactccagcttggcttttgtcctcctctccaggattggtgacctcctaggtcgtgaagctgtgatgagcaaagacacactcctctccattctcccaacttcaggtccctttgacagtgtcagcaggcatttaaatagcagaccacccacagcagggctggtagatgcagtgaactcaggaagatgcctgcatagactctagtgttaaagacagaatccttacaaggaacccccatagttacctaactgctgtctccagtggtcatagaagtgtgataacccactaatcatcattctctgtctctctgtctttctcatacacacttacacacacatacacacaaccttgttgcttaattttcagagagtctactttcagaaaagccttcaggaatacatcatgtacaaaactgagaaattacctgaagtatctttaaatttagtaaaaagttgcattgttttttgaacatcacacttgaaaagtacatgaatacaaacatacttaggaaaaaaagctttaattaatttaaaaaggagaacaatgctatatgctgtatcccacctttctctgaatgttacattttctcccctatcccaggctgcatctaagaaaactcagagggaatatgctatctatcttttccgagcaatgaaagctctgggttttttccttgcttttcagggcacaatacttctctttcttcctggttagacaggataagttctgagtcccctggtatcatcagcttacttcttctctgttaaatattcacaaaaaatcactaactttcatgcctcagcaaacctccactgcctaaaatatagtgaggtcattcatcttcggacaaattgccccaactacggtgggaaaagaaccaatgtgttggactatttatctaatttttgtttagttcggggatacaaataaatgcatagatacatacaaacatgcgtacataatagcagcagcagcctgtgaaacattgacaagacctggagttggaagaggactttgccatcctccagtccaacagttgcctgtcacagattagacgactgggatgtgcgcaggcgattatttgcaaacggccctgagtcccccagtttatgtcttaattcgcagccagggctgattgtagaagcaaatttgcaaacatgtgcaagaagaaatcacacatcctagagcttggatttcctcgtttcttgctatttctatccgtagacagaaccattgctgagctgttaaatttgtctccttcccctataccagtcttgaaaaaggaaaggaagtggagcaaagaaaaagaaattaataaagccggcagatcctaggagaatcttatttaatccaagctttgtaaagttttgctttattccatggcaacatgggtatacacatcccaccggctgtttcagtggctcagagcaggtaaggcctgtgccaaacgccgctagcaggaggaacaacgtggagacagccccagaggtggaacgttggcccttctgtggctccggtgtctcaggacctccctaaagcccagccctgacactgagcaagtttccaccactgttaggaagaagtagaaaggaatttggagggttggtgttactgttcaagagctggaaggcttctgcccccattcccattccattaattgcgtgaggtagagaactcatagaagataggaacacatatgctgatttccaaaattgcctttgtatattttcacgtgaagactttaggggcaaaagaaaagaagcaagcattttgaatatgtgtttcaatttgccttctgttatataaaattgtattttgcctattcttttttcattattcggaaccttcaagaaataaattaagttctctcaaaaatgtgttttttgaaaagaggactaaaacagatggcctggctgtgttaaacacagggaccagaccagcacccacctctccacctgccctgccttcactggcagaattgtgatccatcatgttctctgttcaatgtcatcatccctttcagagcatgggtctcttcctttctaggcagtcttaccaggatgcatgggtgtgcctgcgtaggcacacgcacagctcccaaggactctaaaaaaagatatttttctgcttatatactaataatatgttagagatttatgtttcaaattagtacagaatcacatggttctctccaaattatatttgagagagaaagaatagaacaaaatttattttacaaaaatactcagtacatttagggcatatacaaagatgttccagaatgtagcttatctctttaaagacaattaacacagtttctgggcaaggcaaggcaaaatattcagtaacttagcaacaccaacagaagacagccaatattgcagcacatttttctcttggattgggtcagagagtactgcagagaaaatggagtagagagacctgaaatactttcgcacacactgtggtcagtgcagcgtccactgtgtgccacagtaatactagaaactccctggttaggccttggaatccagctctcatttcgtatgtgacctgcagggaagtaagttaaatgcacacgttttatcaagttcaaatgcaaacttaattttaaatgtatgcaacatcagtttaagcgttgtagctattactagcaattgtacctattactagtctgtactctgcacaactttggagtatactgcctactcaaggtggattttagagctctatttgtggcattatatcacggacaaaagcacgttcatcagagtcagaggaatgtggtgcaaatcccagctgtcccacttaccagctgtgggacttgagtaagctcctgaagcagctgcacctgcattttctggtgggcaccatggagctgtcagcagtgctttcctcagagggctgcgggctggatgaggtttgctggtgcatgtgaagtgtcaatcattgctctcatgagtggtgatgctgatgccgttcccttttttagggaagtgattttcccttacaaagttaccaacagtttcatgttggcccatttttctattaattgtttccactaataggaccaacagtggtagtcccatcattttattactgcttgtcgtagcacaagcagttgcttcattgtgtttagataaatattgacggctgcttttaacagtctgctgttttgtctccttttgaggtccttaaagtaatccttaaaaagatagtgcagatggaaagatgtctggagtcagtgaacctgccttctttcctgtgtgcttgtcagtttctaaaatgccatacacaaaggactttcatgatttctttttaggtacatgattacagttcaattcacttcactgtctggaaaatttccttataatcaggatgaaatttctcatgttagcctttcacatttcactacttttagataaggaattctcaggctttgctatatctgactgctcttggaggctgagcttttggctaactacctgactactttgtcgtttctcttcccttggaatgaagcaaatatctaacttctcactcattgtttctgctattttaccatttagtcatctgtgatttttctaaatactgaaagacttccctcaattcaaactatgtgccggatcaaggaaagggcagttggatattgcagacagcatagtgcaattgtgaagagtgtctgcttaccagccacgctgccttgcacaagttatcaagcctctcaacccacttcctcaatctgtaaaataggtatgagtgtaggaccttcccaggggatttttttgtgactatagaatgattctcagaagactttcaggcagtatgtgggtgaggcacatgctggaaaggcttctgcaggtgcagtgatcaatgcttttctcagtgtgtacatcccataatacagacacgttaccagaaactccctagccaggactttgattgcagctcacattttgtatatggcccatagggaaatgaagtgtgtattttttataaagttcaagtgttaacttaatttggaatttactatcaaatctcagttgttatgggcatttatagctattaatacttcgtcccatgtgtcccatgaggaaaccaaggaacagaaattaaagttctttctggagtcccctgaatctcgttcctgttcttttgcaccctgttaattacatagagacattcacagctcttctgaccttatcagcgttaaggaaaacagaaaaccagcgtgctatttgttctgtcccttagtcaagccttctcaacatatatttttcttccaagattttgcatgtgcacagggatgcctatcctctacaagaaacacattttaggcaaattataattaaaatgctgtttacatctcttcacctttagaatttaaagaatgatcatttcttagattgcatctcagacacacccttcccctagtctggagagggcgaggcccatgggtactgcaaacagcctgacgttgtcaggggcggtctcaacggctcattcaccacatctgcctcgcgaaggctaagccatgtgctgttacccctgctgcgctctggctcattctaaggtacacgctattaaccttgtgagaaaacaaagaggccagccccacccttcctgctcactctgagtcacggtgaaaatgtttcaggatctcgggttcgaccatgagtcctgtccaggtccaggaggaaattcggaaggaccacatgttcactctgagatcccactttcatttccctcctggttgagcagcattaatactctggctagatttaaattctggctttctccagttagaactgaaagttatgacaatgtaatcaaaatagaatgtgggtttacagctggccccctggcctggtttgtgaacataaaacagaaacagaaagtgtaagtggtgacatcatattctctcattcaatgtgaaaggccaccgaagtctttccagaattatttttgagaataatatgaatttttaaaaaatacctaattattttaaatatcgtcttgcttgctccccaaatacctactgttttcaacttggatatacgacatgattaaagaatatctaatatttgggaatgcatactttaaccttataaactaccactgtaaatagacagactcattaaagtgaaaggacattttaaatcaattagtaagcaaatcaattaggtggcaaagacaagattatttttccttatggtagttgaagaataatgcttaacctgtcattctaattaccaagcacggtgttctctttggaagatcatttcaacaaaacattattttcatccagaatttgaaccttgagattgcatggtattttagaaatctattttagaaatctttggcaaaggttactattaaaacaatcacattcatggaaaatcagtataagagcaactaaaataactcacaataccagtaaaatcactttgtcatcttcttaagacttttaaagagcatttgtaagtaactgaatagaaggccaaagggtgtgtaggtagcccagaccatcagtgggcagccagggccagggcaggggccacggttgcagcctgcattcttctaaagggcagagcaaattaaagttgaagcaggagctaaaaaaaaaaaaaaaaatgtttcaaagaattccaccaaccagaggatactacctaggacagtttgggcctaacttatctgtgaaggcctccagcttcctccacaccggtggccacttttcattcactctgaacccttctttgtatggaggtcattttattaattgagctgtgaccaacatgacagaatttcctgttttagggcttttataatatagatagtttatatctaatttcagaatatattcactggggaatggacttagcaaccactaccacaacaatgcaacaatgtgttttggaacaaatttaccaatctgaatttccccctagattaggtcacaggaacattgcagctgatgtacagctatgttcctcctgaaacttggagacacatcctcttgagctgggttataatgggccacccaaagctcgagttcctgtaatggatacactcaggcagcagaacctaccaccgtagtgaggacagcacccagagccctcagaggccatcacaagtgcaccacagctgccttctctggcacgctcagagctacacagtgtactctgggattggaactctttatttttttttcagttgatttgtaaataagattgcacaaaaatccatgcacatcaactctccaaatcagaatttgctgagctaaaaagagcattaaattagatgggctggctttcaaggggtgggggtgcaatagtggaactctgcacaacagttctttacaaagagacaagcaagcacatcgcgtggaaatttccattcaactggaaatgtccaagcctgtttacctcaattaattgtccttgttcacttgtccagcctagcaattgtccattagtaatttgttataaatgagacatttggtattaaagcatctctttgggatactggtatggtttattataacattctgttagtagtgttgtacaagcttgagatgtattaatacgaaatccaagctgcatgagggctttatttttcaagcctacaccttgctgaaattctgaattaaaatatgattctcagtacaaatgaataaatcaacagaaatggtaacgcatgtcaaatattcttaaaacccaagaaagccttgtaacttccttcaatctaatgggaaatgcaggcaaatacaagactgatgtccttgagttttattatcaagactcaagggcaccagtaaaatctagtttcattggttggaaaaaaaatcctgataagcactgttaggcatattaactttaatgattacaatttttaggacactctgtggcctagacttagaaacacaactaatgtccagaaaaagattcctctttttattccatcatctgataggcctatttttacacatacacaccaaccaaaagtagccaagcaaacaaaacaacatactcacaccccttcgcctattatcatctaggtgattttcaatgctcattgcaatgaaacctacttattgtgcatggcacccacccccactgaggaatactgtagtttctttccctttgaacttcattagtagagcacatggttcattcactcctgaagagttcttcgtatgtcagaatatatatactacaacataatttccatcagagctctgaccacccgcttatctattttcataatgcctgccactccatcattagctgttgtcatgtaggctatcaataaatatatgacaaataaaacagttagggaatgagggaaattgactagcagccaaagacctaagccatcctctgcttggacattagaaaactgagttcactacagtcataagatacacaaaggcagaatgtaagccatacaaaaatccatgtcaatcccaatatgtgagtacaactattgaacaccatgtactaatggatgagttggtaaatcattcaatgtcttcatgaggtcaattacagattattatttagaccccaaagattccaaagatggtatttcggtcagatcttcatcctttgtaagcctagcagaaaatatggcagttttattgactactattctttgctgggtgtggtatttttaaactgagacatcagtgtgcctagcacagggcctcaagcacacagaaaaattccttgataataattaaataaaatttcagcaaaaaatatcatcttaaggctgtgaaattatcttcctgtgtggctaaaatagtgaataaaattcagcgcaatataaatcatagtacaatttcatcactaaattttctgatcttgatcttgtcattttacattggaagtaaaaatgtgtcctcctttttttctctgacagtgaaaagtgtgtgtgtgttgtgtgcccttttgcacaccctgcctcacacttgctggtctaattccttccagcatgattatgatataattaaatgacagaaatgtttacttccaagtggaactaagccagggtaactcagggtagggcagctgcttgcaccgaaagaccaagactgctagagaactaggaaacaggcggtgcaagaactccaggctctcatggaagagcgggaggcttctatggggctgcagaaactctttggtgcttggggaaaaaatgggttaaatgctcttaaaaaagaaacctgggagaggtagtttccagatgcaggcccgtcttttcttttaaacagaggcagctccgaagagctggacattgaaccctgagcaggaactggaggccgtcagcgcagctttgtttggcgagcggagctttgcaagggtgtaatgctgcaccagggagacgctatctgcagggaccggtgacgccgtgggtgtggagggggaggcagtggctggccctcttggggtaaggtacgcccaggaacagtttagaataacgtgcgcgagtcaaagggaagaagaagctcctgcagaccttctgggcactgtgcagggtttgctcctgtccaccgtgccgtgttcctgtcctggggtatttgggtgtgtggcgtgtggggaggggagaaggagcaaggcggcagggaggggatgaggaccaccctgtccatgggacaggccctgggccccgcacacaccccaagccccgcgtcccgcgtcctcactgtcctgggacaccccccaccccaccccaccgccacagcccagagcggtgccaggaagccgcctcgacgcagccgtatcttgaggctccagccccatccccagggtaccacgccacgtagagacactatttttcacttcgtgtttgtcactcctaaagcatgtgtgctagctgcaccaaccctgggatgcctcggtgcatagggtttatgtgcgtcctcctccttccctctgagctggtcccccgtggggaactgctgcccagactgacctgcgtccttccgcacgtgcaggaaaatgtccacgtgcacttgtcagggtgggggccacacgggcaccaccactgatcatctgtgggatcgagttactgcccatgcagatcccacgtgcagggcccagtcgctttggtgagagagtggacgctgtggtgactccacggtctgtggctgtgctcaggaggacagagaggggacatcctgagatggtttgggcagcccgcggatcctgtgcatgtccccagagcgtccactttctccatggagcagtggagtggcgttgctgagacagaaagttcaggttctccactccccatgcagcccccactcccctgtctccggccaggcacgcgtctggggtggagactcccggtgcccggggccctccagacctctttccccaccccagggagcaggcgggtacttctattccgtttggcttcagaagggaaaagagaacgtaagttcagggagttctcgtccattcctctcccgtgggccgggcaggcagcagggacagccttcaggagccaggaggggctcgagctgcgaggccctggaatgaggcaggcatgggctgaggctggagggaaagccccgctaaggctgggcgggggcgggaaaacttaccaccaggggactcgagatggggaaggaaaggtcagaagaggagaggccaggcacggggtgtgggcggcctgcagagctggagcaggtgctccgcccagagccaggcatgcacactcagagtaggtggcctgtgcagcggggaagaggggcgggtcggcgtgctgctgaagatgcaggagctgcggcctgctctgtgcgtgctgaaggtgtggtgagaagcacttacaaaaagaaatggactgtgttaggattgcacattttactttgtttctcccaaatacgtgttctttgaatttttttccttccagggccaggactggagtgatggttgagacaggcacgcactgggtcttgtctgcatttacattttgagattttgttcagcatggattttatggcgtttttttgtttgtttgtttgttcgttttcaaaatactgcacggtttatcgtgaagacagggtcctttgctgccgtcttaagttttgggcccaagaacgtgccccaccctaggcccgggcctgctggcttcatagctctcatcattcccacggaaccttaagacctgaggacagaaaggaaggaaacaagcccagtagtccgtgaaaatccagggtcccgccactccaggtgtctgcagcagagctgaacacacgtaggctcttgccaggaggggcatttgtatgtgctgagcattccttatattctcaatatgacgcctttgaaagatctgtggtttgcaaatatttactctcagtccataacttatctttccaacctcttaccaggctcttttgctgaataaaagttttaaattttgaagtctaatatatttttaatttttttattttatggatcatactttttgtgtcaggtttgagaagtctgcaccaaagtatgtcctgtggttttcccttaggtcatcttcaacaagtttcatagtattttgtttagatgtaaatctgtggcccattttgagttagtttttgcacaagagttgaggtcaaggttctttttttgcctgtgatgttcagtggctctggcaccatttgttgaaaacatgatagccaatgtcaagacttaatagttataataatcaggagcttttgtttctttttgttttgtttttagtaactgccagtcactgcttgtggtatacatacacaatggaatactattcagtcttaaaaaaaaaaaaagaaggaaatcctgtcatttgcatacctggaggacattatgttaagtgaaataagccaggcaccaaaagaaaaacattgcatgatctcactccttcatggaatctaaaaaattgtattcagagaagcagagagtggaatggtggttaccaggggctgggaaggtgtgagcttggggagatttggtgaaaggacatagaatctcagttagacaggaggaataagttaaagagatctattgcacatcatggtaactgtagttagtgacaatgtattgtatacatgaaaattgctaagagagtagattttaagtgttctcaccacaccaaaaaaaggtatgtgcagtaatacagtcattaattagcttgatgtagccattccacaatggatacatatatcaaaacatcatgttgtataccataaatatatactgtctctttatgtaaatttaaaaataagataaaataaatgttattcacttgtcgtggatgtggtggggacaggtgtgggatagccctccctgtacaactaggacccaggggtgatctagtgacactagccatttatcaggacgtatgggtgccagtcaggatgataaagcttccttttggccactatactacttagaaatgccctgcaaaaggtgcacatcaaagattgaaagctcaatcctggattttaagtgcttcaaaagtgcacttaattgccacatttttgtcaaacattttcccaggtagtatttttcctcatgtaaaacaacagcaatttaatttgaacagaaagcattttgaaacatacttttggcagggttccttgcagatcagaatggaaatgattaacagggcaattatcaatcatggacttttggcggcagaaggaactgtattgtttggtacagtctgggccagggccacacaccgtaacggagatactctattctgtggacggttggagggggctgtgctgagcagggtaactgcatcttttcctagactgttcacactgctgccacgaaggagtcttgtttagactggacctggctttcttcttcgcaatgagtgttgcagactcccgacaaaggccaggtggtaaagtgtggtgtctgtgagcgagagcctgagatgcctgagctgacctgtcctcagccacctgccatcgtgcagaggtgagagcagcccctgaattctgcccctcggtctctccatagctaaagcaaaaccatccttccgtgctcccaggacaagcaggctattaccaaatcacccactaaccctgggcgaggaggggccatcactgcacaattcatcagtgtctgtgacaggaagagattgttttagactggtttttttttttttatttgcaagcttttttctctctccaaaacgtgctgtcagtgtgttctaatttactctgtaaggaattctggagctaatcataggctcacaaaaagcagcacaggaaagtttcccagataacatctatttcagtggctttcaaacatttttgaccttaccaaagtaagaaatacattttaatatcatggcacacatacagctgtatctaaactttcataatactgcctttacgatatcactctgatattgtctattcttttctgtttatttttctttttgttccttgttatgctggttgtgacccactccagtgatttcacaatgcaggctgggtggtgtcccacagtttgaaatcccaatctagggccttcctctcactgtacaaagtaggtaactggggacattagtggatcagtgatcaaaccaaagttatttgatcttaccaagtgatatcaggatgagaaagctgttagagtgtcagatatgtgaaggaacttgggtcattcctgatacctcaaagagaaaaaaggtagtccttgaacacctcctacttgtaaaggatgcacaatcctacatgcccctccctttcctttcctcccctctgtaccccacccctgcccacattttcttcataagcagctttggtgttttggcttgtttgtttcccttgtctcctacctgtgactttatagccttttggagactcacagcaatagttgtatttaaactcagtgggtggcatccaaggctaaaaaggagattgcctagacacaaaaccacccaagggagaaagcaggacagcatcttactatgattgtttcttgtttcttcctgtctcataaggattattacccagggttttcatttttttcatttcatggttcattttcgctccagtgtagacatacaatagaccactcgtccctgtggctccgggcagcagcctcatctgagaccctcctgagacatctcgtgcagggcagccgtagtgtgtggcttccccagggctgctctaacagatcaccatccttgccatggcttaagaagctgcagatttatttgcttacagctctggaagccagaagtccaaaatcaaggtgtcagtagagtctctctctctgaaacctgctgaggatgatgcccctggcctctccccagcctctggtgttcccagcagcccttggcattccttgccttgtagatgcaaaactccgatctccacctctatcctcacagtgagttctcctgcatgtctgtctctgtgccttcacattcctctctgtgtgtctgtgtttccatctccttatgaggacacccatcactgaatcagggcccactctataccagtaagacctcatttcaactccattacatcttcaaaaaccccattctcaaataaggttacttcacaagtgctggaggttaggacttgaacataccttattgaacaatccaactgatgacacatagtaatttatgcactcgttcttggagacgttgactttatttagtagcattaaccatggcaatgtcaccagcatcgctgacagcctgaagcatatgatctccagaatgtatttcaatcatcatgttcacttccttggtattctttagacaataactcagccttgaactccagtaaagggtttccctgggattttcttcttgactcactccactgtggcctccctcatccaggactgtaacagacgcctgacgtcagtggtctagacctctctgctgaatgtcatctttggtgaatgtcttatgagaaaacacatggttggtcactcttagaagggcatgaaagcctgtctgcagtataaccaaaacaggcacatggcgaggcacactgtgcgcatgtgtgtacaattaatatcatggttttaaattattttcaggccaaggggagatctttgctgcatctactgaagaaagcgaatctttttcttcctgaaaaaaaatggctacttattagtcgaatttgtgttttaaaaatatgtgaactaatataatgcagacatgcattaatgtttaaatatactggaagtttttggtaaaatgaaacccattgtctctgttgattactttgatgagtcaagaagtaacatcctgggaatgattggccagtttaaatgagtgcctcaggtttttggaatacaagaaatcaagaggaagggattagaacatataggttagcaagattgggatcctaaaatacagacccaaatgaatggaacaaaatcagggaatttattaataacagggtcaaggccaaatcagtaacaaatatcctgagtggaagaaaggtggtttaacaaatgcccctatgaaagatagagattggcttaccatgatgagatgtaagcccaagttatgaggttggcacacaaaaccacaaatgtcatagcttaaaacaacacacacttcttatctctgtttctgtgggtcagggtctgggttctcagggactcacaaagtatgttttcatctggagctccaggtcctcttccaggctcataagggttcttggcagaattcagtttcttgaggctgtaggactgaggtcctggctcctagaggccaccctctccataagcagttcttagcatggccgcctgcttctccaggcccagtgggaaagcatgtgcctccaggagggctcagtccattcttcatggcttttacctggttaagtcaggcccactcaggataacttcattttgtattaaatcaaaaccagctgatttgggatgttaattacatctgcacaacttcaactttgccatataacctaaccatgggactgatatttatcatgcatttgggtcaagttgcattaagagatataataaagctggacaagcttctgttgattagaagagttcagttacaaggctacacttgggaggaatgtttacaaactggaatggtcagaggatggggaagacacttgagaaaagtcaagtgacggatgaaggcaaatgtggatatttatctgggagaaaactaagaggagttataatagctgtcttcaaatatttaaagggcttttattaggaagaggaatttggcatattggattttgccttcagagaagtggagtcctgagatgctcttagccattcattccagcctccagggctcacctgctgtcttctgtccaggttctcggtagcagggcagtacagccccatccgtgatcttccatagtcaggcatattgtcacactcagtgagcggagagtcaaccgggaggaaggcacagtttctctggaatgacctacggaatggtacgctcaaatgcaaattctccttcccttccccagtccttgtccttcagatggtaatttaggagctgaaggtcagggcaccagcagcctttggaagcctacaggacaacagtcagcctggctagaaaaaaaaacaatgtcacaggcatgttgtgtttaatcacatgaaggatatttgcattgttttccaactgatgccagcagacacattgtcagtggtatcatgcctggggtatcagagttgacattgggttgccccttctctgaggcattcatgtaaatccttttaagtttataaaacctccatgtggctcctgcatgcttcatcatttgcatgtgtctctttttccaggggaggcagcatggggagcaggatgctggtgggctccaggtgcagagagcagggtgggcgtcagaccccaggtccactgtgcacgccctcttgtagagcccgttccgttgtccatgagatgaggagtgttcttatctctaaagtattatcatgaaaacctaacaatgtagaaagactaaagcacatgggtggtgcttcataaatagtatttctcccactttctgaaaactcctgctgaagtaactgcacaagaatccttgaacatttagaattctggttttagccataccataaagtcagtagtgcgtggtggaattctgctaacgaaaattgcgaaggatcaaggcagagtacagagctggtgtgtagcgggtaccttctgtctgctggcactaggtattttacacattaaatcagctcgttctcacatcagctcttttaaaaataaggaaatgaggagccacagtggcccaactgatgcagtggcagaagtagaatttgagcttgtgcagatgtgcctccgtgttttgtctcctgagcatgctgccccaagtttgacaataccaagatttgtactggaacattccctcccatccccaccccctagaagcccctcttcctcccttagatttgacacatagtttgaaaccactattaactaccttatgagagccactgtttgtgaagtgctgactatgtgccaggtcccgtgccgtgcaatttttgtgaattatctcgtgtctacagtgcctcacaatttctctgctcaatacctccatgttactgccgaggaaagggaagctcagagagagtaagtaatttgctcgagttaaagagctggccaggacagccaggggcttgcaccccggagccttcatccactacactgtcagctggtatctcaaccagccattacaggctgtaaaaaaattatataagatagtctatggtaatgcagaaaagtgaggttattttgctccctttccctttgaagaaaaaagccctggaaagacatatcacttgagtatgggaaaaaatgaagctgtggcttttctgtgagtcaattctttcctggcagcttcttggaataagaccaagtatagcagcagagttttctgttttaatttgagctgcagggtgactttttttcttctatgctttcatctctctgtggcttcttttgcctcgttaatttcatgccctgcccaggcgggctactgtgctgcccagtcacccgggtctggggcggccaccgctggccagcaggcaggccctccagaggcagaggtggccacgcttaggtcgctcccgctgtggaggcggcacacttgggtggcagcacagctgtgatgtggcggcagctggcagccccatgggaaagatgtgtgaagtgtggggtttgacgacccatgggagaacagactttcttcctcttcttgttttcccttcaaagccgtgagtcaacctcaaattctctgtcttttttctccaccccctcgtgcctctctccctcacgctctgcatctctcattgcaagcttgcatttttttgcacacaacactatcttaatatttctcttttctgcaggcaggaaatgagaagtcatttttcagggtcattcaggaagtcatccagagttataatggcccattatctactggtcagagtttacttaggctttcactacttccactgcccacttgaaacagggaaaaatattttccccccgcgctgtgagtgtgctatttagagctgaccacaagcggggggaagagaggatggctcggatgctgcatttccactgagaacacaaggctggcaaagcttgtctgctgcccagcaagcacttcaggctcacaccattttaggttcactttaagtagtttctcaattgttaaaaaaaaaacaaaaaaaaaaaaacctgtactctgaggatatgcttataatcccatagctaacccagaatttcttagagaactgatcaacatcagcagtggcacttactgaaaatgcacattctcaggccctgcgtagggcctactgagttagaatattagagagcaggtctcagaaacattctatccggcagtcttattctatgcacccgaagggataagagccatgctttcatgaaacatgggttgtgtgtaaaatgtttaaaaggtatggcaaaatgtgtttgattggcaccaaggatttctggttcctcctagaatcattaatcaaactttgaaggagaaataagagagtcggcattttcttgcacattctttgtgatgttgtgatgagttggaaacttcccgattgggtttattagagcatgaacacccaggcacccagcttctagccagccctgtcaggcagagtctcctcgaagatgtggaaaggactgaccaacagctgaggcctacaggaacctgagcaggcaaggggagaggcaccccggaaccaggagcaatggccttcccaccctccctcgtcctctcctcttctccttttggagttgcaggccacagaaaggaagtgacatgagtcactttgggccttcttaattccttcatcaaaggcagcacaggtgtgtatgtgtgttggtggctaattgaggtaggcccacagaggagataacagatggacatactatttcctttcttccattctgatataattcagggtataaacacacacacacacacacacacacattctcacttctttggcatctaccacacctgccccagtgcccatttctctcccacctgaataaaaagcccccacaaagcctgaggtacatggaaaggagcagtggtctggctcccaggagtgtgagaagcagccatgttttcagaggctgtattccacttggacttggccctacgctgaaggtaggagcggatgggggaggcccccttcgcacaaagagccccatgaaagagtgcacagtccagtctataaaacagacgcagaaaatgtgtgtaggacttcttcctgaaaaagagcgtggtgcgtccagtacctccatgttcatggaacttcccagtctgcagtttacccttttgtgcaactcccttttggtaaagccctggtcacacttctggttgttcagattatacagggataattccagagtgattttaaagtcaactgccaggcatccgcacttgcaaattagatgcctggcacatgcttgtgttaaggtaataattcattacaatacaaattacaggggagttcctctgggcatgcgacctttcccgtcatttggctttccctgtgattatcaggggagcttccatcgtgctgctaatgggaccttaaccatgtgtcaacccatggctgtaatgctgacactgttttctttctggaatgaaaggccttcgcaattgaaaccaaaatgttatccaactcagtcctgtccctttgacgatgaaaacatcaagttctggagactggccatccagcctccctgcctcatctcccacgccctccatcattttttgtctctacttacttatttatttggctgtattttacgtacatcatgcaaaaatattcctctttgtaaaaagtataatgatttcaggaaattagagggtaaaaagcaagaaccatgctttcactccactgtcaagagttgtggaagaatccttccagcattttttctgtgtattttacatacatacaaatatatgtacaaataaaggtcgatcatttaggttttgtttatatttttgtatatatgagcttatgtcattcatacatattgttttgcctcttgcttttttttaacttaattttactttgcttgagagctttttgaactgaagtacgtgtaagtcagcctatgcatgtaatggctccctcatcttctgtgaggctgtcactaaaaaggggatttagcttgttctgggctttgcagcccgtacactgggcactgttcatacgtacttctctgtgcacgcaaaggagggcttgctagggaggcctggcagagggtgccattcaaataggattttcaatggaggaatttttaaattttcagttatttgaataagttttaatatatatccagaaccccaaatcatcaagtttgttttcttccacatctgtccttccatttctgaactattttaaggccagtcatgtctcatccaagaaatcccatcctttcacacaacactatctccgtttcatggttatgaatctctaaaagcatgatttttaaaacataatcacaatgctgtcatcgaacttaaaaattagccataaatctcttatgttacccaacaaccagcctactgacacatctccagttgtctcaaaaatgtgttttccattgtggtttgtctgaaacatgatccaaaagtcagacccacctctcacctttccctaacctgccggagcccatgtttctttccagccaggcttggagaccaccacacgggatttgcttcttggggcctccctctaaccagctatgcaggatgccctctttcctgtcaatacaagctgctcaaaggactcattcagttcaaattcacctatgtgagcctaggtgatgctacttatttatttatttatttatttatttatttatttatttatttattttgagatggagtctcactctgttgcccaggctggagttcagtggcataatctgggctcactgcaagctctgcctcccgggttcaagtgattctcctgcctcagcctcctcagtagctgagattacaggcacgtgccaccacgcccagctaatttttatagttttagtagagacagggtttcaccatgttggtcaggttggtctcaaactcctgacctcgtgatccacccacctcggcttcccaaagtgcttcatgttttcaggagctgtacgtgcatttttagttttgatgaccaggtcctttttctgttttttaaagaacttcaaatgatctccagggtacacagcgcttgtgtgctgatgaaaaagctggcagtacaaaggccaccagccaaggtcacacagccaaaaagcccctgacctcgggccccttcccagaccctgggtcttttgctgccacatgaatcttcttcaaggtcctatgtgtagattttcttgacttggccatattatttaggattcagatataataacaaaatagatgttaaagcataacatgaaggcatttaaaagggtagaaagcacatgatttactaaaaccataaatcttatgacctgaaagtttcacctaatctcttaaaaaataccgtactaaaccctgattgaaaatcagagctcagacatacagcctgagatgccaaaaaatggccaggcttgtctgttgagaaagccatatgtaactaactgtttggaaattcaaaatatatcttatcattttaaaaacatctttcttctaaagacaatcatcttggcttcaggaatgaggctagtaaaaagtgaaatactcctacttgtggaagaaatcctcattttaaccatgaagaactgaaaaatgcattctgatgttgatggacccaacctatatttgggtattttatgatgtacacaatatacttttgtatatgagattgttattaaatgtgactttgctttttcaagacatacaatgttcctccgggggtcaggcactgtgtttagcactttgtcctgacctcatctgacttctcagctgtccctgagaggtaccagtgtgcaagatcgctgagttggcaagtgatagtgacaatattttcaccccaatttctaatttaaagaccccgatttctagttttgttttgtattggatttgcacaatttcacgttctgaaagaggatgccctcaactttgcaaaatgggccttttgaatgaaaaggatcagtcatgtcaggaaaagcgctacaatgatgaaatatgataaataagtcagtctttcatctgtaattatctactatggggtaaaaagtgatgaaaactaccatcttgaaaggttctggtgatagtggttcctaatgcagtgaaagatgtgtaagtcaaagatttgtaaccagccagggaatgagaggcgaagccatagctggtggcgggggccacatctgggtgtggggaggccacagttgggttgggggtggggcctgcagttatccacacccctcccacctcccttcgacagtacaggcttcctggttaccttccagagagtaaggccagggagagttgaataagttgagaaatgtcatgtcgaagctattggtggaaagagttccattaattgacaatacaagtccctactacattctaaaatctggtcctgactagtggcaagccgggcccaggagtagcacttaaacaatggcaggcttgtgttgctggcaggatacttcagcctcagaggagctgtgtgcagctggggagactcacactcagaggatttcaaagcagagggcatctcgtagagcaacttatccaaaccctgacccactgtaaacacacacacacacacacacacacacacacacacacacacacaccctgagagagagaaagagagagagataactaaagagagagaactaaagtttggcaaaataatacatgctctaatgaaggtttattaatgattaatctactcctagcatttcctagtccactctatctccttaaaaaaaaattctggttgcagcccactaacttgattgtacagctgcttaatggatagcaggctgtaattttcagagaactgtttaatgcgggctacctctgttcttccatgctgcttgtggttcctgctctgctcaggacagaatggggaggaaaacaggctctgcggcacaatattggcaagtgaaattttgtaaaccggccctcccttccttttgcatttggtctgaaaattcaattagatgctgagtcctacaatgtatttgagaagcccaggagtgccctagaggatgagactgggtggctccctgtcaggttgaacatttgccttaattactttggcaagatttgcatcagtggtattagtccctgcctcacttggaggcctgcacttaagtggccacattcaggctccaatttcctggtgatttcatagtgtagggcacttgcaatcaaaactaggcttaaagcccaaccctcttacattttacccacccccacaaatgcagcaaataaaatgactctgattttcattccctagacctcttttctatatttattacattattgttaagacagtttttgaagaaagctgttttatttaacaaaatagctttatggaatcaacttcatatatcttctccgccagatcaaaacaagctcgtagtattagatgtcaccgagcaccatgacaggcagatgaacatcatccctgtgcccggctaatgatagctcggcctgccccggcgtcagccgctcctggcagggccagcgggcggtgtgggaccggcaccgtatctccagcaattcgcagataacaaatatggttctgatgatgttactaaagatctgtccctttcaagattggattagacattaggaatttggagggctttttattgctagcatttttaagaataaccaattagagtattgattctaaagtctgaaagccacatggacagagttcatgtaattggctactttatgtgcctcttcctagattgccctgcattttcaaaacaagagcctttctattttaatcaaaagaatccagaatgaaatgaggctttgaaaactcagcctatgtttgtcttgatttccttaactgacatctagaagaaaatatgagctcaggggtccgctgggttccttccagcgcctaagcctgtaagctcttcctgctggaaccaagctttaaatgcacttgtcagtcatgtcccatgagaatagatactgccttccatgtttttttgttctgatttccgtgtttgaaatgatgaaaatcatttttctgtgctttttaaaaatggaattgcttttgtgttgggaattgtgctgttcatttttactctacctcgttttggaatcactaatgtggccaatttatagccaaaaatcagtatcgtagagtgagcaatgaatggcatggtgactgtgtgagcgaattcatgccctccctccccaccgctcgccccgcgtctcagtcctcagtgatggtaaacagaatgaggaccttctcccgaccgtgatgcgcctcagccctacttcccttgtcctttcctatcataaaatcttctttcatagaaatggtcatttctgttcatatctgtggactgtaaataacaaggaagtcatttttgaggtgaaaactgcacttagactcattccaattttgatggaaacttttagctggtggatggcattttgttttgtcttagttttgcaaggagttatcttaatttagggagatgaaactagtctgtgatccgaggtctcacttccatacatttctctcgggcagtgtggctgcctgaatcatgcctggatgccacaggtgcttagccagctggtcctgtcgtaactgtcactggtagctcagggagtgcagaggtgccagcagacactatgaaattggcctcgtaaagcatcagttatgttgtgatggtggcaaagctgcaggcgagatgggaagtgcagccactgagaactcacagtagagcgtgtgtaacgtaaaaagatgaaacccattgtacacagctgtgtactgcctccttgaagtcaaatttcccccattaccaaggaaaagttttttctgaagggggctgcttgacaggatgacatctggtgatatcatttattcctttggaaatcaatctgtggaagtgagtttccactgactgatgaggagaaaaatgaattggcttcacccagcatccagcttcttatcctgggagagatagctcttggtctgtcatccacgcagctgcctggtgcaagagccaagtttgtgcagcctgcagagcactcttcctgagctgtgggctgccaggtcggggggcagggggggcctcactgtgcagcctcctgccacccactgatcatctggggagactggcctatcctgtcaggagacgcagttgcccagacgttttcaagggcctaagatgtaggcagttgatccacagatttttggagagtccttgagttggagattacaggtgacctcagaggagggagtgagaacatctgggtcatgggtttctactaggagtccacagtgaaaacaagaagaggaatttacgacaagacagtccagcaacttcctttctaacttctcctttcacatatgctggatactccaagactttgcatttacatggacatcacagatccactttgagagaagtagggtaaaaagaaataaatacatagtgctttaggtgtatttctatacatcttaattgatatgggattacattttcacttgtgtttactgtacagactctagacagatcctgctcttttgcaggtaaaacaaatatttcttaaaacctagaaagacccaaaacaatttaacagaaacattttggaccattttggaccttggcagttaggccccagtgcagcagcggcaaccataaacctctccataggtgctgaacccaggtgatccctggcaccggcagccttatgtcagggctctcttatcgctggtttttatttctcctaataaaagtgattaaaagattcatcttttaaagaaagcaaggacacagaggtggattctccctgacgctagcacagctcatgcccaagccactcctgcagggctctggtctaagtgcaaaagctggaaaagctgcaggtcccgcaagacacagagcaaccctgcaagccaggtcaccttccctcttctctgctgtccgactggccctccaccatgtgacattcaaaagctcaagttacttaacctctcaaaactcagcatccttttctgtacagtggggaagatactggactgttgtgaggattaagtgaggagagtggcccaatgaggttgacagttattactgtcattgtcattatttgccttctcacaggcaggcgtgccacagtcattttactgaagctgcttcagtgggtcctgaattaggccctgtcctttgggagagacagtcctggttcaacacacagctccctgcccagggcagcttgggagtgtgggccagtttcgcctttagaaccacaattctctgatatgtgcaatgagagaattaattatagactcaaaggattgcatgcagacacacacagatacaaacacatacacacaacacacagagttacacacagacatgctcacaatacacagaaatacacacagacacacgcacacagcacacagagatacacacagacacacacacacacacacacagacatacgcacagatgggcacacacagagacacactcacagagacacacagatacacacaggcacacacacagagagacatacacacagcccacagggatacacacagacacacagagacatacctacaacacacagagatacacacagtcacacacagagagacatacatacaatacacagagatacacacagagacacagatacagacacagacagacatacacacagacacgggcacacacagagacacacagacacacacaggcacacacgtgcagataaggtaatattagctagttcaggaggagaaagagataaagataaagtaatattagctagttcaggaggagtgaaagaagccttgtttttctccactttttatagaagagaaagtgaagattcgatttgaggtgagttcagcacaaaagcgtatcccaggccctctggctccaactgcagccctttctacctcattcccagaccccacctaagccttttctcttcaaaatcttctcaggcacactgatacacatacctcagatttttaattctccggttgtgttcaccaggtgcttggtcatgattaagaattccgtgatgtgtaccccatgtgtttaaatttgctgctgagttaactttgtggcggcctgtggactagacctctgcacatgcaatgcagaacggcagggccagatttgaaatcctgctatcttttcggctgccttgtaaaaataacatcaggcgatggggatacgatgccagaggtcacctgtgataagttctgtttatggccattttacttctaggaagacaggaagtgtcaggatctcagggatctaggaagccaaaatgtttttccactctgaaataaagtgactgaccaggagttcccggccacgcagccctgtgggaactgccgcacggccacttttatgaagtggacacgtgttggtcccactgaaaagaaactccccacccatggctccctcacgctgcagcagaggccctgccacagcacctgtcagcccctgccagcttgcaggggcgcaggcgcagagcggtttgtgcccttgctggagccagggaagggcacagggtccctcctggagtcatgggaggtgcagccgaggttctatattaaaatacagaggctagcacatgtgcttggggaatgcagctacagtagtggaatgaaagtgctgtccgttccttacccccccagctcctcacctgtcctccacacgcatatccctggctccctttccctagtaaggagactgaattgaaattgtggcttgcccgaggctgcatacctgtgctctttctgaagcccaagtcactggctctagaattctaacctgtgaggaagccactgaggatgtttgtcaaaatacatatttctgtgccttgccccagttccacggcccaggaatctgcagttttcacaagcacccccaggtgattctggtggtgtctttgcacttcttcaaggcagtactgcctggaacgcagaatcccagcctcctctatcctccttgcctaatggcctggatgctctcagatctacaggggaagggaaggtcacacagtcatcgcaatagtaacctcagctgataaatcctcccccataaaacttattccccagtgttttttaataggaaacaataaaactgtaaccagcccaaatatccatcaaagagaaaatggagaagtaaatcatcgcacattcacctggaccagatctattgtaaagccaataatactgaagccccttccaaggccctgggagtcctaacagtgcactggcagtgtctataatttatattatgaaatttgcataaggaaaacattttgtctcatttgtgcaatttctccttctaaatatacgtgtcactttgtacctgatttctataagacccaggacctacaaaccctgtgtctgcccctgcagccacccagggaaggactgcacagcagcaagacagattgccatggagcatgttgtgcccaactagggacagcgcagatagattctgtaatttgcctaacaatgtctataggatgatcccatttgtcaaaaaaaaaaaagaactgggctttattgatgtcacctaaatgcacctaaacttcttttttgccccatgctcttctgtactcttgatctttccccaaatttttaaaaacatgacactcattcccttatttttcctacttagaaaagtgtagatggttttatcataggaagttcaaaaaaattaaaatataatgaaaaatactcaaatagtgcctcacaacagtaactactgctaacataaataaaatccatatttcctctcatacagaccccagagttgctttgcctgacagtgtagttgatggagaaaataatctttatccttagcctccatctggttgcagaccataaagacagggaaaaaatgagggtgttggtagcttcgttagaaactgaaagctcactgattttttcaaaacctaaatagcctgtgtttctccaaataactaatttgcagccttcggcagccaggactggcagggatggggctagggggactggggagaactgctctctcctgagggtggtctgacccgacagcacgcatgaccttcccacagtcaggaactgctcagagacgtgatggcaactccatagaatgaaatactcttcagccagtaaaatgtatttttggataaatatttgctttaaaaaactttactatatgttgttaaatgaaaaaaaaaccttaaggcatcagaaattatgtgcagtaaaatctcacttttgtaaataaatatacctgtttactacgtatgcataaaaagaatcctgagaaatataagtactgtatgcatattgttgttaagtattttttctgtttgcttatctataattctaattttgcttcaaagaacaagttactccggcaatataaaaataaaataactaatttgtcttgtcatcaaacagatagtaagaacaggcaaacctggccctccacactgccagccttttgtgattcaaggcttcagtttcctccacttgttaaaaagattcaacaaagtagttgaaatagtatgtgaaccagtaaaccctaaaaggtgtccagtgttgtctgtgagctaattaagtgatttgattctgactccccgagtcttctgatttcgaagcagtggggagtcagacaggagcctcaggtggcctctcctgagaggccctggaaagtgatgagaacctggcctctggcagctcttcataaacgtccatgttttccctctactctctcactcttttcccagggcctcaaacagaagatgaaaatcaatttctaaaacagccctctgtgtgctctctcgtatctctccttttcacacatcgtggtggtggctttctctgtgttcctctgttgattcagtctctggaattaacggatcaggattccatgcccagaatgctacaaagactgtgcttgagttctcccacatctcactcaattacacagaagtttcagattatgtaacagatgctgtgctgggttaggcagagccatctgacttgttttgctttattttagaccatgagatgggtgagtttttctttttaatgccacattcttttaagaattaaaaacctccacttggctgtcagcattggaaatcagagtgatggtgcaagccctgatgaggacaatgtccttgtctatgaaaaggtgaaatcattgcttgaaatcgctaagcaggacatgcagtcccagatggaggggggaattcgggagctggttggaaaagagtatttggcactttgcagccttgagaggtgcagaagagacaccgaggggttcaccaccagagccaccattgtcagagaggcgtccagctgtgtccacctgggactctgccttcagggcttcttgcctggctgggagctgcacaggcagactcctgggacggtgtgccgacagctctgggcacccccttctaggatctgattcctgaggaatcacaatgtggatttcacaatcacttccagtgtcttttgccaacctctgtgaacagatgtgcaattaaaaaaaaaaaaagaaaggggcccaattctcaacactgtaagtggaaactttttaatggaaaaggataggctaatgaattgaatttgaaatctgagacagaaccgatgcatcaaatgtgctggtgtttacagataatacaaggggggctgcatcttatggtttcaatccttttttaaatttttgttctgagagacccagccagcagactgccgccagtcttgtcagagatgtcagtggtggccactctgaatggaaagcagcatctctcagcatctctgaggcactgctcctcagcggagactgtggtggctttgcctttcagcacgcatcctttctacgatgcctgacagtgcccagggaatgggcagagctgggagctctgaagccctttcacctaaaccaccctgggtcacctgacctagttttcctcccaattttaattatgtcaggcacttcacaaaggcctccttggggacaccatgagctcactgtcatcagattgctccaatcacagctgtggcttgcacacaaccgccatctctgccccagcagatgctgtgtgtaaacagttgtattaattacatctcaaaaacatggttcttgccagatcctcaggatttgggtgcagcctctgaggtgggtgggaggccctcgagggagaaatgtctgcaggaaattcttcccctacgagaggtctgttttctaagttatctaagagctactgcagctgtttactgcagagtgaccctgctcaaagctgtggtcacccaaggctttgaaaggggacctccacttccgccctgggtggagcaccgtgctggagacccacgcctgccaaggcctcattgtcatctccacacgccgtccttggggtgggccactcctgggacacgcagacaggaagccggccacctgagccactcggaggctctatccagagtcagctgccaagcctcacgtcacacatcactgttagtcttggagggctggcggggccctgaagtcaattgaacacttggatgacagggaacttgccactgccagaggcaatatgctccatttttttgacagttccaacaatttttctttaaactgtcataaaaaattgctgctgtgaataccagtgtcggcgtccctgcctcacctttacctggtgcttttccaccacacaaaactgtttctcctcgtgctggccttgggcttgcagacagctgattcttctcctcccgcggctgagcagcctcctccgagcaaccctctgacaactctgctccttctgacaacctctgcaagggctgccagatgtgaacaaggggcccgggcagaaggtatccaggaagactggaaactcgaggaagcctgccctgtcctgtccaccagactttacgcttgcgtcactgggctttgggacctaagtcctcgtcatttgttccttttgcagttcctactgttctcagcacttccttccagcttactgaggtacactcagatgtgatatgccatcggtacagacacagttctgctccagcatttccccgtgttctttctgtcgctctatttactgaattaccgtgaggatgtggagcgaggctgagttctgtattttaacaccattttaattctcacctactgagaaatccatcctcttatcactgtgctttttttaacctgtcacgaatccatgaaatcctatcagccagcctgcatacttccttttaaggtgcagttgaatcaggagaaacttgccgcacatgctgcgtccgggcacagcattggctgaggctgctgccctgacctgtccgctttgtagtactgcccagctatgaaacaggttagccacacatgacctgcatttaggagtaacaagtctgtctgtacatgcacatacagcaacttttttaaactgtctatattttttcctgagataggtatttataatatctccatcttctttcccattttgaaacttagaacaagtttgcctgtcaacagttctccacagcatactgtgtattctaggattttctaaggttgagcaacggaggttcagcaattttgacttaatttcttcccatcccttttccacgcagcccagaagccttggatcacgtggtgaggggaagaggttgtgctatgtcgggaaactctgtatcgaagctcggctcagatcatgacattctcttgactaaaaccctcagtttccatcaaacttgtcactctggcattaaagcctgtcactgtgtggctctgaaaacctctctgaacgtgttccctgcctctgccctgcaggtccctgtgctccacagaagcccacttatgtgacccacccccactcatcaccaccttccctcacccagagcctcagctccccactcccacctgtaagacccctactggaaagattcccacctgcccctcaagattaatctccaaggacatttccaaattcctctccccatctctcagccagatggctttgctccctccaggaaccccagccaccttcgacctccagcagggcactccactccacattctcctggtctgtctggctcatcttacctgagccatgctctccaggtgaaggactatgtctaactcaactctgctttaaaagcagctaacacattgctctttgcatattgttcactcactaagttgaactggacttggacatgcacactgaactgcagcgtctgctgcttcttggtggcccagctcgtcaaaagaataagatttcagcaaaacaatgtaacaattttttttaccaaaagtaatgttaacaatatatggttttcccctgatgtttgcgtcaaaatgctttttggaaaaaacatttttcaactctttagggtcagaattaagcaatgaaatttatataccacatgtataatgtgtatgtttatctaagtatctgttcatttatatatcttaaatagaaattttaaaaatttttttaaaactcctgataaacattctcaggaggcacactatgtaactgttggttgatatacctagctagatggtgaaatcagattttgtttaaagcatggaggagagggaaaaattaaatcttgcagattctgcagtccttaacatctttgaaagaggaacatttcagacaatgtaataagaaggccacgtgctttgacttctgtagattttaaaaatacttctgtatagtttcttcttcctttgaagaagtttggggagtttgggaagatggagaaagatataagaatagactccccatatgggtcatgaattatctttttgcatcagaactcttagtgcagtttcagtattttcttcctcaggagggtgagctgcttccgaatgtcctccccttctttgaggcatcctctgttggtgaactttgagagcatccatttatgaagttgatgacctttcccagtctctgcaagcccttcagtgtgtgtcctctctgagcaaatctgaattgtgtgcttaatacatggaaagggatttgggagggttgctttttaaactgatttcttaattaatattatggtttagttaactagacagtctcattgcagaagtgcataaccataatatgtcttcaaatatatctcccttcctaacaccctgtaatatacttttgtaaagatacccttacagaatgtgatccaccatttatgaacctgcagcattgcattcagagactaagtgaaaagctggcagattttcatttaaagcacaagctaaggaagaaagctggtctagaaggagctacagaagggtaatgcttagggagggaatgatgtgcctgtgggtggtggtagttaaatctaaccaaagaatgatgtcgtgggtgtttggatattggatggtccacattgggccacattctttcaaacataagagtctgtagaaatatgacctgtaaaagactcttaaatattctggaaactgtttcttccttgtcacatccttatatatacttgaacctatgcctaccagacatgacatgtgactattcatacagatttcatcatctctggtttaagaataaaggatgctgcatagaaggctcacatcttttaattcacaagactgaaactgttctgaaatgacattgtttctaaaaattcattacttgcattatattcatttttatttttccatgccagaagggtagaagttcctgtgctcatattaagaaacagcaatgtcaatcgaggcccaactcaaatccaatttataggagttataaagggcgtgtgcctgttttgtctagaagcagtgttgggcagcactgagtaggatagaccacctgttgctaccgataaaggagcagcttctcgaatgctcctgtctggtaggcactatcccgagtgctttggcccctcatccacaatctgtgtggcaaaaggcattgcaggcaattcagtgaggagaccgaggcatggagagcaagtgccatggaattccctaaggccgtgcagggagcaggttgccaagctgggttgaaaccgtcctccgtaggctcccaactccgccgtcgctgctactgtgctggatgatgcctggtagatgcagatgtggagccccatggattctgagacaggccgggtttcagtcctgccctagctgcctattggctggatgaccttggcaagttgactttcgtgagcctcatttgtctcatctctcaattaagaaaacctagagcctatctgtgggggttatctgaaggattccagggatgcatatggcactgtctaccgcatgcggtaactgtttcacaaatgatgaggagcgatttatgttcttagtggaaatatgtcggcgtgtgaagtcccaaagctctgccctgcctggcttgatccagtgcctaggcactgcccctcttcccctctctcccaacccactgtaagaggctaggctgcctcagtaactctgaggggcattgactcttttcatccaaaaattcatgttactgccccacattttttctgttgttttacaacgcagtaggaagtgggcagactgtcaggaaaagtgatttatagtcatgtattgcttgtgctttggcttcatttgatccaatgcagatcagctgcactcagaaaactactcaagtgaaagagaaaaagtaactgaagggggaaatctggatgagtaagaattccagggataggaatattaatagcaagctttttgcctgatatagtcactttatgctgcaggggtgcccctttataaagtgcttgtacaatggatgtttgcttttgattttggatttggagtctaatgaatgttctaaattattattagaggagcttgcggttgttacatgtctgcctttattgcttatttttagccatctcccctgatgtcaaatgctcaggcaagaatgatacattcatttataatgtggctccttcagaaatataccacataccttttggtgtggtttgtggctgagaagagtggggaatgcacaagtggaaaactgcagaaagattatgccttcatcacttcaagtatttgagatgaaactagatcatttgctgttgctttttattctcattctaagtgcttttcaaagtcagcgctaagattttaaaatggttttctgttgttggcagagagggaattactctattactttctgataaaacagagtctttcatgatcaaagagaaccaggctctagtagttccagtatcctaacgtggacactaattgtttccctccttttcttcatgaaaacagcttctgcacaaatgatagccttgtgaactagccatgggcacaactggagaagcatttagggagctttagtgcaaattgagaccacctacacatctgactctacagggtttgacaacatccagggtgaatcacaaaacatcagtctaatcagggcttatatagaaagagtgaaagaactctgatttcatcctaaagattatttatattaaccattgttccaaatgcattaactattttaatttagttgttttgattgttaaaaaaaacacatctgtttggtagataagacataatttaagacaaatgttctatttgataagcttttagaaacaacttatttttattctttcctgtgagataactcagatgtggagaatgtgacaaaattttaagcataacatgagaagggctgacacacatagatttctgtgtgcttacttgaaaacaacaaaatttaagaatttggtataggagttgtatcaggtagtgcagagtccccaggagacctagagacccaggtctgggagcctagcggcaagggctgaatgtgggatgacatcagcagaaactcacagccactgctattccaaaaacccagcagcagctcagtgcagggcagtgctgatagtacagtgcctgcaatcctggagtggatttggatgtgtcaggtacgcacacgctcactgctcccccagcagtacgttgaacagtgtgcgtccaggtgtctgtagggcccctcgccctaactcacaaaaccattctgggtcagaagccaccaatattgtcatcatcctcccttttctgagaaccctagtaagtccctccagtggggcaagcccaccttttcccttcattctgtggcaatatgccttcatttcctaatcagttttgccctgctcattcaatgcaaaatggatctgctttccttgggcaccaatatgtccagggattgtttatcaatcttcagttctgtttcctttacatatccctccaaaaatcaggcctgcactgcctgtgcactccacaatccacaggcctgaaggaaatgttatctttgatgtagagacttaaagtaaaactcttcaaattaattatttcatgcaaaaggctagtcctgactctaattctaagacatgtctcctaaactctggaagtctgatgtatcctattatcaacatttatccttaatgtgatggtttatcatttatcctcaaagctgcattgtaaaatgtacactgtaaagtgtacattttaaagtcggttttaaaaaatcatatttagagatcctggtaaaaatctatcaagtcaagacattaccttattacccatggaattgtcttcaactcttacagttcaaatattcctgaattggctttcacaataaacatcctaaatatgtaagtagaaacatatatattgccaactttgtgccttcccaagcaaaattaaaatacaggaaaagtcagtttgttttgcccataaataaatatatgtgtgtgtgtatgtgtgtgtatacacatacacactcagaaaagatagaagcagcagcatattttggcagcatctggtttattggaactcaaacgttctgattgtgcatacagactagttaatgtggtaacaattatgtatttcttccctgctccttgccttctttccctccccagtttttttcttcctgatagtaggtgtgtacttttttcctatttccattggcaagccacatgacaagcaaaacgatcactcgaagaatattgttccctcaatcaagaaaaatgcccattgggttttgttatttgatgttatttgatgacagagacctattgtttttccatttttctttttttgttttccgtggcacctatggaattaagcaatataaaaaatctattatttcagatgttcacgtctaatgaatttcatgtgaaatactggcagtataaccccaaatagaggaaatttgtgaagagtggatgctgcagggcatgagacatctgcacagagttcatctcttccagcatcttgcatgtcccaagcactgccctgccaggcagagaatgctgcagatcacggcagtgaattccagttgttcagagcacatttgacttccaaattctcaaggccacagatttgaggacagaacaatatttgcatttgaaattggaagattattttttgcacaagtgcctatatgctatatagagtttgcccactctgcattatcttccccctgttcccccgttatctggcacaagctattcaaaagacacgcctacttgtaaaataaatggtttgcaaactaaggaaaatacttaaatctcatgtaaatggtactatactatgtataaaaatgtgaagaaacacagaacagctcatgaacacctccactgctgtataaaagaaccatcttttttctggctcctattggatgccttagaaaaatctgtatttcctctttagttattgtgtttgaaagatgaagttgagacaaaagttctattctttttaagttggcagaacttctgaaaggtgatttttagctgcagtgtgactcattccaaatgcagaaatctctgaccctgagttagtctatttgtcatgcaagagcctagaaaagccctgagtgataagaaatggccataggccattcccacagaattttcaacaaaaatagaatcatgcttatgttctagtcatgacttagaacttataactcatgttcggaactgtccatgttcacgcacaggggccgtatcactccgccagagctgccctgggtgccggtgtgcagaggggtccgagagtgactgtctcttcctctgttgtcgaatgtgtgggttatctccataaatggctgccatgagcatccttgttcacacatttttaggtacttgagtgagtgtctgtggaataattttgggaagtgaaatctgtggtcagaggtttgtgagttttacatgctacattttcagaagttgagaaatagcagtaggctgaaggcaagtcgccatgcctggaattcatgaacactagttgaaagaactggcgtgagttagtcatgacaggagagatggggaagggagttgcaggtaggagggccatcttcaaattctcaaagtatagtcactccaaaccaaaattcgatttaatctgtaggactccattctcaaagcacagtcactccaaaccgaaattcgatttaatctgtaggactccaggtggcagaataagaggcaatggatgggtggaagcgaaacagggccaaagtttgacttcatgtgcaacttcctaaggagtgatttgaactccacaaacatgaactaagcacctcaacacaggctgggcaagttgctgttcttttggagcttacatcttagtggggaaagagaaatgcctatgtaaacatataaatcagcaggatacattgtgaggacggtcattgctcagtgagactgcaatagagtgatacgctggagggggctgcaagggagaaggtgggagggacagcatttagcagaatgagcagcacagtcccataggaagaagaatttattgcctccttaggcaaataaattcccaaaccttgaacatcagaaaggaaatagattaatgtgcacagaggattaaattatgtgatctgcaaagtcatttaaaatctatttccacataaaacatattaatgcaacctaaacaaaaggggtctggataccctcatcttcttcccaagcatcaagtctttctatagttaaactgagatgcttttattcttggaaaattttaaggactatctacagcaatggaagaatcgggtgttgggatgtgttcccaggtaataatgactgcaggctgatttggcccttgaggtgtggcctcatggccctctccaaaaaaaatcaaggacctgctacaaagcacaaagccgactgcaatgcttgctgcttactggttagggcagctcctctttgccagcgaccaagcagaaagcaagacaagacaggttctgaagcagtaattcaaagccttcctcgctttcccatgtgagtcattgctagtcagaatattacctttgcagagaggcttaattccaaatttgctcttaaagggatatcctctcctggtttaggtataaacttttgactcacaggacaaattctatcattcctttgggcctaggattgcatttatttccatgacaaaagggcctgtctggtgtttcagcaaatgaaaacaaaaatataaagcccatctccttttgaatgagctctaaaacagttctccactggacttcagaacaagagggagctctgggctgctggctggttgtgcatttgctgtgggttccctccggcaggcgacctctccgcgctgagaaggttatccggataaccaagtaagaaagtacatgaggaggcacagaaagaaaaatgtgagagataacagcataaacacacagtgtatgttgttatgaggcatcacatgatgagatactgctggggagggaagaagtgaggagattcctaggaatcttatgagaatttccagagacaacaagttttgagcttttttttaatttagaaaatttaccttatttttaaaagaatatgtaacatatcccatgctataaaattctagacatagtagatttaaaacagcataatggaaaatataaatatctattttcttttcctatttatgtattctgtgccagtaggaatgtagccaaaaagagagaaaaggggtctctgcagacatggatgtctctgtgacttgatcactgctaacccaagaagataataaagcagaagcatgtatccaggttgctgcagccaagcctgcccggtctgcggggcgtcctcacacatggggcagctctcccaccccacacactgggaaaggcggacagaggctgggcaaagcccccaattttcgttggcactgaccccgatgatttataggcctttgtttcccatgttaaatgtcttacgatcattaaattatttatagctcaattagcatgtgtccaaaaccaggaagttcataggagactgtgtgactgggaattaaggagcaaagcaactttccagtctgtgatttactgggtttccattctgtttcctgttcggatccggaagtagaatttcaaatattgcttttcatgctttatttgggaccgattttagccccgctctcctttctcttgccattcgctggccattagccaccagcctctgcacaatgaccagctggcccctggcagatcttgggcccaggtgtgaagtcgctggagaagcatttcagggccaagatgggagtgatttcattttccattgacactatgcagaaatgaaggggattcaagtgccttcagaaaagcttccttccagcgaatggagttttgggggttttccagacttgcaactgcttttattcttggaagcatcattgttgctttttccccccttccatttatatcccaggaactgattcagaaaccatagaaattggatttggaatcgctgaatgctagcagacagctgactgcactcttcccaagaaaccctgccagctgggttcgggtatcgcgcggtgtgtgctctctctgcctggcccgctgagtcctctaactctaatggattccttcttacaccaaagtgcactagaactaaagtgttttgcttcattctttagacattttgtggtttagggctcaatcagccagggtatgatttgcaatccacagtaaccggtttcagagcagctgcccagcgaggcaggtttcatctcgcttgctagacgttttgtttttttttttttctaaacctcacaccttttatttattagacttggattccagtttcctgagcctgtttgtgccactgattagacaggcttgaagcagaacccaccaggcttcctgaataaaatgcagcagtgattgtattagggggttttaaattgctcaaaatactgtctaaaaaacactaaaaatcatgttactttctagattgaataaaatcctatagaaatgaattcctggacttgatatgtagcaagctggcattggctcgggagtgagtgggctcagttaagtgagctaagatgagatggtgcacaggcgagcacccacctgaggagtgtttggatgttatgatagccagctcctctgtaaagacctgtccttctatgtcagcagcccagcagataaatgacgtgtaaataccacatttaggagggcttatgatgatgccaattaatggagacctttttgaaacaggaaggaggtgaaacatattcctttgcttctacatcactgtgtgccaggcactgtttacagcatctcgtttaaccagcagtcaccacctgacggatggctgatgtggggtggggtcccagggtgggattgcgtgatgggcttggggtctctggctgatgggtgccagagctgggactggaactcctggcgtgactgaggcagacacctgggctacccagcctcacccacgacgccctcactaagtgacccacaggactcaccggaagcagggcagcaaggtccccctacagaggtccccactgcaaaccgatacccagcttagacagcagttctgcagtcggcgtctcaccccttcgggtctcattgtgactcactttgatagccacacgatttaagggtggttcagtagtgatttgatgagtgctgtggctcagggtcattcccctgcccaagcatttcaaattccagaagttcatgccctgcatggtgggtgaaaagtctcaggccaaccatgagcacacagcagccaggcgactgaggcagctgcccggggtggcacgttgctcaaacccatcatttggagtcaaaacaaacagatgattagctggggtggtcactttcaatcaagagttttcacatcgcctagacatggcctcagaatcaggcctggtgtggccaggggctgatctcacagtagacaggaagtgtggcccgagggccatggctgccccctcagaaggccctgtggagtggctggccgagcctcagcagcctcctgtgaagcgaggaagggtcttcctgccggcctctggagatcagtatgggaatgcacaagtaggaaacgctggatgggaatccctctgccctgtgataccaaggcagtgagtttgtagactatggaattgctgtcggagggctctgtaaccggccaaggtcacacaggtagccattggtagagcagggactggaatcccagacccccaacttccaggactgtgcacctttctttatcccatacagccttacagtcaagtgccagtgcaacacctgattcccaggttccagcctttgtcttttataatgggaatcaaccttatcttgacgatccagagatagtcatcaaggaagattaaattatccccttagactcagagtgaccatatcattttccctccacacaaggacacttttgagaatgaaaaggaggagatgtctgtaccagacgctggatgacaggcaccgacaggctgtctgccaggggagcagcgattcctgtatgttgtagaaagtttttcaaaagtcaccttggaaagaggttttgttccttaaccttctgttaaataggaagctccgtgaatgaaaacaactcccttccctaaacattctagtaatgacccaacactgccaagcctgccagctctgcctcatggtcgtgttgactgtgtgagactatgtgagtgcctgctacacagtacgctttcagtaaacatggtattgcctcgataatcccacaaaaatgtcctattcaaatcacctggcacccaggaaatttccttcttttttttcccaggtgaaatatacagttgaaaacacctgacagcaattcccctctcccatgtgtttgcaggatggtggttttggttcctccatctttgatgtgtacaagtgtgatgttttccccccacagacaagtaaaccacattctcttcacattcccaatgttttgtcaatgtacctccttcaatagaggatcgataaggaaaaaaatcattgacaatctcaattagattcactatttcatccaaaagcatagcttagaactctagtttttgttcaacactcttgccctatgagtgcacagaactttaattctgatacaaacatccctgaatgtttagctttgacagagattccaaggtgatttgataagaagcagggctgtgtttgggctctgggagtttttgatatggtttcaagccccatccaaaacccacagacctctagaaagtaggtgcctgccttcctgcagcagccctggagcctgctgggggctttgagcagctgctgccaagccaggcctcacccgacactctgatgggcacggccatggtggcaggggcttggacgctgccaggtgactctaacttgtggccagggtgggaagcactgctccacagaggtgccaaaaccaggttccttcctgtgttctcacatttcacagcctcaatgtaaaaagtaagacatgggcactctggaatattacaaaaatatagaaaagcatgttatagtaaataaaaggctcacagaattttgtcatttaggaacaatgattattaatatattagtgtgtgtttttgctcattaacagtatatcctgagatatttcctataccatttaatattttaaaagatgtttacactggccacagtagctcatacctataatcccaacactttagagggcaaggcaggaggatcacttgaggcttaaaaattagccaggtgtagtggcacatgcctgtagtcccagctactcaggaagctgaggctggaggatcacttgagcccaggagttcaaggctgcagtgagctataattgcaccattgcactccagcctaggtgacacagtgagaccctgtttctaaaataaataataaataaattaaaacatttaaaaatacatgatgtttaattattagaggactcaattttatatctatgtatacaataatttttaagtttcttaatattggacttttagtacctttttaaaaatactatttttaaaaaaatctgtatttctaactttttataacaaggaacctttggctttgagatgactggggaatccattctttcctatagtatccatgtccaatggacttaaagtattaatcaatgtgtttatgttttgttatttttctggcattacaaaaaattctaaatatattgttaccgcctgtataaatatcagcttttgagagaaggacattgtgtagaaataatgaaacactgcaacttgtatttgtattattcttttttttttttttttttttgagatggagtctcgccctgtcacccaggctggagtgcaatggtgcgatctctgctcactgcaagctccgcctcccaggttcacaccattctcctgcctcagcctcctgagtagctgggactacaggtgcccgccaccgcgccgggctaattttttgtatttttagtagagacggggtttcaccatggtctcgatctcctgacctcatgatctgcccgcctcagcctcccaatgcactgggattacaggcattatattattctttaaattcacatgagaatttagtatggcttcaaaaaataccataagttaaaatatcaccaagactctgttcagacaaaagtatcagaaaagtgagccaggcactcacatagtttatagtttataaaagtgagacaggcatgatctcttaacctcactatagtcctgtgaataaggtttatttacatttcattttacctgccaggattattgtaaaaacgccaagcacattgcctacacaaactaaatattcagtcaatggctgctattttcatgagttcgttttaacatatatttattgtcctctactggatttaagaagttatatttattatcatctaagattttagctattccttctcttaaaaatagattttataatcaatggcagtaagggagagtaactcgcagttctctgaatctcaaggggttcctggaagccttcctgaaggtatagtgaaatttcagcttcacattcccatccatgagctccctgcaaatatcccggtctgctctcaggacccagtgacttacctatgcagaggctgtagatagcacctggagcttcctgtgtgccctcctcaaactcagccaatgccgtcatacagtagcaggcaggtgtctttgctgggtagttggactggatgtccctgggattgcagaactggaatggggagtgacatcaggaaactataatcatcaggacaacatggtttgccataactttaagttttaagcgaccgcagattatgcggagagagatgcatgcccacagccatgcttcccatgtaactggagaggggtctgaagtttgaaacaagtgttcctaggcacgggttacagtgtttgttatcatcatacttgatttagaatggggcacaacatgtggattcatggtaactgttacaaccttactcattttaatacctgaaaacatgctttccccatgctgggaatcgaaagattctcctaggaaaagaaaggcttgacaacatcgattcaaaaagggcatgcattttcctcatttaaataactctaatgtgcaagtagatcccctgacctcaagctcagaagagtccaggccttcacaccttctctgcttctgctctggggccagctattgagattcctgtgcccacgcaatgcgcacatcccacccctggccgctgtccacaagaaatccagttgcaccaagcaccccactttttgcacctctcatttatgtactcctaagagcctcaccacaactcccttctaaaaacatgagttcctgactgggaattcgatgctgcccaggcagctttgctcagagggagcagccttctagaaatgtttcaagtaaactttcaagtataactaaattcaaaaaaaacacatacacacacacacacacacacaagtcaaaggtgtgtaatttggccaatatcacaaaccaattagccctttgtaagtggcacccagatcaggacagctgaccataccagcaccctagaagcaccccgtgctgcctcctgggacagggctaccaccatcctaaggccagcacgatgggccagctttgcctgctgttgaattttgcttacatagaatcctccagtaggtactcctttgggtcaggttctttcactcaacattatgtgttgatatttttccatgctgtgctgcaaaattgtatttcttgcattccataactgggcagttccatcataggagaataccacactgcgttcgtccattctaccgccaatggacatatgggttctttctcttttcttgcagttacaagtttatgaatattgtcccacgtgtccctggtgaacttttgtttgcatttctgttgggtacctcagagtggcgttgctgggtcagagggtactggtcgctttagtagctttgaaagatattgccaaaacattttccagcgcagttatagcaaattatacaccaccagcagtagaaaacatctcctaattgctcacagtaaacccccaaagattgccacatacatcttccatatcaattacttaactattcagcaaatttgaagggaaatatatttaatctttttattcaaatagtttataaagtggaatagagatgtgggtaaaagttgtcttgccacctttttagatcggtaaaagtttgttgaatgcaggcaagaaaagatgagaaataatggtacccaatgaaagacatagcagtctacaaggaggggcatttcccggggtgggggggacccacactctgtaactcccacattcaattagcatgttataggtaagctgcagaaaacgaggcagcttgtcaaagaggaacggctcttggccatggttgctgccctaggaggatatttgatactagcagagctggggcaaccctggaggaaaccacctggaatgatgggagaactcctccagggaacatggccctttaatagatctctgttataaaaaataatcccaaagcagccaccagggcatactgctgcgatcaagtcctaggcggtattcccttctgcgccatagaccctgtgcagagtgccctcaacgaaggagcaaggaagaccaagtctcccgagggtttgcatatgtgtatgtgattctgcagtcatggtgaatgacacagtcagggctgcggaaaagcattggtaaagtgtatatttgaggcttcagaagtttgaaaaggctagatttcctaggccaaaacactgaaaatttgcaattagaacttcagtgctgatgctgggaagactggagttagtttgagacatgcacctgtgcagaactgggcccccagaaaaggagaaggaagggaatccagaccagagtagggcctgacaccactcagactcggcgtgtctataaattagaattgcgttacaattacactttgacattttagtggtttttaaagtgcccagcacaagttaatttttcattaatgaatcctttattcataaaatgcttagatggagattacccttttgagcattttgccagtgcttctgaaattaatggggacctcctgttggaggacacagtctgttgcaataggtgaccactgctctgaatctatgtcacctctccaggaccacgggcacaaccatcacctgaggcatgttggagatgcagatggtcaggccctcctagaatctcagaatctgcattttagcaaagtcctgggtaattcctatgtccattggagtttgagaagcactggtaatctcaaatactttaaaagattactagagtaagataggctcagtaggtacctgaaggcaccatcccaaagaccagagtggtagaagcaggtggaccagcctctgaacacatttctcccccactccccggctgtgtggaaggttgccacctttggggtagtcattcaacaaacacgtgtcaactgtccactatgtgtcaggccaccactgggcactggctgtggctagctggatagacaccatttctgccctccagaaatgtcatgtccactggcacatgacaagtcactaagtcattcagagccatgggtgacagctccaggggccgacaaaggagctgtgatctcacagatccacagagaagtgtcccagggcgggcgggaaccaggactgcacagggaggggtgaagtgacacataagaagtcagcccatcagcctgaaatgctcccccaaatcttcccattcagtgttttctcagtagcaaactcgtgggaaaattggttattttacttaaaaaactcatactagaaagctagtttaactttaaaaataaattttaaaaacatttttattaacaaatcctacctttcctccaaagtcaaggagaaaagaatagaagtgaacaatggaccaagtaagcctaaaactctgctctttcccctgctcattttacagttcaagtgccattcaatttatcctggcaagaagaggaaggcatcatcaagaccttaattttctaatacatctgatctgagaagaatgtgaaagctataaaattaatttttgatcaataactacaggccttttgagagagtgccctcctaatgaattgagtacctatttctccatacacagtgtctatcatgacctacaaacccttttcccatgaggtgtaacagagagagattacagccttggaactggatgtcagactctcctggtttaagacaataagccatgacatagagcctgaaaccaacacaatcttccgagtggttccagaaacatataggggataatgttggctctgatgctgtacatccccaacaaccatcaactatttggaaactagaatttcagcataattggagttggtgttaccctagcaaatgctgtgggaagagagtctcactgtgtatcttctcctgtttaaagcctgaatttgttcagaatgtaatatctctgtttagccactctactgaaactgatctaggaaatgttcaaaaaaaggtatcccaaggatccctttgtagctacatctgtgggattcccctcgctctggcgtggcctggcccctctgcatttgacaatacggtcctatgcttttgtcttcctgggctgcgtgaacccaccctgccctggttcacctctcctcttgacccatccttatcagtgtcttgaaaggtccttctattggaggacacattctgttgcagcaggtgaccactgccccaaatctgtttcacctccccagggccatgggcacaaccatccctggagtgtgttagagatgcagttggccaggtcctccaaaatctcagaatctgcatttttgcaaagtcctgggtaactcctatgtccatgagagtttgagaagtactggtctcatgagttcctgacatacaaatagtgctgaggccagtatgctgactgggtagccagatacaagtgaaaaccttcctgttttttgcaaacctggatggacccgaggccgctgacgtgggccaggacaagctactctttttcagtgtttctgttgcatcgctgtgtctctctgtgatcaggtgctgccctccctggcaggaggactgcagacaggatgaccaagagcactctacacagcctgctctccagtgttgggggacgccacccaccctcgtggttcctgttcatctgcctacacgtggagggcccaagagggctaatatgtgactatctccacttcctggtaccctgtgtgaataacttcacttactaaagggatgttgagcaactttattaataatgaagaaagcactttggtttgacaaataatcactccattttttcatttgaaagttaactcttgttagtagagaaagcaatgtattacaaccacaaggacgtttacatggaaatgaaccatctgcaaagcatcccccattttccttttaaatcagccaatgggtggtggtgggagaaatattcaccagagtatttaacatctatcccccttcctagactgtcagctccatccgggcggagactgttggtatctccacagcacacacagggcctggcacacatccggggctcagtgagcacttgctgaatggtgaacagattagctctcctgggaacgttgttgacacatctcataacactggtttggagtggagggcattcatcgggctgcatattcctatttttaattgtattctccactggttacagcacctacagttataaagacattgttaacattgcttataggaagacatttgatggaaatgagtccaaaggcattacggttagaaactggccaggtgtcatttttgagagattagataactgttttccggtagagtgaattgcctgtttgttgcaagttgggactttgctgggctggtttacagggccaaggggaaagagataagtggatcttctagtgagaggtcatctgttttgaaagcctggaagattccatgaactaaatccaagtcttacaacacagggaagtgtgtcatactgtgcagggatgaagtctccaatttagcatgaaaacaagagctcctcacactgtcctcttcagaaagcccatacaatccaaacttctgaatgcttagctgcttacaaccatacatagattgagggataaaactctgatatggaagagaaggtaaacattttttggcagacattcccaggaaaaggcggctctcttctctcattgctgctgctctttcagaatccatttcaacagaggaggagtcaatgggagccccgtgcctctggcagatatcatatggcgtttcagtggcattgtgtgttacccttcttaggtaacagctcagccattagaagaatgtcctacacaccttctcattttctgtgatgagaggaatgtgaggtactgcccttcgagagctgtcatttgtcctagtagccagcagcgtgactgtgctgtcttctgctctgtctccctgtcagccttctgcccagccaccaccactatagttttgttctctccattggaactcctggttcagagaattaccataaaaaacagacccctagacatacaacactctatcacataatggtgactttgtcttctattttggattactgagctttcttgggtaacttccactaaatcgaagttaatattagaagaacttcctcttactagaatcgaaaagcatttaagtgatgcagtcaagtttgtaccataagtaattcagtcatttaacaaatatatatggcctctgtgcgacagtgaccttgactgggaatgaagctgtcccatgtggggcctgttcttcaaaggcagttccctgctgcccagttcagtccagtggatctgggcatctctctttaatccgcattaggggctctttactgattcttcactatccaaaaagacttggaggggagacctgagcccacttctggaaggaaatgataacaatttatttagataatctttgtgcaacaagtcaattcactgaagagatctgctctctaggagcctctgtgaccccaccataactgggaaggctctacctctccagtcttcgggccacatttctctctggcctgctgtcttcccagcactctcagccttgctcatggagcactctagtcctccgtcgaccttggcctttggtaacgtgatttttcacctggcagctcccatctggtctcactccctctttttgtccagtctgcatgacacagcctcacatcgttagtgttccctcactcccctcttactgcccaacctgcaaagtccatgcctgggccagtgcagcatgtgtcctcaatgggctgctggtggcagtggggggaaccgcacagccacgctgtgtgctgctgaagaaatgcacagcctcctaccctcgccctcaagaggcagccatggctgcgcatttctgcccttctgagctccgctcacttttggcagcagccgttccaacctgcatgggatcttcactctctcacagatgtgctgactcctcctgctgcctcccctctctgtgccttctcactctctgttccctttgccctttctccccttttctcctctgcctacctccaagccatccatcacaggacagctcaagcatcagatcctctgggacactttccttagttgttcagtctgatgaggtgtccctcatcctctcttagctgaaaatcagcagctgcctcaacttcttttccagcatgtctcatgagtattgccacaacagcatctgtcacaatgtggggtagtggctgacttgcttttctgccattcaactgagttccctcagtgctggggccagcgtgcagtgtcttgtattcagtatatagctgattaattgatgaattgattaattaatggttcacactagcacagtgcaaccttcaatgcaaagatctcatcaaaataattcacatggtgggatattttagaaggatgaccaggctagtttgtagtaagaaaaaatcaacaagactaggtcaggaattctttttttgtctacaggcttgctatagaagatattgaaaatcatctacctaattacctttattttatcaggttgtgtattaaatatcacgtctgggggaagaaaatgtgatatgtgattacagacctttcctggtacaacatagtacgtttcagattaactcaaggtattgtggtgatattgcggtcaaagccaggtgattaaagagtcattctttgaaacaaatatctgtgcaatcaattaagaaattaatttgcaaattttatttgcttagagtaattgatatatcattccttttacaaacaaatataaagaaaacttaactaaaaatactgcatatctctttcagattatatatcccagaaaggatatatttttctcctttctggtcttcctttttggtgtagcatctgtaggaaatgcatttcttcatagctaagtgtacctccttgtgaaatatcttcagagtctactggtgcacataagcaattgctggcagcagcttgagggtctccatctcacatttatcatatgccttattgcatgaggctttgcaagaggaggtctagagctacaatatctcatggatatgaatgtcaattcaaatcccagtggcagtttatgagggggaaagcctagaagagaagaaacctagaggaatcaagcaggaggggagagtaataaaagactagagcagcaggtttttcttaactcaaactagaattaaatctctgtgtgtgtgtgcatgtgaatgtgcccgtatgtgcatgcatgcacgtgtgtaaatggatgtgtgtgtgtgtgcatgtgtgtgcaagtaagtgtgtatacgtgtgtgggcatgtattgtgtacatgtatgtgtgttttatgcatctgtttgcaagtatgtgtgtatgcacataaaagtgtgaatgtacatgtgtgcttggtgtatgtgtgtgtattaatgtatgcgtgtagttctagagtctagttagagaaagtgcataaagaaatagggaaattaacaagaaagctatagcttaaattataggaaaaacttttctccctatcagtcatggttttaaaatgttcagacttgatatgtttcccagtgctattgtcagaaaatgtccctatgacattccatactacttcaatcaaatctaaaacctttgttccaacatgttttattgatatgagtatatttcaaatttctaccaggtttttggagaggtattttggccataaaattgactaaattattcaaaataaaaaatgaataagcctgggccaaggcttggagacttgcttaactcagttcttaaattttcagattttcaaaattacaaatttaagctctaaaatcatggtgctgtgtatgatattctttgattgcaacttatggttgaaaaactatagagggctttatgctaagagttgtggatcttaggattttcatgaaatctgcattatcatcatctgcaagtttagatggggcataactgatccaaaggatggatccctcgggggcaattcaactggctgattccagccaagatgacaacagtcaggatccgttcccttctgatcatccattgggtgccctgatttcctctacagccctagctgaaagaccagacactatctcaggctggctgccccacatgccttgctccacaccaaattcacagtctataaacctgagcctccagtgctcctactaccatactcactcgaacattcccgattctgacctggagatgtcaacagctacttgatgccactctcttctatctttctgtagctaagccatccccaagtttgtcgattcaccctctttaacccctgtcggggtgtccattgtgccccttcaccctgccatctccctggtgcactgttttgcaaagttcagcatacatgagcgtcacctgggaaccttaataaagtgcagatgttgattcagcaaatctgggatgccctcgggctgcatttccagcaggctcctggggatgtccccgctgctgtgctgcagatgacactctcagtggtgggactccaggctctgctgtcgcctcctaggggtttctccacactccctggaggcctaatgggcccttctccacatggcagtaagatctgtttttgtgtttgtgtttcaagttgggagaaggagattatttaatactaaaatgtgcaacatgggattgagaaaactaattattagtcataagttgagtatgcaacattgaaaccacatgctttaaaaaattataagaaaaaatcatagtatttgaaagttacaagctattatggctaactccatttatctcagttagagaagaagagtcacctgtcaccagggcactgccagaagccaggctcatttccaacagcactgggtgctccagctttggggtgccagctcctcccataaagcaaacacatacctagggatgatatttctttgcaagggctctgccctacagcttgtacatctcaagaagttatgtaattaaactgtctgttttgagaaaattgtagattcacacatactagctgtaagaaatgatgcggataaatccagcgtaccagctttccccacggagacgtcttgcagcgtcacagccaggatgaggcattgacccaggcgaagtccagagcacctgtgcgctacagggccccttgcactgtgctgtcacagacacgcccacttccagatgccatctaggaccccctccaaaaagcagaggcattcttaaaaacacacatctgcacatgttcctcttcatttgaatctgtcagtggcttctcagtgcctttcaaatgaaatctaaagtccttacaagccttgcagcaggaacctctccatcccacttcccctcacactctcagcttcatctctgctaggctctgttcagccaggcagcctttcacagtccctctcctcctgccctgccaggaaggtcccctgcccccaactcttccccacatgtggcggggccccgcttgtccttagaagcccagctgaactgcttcctgaaggaacccctccagaacctctcagaccaggtcaggtttctgcactcttagatcatccccatggcataatcacagttgtgatgttgtgatgattcagtgaatgtctgtctccccactggatggtaagcttcctgagggcaggaacagcattggttccagtcaatgctatgtcccaggactgttcgtttttgcacatactaatcctaaaaggacgatgacaacagcaaccacttacatgacctagatgctcttctgggtgttgtgcaaatattaacaatttaatccttgcaacaatccacgagggaggcattcttctactcccacttaacagacaaggacagtgaagctagtaaagagaagtcatttgcccaaggggaccccactactgttggcagagctgggtgcaaacgcaggcttgtgaagccaggacccatgcattcaaagaccatgccaggtgcccccactgcacacctcatccccacataccagtgagggggagagaaatgctcctgcactgcctctgattaactgctttcctagaagtcacacatataaaagggatttaattctagtgggattgaatctcaatagtttccttattaggttgatttctgttaatagtttaagtactggatatacatgaattagaaaatctagattattagcaaatgcaaactataaagtattttataaatgttatcttgtttgtcaggggatgagtgagatattcattatacaaaaagtagtgtggattttgaggtagaaggtttactaaggatcataccgtagtatgaaatagccacaaacattcagtgaaaccaaacacccccgcttaacctcaaactaacactaaataataaggaatagacttgggggcagtgcaagtgtatttctaatggtgaaaaccattccccagtgaaaactaatgtaccatctagttaataagagctcctctgacccacgcacatcaatacttacatcccaatggtgatgtgacattttgggttttgtatttcttttgcaaattgagctagcatttttgatgagtggcagggctctgctacccaacctttggacagtttccaagcataaaatcacaattccagataattctgtcacaaagatctgggtctcattaggaaggagaggaagctgggagatgatccagtccaacctcccccaaaccaaacatcacggccttctcagttgtttcaccaaccatctaaatgttttagtaattctaaaaattgatgcgctttttccacgaaaggaagtgttaccacattttccaagtgggaggcatctatatccttactccttcatcctctccttcccaccccctcaccccccaccacccacacaacatctgcaattcttaaactaaagcacaaattgttacaaaagttaattgcactttcaaaggaatgcttgtatagaaactttctcggcttcaaggaaaaataatacgctttgaatggctgttcaacagcatagaaattagctgagtagaaggcactcatatagccattaggaccaatcctttctgccgccaacaccccccttataaagacttgacagtgggccagaataaacaacttcaggatgaattcagttgagacacaaagtacacacttccagtttttcccttctctggttactggcctcaataaccaggcagtcaacttaaaaagaaaaacaaaagcttgcttcagattacagattgcagacttcttataatatgtccatttcaccaggccccgctctcagccccgggaaaggccactggaaaccacctcacatggtagggccttgcgggagccagtaataaccttatctccgtcaacatgttctgtcagattgaatggggcagccagagaagccagagttggcacaggaaccaaaacaaaggcttcccatcctcctggagtgagcggttgagcctggattggtgcttagacctataatgggtgcaagcagcgttcattcatagtggctttctagacccagggacttggccccagccctgctgctccactcctcttcttgcttcattaccacgagtctcctagaccaccgaacgatgcctgcatttgaaagacacttctgctgatcaaagcagctgatgtgtccctttgcggttcatttctaattgtccccaaggaggagaaattcaaatagtttattactgagagttaaagaaatccactgaaatattctttggtctaaaattactgtcatggcggagcagcttcaccttagtcattgcccttaaatatgaaagctatttaagaaagtttgcccttaaatatgaaagctattttaaaaagtttaatgaaagaagagaatcacaaaacattttcaaaaagcaaaagaaaacctaagagaaaagttgaaagtaggaattttttaaagaatatacgacgtgtgttctgtgactcacccctgcaagttatttgtgtgtattcccttgcatagtaattaataatgaagcaaagcatggcaatgatatcttttcttgtctagtattctagaagactccatgtttttggaaaatatcactctagttagatctcaaatatattcaatcagaaaatgggttttctacaagattctatatctgtagtcaatagcaaatataattctattaagctagtaggatgtgataggaaactaaaacctaggggagaccaaagcaaggaaaaatacttcctcatccaaacttgagagcaatttaccgtcaggcctactattaatagatggaatacagattccattttcattactcaactgccatattcattattacactgtacagaaaagggaatcacatctgttgaaaacttatatatgatgttcatgcatgcattccagtaattcaacaatttttatttatctttttattgcttgctaatttttcaaaataataagctaaagaaaacaaaatgtttgtgctgttctcagatgacatgttatctctttaaaggacaaaatgtgctgtgaaataatagaatgctttcagcactcaagtgtgagtgagtgctcatacatgagagaaagccgtggggactacagaagccaagaagcagatctagctggggaggcctttgcagaggatgtagttgtgtggagaggccacacacgtggaattcccaggagggctgtggaggcggggaatctgcaggaaagcactggggtgagaaacgtgatgagaaacaattattgtcttaaaatatctgcagggctgtaaggtagagaagcaatacgttgcatctgtgttaagtcaaacaaaattatcaagggactggtttcagcttaacataaggaacaattatgtgatagggttgtcaataacaagagtagactgcttcttcacacactcctagtcactcagaatggtccaggaggagtggacaaccatttggtagagtatgggaaggcaggggccctgggtgggagtggtgagggtagggagtgagtatcccaatctagaagtaaattgtgcccagcacggagctgcaacactgccctgcacacaaacacacacaaataacaatccccagcccctgcatttccctctccggtttcaggaccttgtatcttacttcaattcctttatttagctgatgatgaaataggaagagcttagcactaagaaaatccttttggagtttggccttgggggaaaatgaatcactccaaccaggtctgtcttctagaaagtataggatgaaagggctcctcatcacatacttcctgacctcctgctaggcctttccctaaaacaggggctggcaaagcacaacctgtgggtcacgcctagcctgccacctgtttttgcaaataaagttttattggagcatgactatatgtatttgcttacagtctgtggctgcgttcacactatcccagcagagttgaataattgggacagggaccatatgatgggtgaagctgaaaacatttactctctggctgtattcagaggaggtttactgagcccttctctgagacatggcaagcgctgcttcaggctcatgcttcactagattcaggcctggggcagtaaagagccagctcaggatagcactcccgactcactcattttttcaggcaggggagccatctaatgtcaagtgcctacgtgcaggaactggtctgttaattagcagctctcctcatggaagggataatatattctagaaacaggagtgcggccctattgcaagaatgtcctgagccaaaattaagattcttctatggcagaaacttggctggggcttctcctgagttaacttggtagttgttagtgatttttgagtcagtttttccttgtcaacgaccccaggaatgagtttgggattacagggtagccagggaaagggaaagcttcacgcccgcccccgggacaaggtctgtcttcacactgctacatcccttcacccactttaaaatgaaacttaaaaggaggatttcagttgagtaggaagtgagaagagggctcattttaaaacaagcgttaaatgaaaacccacacacactcagagcacacaaatccaaccacgcttacaaaaccatcacagagggtcaggcgaggcccttttctaaatgaaaaagaacaggggtggagactgttctgagagcatgctgggttccctgaagggaattctcagctgtatgtgccccgcacaggatccctgctagacacaaggccagctgccttcctttcaagccgcagacgcatccctgtgtccaggcgggctggtcagctgcggtcagcaccagcttccccgctccatggtgaggtcatcacaacatgtgagcaggagggcaggccggcaacctctgagtgcttagagaaagggacgggattcctcctgtgcaacccctctagtctcactcagactcaagtctgactaaggggccaggtgctttgaccagggactctcccctctcacttccctcccaggagtcacaggtacatgagtccttgttttacaaatgaagaaaacagacccaacatgattaagatgttgccttcataggggtggcaccaggattccaaaccatggactccactgagcccagtgcccactgacatgtgccagtaacagtgcagctgcctgtggttctgtcgactaaactgccggcagaggctggctttccaccttctttttttttttttcactcttcaaacactttatgacatgaacataaactactggctgcatcgttctgctgacaacatgacatgtttctataacttgaaaaaagcaagcagtggactgctcattggtaaaattgagtcagtaatcttttaggaaggttatttttcttccttttactgcttctcatctgttccccgcagtaaagaggacaagatgacgacgactcagggaacacctccagcctgaagcagcaccatgcgagcttagaccttagggtcggcttagaaaccacaggcggggcggcttgggcccctcggacactccctctcgaagctgcttctccccaagctaccccaaaggcactgagcgccctctgccccccagcaattcaattcactggctgtcctgctcctgtcagtactgagagttgcatgtttgaccctcgggggaaaagtccagaggccctggggtgtccagcatgctctgaggtccctgctgctgaccccttgcgctgtcagcattcagagacattcacacagcacagcctcccaggctaacagctgtcatggaacagtggagcagctagacgtggccattctgtggcccagtgctgcagaggtcaaagggacaagcgcagggagcatctttgctttcagaaaaaaaaaaaaaaaaaagaagcacactggtgcactgacctgctcctggtgtctttgtgattgctcttttctttcgatttttggttgtctttttttttttgaaagaggggcttttatgcttttttcctaatgttcatgggtaaaccaatgtaaatgtgtgtatgtttatagagatggctttaaatcgcaattctgcagtagagattgattttttaaaaaacatgggtaaaaattgaagaaaaattttaaaagaacatttaaaccatcttgggctaggggtggatatgcaccaccccacggaagccaaacaaaatctctctgcagataaacatttgcaaaaagaatttccaatcccaatttttgagtcagagatcttttatttccttgcaaattacatatctgtttcaggatttttgactataagaagaatgaatgaagatgtgtttcttacagataactatgaacaaaccaggaaggataataacttgtatcccccaattcgaatccagaggatgggaaggcataaaaaaaagaaatggaagaaactttatttttagtggtaaatggtgggactatgtattttacgtatggtgaagtcaccaagcccaacacttggcacttgtaggcaaggtagtcttctaatctgaatgtgaagtattatgttttcatttgcttggtaatgaggaatattggtgctttcgtcccagttctcgagctgactgacttctctttctgacgtgtgttcctttagcacacctctacactgcatggctctgagatgtcctgtgactgtttcatgtgtaaagttgcctccccaaaggactcacatattccttcagggcagtgagtacttctgattcatccttagcagctaccttcgcgctactttactagatatgttgtagttgaattaatgaacaaaagaacaagcaactttggtgcctggtgtgcatctcagagcagggtggagtgagcctggccaaagggtcatcatgcaacctctgtggctgactccatctggccacggagcttctcagccatgcttggtattcacatgacttctagggcgacagctcaaccagcaaataaacagcttcatatgggaaatattactcagcctttgtcatcaaggagtgagtcacgggcctgaactgaatagaagatagaggagaaaaggtgtgtggactgggtgagacagcgcccagcgaggtgaactcccggcagccctgcctgtctttacctgcacatcaccttgctagggtgccttcggttgtgagggcctgtctaggaagagaagagttgcaccctggcaggcagcactgagctgtctcatgcaaagctgaggaagaaagagtgagctgcccagtgagcctgctggggtggtggaggctgggctgggctgtgcagtctgcagcccccagcagcccttggcacctttctactgcctggtgctcaccagctctccagtaacaaagagggacgtgaagtcagaggggaagggaggtagcacagggcagtcttgactttgaacaaagagctggcttcctgaagtcagctggccgggttttgaagccgattttccagcagtgatctttgatgccaaccccatttaggaattctgtatctccccctaccttctaccagatgtctctgagctcacctttggtgataatcatgcaatctccgtcatccccacgtccacactgccccattctgtcccaccccgggttctgtggtgctgtcggctccccagcgagccaggaagggagaggccagctctgctggggctcctgccgccctggctctgcactgcccttctctggcaggtctgaggcgccactggaggagccacacggccctgaagcagcaaggcagatgccctggacacagtggaggcacagagtgcaagcaccggcctggcccacagacttttggaggggaagtggtattattcagttcaaaagtatgcctgtgtgtaaagagagagcccctgaacatgagtaagcaaaagtctcagcgcagagattagacaagtagaatgctggcccgagaggaggcgtttactcaccctctgtctaggaaggaaagccaggcccagcacgctcactgctatctatcctctcacacagagggattttgaatcgaagccagcatcctgtcctttctccaatgtcccctgctcaggagtcaggactcagcaaggcccaccccagccacacacagatacagttccaggactcagaactcagcgaggcccaccccagccacatgcaggtccagttccaggattcaggacacagtgaggcccacccgagccacatccaggtccagttccaggactcaggattcagtgaggcccaccccagccacacacaggtccagttccaggactcaggactcagcgaggcccaccccagccacatgcaggtccagttccaggattcaggacacagtgaggcccaccccagccatatccaggttcagttccaggtaaatcatctgccttcctccgtccaaaagccttgtttcctgtgtgtccttgtgtttaaaatggaaacgttatgagaaactgcctgccagggcaaagggtgctgcccggcacacagtagggactcaaaatgaaactattgtattgaatacataacagatcaacgggtattgctttctgaaatcttttttagcccaattttgtttcttatagtccaataacaggtcaaattcatttctgatttactagccattcagttgcccataaaaaatggaaagtgatttaagattattagtttaaaaaccaatgaaggtaaaacagttatcattgaaggcacataggcagaaatagattgcaatagttgctgccatgtgaagcctcagtgtcatgctccatatttagagagatctatgatttctgaggccctttcatgtccatgatctcagtactgctcacaactgccctgtgaaattcgccgagctggccccatgtcaatcagagtacactgagcactgagacccagcatgttgagataactggctagagatcatcccataatggtaccatcacaatcttcacactgtagaagtttgatgatgtcactggaagcatattccacagtcccttgtgaactggccttcctgtgatcagaagcatcagtgaactcccaagagggtgggaactcccaagaggtattctcactctacttagtgtatattttacaaatcacaagcttggctttggattcttttaatggctagaaggagaatcatggggttggaagtccaccagtttgggtattctgttccctaactcaaaataaagagatgttattttcaagtcttctgcttgttaacttaattagagatacatgagtttgcagctgtgctgggcatgccgcagcttggcatgtttagtccagaaggcatattataatgtacatggaagattgtcagaaattcaaaaggactttttgagtatcacatgtgtattttcaagttccaatatagattcacattcagtttgacaggtatctttggatgcctatcagttaagaactatttattagttgtggaataaaatagggtaaaataaggaacaactgaggaaaaaacataaaatttgctttgtgaataaaagttgtcttcaaaattatgactttttccatcccacaaaagttttgattaaacccacaatgaaaatttaaataagtgtatttactttggtttaaccacttatttcattatgactcacaactataggttttctagtttccattattacaaactattgtgtggtttaaatcaatttcatagactagtctagttctatagtcacaatttataaaatttttttatgtggtaaattgagtgtcttcatagatgtacatgattatttctcaatttttaaggaatgtattttttaagatagccttctttagccttctttaacactgatttttgtaaattttttacagatttttttaaatttttggtaattttttagcataaagtaatacatggtcactatggaaaacataaaaacacaaaaactatgaagagtaaataagaaaaacacccagaaatttaccattcagaaaaggtcattgttaacaacacggtgtatcttcctcctgtcatgcttccgtgcatttgagcacatttgagatgtgtatacatgttcactttgagattttagtatagcaaaagaaatgaccggtcctgattcaatgaaacctctggcaaactcgctatattttccttacatatttttaagttcatcctataaatgaactatccattcatcttatttgagattttcttaaatctttcagcaagaaagcgggaaaaaaatcctcctctggcctttaaagcctaattaaatatatgactaagctagaaatattttataatgaccaaccagaaagtggcaaggactgtcactcttcccatacagcccacctcctcctctatctccctcaggcacacggaaacgagaaaggcagagaaacccaggacaagtcatccaagactttggtcacatggccatccattgctttcacaacaaaaatataaatccaacatgtgtgtgtgcatttcataccagtaggtccaataagctatctatatatacacatatgtgtacacacacacacacacatccttacagacactccccagcttactacagtttgacttaagattttttgactttacgatggtgtgaaagcaatgcacattcaatggaaaccatacttctaatgttgaattttttatcttttcttgggttagttgatgtctgatatgttactttcttgcgatgccaggcaatggctgggagccagagctcccagtcagccatgcaatcaagaggctaaacagctgatactatacagtggactgtgtcaccagcattttggggatattgtgttttgtgtttttgaatcctatcatgtctacaaaatgccattttcgactgctattttcaatttagggtgggtttatcaggacataaccctatggaaagttgaggaccatctgtatatctggtagggaaagatggataacaaattcataggcaaataataatttcatgattattattaagttattcctacttaataataagtagtgatcactgccagggagcagagaatgcaggataatgtgacagatgtaatggtgggtacttaagctaatgtagttgcagaacaggcttttctagagggtaggcctttaagcgtacctcgaagatgcaaaggaagcaaagatgcgaagatctgggctggggatggaagcagagacaacttggaggccaaggggagagactgacaacagcccagctcatacctcagcagcctttaatgcatagctaagaaaacaacaaattaaaacaattatagtttacttagacgattctaagtgtctaagtggatttgggcaaatctggagaaacttgttctaatactgtgtcttaataagtaatatagatttgcccaggcttgtgggcagagtggtatacaccccataatagcagaggaaggccacagggcctaccctacaaaaccagaggcatttaaaaacttaaaggaggcagattgcttttattttcagttaaaataaagtgaggagtttctcaagaaaaataataacgagaccaccggcccgccctagatgtccaacaagaatgcacagataacttcgtatatccactttcctgaacctgcccctgacagccaagtggagcacaacaacagagatgaacctcaaaactactgtgctgtgacataaggcttgctcaagaggacagtgtggtgtgagtccatctatgttctaaagcaagcaaagctattctgtagtgaaaatggatcaggacagcagttgcctctggtgtatgggggcagggatcgactgggaggggcatgagggatgacagttagggtttcgatcatgacaggaattcagattactccagcatgtgcatttgttaaagctcatcaaatgctacacttaagattaatcctctcacagtttgtggatgttaccttaaaaacaacaatgatgactgcaaactaatattgaactctggttagtgatataccaatgtgaagtatagtgatatctctactttactttaaaatgcatccaaaggcagactagaggaccatatctgacagacagaaaaatagatatgtgataaggtgaatgtagtaaaatgctaacataaggatgtttgcggtacaattctttcagcttttctatacatttataaatcataataaaattttaggacaaaaagttagtgctttgaagtcctaagtcatagggcctgctgctcttgatgcagtagaatttgtcttcagatttgcaaagggtaaggcaaaccactagcattttgtatggaacttgatgcaaatacttttaattgtctggttttcaaatgtatagacttaaagtaatatcaactctttctttgaatcaactactgaaatacctagtcttaaataaatatttttatgtaatccttaaagtactatgtattcatttttctttcttctttcttttctggtttgataaatattctataaagtaactgtgtttaatggccaacatttgagtaagtccatatgcagatccaaacatctcagtttagacaataacttaagacaatatagagtggctgacatcccctaacgtgggtccagatgcatgttatgttatgtttctgttgcattctcaatagttaactttaataaaagaaagtcaaaagcttatatattttttcaatcttcaaaacatttctgggaggttgtcttagttaattttatgttgctatacccatatcacagactgggtaatttataaagaaaataaatgtatttggctcatggttctggtggctgggaagtccaagagcatggcattggcatctgcttggcagctggtgagggccttcatgctgtgtcaatctatggtggaaggtcaagagagcatgcatgtgaggtggtggggaagagaaaaagcgggtttaactcatccttttatcagggactcactcccgtgatagctaacccattcttacatgaatggcattaatccattccttagggcacagctctcatgacctaattataatacctcttaaagtttccacctctcaacactgttgcattggtgattaagtttccaataaacgcactttggaaaacacattcaaaccacagcagagatcaacgttattgtcaccattttcatatttgaggaaagcatggcacagagagcttggagaagtacttcaaggtcacccaatgaggaagtggctaaacaaaaaccttatcttaaattaattaaaaacctcttgctctttgcagttttgtcttaaatctacctaatttgtgactgtaatttttaagtaatttactcatataagtggtctcacattaaattttctcattgctttatatttctaacatgagatatttggtataaggatggaaccaagatcataccttgttttaattagaaaacctagaccaagtcattgtgatcctcatcctagatttcagttaaatgctgctgtctccttttgggtatgtgacaggggaaagcctcagaagaaacaaccttatgtgttttcttttgatactttagtaattaacccaggatagtattcaagattgacatgccttatattgaatcaaatagcatatcaactgccttcttattctcaagtatagacatgttgggtaattgggcatttaagtttctttgcaattttttccattattaacaaaattaatgagcaacattctgcataaggtctgtttcctcagaatacgtttcccaaagtggaatcatcatgacgtagaatttaagcatacttacttgtttaaacaaattgtccagttgcttcccaaaatgttttgtgaattaagatttacatcaagaatatgtaatgttgttactgtctcccaaatacaggatctttttctgaatataaaagttatacatgctaattgtagacaatgaagggtcattatcctcatagataatgaagtgcttctaatacttgtgcttttattcatttattcaaaaagtgctaaataagccctgaaggggcttttggggggtcatttggggcttatttagcactttttgaataaataaataaaagcacaagtacagtttttttaaaatactgttttctataatagattaatcttaaatggcatgttttcctttattttactgacaaaagttacttactctgtgattgaataataaaaattctttggttcagctgagagaaacttgcaagctgacgtccttgattatttaaaatgaaagcagctgcctgttttcatctctctgcatcctgaggaaactcttctgcaacgtgttccagccctaggttctagctgaccctgttcatctgtttggcacgaggggcccaactaacacttgcggctacctggacgacagccaatctagttggaatgagagttagaggccatagtctgtcagctgggaaagcagcttttattccaaggtgtgccaaccgaaaggccacatgttattgtcacaacctggtacctacatcagtgctgacatctttaagaaccttagaattgggaaatcagtttagccctatctgcatgtgtagccgacaaccacacaattgttccaacttgaggttgcattcagagcaacctcatttcccccatactcctgaggaaaagcagaccagagacgctgggtcaatccagagttatggttggaaaaatgatggaataattctgcccctggtgataggagagagggactccatcttgtcaactgtcatggttcccatgtgaaagctatcattatcactgaaattgaatgagaacacagaagggaagaacagggaaatccccacagagttaaagaggatgtgaagattgcttcatgtttaatgtttgtgtaagtgctttgggttggttatgtgctgtctgaacatgtgctcatttccatggctcattgagagggcagacagtccaatgatactctttagaatcattcccatggggaaggaacaaagaagcctgtaaaatagaaatgcacatgtaaaaagcattgaagaaagtgccagtgtattgattttggccatggtttgtgctctaccacctggttactgtgattgcagaagtgcctttgcagatgaggaagaacctggccaaggctcaatccaacatccaaagccagaggccatatttcttcactcttaagataatttgggttcaaattatagtccctttacacactctctgcctcaaaaggcccaagactctcttttgttatgcttgcctaaacatgcctttcaaagaactagttctgtaaatacaactttattataaacctctcctttgcttttaaaaatggatcaccacgtccatttctatggtccaactttgtcccttaatttaaaattttttcttggattaagtttgatgccttgaaacattaggaactcaagcatacaagattgtatgctggtggtgagggaagtaactgtgcctccgcctgtgctgggtggatcaacatggagtgtggacgagcatagggatgtgtgggtttctcactagctgagagtgtttttaaatgttgtattttgatgtttgttattttctgaatattctacagttagacctttgatttattctttgatgcattcatttgaataatatttttaatctccagccagttaggtttttaatttacacttttgtccctgattttaggtgtagtgttgtgtacactactgcccagtgtatgttatgtttgtaaacattcattgcacgcacaacaatgtgactcacaatatttttgagaagtaaaaagttcattatatagttattaactcaaccctacagttatattcgtgaaataccttgtgaaatttattttttgcctactggagctcttacaggttaatcctgtcttcaagattttcatagaattttcatctaccacccacccctttaaatttcaacatttttttattttggcattttaatgcaattcaatgcattatagggacaagctatctcttattatgaattgcaccttatataaacttaaagatcttttatcacaaatttctttgctgtgtcctttagtgagaatttgtattatcagtcactaaagctcactaagttagtaagctttgcgcccagatgacctgggcaggaatgggtgagtctctgtgtggagagagtgaagaaactgctacccttaatacctggaccttgagggattgttttattttagtttttctgcatttctcagtatttcatgtgatatctgtctttttcttccagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaaggttggtgactttgattttcctacacaaataaaattggagaaaatctaagtggagaaaggcctgggcagaattccacttgaagtgtgtttatttttgctatggcaatgacaagtcttacagagctacaaacgagagttttatgagaaagccattttaccagctaatgtcaagtaataactagaaaaggatatcaaatagaaacaggctaatctggagttccatgtcatcatagacactgacgtttatccctgaccattacctcagtcatgatgtgctgccatactcgctcttaaaaactttttttaaaagccctgctttgcaccatttgcctattcccttagtgtaaatactcctactatagctgatttcaaggtaccaagtttcactcagctggtcacagaattcttatttcacgataggcgctaatgaccccataggagccagctctgaaggcttcagagtttcactgaattttggatggggtttacttagccttcttctgtttttcttttacctttcctttttaaataagaaataatgcaagacagatacaaagtaattctttttaatttccattttcactggagagtgttgaaccccgtgaggcatgagagcacagtgttccagaacaatgcttactgctcattatcacaggggtcaaaggctaacgtgcagggattgttgcagatcgtggacatgctgcctcctgtgtccatgactgcaatcgtctacctattttacagttgttgagcactcgtgtgcattagggttcaactgggcgtcctagggctccctggacccattttagaccttgagttcttgagttcctcaaaagagaaatcacgcatttatgttttctcttcttagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacagggtgagaggctgggatgccaaggctgggggttcataaatgcagacagcagttccgatggctcccagcgagcttgtcactcaattccacctcggagaaggcttttatttttacccagtacacgtgcactgagtgccggctgtgtgtaagatactgcaggggaagttactgagaagatggcagatactggaatgggaagatttaagcggggtaccagtgtttacatggacatgaaaaaatactgagagatagtaagaaatcgtaaagattctgagtaaaagagagtatgaccaaacaagctgagcaggaatcgtgaatctatgtgtgtaggcagtgaataaactgccagtcttattacctggacctcaaggataaaagacatacagtaaaaatcaacccacattgaggacagtttcgagagtcgcgctgctacacagaaagccctgtgtaagttaaggatagagaatgaggtgttctagaactttgaatttttgtgagcaggactcgtgaggttcctgtgagaggaaacaatgaaggatgatagaaaagaagggaaattgattttaaaaaactggagatagcagtgattgtgcctcactgtgcagtgggtttggggccaggaatgttaaattggtaacttcatttaacgcccacaacctttcttcaaagtaggcactgtacagatgccccttgacttatgatggcatcctatctggctggaccccgccgagggtgaaggcgtcattaggtcggatttcagggctaattgaatgtatattgccttcacaccatggcaaagtcgaaaatctgtgttaaatcatgctaagccggggactggctgtgctctgccatcgtacaaataaataaatggaagtcaagtaactcccttgagggccccagctagtgaatggagaggccagctatggccaccactctctgccccagggcgctcaacgcccctcctgtgccatgcagttctgacagggaggcagtgctggtaggaaaggggtgtgatgaaaggggtgcccagcagagggagtcatatccggagtgacaggagcccaacaggggtgcagcgctggaacccaagccagcacctctggtcatggctcctcagttcaccgcctataaaattgtgtggttcccccacaccccttgctgctcagagcagccgcgcacatgcttgtgctgtgcgtgcctcctgtgagatggcctggtacaccggttcctacagtgcgcctcacacgctgtctcggagggaggcagcctgtgcgggtgcctggacctccgagccagaccctctgggttcctgcctggccccgtccctcagcagccagatggctcgggagcacattctccaatccctccgtgtctctgtttcgtcatcttcaaaaatgtggatggcatagctgctaaaaaatggtgacatacttcctaggtggtgcagaaaattaagtgactgtaggaacaggcctcagcagctccttccacttccttggtatgattgttttttaaaccaaggctgggattgtatagatgcagattagttaatgtgataccattaatagctaacctagtgcctgctgcagggtgagcctcccctaagccaccgggaagcggctcctgcagcctccctcacgtgtgctggccctcctctggcagtcattgcctgtggtgtgctgaaggcccagctctgactgtgcctctgtgctctcctcgccccgccccctgctctctctcaggtctttggtctgttgtccgagctgccacagcagcctggacatccctgttggtgtttccagccctgtcctctcctgagttccatccacctgtgcatggctttttcatgagtgttttcacggatggttctgctgtcatctccaacctgataaacaaagcaccacgattcagcccttatgaccccaagcttccttcctcagttccttgcttctgtgcatccactgaagaagcctgttccactgtttccctgcactgggtctcctgtctgcaggaagccttcagccctcacttccacactcctctaagatgtgtgcctgtgcccttctggggaagctcattttcctagcagcctccaggatcttcaggggtgaatccctcctttcccacgttggtactctgtacacacaacatgcccattccctgcctggggagctgggcattgcttcatgaatcagaggtcaattttttctctattaaagtcacagatgctcattgcaccattgtgagaatgaatgaagatagtgcttataaatcagccagcaaggtacccagcctcactgtgtcagggtctccctgggcatgaggtggttagagtgtgtgacatgtctgtccccaagcctgtcagctcccagatcgaagccagtggatctcattcatcctcgcagcgcccacagcacttgcacagggttttgtacacataagtcattctgtcaatgttcatgtttaatgtcatcagtggaacactcccactttgtaaagacttgaatgtgttcatccctgacttttccacatcttgttagttcttctttggaaacagctgtacagtttcaccatcctgtgcatccctggagtctacctgtctctgtcatacattcagattcttcttgtttcgtgtcactctcatatccttttctctaatgaaaagctccgcctgggcatgcaaggtggagccctggatgccagcccctcacctggcatccagggctgtagcactcaggaactgcctccctgccctgcctaccccctacatcatgcgaccattccagtccagccaatcagccccttgggacccagcttaccacatgcatatcatttatgctgtgaccactgactaaaccattctcttccttcctccccatatttctaaatttctaatcattgctcaaagcccaattcagagaaaaccctagctcctccatggcaccatcattaacaattttatctggccgccccccgggaagttcactgggctaattgcgggactcttgttcgcaccatggcatctctttagcagaacataaatgcgaagagcacatgcatccttcatgggaatttaaaggagctggaaagagtgctcaccgcagttccattctcccgcagaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgtaagtgtcgcatacacactatctctgcctccagctcctatgggggacagctctacagcactggggcaggggagagaagccatgtttagtaagtcacattaatcagaaacaaaaagtagtaagcaaaatatctgaccactagaaaagcatgtatttaccacggacatagagatcgtttttttgtggcgggtggcagcccagctggttggcagtgcaggccaccggaggcagatcccctgcagggacagcagagcacttgtgtcctgagaagagctgctgttcatggggctggcagcaccagggcctctcctagcctgccctgctgacactggccagactcctacatgcttctgagtctccagaggctacccggccctcctgaagcaccagggctgaatccacccccagctgagggcatgaacactgccacatggagtcacacacacagctgggcactgccatggagaggaagtctgtccatgtttccttgaatactggtggcctggtccctgtcccattccccagtgaggcagcctgtggggaagcctggcagggaaccaggcgcaggtcagcgtggcgccctgactcaggccagcactgatgggggactctgagacgcaagctcacactcacccagctcccctgggctgcgcccgttcctgatcgcttggactttctgttctttagagtaagaagtgatcaccatttcctgcttctttgtttctccacaactgtgcagtggatgcctgtttgttttctgccctcagaacaaaaaaaaaaaaaaatagagctgacgtgaatcttcaaaatcatcaactacagggctttggatttttgtgtatttgttttattttcattttatggatggattgtgatgaaatgcccgtaatacaagattttccatcttaaccattgtaagttacaatgtcagtggcattatacatccacatgggtgtgtggccatcaccaccgtccacacacagaactcttttatcttgcaaagctgaaactctacccattagacagtaactctctgctctcccttccttcccagcctctggccctggcaggcaacagtccacttgatgtctctatgaatttgactgctctggggctctcatacaggtggaatcatgtagtatctgtccttttgtgtctggcttatttcacctagcaaaatgtcccgaaggtttatccatgctgtagcacgtgttaagaatgtccttcctcttcatggctgaataatattccattgtatgttgacactacattttgtttgtccattcacctatctacagacactggggttgcttccatcttttgactgtttgaataatgctgctgtgaacatgggtattgaggctctttgttttatagacatattattccaccagatacccatcctgacacctactatgtttgcaagaaactgaaagctttattttacattgcaaaatttcatattatgagatcaaggttagcatttcctcagctgtctggtggacaatggggaggttaaactgtgcacattttatttttttttaatgaacctggaacggttatggggccagtgtttgccatggatcaggtcaggcagcccacaatggcaggtctccatgttctgtacaacaactgtgggaaagacccacagagaaagtgctggaaaggggaatgatgggtaggttcatgcagtaaaaagattcaaatactacagggcattgaactataggccaatatagcattgctttaagaataaacaaaaaataagacagtaagaataagcctagcaaaatcaaaagtctataaagaactgacatttcaagccaataagagaataattccttattcaataaattgtctggaatgacttaactattaggggtgaaaatatcaaagtgagagaactataaagggtttttaaaaaggaattaggtatgttgggttagtcgcattggagagtgcaaattcaccatcgacctgatacctgaaatttcctccttaccatctagaggcaagttgggaatgctgccaggctcctgtggtaaaggaagctcctctcttgactggtgctttatggctacacgttcctgctcagaatggatctcatttagtcttcaccaaaaaaaaaaatctcatgagatgatttaagtgttttatggacaagatgtctaaaactcagaaaaatttcacagtgtgcctagcttttatgtttatgttgaagttgggcattagaagttagaatgaatgggtttacttcagagaaaattaaatccatcacccactccttgtactatgaattccaaatacatattaaatacatataataaaatatttaatatatatgtaagtgccagaaggaaacataaatatgaatattttgtaatatcaagttgaagaaaagccaaaatctgacatcataaaagaaaactttcaagtaaaatatgttaatggctaccaggaaaatattgtgcaatgtctgattgccatgaagagggttaatatccttgctatatcactctgtgaagtcatctttaaaagactaagaaaaagatgaatctcttaataaaaacctggcccagaacatgagcagcctctctctctcactctcactgtctctctttctgtcacacacacacacgcacacatacacacacacacacaaatatggccaagaaataaagtaaaatgttatttctaatgtaataagtaggtcaaaatagaaaaagaaagcatcacaccttcctttgcaaagtatttgggttccttttgcttttaaacacctgggtcagctggggtgtcgagaaacagaaattctcacgttctgcttgtgggcatatatgttaataaaaccaagcttggcaatatgcctgcaatatgtatctaaagcttcaaagtatgtatagctttgaccaatcaatatcacatttcggaataagagaaaaagaaataatgaaagtgaaaatcataagagatgtagaaacatattcttatacaagaattccttgcagccttatttataataaattttgtgaacaaattatatatctaaaaataagagattggttgaaaaaattatgcagcagccatgctattgataatcatgttagatagaagcatatttaaaggcatggaaaaattgccatgttttatatgggtttttaaggttataacacaatgtatagtgggattccaattcctgtatatacatagacttatatgtctatattgattaactctggatgagtctcatgtcttctttttgctttcttctattatccatattttatacgatgtgcctgcatttcttttttgtaacagatggtcaatactagaatcataaacagatcttgtttgtttattggcaaatgtttcccgttagaaaaagatgcatttttcttttaaatatttttattttatacaatgattacaagcttataatagaaatttgaaaattatatgtgagtacagggtaaaaagttgaaagaatgggattgcacgctacagatctagctgcttttagcacgcctgcgtaggaccttgctttctctagacctctgttgcagtctctctgcctacctcctcacaacgtccatcccccgcggtcactgtcgtgatgccagcctccccggccttcatgtctctaaggagcaccagcgcggcaattagcgccctttgccttggtggtattctggcttcacagtcacatgggagatcaatcgtcagcttttctgtttgaaatctaaattcttcctgactgcaggggacctcgggacccatgaacacctctagtttactatgtcttcacagtaaaagatatctgcatgactggactctttaacaaatttggtggttaacctactctttctatatagatatagcacttcgaccttcagacttctcaatactgataaaaagaaaacacgacagatgacaggaaaacctttgcagctataatttgtaatcggccaattataaaaactgcaaaaattgaccagatagctaaggttttacacagtcatgaaagtgatctgcactgttaacatttcaccctctgtgcaccattctgtgcttctctctggtttggagtctagaaggttttatttacaggctatgacttaacaatcccagaacggctgacacatgcagtcactcaagactggacacagcaaggaagtagtgggtccatgccaaaggctcagccagacgagacactctagctgtggcaggagatgccagggaatgctccaagcctaagcagattgtaaacaaggaacctcaaattcatgaaaaattcttgcttatgtggcccatgtcagtaattactctctgcctcagtttccgcagctgacatgtaaataaaagcagttcatggttcatcttcttttcttatcggggtctcaagtgattctacaaaccagccagccaaacaatcagagaataagttgaaaagattgtcttcatttattgaatgtgcttaactcaggcccgggaaagggcgtcatcagtttctcatcatttcactgagatatgcatctattacttttacatttcaggccaaaagtgtgatccaagctgtcccaatgggagctgctggggtgcaggagaggagaactgccagaaacgtaagtcagtgaacagcctcagacccatgtgtgaccgcccctctcttccttcacttgcttaggtgattggatttgttttccctctgaagactccaaagagttactttattacagggtcagatgtgaaccagtaggtgaaggacagtcttgcaaatctcaccgcatgcagttaatccagggtgggctattttgggagcttcagcctatcacaaataagtgaacatcagcaggggctgggcgcggtggctcacccctataatcccagcactttgggaggcggaggcggtcggatcacgaggtcaggagatcgagccattctggttaacacagtgaaacctcgtctctactaaaaatacaaaaaattagccgggcgtggtggcgggcgcctgtagtcccagctactcgggaggctgaggcaggagaatggcatgaacctgggaggcggagcttgcagtgagccgagattgtgccactgcattccagcctgggcgacagagcgagactccgtctcaaaacaacaacaacaacaacaacaacaataagtgaacatcagcaagtaccccagccctgtcctctgaacacagcacactttcccaggaatggaagacttgctcctgttgacagcagtcaccagacttcttgtttcctctccctccctggctttctttggtacccacctacacagaagcctgagcacgggttctcatggggacttttccatgtggaccctgctttacgatggagagggccattctcctaggtatggttgtctggctcagcctctcagtggccaaggaacctggggacatgagctcaaaaacggacactatgtccttaagctgaattgtgggggggctgttaggcccttctaaacactacttcccagcaggtatttttgttctttgtatgtgctttctgcattgcccaagatgcatctaattatttagcaggtctcaaagtctagacttgatctcatgagttctcttaagtgattaaaaataaatcaggagaaaaaagaggcaatcagaaaagggcatggtttgacttagtttgaatgtggtttcgttggaagcaaatgtgtcttcactttttcatgaaaaagtctgcaagtgctctgcgacatccctgggaaatgatcctaccctcactcttcagctcacagggaacctttgctctttttcagtgaccaaaatcatctgtgcccagcagtgctccgggcgctgccgtggcaagtcccccagtgactgctgccacaaccagtgtgctgcaggctgcacaggcccccgggagagcgactgcctggtaagatgcccctccagcagcctccctggagcaggctggggctgcacccgccccacccacaccaggacagaagacttcctgtgggggagctgtcaattagcatttgtcataacagacaggatattgccctctgcctggtgacaaagtatctttagtatcctgcctccaccactcactgagaccttgggaaaatgatgggactaccatgcctccatttccttacctgacaatgatgcataacaaagtctctcccagttgaatgcttaaatgatgagatgcctgtgatgtccgtcattaggacctgggcacagaacaagcactaaatactacatgcaagtatttgtcatgaatgtgccttgttgccagcagcacactctctttattgtttgacttcggctatacctctagagacttgacactgtgaggtccctaagagacccatggagagccacacaggtcttgctggctggggctgggttagggcctcctgacacggatccctcggctcctccaccactgctcaggcacctcctgagctgcaccctgccctcaaggggtcctgaagtactcactgtcgccccattgctccagaaagtgccagcagaagccttgctgccccagcgggctctgagcagcactggagggtacaggtcagaagcgtcttggaagtcctggagacgccaaggctggtggatgtgactcctggagtgggagctggtgtgacgaagcccttcctaagactaaatccagagcactctgtggtttcagagaagattcctaaattccagagtttggacccagacccaggaattgtgacttggttggcctgagctgtttctaatgtgagccccagggagaagactgtgcgtggggttggtcctaggaaaagccctcgctgtattgggtctggctcctttacacggcattgttctagcaaggctttctgccattcagcaatacattataaaatataccctcaattgtactttataagggaagcccaatgtcctttataagggaaattaaacataatttcattccatagtcaccgctataatgtgtgaactccatcatctatacgttagtaaacagacgtatttttatcataatccataaattatgataggtgggacagtgcacctaagaaaaaaatggactttttagagaagggtctttctgactctgcagagggcgccagctgggttttcccacactagtggaacactaggctgcaaagacagtaacttgggctttctgacgggagtcaacaccgtgctgcgcttcctccgtgtgtggcgctgagtgtacttacctcacttgcccagcgtgtcctctctcctccataggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtgagtcctcctctgtgggccctctaactggtcaggcatccttgtcccgctctgtctcctgctgagccctggagtatcccatcttggagagtctttgggtggatgtgtttgccttgcttggaggaggcgaccctgtgcccgtccaggcacacaggcgaggggaggggctggcttgctaccgaggagcgggcaggtggtggccatctccacccatgggggctgctcagtgcacagggcagatctgggtggccaggccacctcacaggagaaacacctgctgctcagccctcaccactcatccagcagccacagccgtgggtattcagttgtctgctgggcacaaagccgtgggcatgccactgtttagtgcttgtgccaagcaggtatttaatacaccgaaatcagagagtctatcagaagacctgccttcttgagtggttaaaattctagtgaaagttatgcctcttaggagtattgcagaggttttgtttttgtttttattttgttttgttttaatggtttgggtttgagttttgcttgtttgtacttacatttgtactggtggctccagggtttagggaaattgtgacataaaataattcctgacagagaaagcaaaactttgtctaatgaaagagttttagaagccactcttgatctctagaaggggagattaactgagaaaaaaaattgaaagaacaattatgagggggagattttaccctgccagatttgtgtacatgaaaaattttacattccgtatggaaaaaaaaaacacaaaataataagccattataaggtaaatgacaaacaaagctaaagaaaaatgtgccacagtgatgacacagatatatctttgagatagggcttaacagagctttaaaatccataggaaaacacttcgagcctgagataccaagagcagatggttcacagaagaatcatcaatgtcctataaatatttttgaggatcttcttggggaacttaaaacaggaacaggccaggcacagtggctcattggctcatgcctttaatcccagcactttgggagactgaaggggctggattgtctgaggtcaggagtttgggaccagcctggccaacagggtgaaacctcgtctctactaaaaatacaaaaattagccgggcgtggtggcgcacgcctgtaatcacagccgctcaggaggctgaggcaggagaattgctttaacccaggaggcggaggttgcagtgagctgagatcacaccactgcactccagcctgggtgacagagcaagactccatctcagacaaacaaaaaaggaagacatagagctcctaaaaataacgcagaagtctgctattaatacaaatgaattactttaaaggtgagagcaggtggaggagagggctgaggtgcctgctgggacgcaaaacagctggcccctcaagggacccagtgtttcctgccatgatgaaacacctgtattgtccacattgcggcctagaatgttattaaactcttgaacgggattccttctctatttgcaacctttcattctttgtccttaaagtaaataaagccaaaggaggatggagcctttccatcacccctcaagaggacctggaccgcctgtgtgaggcccgagcacctggtgccaccgtcatcaccttcctttcatgctctcttccccaggtaattatgtggtgacagatcacggctcgtgcgtccgagcctgtggggccgacagctatgagatggaggaagacggcgtccgcaagtgtaagaagtgcgaagggccttgccgcaaaggtaggaagcccgccggtgtgcggacgaggcttgttctcggctgctgaggctgggctctcatgccacctccaaaggaacacatcttcctcttctcattaaaaaacaactatacatatcgtttctttaaaacagaagataaagctgtaaagctaggttaggcaatgggaaggcactgaaggttgtgacggggtggggggctctgatgagaacagtcacagagccagccccgctcagcagctgccaggtgcccagccctggggagaatccagggaaggcagagctggaagcagtgcagctccaagcggcccatgggaaataatgaggagaacgcaaggtcagtgtgaggtgacagggatggcatctcctacaccgccgtagccccaaagtgtactataggtcctggtgtccccccttcccgcctgcactctccccagccccttcagtgtttgttgagtgaatgaaggatgatgtggcagtggcggttccggtgaccggaattccttcctgcttccctctgcctgtggatccctagctattcttaatccaacaaatgtgaacggaatacacgtctctcttatctctgcagtgtgtaacggaataggtattggtgaatttaaagactcactctccataaatgctacgaatattaaacacttcaaaaactgcacctccatcagtggcgatctccacatcctgccggtggcatttagggggtgagtcacaggttcagttgcttgtataaagaaaaacaaaatctgcctttttaactggtagagattggtgatcaataatcaccctgttgtttgtttcagtgactccttcacacatactcctcctctggatccacaggaactggatattctgaaaaccgtaaaggaaatcacaggtttgagctgaattatcacatgaatataaatgggaaatcagtgttttagagagagaacttttcgacatatttcctgttcccttggaataaaaacatttcttctgaaattttaccgttaatggctgatgttttgatatttttcaaaagtgcagtttctcctgcaggcaaaaggggacacgttaagtccaggcttgggtcattcactgcggtgtaaacacgctttctccctcccgcccggccccagccagctgccttggtggcccataacccctgagggtagagggaggggacaggggtaggtgacaggcagcctgggcctcaggcttttgaaactggacgccagagccttgtggggccacgggcaagcctcgggtctatgactgccgcctgagctccgcttccttcctctctaaaatgggaagattagaccaaaataacaagactgttttaaggttggaatcaaataaggaaaatttgtaaagctccttgtatgtgataccagatccacaattggcagataatcgcagcaggagcctcttcggggtaatcagatacgcggcgcagcaggggtctcagggccacagccaggggggcggcgggagacatgcggaatcgcagcggaaggcgggaggcagctgtgaactgtggctcggcctgcgtccgccctgcgcatgtacactcagagaagatgataatgaaaaagaaagcaaatccaattttcccacttactgttcatataatacagagtccctgagagtctagagtaatgtctcatacaaaaaagaaactcctacgtggtgtgtgtctgaagtctttcatctgccttacagggtttttgctgattcaggcttggcctgaaaacaggacggacctccatgcctttgagaacctagaaatcatacgcggcaggaccaagcaacagtaagttgaccacagccaaagcctggtagattacatttgcctttttagttggaaattaggcttaacaggagagttgctaagatagggcacagagctcctgcatctctcgccggcattcccaaatgctatctcacatgagcaggcacagggagcaagactgcacgaccactggcacaggctgtccgctaaaccacagacttctcagcgctcgccagtgcttctgcttctgtgtccactccagatcccacattgcacttagttgtcaaatcttttcagtccatttctaacctatattagctcctgtgtctttccttgtctttcacggccttgacacttacaaaacgtgtgggtcaggtactttgcacactgtctaaccatgtctgttcagctggtgttttctcaggatgcaattgaggttatgcacatcttatcacagggaccagagagactttttagcaccactcttcaagaatttccactttttcagctttgacagtggaatagacatgcaggtgctcacacacaagcatctttaatatggtaatggtaatcatcagtttagtggtgtggaggaggagatgggaatctcttagtgaaacccgccttggaagcagcctcgttatgagaactgctgcccctacttgactcttaaagcactagataatactgtgcaacattaaagagaataagagtgcgtgaaatatgcattgcctcccataaactcccttggctctgaatctctgatactaaatatgtggctaccgttgcttcccagaaaggcctttttgctctgaattctctggaatgctttctttgaccaagattcttataaaaataagagatttagagcaattttcttggatggctggtatgagccagttggcttagttgtagggatttaaacaagataagggttacttacttttcacatttaatgagaagtctggtgattccagctcctactgagacagggtggccacacgttccagggtgtgactcactgaggccccagacctgccctgcaaggaaaacctggctctgccctggtgtcctggcctccctgggcatatgtgggggagaattcctaatggtattggttacaggctcctatgcgagaccactcatctgtgtaggagaaaggaaaaagatgggggaaagaagagcagcagggagaggagaagcctctggatgatactctaaccccctgccatccaacacctgaacatcagtctcttcatccagtgctctcagctggcccagcccccagcctggggtcagatgagagcttcctgcaaatgcagatctctttcctgtggctccttctcaattacagacagctcctccacaaggtgcactctggccttgtgctccctccccaaaccagcccagccctcccagcctgcatcatcgtggtcctgtaggggctagaggttctcacacccatcgtggtctggcagaggctggtggttctcacacccatcgtggtccggcaggggcttagtggttcttatacccatcgtggttcaggaggggctagtggttctcacacccatcgtggtctggctggggctagtggttctcatgtccaccgcgtgctttcctgctcctccaggtggctgaggacatccccccttcggtctgaatgacttccatccagtcatctgatatacacattggaccacccaatagcatcctagtgtcatgttggatggtgaagaaaatgccacagttactgctttcagggcctcacaaccttgggcatagctttttggaggaaggccccacttcccaggcatccctcccagacctggtcagaggcccctgctctttgcttccatgttgcccacactcactgtgctcttcacaccggctcaaaatgatctgcttacggggttgtgtcaccaccagatcaagcgtcctggagaggaggaaacatatttaacctgcacagaatttgggacagagaacctctagtgtttgttcaataaatatatgaatggatagagggacaggttgggtggtggatagatggatgaacccacacctttgaagtgtatttggctgtttgagaggttagaatatgttctcaatttccaggcaaaatgaaaatggagaaaatataatgacattaaggcattttattcatcctccccatctgccactgggttaaagatactaaataaacaaggaactatcttttgcctggaggaactttaaaaacacctgcagttttcaaaaggtgcagtgtgtgcctcccacagcatgacctaccatcattggaaagcagtttgtagtcaatcaaaggtggtctggagaaacaaagttttcagggatacattgtttttataatttttcaccacatgatttttcttctctccaatgtagtggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcagtaagtcaccgctttctgtttagtttatggagttggttctaatgggtcctttatttgtatttagaatattgaagggctattcccatttaaattacttttttcagttccttaagaagcaaattaaaatcttaagattcctaactgtgaaattaccatgtgaattccattaaaactttttccagatcattaccattcaatgggatgaatttaccctgaggtttaggctaccaattatttgtaatgtaagtaactaaatttagtattagttatattaccttttagttgtaggtcactctctgctcatttcagcctgtaaagactacagctacacacatacacacacagaggaatggaatgagcactttacatcaacacttcctgttctggctctagagcctcagcttttgaagctggtgagagcctggcctgtgctgggccttggccacgggcagcgtcagctttgagtcaagtgctggtctggcctccctagctttgagcctctgtcaattcccttaatctgtttaggctttggcttcctcatccatagaatggagatatgaatgattcctacgccgtagtgctttgagagaattcagtgaaattcctgtgtgtaaaacccttccatggtgcctagcacacagcacacagccaatggcccaatggctcctatcagctgtgggatttgtcatcagaacaccaccagctctgctccaggctgccctgggtaccatcaaaacacaccctgtgcccagcagcacctgctcctctgcacacctggttccttcagcaggggcagtggccgtgggagcacagaaaacatggagtcccatctggtttaattgatgccattgccaaaggggaggactcacggcaccccctctcgggtgccagggtgcctggctcccaccaggaggaagacctgtcctccactgtcaggcacatttcagtcttcccagcagccagcacaactactttgtccttccagtcacggtcggcctctgggaagcccagtctgtgtcctcctccttcaggggtagccagcatgtctgtgtcacccaaggtcatggagcacagggcccctcccgggaaggtgccgtctcctccggcccctcgggtccctgctctgtcactgactgctgtgacccactctgtctccgcagaggccacaggccaggtctgccatgccttgtgctcccccgagggctgctggggcccggagcccagggactgcgtctcttgccggaatgtcagccgaggcagggaatgcgtggacaagtgcaaccttctggaggggtaggaggttatttctttaatccccttgcgttgatcaaaaataaggctccaggttgttgttatagctttacaggcattctgtttgattttctcttccttttattctttgcccttggcttttggaggttttgggttttctgtggggagacgggaagttgtttgattgcgttatttttggcaaatttaagcacaataggaaataagcaagtattattgcctaatataatccaataatttatagaatctcttttcctggaagtatcttaaatttttctaagctacaaaaagttcctaagacaaatgagacagtcatcaatggttcatctagccaacaccgtggccatttgggcttttctttgtagtgcccgattcctggtgtgtgaaaataaattaacacaaattatattgccaagttaatatctgttttatgtgcccccagcatgtgttgaacatcaaacagtaccagggactttaaatatacccacggacaaagaaataattcataatgatgtttgttgaatttagttgcaatcaataaaaagtgcagtttgtgaatgctctgaggttcttgatattgatgtaaggctttgaacgacaaatgaggacaaaacataaataggaaagtaaaactgaaggatagaggccaaggccatgttttagaagatttaaagaaaaagggaaatttggtgagcaccataggaattacagatggctgtaggaattcttcctgttttactctctgggcatggaccacagcttggatccagaaatatttaggagcaggataagaggaccaagttcaattctataggaatcctttagctgataggctcagaacaaatcacataattgatagtgctgcttcaacttcaagtaaggaatattgatgcaatccttacagctacaaatggacagtggtctcatgttttcagttttcaagtgtttcttaagaggcaaggtgatgaaaacgcccacgtggggagccccatgtccttccattagtgtagagaaacctggtgtccagcagcacctgctccctctgcaagcccagcccccttcagcaagggcagtgacccagagaagaagcacagaagacacaaccctgtatcacattttgtttaatggtgccattgaccaaaggggaggatgaaaggcacacacttttttgttgttttttgagacagagtctcacgccatcacccaggctggagtgcagtgatgtgatctcaactcactgcaacctctgccccctgagttcaggtgattctcctgcctcagcctcccaactagctggaattacaggtgtgcaccaccatgtccagctaattttttgtagttttagtagagacggggtttcaccacgttggccaggctggtctcaaactcctgacctcaagtgatctgcccgcctcggcctcccaaagtgttgggattataggcataagccactgcacctagccaaggcacacactttggagaataaacactccttgttcgctgctggagggtagaactatgcttgactactaggcagagtccagtcttactgacaaacagccgtacatctgttctgtcttttcaatcaaacatcagcttcttgcttaacattgatgtgtacatcttgagggatgtcaaaatattgtaagctaagtttttcatacctgtgttccacactcaccatttttagtaataaccattgagcgagttcattctccctccttcctttttctatcacttaatctaaaattatcatttttccagcttaattttgataaccatgaatctggtattagaggcagggaacacctcctcaggactatcttttcttttatcatttggcttgcttacccaatatgcaaaaactatgctgtagaaaaagcagaaaagatatcttgattatgaatgaagctcctgtgtttactcagagagaagatgacccaggattcagttaacaaaatcagctgattatattactatatagtcctggagtcccaactccttgaccattacctcaagttatttggaattttgaagaggtgatttgtgttcctgcaataatgtctcaggggtgggctgacgggtttcctcttcctcctctcagtgagccaagggagtttgtggagaactctgagtgcatacagtgccacccagagtgcctgcctcaggccatgaacatcacctgcacaggacgggtaagagccccttgctgctatccacgtccatttcatgggaagggccttcacagaagccgaacagtgatgatggcccagggcatcctgtgtgggcaggacggccatcagagccacttcccagaggagacggcaggcgctgacagcgctgtccgggcagggtgtcggtgacattagcacacacattagcctgcgatgaacattcactctttctgctgacacccccaaccttatctaagcttatcaaatcctcacatttaacggaggctgttttcacctggtttcccccatccctgacctagtcagcattgctttatcgctttcatcaaacatcctcaaattcttaacattagcttgtaattaattgaagaatttttaaagaaattgctagcaaaactttttaaactgcacaactttgtatctatatgttcaataacatatagatacaatattctttacaataatcttttaaagaatatgagtgagaattcgggcccctctcacaccaaatgtcctgatgttgttaattctcaatgttattatatagggagctctgttttcttgtgagcttcaacagccagttctaaatctactaactgaaaacattttttagacattctctaaattgggcagaagatgacaggactgtgttttgagggataggctgccagcgtggctgcttacaaagtaaagacttggtttataggtttgcatggtgttgggttaaatttctgtcattaaaataattggcgatattgacatagtcatctaattatgctggctctgggcacacacagcccttgagtggacaaaaccaacatgagagaacttagccaaggggaaagcctttccctgctggttttatttctgctacttctgaagtgtggggcacacaacctgagcagtgcttttatttgagtcccaatgcttttatttgagttttgcaaggttattccaagttttacaaatagaaggtagcgtatgactcagtccttgatatgccaaccactgcacagagacttgccaccttcctgtcactggagaaacactcatgtgggttttcttaaatttgcctccctctgagcttccctttaacttcaactataatatgcaagaaagactatctgaccataaatacacatttgggccaatcaagatggttttgccaaggaaagatgcccacaatggttaagcagaatgcaataatgtagagaatatcatttctttcatgctggtgtatatcatatgcattcaaaaacagggagaacttctaagcaactaacagtgaccatatcaagcaggtgcaatcacagaataactggttttctcctttaagaatttttctatcatttggctttccccactcacacacactaaatattttaagtaaaaagttacttccattttgaaagagaaaagaaagagacatgcatgaacatttttctccaccttggtgcagggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacgggtgagtggaaagtgaaggagaacagaacatttcctctcttgcaaattcagagatcaaaaatgtctcccaagttttccggcaacaaattgccgaggtttgtatttgagtcagttacttaaggtgttttggtccccacagccatgccagtagcaacttgcttgtgagcaggcctcagtgcagtgggaatgactctgccatgcaccgtgtccccggccgggcctgtgttgtgcaatgctgcacatcacaacaggagggtagggggacaaaagagcacaggtcctggcagctgccacagtctccaggggcttttgcgtttctctccagatttctaaggttaacatggggattagctgttttgcaatgaataaaaggtaacattgcctggaatgttgcttaaagacacttttttaaagctagttgattgttaagctgttgctacttaaattaaaactactttgggccagacgcagtggctcacgcctgtaattccagcactttgggattccaaggcaggcagatcacttgaggtcaggagcttgagaccaggctggccaacatggtgaaaccccacctctactaaaaatacacctgtagtcccagctactcaggaggctgaggcaggagaattgcttgaacccgggaggcagaggttgcagtgagccaagatctcgccactgcactccagcctgagcaccaagagcgaaactctgtcgcaaaaaacaaaaacaaaaaaaaaagctactttgactggaattagcagaagcactctgattgtgtgtatcttatttactggaataataaagctgtcaatcaaactggatcccactcaacaatcagaaagagaagttgagctgtcatatagtagttcacacttacttctgtttctcaaaatcctcagctttgtttggaactgttactcattctttctctgaatccatctgtatgagttgtgtgcccttgggcaagggtcttaccttctctgtgcctcactttcttttctgtaaattgggataataatgctgcatagctcacaggatttttatgaccatgagttaagatatgtcatatacttaaaatggtgcctggaaaatggtgaatactgagtcaatgatagcatcattgatggtgggatggtgatgaggaggtgggagtcacaatggtggtgttgatggtggtgatggtggtgaggaggtgggagtcacagtggtggtggtgttgatggtggtgaggaggtgggagtcacaatggtggtggtgatggtgttgatggtggtgaggaggtgggagtcacaatggtggtagtgatgatggtgttgatggtggtgaggaggtgagagtcacaatgttggtggtgttggtggtggtggtggtgaggaggtgggagtcacaatggtggcagtgttggtggtgaggaggtgggagtcacaatggtggtagtgatgatggtgttgatggtggtgaggaggtgagagtcacaatgttggtggtgttgatggtggtgatggtgatgaggaggtgggagtcacaatggtggtgatgagggtggtgatgatgatgaggaggtgggagtcacaatggtgtcagtgttgatggtccgatggtgatgaggaggtgggagtcacaatgttggtggtgttgatggtggtgatgatgatgaggaggtgggagtcacaatggtgtcagtgttgatggtggcgatggtgatgaggaggtgggagtcacaatggtggtggtgatgacggtgttgacagtggtgacgaggcgggagtcacaatggtgtcggtggtgatggtggtgaggaggtgggagtcacaatggtggtggtggtgatggtggtgatggtggtgaggaggtgggagtcacaatggtggtggtgttgatggtggtgatggtggtgaggaggtgggagtcacaatggtggtggtgttgatggtggtgatggtggtgaggaggtgggagtcacagtggtggtggtgatgagggtggtgatggtgatgaggaggtgggagtcacaacgttggtggtgatgatggtgttactggtggtgacgaggtgggagtcacaatggtggtggtggtgatggtggtgaggaggtgggagtcacagtggtggtggtgttgatggtggtgatggtggtgaggaggtgggagtcacagtggtggtggtgttgatggtggtgatggtggtgaggaggtgagagtcacaatggtagtggcgatgatggtgttggtggtgaggaggtggaagtcacggtggtggcgatgatggtggtgaggacgtgggagtaacaacagtggcagtgacggtgattgagacatgatgatgatttgtcaactttctaggaaaacaatcatataatctccaacagtgatatcttaatatcttttccaaaagtatcagatcatattataagggccaagtttccagaataatatcagacataatgacagtggacatcagagcttggcatctaaaggtaatgggaatagctctaatgtctcagcgtgaaaaacaacatttgctattagtctgagatactaattatctagttaaggaagtactcacctatacctagtttttaactgttttttaaaatctggaattgattttgaattttaacaaatatttccctgggaacaatgtaagattcttcatattttcgcctttgggtataccaacatgccagctctgttggccactttgtgagctcgatgaagcatggtataaaagatgctttgctagtgtttcacgtaatctatttctataagcaattttggagctaagcctctgaaacagaattatattatctgtatagaataaatgttttatcttcccccttttctttcttctggaatagatgtgcatcagtatctctgcatcaatatctctatatcagtatctctgtgtcagtgagcatatgttgctgggcttaggggaggtccagaaagtgattgggttttggcattttcaatacacttactttgtataagaaatagtttgccaaatatagaaagaggggatttagtcaagatttaaattaaaaatgttagtggtcatttttctaatgtctttctattttttcccaggtcctaataaatcttcactgtctgactttagtctcccactaaaactgcatttcctttctacaatttcaatttctccctttgcttcaaataaagtcctgacactattcatttgacatatggaattttataaatattttctttagtatgtgtgattacattcctgattctgagcctttttagatgagtatatagtttgatataatcttgttattgccacctgtgtcttctcccaaagccattaattatataggaattacacgatagaaatgggtttaatttttaaaatacggccaagtgttgatgagagggaaaatttttttaatttctttcactgagtatttatgacgtgcacaacattcctgaatatattgtctctctcatttctcagatgggatgtattgccttctccatttctattgttaaagaaacacttacaggggtttctttaacaacttgtgaacagcagcatcagagcccagactacagcataagcagctgctgattccaaaagccctaccttccaaccgggcaggtgcagccacccagacgagggggaggaaccctggaggaatagctatttcttttttttttttgtcgagacggagtcttgttctgtcaccctggctggagtgcagtgccgtgatcttggctcactgcaacctccacctcccaggttcaagcaattctcctgcttcagcctcccgagtagctgggattacagacacctgccaccacgcctggctaatttttgtatttttagtacagacagggtttcaccatgttggccaggcttgtcttgatctcctgacaagtgatccacacaccttggcctcccaaagtgctgagattacaggcgtgagccactgcgcccagcaggaatatctatttttaaatggaactgtgttttcatagtacacggtgaggagaaagttgctttgaaatctttatcctaataaaccaaataatatgaaaatttgcctattttaattatatgtaacaaagtttagttactgctataattgcaaatatgtataaattccttaccaaaaaaaaaagaatcaagtgggagccagagaataatttttctgacagaattaaataacatgctatagctgcttgagttcatactcaatagtcatttctgcagagttaccgagggcctcatcagcgtcagcaggagcccctcgccttctgacgctctcacatccttctctcctgcagccccgtcctgccactgtccttgtccagcttctcttcaagggtcaactggtctacctttccctacaagtctgtcacagcttcttgttagcaatccctatggttgcccaaaagcattttcagagcctgcataagactgcatcttgtagaaaatttgcagtttcaatctgccctccctctgccgggtgttcccattgtattgcattcagcaggcagggagagactgctattaggtctgttcctgagtgactgctttctgtctcagactgtttggtgtctgtaggaggtagtggggtgggcagtaacgaggtctcctgtatattccacccctacgaagcctgtgtgtttggtttatgaactaagctcaaaagcaccacaggggtaagactgcagtacatgacaccatggaaaagagggagcacccagacccccaaattaagaagagcagtgtagagaacagagacctggagagcagagatagaaactgttaggatcagattatagtgttacaccagggctccccaggcctctcacatattgaaatgtacttgtccatctttctccaggccaggaaatgagagtctcaaagccatgttattctgcctttttaaactatcatcctgtaatcaaagtaatgatggcagcgtgtcccaccagagcgggagcccagctgctcaggagtcatgcttaggatggatcccttctcttctgccgtcagagtttcagctgggttggggtggatgcagccacctccatgcctggccttctgcatctgtgatcatcacggcctcctcctgccactgagcctcatgccttcacgtgtctgttccccccgcttttcctttctgccacccctgcacgtgggccgccaggttcccaagagtatcctacccatttccttccttccactccctttgccagtgcctctcaccccaactagtagctaaccatcacccccaggactgacctcttcctcctcgctgccagatgattgttcaaagcacagaatttgtcagaaacctgcagggactccatgctgccagccttctccgtaattagcatggccccagtccatgcttctagccttggttccttctgcccctctgtttgaaattctagagccagctgtgggacaattatctgtgtcaaaagccagatgtgaaaacatctcaataacaaactggctgctttgttcaatgctagaacaacgcctgtcacagagtagaaactcaaaaatatttgctgagtgaatgaacaaatgaataaatgcataataaataattaaccaccaatccaacatccagacacatagtgattttaattatttaagagtagtttagcatatattgctttatgatttaattaaaaatctccaaaatatatgccaaagaagtagaatgagaaaaatgtatatttctctttcacttcctacagatgcactgggccaggtcttgaaggctgtccaacgaatgggtaagtgttcacagctctgtgtcacatggacctcgtcaagaatgaccacactgctgtgggtgaagatgctttcctgcatttctgactgtcctctgtcctgatcaagtttctatggctctgggccagcctaccctcagccagggtttctgcagagactgcccagctggttccacgtggctccacgtgccaactttgtcctcagtggagggaaagttggacacacagtgctggggctgctccctgctccgccgttgctcgatgcatggcctgcctctgaattccttggttccactggttttgctgggtccttctgtgcctctagctcctctttttttctgtccacttaccccattggtcccatcacaagcctgtgtgtgagtggcctttctgttcgatgacaacctccagcataggggagtgtttctccttgctttctttcccagacacactgcccagcaaaggcaaaagggcttccttcaacatcagctctggccagtttgccagagcaaagccctgagaaaagcaaggttgaaaagtcttattcaaactcaccaggaaagagtggtgttactctcgatggcgtctagccaggaatcatggaattatacaccgagcacctgtttgccattttggatgtttccaaacatgaaccaaacttccaggcccctctgccatctctggtaacatttacaaagtcccttcctcaccactgcccttccttcattttggcatgctcctccgcccccgagttgacagccatagctctctctcctgccaccagtgtcacatgatcgaggaagaaggcaacttcaaaaagactgggtccccttccactcccatctcttcagtgagctgctaggacacccagcagaacttccccactccacactgcaatctcagggatcttagtcacggggctttccaccatgtctccacctggaaaccagtcatggccattccttcttacatctgctcttttccatctttttcttctcctcctgttcacccgcccttactcttgtggcgccctatggatatgcgctccatagcaaatgattctttatatcttacggtattctagtgagctggcacatgtggcttctggtttcctctctctggaactagacatgacctctgtgggagggaggattaaatgcaccctacagtctgaggctgcatgatgacatcactcatcacaatgatgctttctatgtctgaatcctattcctttataacccctttcaagctcgttcagagagtatttcacacaatccatgtgctcatcttaaaagccaaggacccagaggagtctcagcattgccaaaaagtcccttcacccagcctggccagaggcagtgcctggtccatgtgtatggactatggcacttcaattgcatggaaatactcttggaatgaacaaaataccaatccatgaaaaagcattattgaagtctaagttattttttgaatcatattttgttaatcaacaaattgaaaaatactcattatatggagaggtccagataaagcctcaattttaaaaaatgaggaaaagtgtgcctggtaggggactggggagagcttgagaaagttggaaacgttgccttagaagcctgttttttctccttttagaagctacatagtgtctcactttccaagatcattctacaagatgtcagtgcactgaaacatgcaggggcgtgttgagtgccaaggccatggaatctgtcagcaacctcacccttccttgttcctccacctcattccaggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggaggtgagtgccagtcctgggtgggctcaggagccctcgcaccccgacaggaacaagggccagccccgagaacgggccattagcagttgtgtatgttagatacataattgtattatgatgcagaaagaatctctgaatgtgcagttatacccagttggtgacatgttggtacatccatccgaggaaatggcaatgtttctaggctgcacccttcaatgtccacaaagctgtgtggcatctgcttaggacccggtgcctgtgtgtgcataggagggaggccaggaagcctggctgttgatcccatgctggcactgtggcgaaggcgagagattcctgctttggaaaacaccattgtccacacagtggctttgtccatgatggacttcgccacagcccagtcctgtgctggaagccatgttctctggaaagagcaacccagcggctcataagcataagcgcgtgtgatgtgccccaaccaaacgaccgccatgcacaacttccctaccggagttttcaatccagttaataggcgtggaaacagacatagaaattgtgtttgttgaaaggtagctgttcagttaaagaacacctgtatcagagcctgtgtttctaccaacttctgtcaagctctgtagagaaggcgtacatttgtccttccaaatgagctggcaagtgccgtgtcctggcacccaagcccatgccgtggctgctggtccccctgctgggccatgtctggcactgctttccagcatggtgagggctgaggtgacccttgtctctgtgttcttgtcccccccagcttgtggagcctcttacacccagtggagaagctcccaaccaagctctcttgaggatcttgaaggaaactgaattcaaaaagatcaaagtgctgggctccggtgcgttcggcacggtgtataaggtaaggtccctggcacaggcctctgggctgggccgcagggcctctcatggtctggtggggagcccagagtccttgcaagctgtatatttccatcatctactttactctttgtttcactgagtgtttgggaaactccagtgtttttcccaagttattgagaggaaatcttttataaccacagtaatcagtggtcctgtgagaccaattcacagaccaaaggcatttttatgaaaggggccattgaccttgccatggggtgcagcacagggcgggaggagggccgcctctcaccgcacggcatcagaatgcagcccagctgaaatgggctcatcttcgtttgcttcttctagatcctctttgcatgaaatctgatttcagttaggcctagacgcagcatcattaaattctggatgaaatgatccacacggactttataacaggctttacaagcttgagattcttttatctaaataatcagtgtgattcgtggagcccaacagctgcagggctgcgggggcgtcacagcccccagcaatatcagccttaggtgcggctccacagccccagtgtccctcaccttcggggtgcatcgctggtaacatccacccagatcactgggcagcatgtggcaccatctcacaattgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtgggggtccatggctctgaacctcaggcccaccttttctcatgtctggcagctgctctgctctagaccctgctcatctccacatcctaaatgttcactttctatgtctttccctttctagctctagtgggtataactccctccccttagagacagcactggcctctcccatgctggtatccaccccaaaaggctggaaacaggcaattactggcatctacccagcactagtttcttgacacgcatgatgagtgagtgctcttggtgagcctggagcatgggtattgtttttggtattttttggatgaagaaatggaggcataaagaaattggctgacccttatatggctgggatagggtttaagccccttgttatttctgactctgaaacttgcattcaattcactccaccaagttatctcatctttgaaatggctttttttaaaggtgcctagaatatgatggcgtgcagtctataaactgttgcccaccttctgtactttctctcagaataattcacattcttctccagtgtctgttgattgttactttgtggaataagttcttggaaaattccacaagattattgttatcttcttactaccaattctattgaactttctccaccttctctgggccttccccagccagtggtgggaagatgctggctggagtctgacagagcctcttctacactggcctgggcttgctgtgagttggtggaaacctttgctcttgtcccaacacagagcaagtgaaagaggaggtcaaggggctcaggcagcggactagggaagcagaatcgaggaaaaggaaaaatggctgacttattacctcaaaactctagagaatttagttgatcttacagccaagaaggacaaaagccagagagtaatatcctccgcctcatgtctaacccacagaatacatagcaagtaaagagaacatgggcctttataaaaatgtcttaagatacaattttttaattggaggaaatctacagtttaattttctctgggcagcttttcttccttttattatagtaggggaaatcccatgttgatatacttctaaatgaaagatgatgaattgatataatacaataaaaaatctgtaaaattgatgatatacttatcaagaaaaattagctttcattttaacggtttacaaattgagtcaagtcctagtaacaaaatgttaagtctattaacataaccacaagaaatacaggaagacgggcaatctgtgaagcctttcacttacaatctctggcccctcacctgtgctgtgtaggaaaatctttgtgcacaatttgcttccttaattcattttttattcattcaacacattctaataaattatacaaaatcatgttgaaatgtgaatttcagtggtatttataaatgcagtgtgaggagggtttggatgtattctaagacaatagttgtgctttgggaaggaagcagtgttcactgaaaagtgcccccaggaccttttaattggaggaaatatgcttctgtggagttggaaatggggtagaagatagataaggtcaaggcttaaaagttaagtgcacccaacatctgaagcgtccatgggcctggcatggtggctttcgcctgtaatcccagcactttgggaggctgaggcaggaggatcccttgagcttaggagtttgagaccagcctgggcaacatactgagacccagtctctacaaaaaataaaaaattagctgggtgtggtgtctcatgcctgtagtcccagccactcaggagatgggaagatggcttgagtccaggagatctaggctgcagtgagctaaaatctcaccactgcactccagcctgggtgacaaagcaagaccctgctcaaaaaaatagttagatataaatattaatatagatacctatatatatctgaatatagatatctatatatactctgtatatagttatttagatatataaatatatatgatatatatttagagagatatatatttagagagatatatatttagagatttatatatattttatatatatttagagatatatatctctaaatatatatctctctctaaatatatatatatctctctctaaatatatatatatccctaaatatattaaataaataaaagaaataaaagaaagctcagtttggcctcctgcttgtcctgtctcctcatcccctcttccccctccatcattttatttccttgccccatgtttcttcactgcggccatgtcccccctcctctccaatgatggatgtcatgtctgctgcagtcagagggcgacaagcctggagtgttccctgaagcctgtggtttgtggtttgtcctgcagctcaggctgcccaggcctcaccagcaatcctggcgggcagggcaccacactgggatggagagggggaagctggaggaggcactttctggtaaagaaagcaaaagccagcagtgcccaggccaatttcaacagggagttaaatagcaccttaatcctgtggcaggacagctcatggggccatgtgtgctcttagaaagactcacatgcacgcatgcacggcagcaatgactccatactcacgttcccctgcagacaccaggcccccacagccggcacacacactgcagccccagttccatgttgctagcagtggcttagtgaatgagtaaagttcttaaaatgcaggggacacctgcccttcattcataaggctggacgtacacctctccttaaggagttcaagagctagtggaatcccaattcatacggtagagccattcacagatgagagagacaagccagaaggaaggaaccaaaagtcatgtcagcagttaggacaaaataacaggctttcaaggtcacaaagcctcagggacactcctgcggtgggactgggctaggagccatgggggctccaactgtgcgctctgcctgccagcctgtgggtgctggggctccacgaagattgttgtggaataccaagcatgcttgctgtaggtcacggtgcacgtttactacttccaagacaaacagccgagaacaaagctcgctttagcttctgcgtacaccgaacgggacacacgactgaacagcgttcccattgtgcctgctgggtggggaggaagtgatggcccagtgggtctatcagatgttagtaggatggggcctggcggggctccaggctctgtgtggccgacacccacgccccccgctctgctccccattcccagccccaggtcagccctgcgaggccctgcagcagatgggctgctcaaactgctctggtttgcagatttttcttccctctcaaatgaatacaatatgttttcaagtctcaaccagatcttgagaaaataggaagagccagagggtttctttggtgttatggttgtacagcttcccagactccgggggagagatgtgatttgtgctttctggcaatcccatggcgtattaaattttcataggctttccagtttaaatttagggtaggcaatggaagggaacgcaaaacagatttctaggtgtactgtgtgtgtgtctcccacgtctaaagtctgttaactggagcacccaacaggccccacaggctgccttcacacagaggacctggggcgcctccgacccattggggtgagcagtgggccatggagggagccagggtcaggagacctggttgtgggcctgacctgaccctgctcagggtggcctcaggtgggccgttcacctcgtcagcctcagcttaccctctgactacagtgacctcagacaaaatacgcttcctggccctgtccagttctgactttttataaacaagcacttatccaagttaaagggatattttcaatatctactgagtccacagatattaaatatctcctctcttctttaaaattgtggcattatctttagaatataaaaggaaaataacacacactctccttgaaaatagagagcctaaacactctgcaggaaatatttaaagctatagtttttgtttgtttgtcttgaatgcaagtggcctggactttgacttgctttgagtctttgaccttcatgacttcagtacagttcaaccctgacagttttgaagtaggtatgtgcctagatctgccctagtccctgctggaatgttgaagaagcaaaggtccaggccctcagagcacttgccacgtacttgccaacagatacggggcggagacttgagtcaacgtaagagcaagtgtgtgccgggtgatccgacactgcagagcgccagctagaccctaagcgtgtgctaggggctgaccaagccgttctttcctcaaaaacttggtggggagggtatttttaaaatcacacaaatatttaagtacagattatgatgactgcctcaaagcagtggctcttcagcttcatcaagcttcagagtccagagggtttgttcatatggaaggctaggcctgtctcctgcatttcaccctcttggcctgggggcgggacccaagaatgtgtggctctaaaaggttcccaggcaatgctgaggctgctttctgaaggaaaaactgcaagataccaggagagtttcatttagattgaagagtcgaggaaggctcctctgagaaagagtctgctaaggaaggaggaggtgggttctggggacagaggttctcccgtgggtaagggtggagggaagctctcctggggagaaggtgggcaggaggaccagaggctggagggaggagggcagtcagcctcggggcttcccaggaacagggacggccagggcagggtttagggcaaggaaagcgtgtgagcatatttgtattttagtaaatatttacagtttgccctccatgtctgcagtttcatatccatggattcaatcaaccacaatgaaaaacgttggggaaaaaaattgcatcggtactgaacatatacggactttttttcttgtcattattccctaaacaatacagcataacaattattcacatagcatttgcactgtattaggtactataggtaatcaggagatgctgtagatgggaggatgtctgtaggttacacacaaatgctgtgccactttatatcaggggcttgagcatcctcacattttgatatttaagggaggtcctggaaccaattccccagatactgagggtccactgtctgtgtcccctcgccccaccttgcctttgtctcctgtctcctatctccaccctgcctcccgccagcctgttgctcctgacctgcccgggcaccctggagcagcaccctatctcagagcctggctcagtgtgttcacttctgcagagaaactaacttgcccaagtccacactcaaaacataggcattgctgagatgtgaaaagcagctgtggatgctttctgctacagtctgtgtgttcttttccatatctgaataaaaggtcaccaccatttgtattttaaagagaaagagaatttatgggtggaaattggggattccctcattctcagtcagacagaaaagagggccccattgtgtgcctgattgcaaataaatttagcttcctcagcccaagaatagcagaagggttaaaataaagtctgtatttatggctctgtcaaaggaaggcccctgccttggcagccagccggaattagcagggcagcagatgcctgactcagtgcagcatggatttcccatagggagcctgggggcacagcacagagagaccacttctctttagaaatgggtcccgggcagccaggcagcctttagtcactgtagattgaatgctctgtccatttcaaaacctgggactggtctattgaaagagcttatccagctactctttgcagaggtgctgtgggcagggtccccagcccaaatgcccacccatttcccagagcacagtcagggccaagcctggcctgtggggaagggaggcctttctccctgctggctcggtgctccccggatgccttctccatcgcttgtcctctgcagcacccacagccagcgttcctgatgtgcagggtcagtcattacccagggtgttccggaccccacacagattcctacaggccctcatgatattttaaaacacagcatcctcaaccttgaggcggaggtcttcataacaaagatactatcagttcccaaactcagagatcaggtgactccgactcctcctttatccaatgtgctcctcatggccactgttgcctgggcctctctgtcatggggaatccccagatgcacccaggaggggccctctcccactgcatctgtcacttcacagccctgcgtaaacgtccctgtgctaggtcttttgcaggcacagcttttcctccatgagtacgtattttgaaactcaagatcgcattcatgcgtcttcacctggaaggggtccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccatggggatatgtgtgtgcgtgcatgcagcacacacacattcctttattttggattcaatcaagttgatcttcttgtgcacaaatcagtgcctgtcccatctgcatgtggaaactctcatcaatcagctacctttgaagaattttctctttattgagtgctcagtgtggtctgatgtctctgttcttatttctctggaattctttgtgaatactgtggtgatttgtagtggagaaggaatattgcttcccccattcaggacttgataacaaggtaagcaagccaggccaaggccaggaggacccaggtgatagtggtggagtggagcaggtgccttgcaggaggcccagtgaggaggtgcaaggagctgacagagggcgcagctgctgctgctatgtggctggggccttggctaagtgtccccctttccacaggctcgctccagagccagggcggggctgagagagcagagtggtcaggtagccctgcctgggtgctggagacaggcacagaacaacaagccaggtatttcacagctggtgcggacccagaaagacttctgcttttgccccaaacccctcccatctccatcccagtcttgcatcagttatttgcactcaacttgctaagtcctatttttttctaacaatgggtatacatttcatcccattgactttaaaggatttgcaggcaggccctgtctctgagaatacgccgttgcccgtcatctctctccgacagcagggcagggggtccagagatgtgccagggaccagagggagggagcagacacccacccggcctgggcaggtcctcctcattgcttgcatccgcctggttagcagtggcagtcagtcctgccgagtcattcgtgaggcgctcacccaactccaggcagatgtaaaaggtgacctacaagaagacaaacaaaaacatctggagcgctcttatgccagcatctgcccttgacaccaccaggcaggctgttgctgggagccgtggtgcttgggtaagctccttcccatggcagagctcctgggacgcattgtagaagcagggaccacctcccaggataaccagatagcagcacaccctgcacagccccttttactccagcatcatcgggcattgatatctcagctgcagccacaggcggcccccagcaccccaggaagtggggagcgctcatgcttctctgagcacaaaaatcactgaatatttttgccattctcatggtcataacccgggccacagagtagaacactcctatcactgttgttagacagtggtcctgggagagggtcttgtgtgcctcggatgccagggcctctttttattgggaggtgcttgttatttctgtgtgtggctgcatttgtttcccaagactgccacaacaaatcatcaccaacttggtagctcaacatagcacagctttattccctcctggctctggaggccaggtgtctaaaaggccatgctcccacaatggttctgaggaggatccttcctgcctctctggcttctggtggctccagcatccctgggctgtggctgcacctccccatgtcaacctccgtcttcacaaggccttttcctgtgtctctgcaaccacaggcccctctcctttctcttaataaagataccagtcattgagtttgaaaattgctaagagagtctgttgtaaatcttcttagcacaaaaaaaaatgacagatatgtgaagtggtagatatattaattagtttgatttgatcactccgctatgtgtataaatgtcaaaacaaacattgcactccataaatatatatattaaaaaagatcccagtcattgcatttaggacccaccctaaatccaggatgatttcatttcaagacttttaactagatttgcaaaaccccatttccaaataaggtcacattctgcagttttgggtagacgtgaaatgtggagacactgtgcaacccactgtcttggggagggggtggtcagcctggggcagatgttgctgggtgtggagctacatccactcatgccctgacctggaacccagacctgcttccccagctctcctcctggttatctgaagcagggaatggagagcactgccctccttgcccaggcagtctctatcacctggttttagtttcttcttagcacatattgccccagaatatctggttggtttatggcttacttgagtttgtgcctacctgtcccaaccgggaggtgagccctggctattccccaaacccggccctgcatgtgggagctgcccttcctccgttcatcagagggggccaacagtccacagctgttcttaatcatctcccagtaacccccagctccacaaaggtgactccttacatggtggagaggtggtcgggccatccgtgtgaaatgtgtatgtgaccgttttccttaaggggcacgtagtcttggcaggtttcgctcaatataggatgagctcaggactccagtggactgtggattcagatctggattctggcgcattcgccgtgtgaacgggggcacgttgctggcctgtctgcgcctcgtctcccgactgtggagtgtgttctgccccttgtctttctgggaggtagggagggcagtgagccccttcgcatcgcccaccacaggcccagcacatggctgatccccactgagtgttcttttcctcctttgatcccctttggctgacctaggttggagcagccactaaaatatacccagaaacatcttcctaatctacatctgtgccaaccctcattccctggcgcagcatgaccatcacatgcccgccattgttcctgatctctgctgctcatgacctgctctccagcgctccttctcatgctcacattccagttggcctgacctagataagtggaggtttatttgaccccaaaaattagccttctacaaacgaatataatagtgtccattacagagaataaacttagtgcgtgtcccatttaagcagaagttactgaaagcctgagtttaagtttccagggcctgaaagttttccatgacagttttctgcataatattacctacaatttcaatctgttatttaaagccattcttgtgtttgttgtactttgattagctttattttgatttgaagtccttttacattacgggcagttaacgctttgtctctgttagatttgctttttagttcacaagagaaacctcattcctctgtatttgaatagttgcaatgatggaacagctgtccctggagggaaatgaaaacagtgattccccaaattgtgacaatagaaatttgctcttgggttacttacaatgtatctgagtattaaaaaattttctttttaaacgtttgaagtaaaactacccagaaacacttagtggctgaccagaaactaaactcctggcatcctcaaaatgggatttattggcttataaatgtcctgtgttgactcacaaaggcacaaactatctaggtaagttttcttctaaatgttgatgggagagctggccactgttatgcaagtttcattgtcctgactaaactgccaaagagattacataaaattatatcaactagacaaaaggaaaaaggaaaaaaaacagaggtgtcttgggaggaatccatatgagaccagtagaccatgagagagacatcccttgccatctacaaggaaaatggattttgttctccatatgcaaaaccatctcaggagcttgcggagacaccacttgcttactagccagaaagagcaggtgcctcctaaattccccacacaggagctcacagtggctttcatgcactgggattaagttagacttaagaaagcctgtctactcttcctgggatttacaagccagctagtaaatcccagaataaatcacacggcacagtcatccaaagatcccgtcatccgtgccgtttggaaagccctgctcctgtgccaccctctccccgtggagcctcccatgcccaggactgcagagtcctgccattcagactgcaactcatctcacattcttccaaactatttggacaacagagctttctcatcacctaatgcagattacagtctcacagaattgagtgttcaggcagacactgatgtggttctgtagtacagcaaacaatatcagtttacagtcctgaggccaggcctggtgaacaacgcacggtagcggtggggcagggttctcagaatgaaactggcttacacatggcactctctgaccacaactgtataagcaccaaactacacttagttccatctatgaggtaaaatttaatgcagatgaacatcaaagaaaacgtcaaaggctcctttttacaagtacgtgggctacttaatttggtccaagtccattttaaaaagccctaggtgctttcacggctctgctactgacaagaagccccagtgcctgtgagctgctaatgggagggagaggaagatgagctgagtgggccgggctatcccgtccacaccgggagacagggaaggagactccaagctggtggtgccagcacattccaggccactcaggcctattcctaggtgccaggtcacgaaaaccacgctgacagatcgtgctgtgtgcgtgtcatagcacacaagcaggactgtgagagagtgaaagtgacactgggtggagcactgaggaagggccacagtgtgttggtggagataggctgtcatggagaagagaccctggcttgctctacattgcttccaatgcaactgcaaggcaggtcccagagggctccggccttcgtcatccaggtttgctccctcccctcatggctttcccatcctcagatgaggactcggcagagcctacccctgctgactaactgtggccccagggtggtgactcagccctgcacctcctgatcccgtctgcactgggccagagaggatgacttacccagcacgttcacatcacacagctttgtggattcctaggtccaaggaccagagatttcagttatgtgagttattttttttatttgttcttgcgtattccacaaagggtcgcagctaaacttaacctaatgatcactttagtatatcactaaaaagacaaagctcacagtgctgttgaagcacattcatcatctttagacattttgactagttatttcttaagcatttacctgctagtgttaagcatcacatgaaatacatatagaagtaagacaaaatttcttatctccccaagtttgccaacaaatacagagcaggaagggaagcaggtcagagcaggaggcgcagctatagtgaggccaccatgcaaggcacagggagggtgagctccaagtttgaatggaatgggtctgtcagccaagccccctggctctgggaagatagcagtgaacaagccagatggcccctcaccctccagagccgtgagtcctgcagaccaaacagcgtgacaggtcctttccctgtccaggaggcctctgtgggtgagagttggctgcggacagggcgtgaaggcacttgagggtggggaagtgactctgactgggagatgctgaggacagggaggaaaccaccagataagggacactggggaggaggggtggacccctcagggccaagcacatggagcctcatcacaaaggcaagatggtggccaaattcaaggtcgctgcaaaaggaatggagaagagagaatagatttggcatttggaggaaatggtgacaatcatgagcacctacccgggactctccatgggtgctatctctacataaactcattccaccctctgattaatccattctacatatggggaaacaaaggcatgcggtgtttacgtcacttgccaagatctcaggatttgatccaggtggcctggttccatggtgcagcctctcagcctgcatggatgccccagctcagagcatgactctcaggacaggggtcccagcagccctccctccctgagcagcagggtgcccgtgctgcaccacttctgtctaggaataggacattctgacactttcctgcctcttccgaggtctagcacttactctatgcctgcctgggaaggtggcaagctggcctgaggaacagactcttccattttttagggagctcaaggccacagatgctctgagatctggagtccagagacaggagcggaggcttctcctggtgaccactctgcttaaaaacttcatcagatccgtagtttcagagcccccctgaaccccatcccttacctctaccagttgcaggtgggtctctggggtggggctgccctccccaccagcaccccaagggctaaaaggttgaggggagaacaccatcatttgtacagggggatcctggaagatgaggcctgagaaagccctgcggggcccctcaccttctccctagctgtggccaagagtgtctggccttgcctgcctcaggaccagcccaaagtggaggtgagaggtgagccccagcccccaggggaagggtgatggtggtcttggtctcagcatggttctggtagaggtgggttattttgaagatgatgaaccttaagcctctttctgatcttgctttaaataaatacttctgaacaacagcaacaacagaatagtgttgataggaaagccctccactccaccagaaccacgcggccttctcgtcctcccctcctccacttccttcctaagtcactgctccatgagctcttccacaggagatttacaaaacagaacacaaacaatccagttcctgcctctcactctgaactcctcccaagactcgtggggtgcggcagcccctgggaacacccagcccttcaaggtcaaacacagcccccgcccctcactctggggtaccctgccagaataagccccgacagccatgtggagcagagccttcttttttgtaagtggaagttccaggctggcttttcaaatccccttttaacctcagtgctgtatttcaaaattcattccagttttcctgtagtaattaacaaaaataaatattttaatttcaattaaagtgagggtctcggagaagaagcaggaactgagtttcctgagaggccccgctgaggctttgttgatatttcttcctgcgacctctgctcggaccctgggagctcacaggccgtatcgcagctcttatctttggggaccagttaaagcataactgcgccaggcacagagttgtcctttcaaatgtgccggcagtgggacggagacccatgcgtcaagtctcctctaagttcacatgggattctctccttgtcccaaagctgtctctgacttaaaaccctccaactgattacctgaattccagaatatgtcctgtgctctctgccctttcccacgcctttggtgaagaccggtgttctgaggaaacagacactgtgtagaaatggctcaggtcctttaaagccctggtgtgaggagtggggaagggctgggccagaggtcagctggatttgttagattgacagagtgacgcggacttccccagaggcacgggaccaaggtgcatgctcacgctgtctcatgctctcacacataatgtgtgtgtgtgtgtgtatatatatatacacatatacatatatatatatacacacatatgcatatatataaaaccccaagcagcctctggcttagcaggtgcatttcccagcagggcaattaaagccatggtcccagtagtggtcttggggtctcagggtatttggtctgtgcagccacatgcttcagtctctggaccccaggtcatctaacgaggtggtcgtgtggggactgggatagaaaaggtgtctgcacggacgtgtgtgaaagggctggcacatcgccagtgctcagcactgtcagctgctatcaccagtcattcaatcattcattcattcattcagttgttcattcttcaacaggccgttttaaaaatgtgcccagtataccaaaatctccgctaagcatttaaagaggcagaatgaaagttagcagtggtggtgaaacgaagctgggaatgtgctctgagggcctccttgtgggcttaatgaatatgtagaaaccacgcattttaaatagagagggagaaagggagaggttcctggtcctctgcatggggacttgtgtgtggctctttactgtaggcctgtgccactcctgctcaacagctaccacagaggacgccttcaacaaatgtgaagaacgaacaaaaggtacaaatgtgaagaacgaacagggtagaaagaaaggagaaagcaagggtgagggtgagaaatcaagggacagagaagagagaagaggagatagcctgggagttcacacagccaagaaggtagacactcagttgaaccagcaagaggctgagcctaactctccctttcgaatgggcaggagttcatgatatttaataaacagaggccttgctctgtaagagacagggtaccaggcagagagcaagtcagcatcgcaggagtcaaacgaggcagacagcgggggcagggagcttgcctctgaaggagacccaggctgccagagtagcagggagtctgggccagtcctcttttgggaagcgcttcctcggcttctgccccccctctcctctccctttccacccaccatcctgacataatacttcctaatctggaagtgttgtccagagaagaacctgctcatttcctcttaagtaggcagggaagcactaacgtccagcagcatcggaaacccgtaggagcgctctcggcagtgcagggtgaggggacagtccatgtagtcatgagacgtgggtgtcaggcaagcgtctcttttccaaaagagaaaaacattaaaggcctcacaaacggcgcccaaagactaattctgcatagcatctttgcgagaccctaggttcttatgatgactggttttgcctgagaaagaaaaaattttaattttgctttgacatgccaattcaacaaatcattttcacataatattcatgcaaaaaaaaaacaatttgccagaaaacttgggaatccatccacatctacagcttttccctgcagtcacactacagtgggatccctccatacaggagcggcagagtggagcaggctagagatgcctgtttgtttctgtttgctgcaccgcagcaagcatttctgtcgtgcccactctgtactagaaagtacatgaacatcagccataaagggaactagaaaggtggcccaccctcttggtggagagagaagagagtgtggtagaaacaataataagaagtctgcagaacttgacccctcccagcctctcccacctgccagcctggcccttgcagagagatgcaggctgccattcttaggccaaagcctgggacagttgggctcagcaaggtaggcatccgtcaagcaaggaggagcaggggtcagcagtgaccccagcagccagcagggagaaaggtgcatgtgacaaggacaccagaggccgtgggtcaggatcagccagggtcagggtagcatttctaggaattcactctgttgggcgctgtgctggctgcttctcacatattattcctttcttactctcagagcagagatttcaattgcagcgagattgtggaggcagccagggaggtggggagggtggtgtcttctaaaagcattttcagtatccatgtggtttcagtaataataataataataaaccagtgaaaagtaaaacaggacaaaaatcttcataggcagtgaaccatatcagagagtccaagaaagcacaatgagagtgtggcttaaaaaccctgaacgacattcctttgcaccagcttggtgaggagggcatggtccccgccaccccccacccccactttgcagataaaccacatgcaggaaggtcagcctggcaagtccagtaagttcaagcccaggtctcaactgggcagcagagctcctgctcttctttgtcctcatatacgagcacctctggacttaaaacttgaggaactggatggagaaaagttaatggtcagcagcgggttacatcttctttcatgcgcctttccattctttggatcagtagtcactaacgttcgccagccataagtcctcgacgtggagaggctcagagcctggcatgaacatgaccctgaattcggatgcagagcttcttcccatgatgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccagacattctgggtgagctcgcagcagctgctgctggcagctgggtccagccagggtctcctggtagtgtgagccagagctgctttgggaacagtacttgctgggacagtgaatgaggatgttatccccaggtgatcattagcaaatgttaggtttcagtctctccctgcaggatatataagtccccttcaatagcgcaattgggaaaggtcacagctgccttggtggtccactgctgtcaaggacacctaaggaacaggaaaggccccatgcggacccgagctcccagggctgtctgtggctcgtggctgggacaggcagcaatggagtccttctctcccttcactggctcggtttctcttagggaccctcacagcactaaggggtgcgcgtcccctgtcaggccctcgaatgccctcccacagccaggcccctctgaggtttcactctggcctgctgggctcctagcagccaccaacccatgatgctgggccctgaaaacacacgcagacctggatgagtgaggccactgggcacaaccagggctcccagctcaccagagcagcctgggacacagagggtgctcagaaacctaccagagcagccctgaactccgtcagactgaaatcccctgttgccgggaggaggcgccgggcctgggggacgggtcctggggtgatctggctcgtctgtgtgtgtcactcgtaattaggtccagagtgagttaactttttccaacagagggaaactaatagttgtctcactgcctcatctctcaccatcccaaggtgcctatcaagtggatggcattggaatcaattttacacagaatctatacccaccagagtgatgtctggagctacggtgagtcataatcctgatgctaatgagtttgtactgaggccaagctggcttttattgttagttaatttacattatatcctctgacatgcaagtattttctttcgagataatgactaatgataatgtaatcattgctgtctatctattgtactgagaaaacacggcagaggaaatcgagtccagctgccgtccaaaagtcactggagattgcaatgagctcgtctggcagggtggggggtatgggagggaaagagcttaggaaacggctctccctgcaaagtccaaccaaactttaacgttaaccaaaccattaatgttgccatgaatttgaagtgaaccagagggaggtggcagaagaagcttaatggggaatagttccggtagagaaatgaggcttaagatgaactaccctggcccttatgtgtcagagagaacggcttgacaaacacacactgaggatgtctgcagggataaaagaagaaagggagatgacccttgcttctcgctctcgggaggaccatctggtccggccctggggattctctgtttcctcttctgaatcccagtgttgcccagcactggcctgtacccatcctcacgagggccgctctcctcacccggccctaggtccctgccctgtcctgagcctacaggggcctcccatgttgagaaagtgttgctgacacattgtctctgaccgctgtgccaggcattttctgctgaattaccgcacttggtccttgaatttcacccagcaacttactgaaaggctggaacccatgaacctaccccttcactgaggaaaatcagttaccccagccatctacagcgacaggagcaagggaggagtcgcctcacctctctagaaatgtgtatttgaggagaacactattgaaatgaatttccaagaataatctagtcagtattacaaaagcaaaattatttgggatatcgtccttttttacttagtattttttctttttcctatagcattattaactttctgattttccaaatacatacacatttttaaatttcctgagtctttatctcttctgttaaaatgtaagatttatgatacaaaggcagagatttgtgtccatgaataagtgaagtttggtgtgcacctgtgagctgagccacctcaattaatggaacagataaggaaataaaggtctgctgatgcattgttatttacagccattttcagaatgtatctcctctccacgagggaactgcagggtcctgccccaagccatttattttgtcctcaagcagcccgcccctcccactccaggcacagcccggtctcctgctggtctcccctcttcccacttgctccccctcatctatgctccagacagaggccacatatattttttaacttttttttttttttttttgagacagagtcttgccctgtcacccaggctggagtgcagtggtgcagtctcggctcactgcaacctccacctcccgggttcaagtgattctcctgcctcagcctcctgagtagctgggattacaggcgcacaccaccatgcccagctaattttttgtatctctagttgagacagggtttcactatgttggccaggctggtctcgaactcctgacctcatgatctgcccgcctcggcctcccaaagtgcatattttttaactttatcagacttttcattctctgctcaacatctttctttggtcctccaggtatgttcagataaaacctgagcacctggccatgactgatgggttgctgggccatctggccctggcaactctcccgtccaccaggtccccctcccgtcacgctccaggcatagcctgtgtgtgccagcgcaatgcccacactccatgcacaagtggaagccctctcaaagtcagtggcttagtgccttgatgtggtcacacccattctcaggaagtccgttcccactgaaaacattgtgtgttttcaacatcattgaggctgccacggcagattataatcactggcctaggcagcccactggaactaccagaccatgagcctgaattttttgtttaaaaatcatatcctgttttctctactctctagtctctagtcaaggtgaattattcaatttaataaattaggggcctagtgtgttgtaccaaggagctaaaaagagagaactcgcaacaccttccagcccattctccacctaacactggctatactggctctcctctctctcgctgtttgttccaaaatctaataacctgtcttcccactagaattcatcatacatgtttaaaaacctagttaaatagtagttaaactgactgcatagatctggaaatgagacagtctttcttttacaaatccatatagactatgagttgggggcaggggatgacacaagaatctattttcttgcccccaaaccattgctttccttccaatgttaagcttgtattctgtgtattaattcaggtggttccgtttgggaatggcctctgttacccagagatgggagggccatcagaactcggggttgtctgaaaaaacactggttctaaaattatcactgctttcacttgtttttaaccatcatagttgtttgattttgaaggaaaaacatgagggtttttattctatgcttgttatatctatattgtggtttcgtattttttagattttagtacctgacatttttttaacttttattttaggttcaggggtacatgtgcaggtttgttatataggtaaatttgtgtcatgggggtttgttacacagattattttatcacccagggattaagcctagtacccattagttatttttcctgatcctctccctcctcccatcctccaccgtcctatagaccccagtgtgtgttgttcccctctaagtgtccatgtgttctcatcatttagctcccacttataagtaagaacatgcggtatttgattttctgttcctgcattagtttgctagggatgatggcctctagctccatccatgttcttgcaaagtacatgatctcattctcttttgtggctgcctagtgttccatggtgtatatgtaccacattttctttatccagtctgtcattgatgggcatttaggttgattccatgtctttgctattgtaaatagtgctgcagtgaaaatacgcatgcatatgtctttatggtagaatgatttatattcctttgagtaatgggattgccgggtcaaatggtagttctgtttttagctatctgagaaattgccacactcttttccacaataattgaactaatttacattcccaccaacagtgtaaaagcattcctttttctccacaacctcaccagcatgtgttgggatttttttttttttttacttttcaataatagccatctgactggtatgagatggtatctcagtgtggttttgatttttatttctttaatgatcagtgatgttaagctctttttcatatacttgttggctgcatgtatgtcttcttctaaaaagtgtctgctcatgtcctttgcccactttttaatgggattgtttaattttttcttgtgaatttacttaagttccttatagatgctggttattagacccttctcagatttgtagcttgcaaaaatgttcacccattctgtgggttgtcttcactctgatgatagtttcttttgctgtgcagaagatcttcagtttagttagatcccatttgtcaatttttgcttttgttgcaattgcttgatgtgttttcatcatgaaatcttagcccattcctatatccagaatggtattacctaggttgtcttccagggtttttatagtttggggttttacatttaagtctttaatccatgttgagtttatttttgtgtatggtgtaaggaaggagtccagtttcaatcttcttcatggctagctagtcatcatttattgagtagggagtcctttattcattgcttttttttttttgtcaactttgtcaacgatcacatggttgtaggtgtgcagccttatttctgggctctctattctgtttcattggtctgtatgtctgtttctgtactagtaccatgctgttttggttactgtatccctgtagtttaaagtcaggtagcatcatgcttccagctttgttctttttgcttaggattgccttggcaattcaggctcttttttggttccatgtgaatttttaaattgtattttctagttctgtgaagaatctcattggtagtgtgataggagtaacattgaatctataaaatactttgggcagtatagtcattttaatgatattgattctttctatccatgagcatggaatgtttttccatttgtttgtgtcatctctgatttctttaagcagtgttttgtggttcttattgtagagatctttcactttcctggtttactgtatttctaggtattttattctttttgtggcaattgtgaattgaattgcattcctgatttggttctcagcttgactgttgttggcatattggaatgctaattatttttgtacattgattttgtacaactgagtcttcactgaagttgtttatcagcttaaggggttttgggcaagactatggggttttctagatataggatcatgtcatctgcaaacagagatagctgttttcctctcttcctgtttggatgtccattatttctttctctcacctgatttatctggccaggacttccaatactatgttaaataggagtgttgagagagggaatccttgtcttgtgtcaattttcaaggggaatgttttcaacttttgcccattcaatatgatgttggctgtgggtttgccatagatggctattatgttgaggtttgttctttaaatacctagtttattgagaattttaaacatgttgaattttattgagagccttttctgcatctattgagatgatcatgtggcttttgtccttagttctgtttgtgtggtgaatcacatttattgatttgcatatgttgaaccaatcttgcatcccagggatgaagccgacttgattgtggtggcttaagctttttgatgtgctgctggattcgatttgccagtattttgttgaggatttttatgtctatgttcatcagagatattggcctgaagttttctttttttgtggatctctgccaagctttggtatcaggatgacattggcctcatagaatgagttaaggaagagtccctccttctcaatttttttggaatagtttcagtaggaatggtaccagctttttttgtacatcttgtagaatttggctatgaatccatctagtcttaggctttgttttggttggtaggctatttattactgattcaattttggagctcattattggtctgttcagggattcagtttcttcctgaggtttttatttttatcaaatggaacttaacctttttcatttccaatttttttatgatctaaaaatgtgcagtttacagccctgttcagaatctgcatcttcctcattctgcagatacaggtccctcagagcaggtgactgagtgtgtatcctgtctggagcataatacttatgctagtagagttactgttgtctttattgttaattaccaaagtttaccacttatcagtcacttactacttgctgggcattgcactaagcatttcagttgtattatcttgttgggtccttacagcaatcctgtgaaacagatactgctattaccccactttatagagaggtagactgaggcttccagcattgaagcaaattgcccaagactacagaaatgtaggtttctaaacatcaagaaacagtaaccagtaatgatgactaaagcaagggattgtgattgttcattcatgatcccactgccttcttttcttgcttcatcctctcaggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgtgagtgactggtgggtctgtccacactgcctagctgagccttggtggctgctcttagccaaacagctgaggcctttgcatccctggagaaatgtcatcacattacttaaggcaggcacacaaatccagaaacatctgtaaataccccttcaagcattcttttaaagacacttcttgactcattgggcagtatgacctgacatttgcccatgtttgcaagcaaataaataaaactaaagtcttccgcaagccattacaccaaaatattctattcgctgagttactcaatgaaataccgagttgccctatattttgaagcctgttaccagagagactgaatgtttttaaatgcatggcagtgagtaacaacataaggctaatagagtcaacatttctgctttgacttaaaccttttaaaccagtggatttatgtgaagtctctgcagtgtggcatttaaacatttcaatctaaataagagtgtgtaatttgattgatgctattattctaccagattcacgagtgcagtgggctctggaggtagcattacatgcatgggatgagcatttgcaaaagaaagttgtatagggaatatgacagagccaagttaatgtaaatattaatgcctttctgaactctaggccacagagttgatcttttttaacttccttggtttgggctaaggaagctgtgatccagagaagccacgtgatttgtctaaggtcacatagcagtctggcctaaaatagcttgatatgctgtggatggaaaataaatgtgatccctcaagaggcatgaggatttccaggcagtagccatacctccaaattgtttaatctggatttagattgttgggtagtcacatgcagcagcacagttaacagtgtgtcctcctgtggaagtggccagcacagccagccctctcacttgcatgcatgcccaccagccttctcacttgcatgcatgcccactgggtatgtgctgtactggagacgccgggggtaggggcccagtcccaaccccaaattctttaaagcctatttttctaagttgcatctggtttcctacctgaaggaatgctaagggtggatgttgagtgaggaccttggtgcagggcaccctgcagtcaggatagttcatggagagcaattgtacagacccacactgctccatcccctcaggcgtaacacaggatgctgaccccaggaagagtgggcgtagaaaaactagagggcattattgttattctgattcaaatgtacagtgctggcatggtctttaaacagtaaccagtactagctggccaagacagaaaagtctaccacaaagacttggttctttcatcacttatttgactggaagtgtcgcatcaccaatgccttctttaagcaatgccatctttatcatttcttccagtgttctaattgcactgttttttctcattccttccccaggctggatgatagacgcagatagtcgcccaaagttccgtgagttgatcatcgaattctccaaaatggcccgagacccccagcgctaccttgtcattcaggtacaaattgcagtctgcgcttccattgggaagagtccctctaatgagcatctcatgtcactgtgttctgtcacatgccagcctggcctccctgtgtcccagatcgcattattaaaccctccagcgcattagagcaagcctcagtaaggcgcaggccacatcgtgaactaagcagcatccgtgagtggggcccacccaactccatctccccctccccgtctgaactctcctctggtgctcgtcctcactgtccggctagccaaagcctcagctgggtctaagagagaagcatggtctattgggctttggtgtcaggcagacgtggcttcacacccctgactctccacttcttcgcatcacccaggcagccgatccacctatctccttccataacacaggaataccaaaaccaagctcacaggattgtctcaaagattcaataaaatatgttgcaaaatacgctccctaacacctcacagcaaggtgcacaaatcgatgaatgctgcagcttcttccctttctgtttcctcagaagctatttgaatctcatgtaggggctttcaagcatcaaaggatggttcatgttttattttaaggcacccacatcatgtcatgaggggaggcagctataatttagagaaccaagggggatttcattataacaaaattggcaaacacacaggcacctgctggcaatagacccctgctcctatagccaagaagtggaatagcatctctacgggccattctaatagcctcaaaatctctgcaccagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctggtatgaaatctctgtctctctctctctcaagctgtgtctactcatttgaacaaattgaattttagggaaaataaccatctagtgaaactcacatggaaatgaagtcaattttaaccaaatggtaaaatcaaaatcaaaataaattaagtgtattaattattttgttgcattgcaacaacttgattgtaagccttttaggtccactatggaatgtaattaaatcaaaactaaacctagttgctctaaaactaacgattaagacaaaaattaaacaccttcacaatataccctccatgaggcacaccacctgcattcaggaaaagtggatgagatgtggtacaagcattccatgggcaattctctgtttctttttccagagtgcaaccagcaacaattccaccgtggcttgcattgatagaaatggggtatgtatgaacaccttataagccagaatttacagctctccactatggctctattttacatggaaaatgccttaacctaaataattttaacccagataatcttgagttttcttcctgtgtgggtttttccctgcacggctgtcacgcctcacagtgccgttcaaagcgtgactcctggaccagtagtagcatcgcctggccttgttagaaacgccatttttcaggccactgccccagtttgaccaaatcaggacctctgggggtggcacccagtagtctatgtttgagccactttccaggtgatgctgatgtctgttgaagtgtgaggccgtggtctagaccgcactgtgccatgcagaaaccactagccacatgtggctacttcaacttaaatgttaatgagttaaaatgaaataaaatataaaattcagtttctcacacatgtgaagtgtccagtagccacacgtggctagtggtgaccgtattgaagagcaccgctcatagcacacctccctcactgcggaaagttctgctgtacagcacccagcaacagccctgctgcccaaccctgcagcctgtggcccaagtagcaccagcacccaccagggtgcaagactctcaaggcctgcccaacctactaatcagaaccagcatctcaaggagatctcgggtgatttttgcaaacactgaagttggggcagccctgaccggagtaaccttccctcatttcctcctgcagctgcaaagctgtcccatcaaggaagacagcttcttgcagcgatacagctcagaccccacaggcgccttgactgaggacagcatagacgacaccttcctcccagtgcctggtgagtggcttgtctggaaacagtcctgctcctcaacctcctcgacccactcagcagcagccagtctccagtgtccaagccaggtgctccctccagcatctccagagggggaaacagtggcagatttgcagacacagtgaagggcgtaaggagcagataaacacatgaccgagcctgcacaagctctttgttgtgtctggttgtttgctgtacctctgttgtaagaatgaatctgcaaaatttctagcttatgaagcaaatcacggacatacacatctgtgtgtgtgagtgttcatgatgtgtgtacatctgtgtatgtgtgtgtgtgtatgtgtgtgtttgtgacagatttgatccctgttctctctgctggctctatcttgacctgtgaaacgtatatttaactaattaaatattagttaatattaataaattttaagctttatccagatactcataacctgctaacacacacacatatacacacacatacacatacacacatatacacacaccacacacatacacagacaccacacacataccatacacagacacatacacatgcacacacatatacacacacacctcaaatacatacacaccacacacacatacatgtatacacacatacacacaccacacatacaccacaaaaaccccacacacatacacatatacacaccacacacaccacatacacacacgtatacacacatatatacacacatacaccatgcatacatacacaccacacatacatacagacacaccacacacacgtacacacaacacacaacacagacacacacgtacacacactacagacatgtatgcacacatacacacacaccacacatacatacacacagacacatatacactacacacaccattacatacacacgtacacatacaccacacacaccacacatacacacaccacacacacatacacacgccacacacacaccacaaaaaccgcacacacatacaaacatatacacactacaccacacatacacacacacaccacacaccacacacacacatacacacaccacacacaccacacatacacgcaccacacatacacacacgtagacacaccacacacaccacagaaacacacattaacacaccacatacacatatgtatgtgcatatacacacccacaccccacacacacatgtataaagatttagatatatataaaacatacgttatatatatgttgatgtaatatctaatatctatatatctaatatgtagtttattagctatctaatatctatgtcatatatatcaaatctttatatataaaaatatgtagaaatctttatacatatgttatatgtatataaagatttagatatataacatatgtaagttatatatatgttagtgtaatatctaatatatagtttattggctatctaatataatataaacagattatcaatattataagctattagaaaaatgcaagttaaggcagatgatatacctctttcacaccaactcacacaccaactacacacacacatacacacagacacacacgacacacaccatacacatgtacacacacaccacatatacacaaacgtacacacacaccacacacacatacacaccacacacacaacacacatacatacacatccacacaccacacatgtacacacaccacacacacacatacacaccacatacacatatgtatgcacacatacacaccaacaccacacagacaccacacatgcataaacatatagacatatacacaccacacaccatatgtacacatgtacacacacaccacatatacacacaacacacacaaatacacacaccacacacacaccacaaaaaccccacacacacaaacatatacaccccacacatacgcatatatacacacacacatacacaccacacacatacacaccacacacacaccacacatacacacacgtacacacaccacacacacaccacagacacacaccacacatacatacacatacacacaccacacacacgtacacacaccacacacacacagacacacatagacacaccacatacacacccacaccacacacacacaactcataccacacatacatacacaatagacacatacacaccacacacaccatacatacacacgtatacacacaccacatatacacacacgtacacacacaccacacacacccacatgcacacaccacacacacatacaaatatacaccacacacacatacaccacacacacggtgcacatacacacacatatacacacaccagacacacataccacatacacatcacacatatatgtatacatgcatacacatacacacacacatacacacactctcctcaaggcagtttatcctctgagaactttaaatttacaaaagacacatatgtccattactttgagaaggacaggaaagaacccactttcttttgcagcaacagcaagagggccctcccaaggctcctgctccctgtcataagtctccttgttgaggacattcacagggttcagaacccagggatcctgcatgggatggtgctttgctgattacttcacctctgatttctttccactttcagaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatgaattcttccttagacttacttttgtaaaaatgtccccacggtacttactccccactgatggaccagtggtttccagtcatgagcgttagactgacttgtttgtcttccattccattgttttgaaactcagtatgccgcccctgtcttgctgtcatgaaatcagcaagagaggatgacacatcaaataataactcggattccagcccacattggattcatcagcatttggaccaatagcccacagctgagaatgtggaatacctaaggataacaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtgaattcaggtagtaaatatgaaactagggtttgaaattgataatgctttcacaacatttgcagatgttttagaaggaaaaaagttccttcctaaaataatttctctacaattggaagattggaagattcagctagttaggagcccattttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtat

SEQ ID NO: 3 - DNA, 123 bp, Homo sapiens
cttgtggagcctcttacacccagtggagaagctcccaaccaagctctcttgaggatcttgaaggaaactgaattcaaaaagatcaaagtgctgggctccggtgcgttcggcacggtgtataag

SEQ ID NO: 4 - DNA, 99 bp, Homo sapiens
ggactctggatcccagaaggtgagaaagttaaaattcccgtcgctatcaaggaattaagagaagcaacatctccgaaagccaacaaggaaatcctcgat

SEQ ID NO: 5 - DNA, 186 bp, Homo sapiens
gaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaag

SEQ ID NO: 6 - DNA, 156 bp, Homo sapiens
ggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaa

SEQ ID NO: 7 - protein, 1255 aa, Homo sapiens [sequence omitted]

SEQ ID NO: 8 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 9 - protein, 1308 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 10 - protein, 10 aa, Homo sapiens
CTGPGLEGCP

SEQ ID NO: 11 - protein, 10 aa, Homo sapiens
GEAPNQALLR

SEQ ID NO: 12 - protein, 10 aa, Homo sapiens
PGNESLKAML

SEQ ID NO: 13 - protein, 10 aa, Homo sapiens
SVIITASSCH

SEQ ID NO: 14 - protein, 23 aa, Homo sapiens
MELAALCRWGLLLALLPPGAAST

SEQ ID NO: 15 - protein, 8 aa, Homo sapiens
MPRGSWKP

SEQ ID NO: 16 - protein, 10 aa, Homo sapiens
AYVMAGVGSP

SEQ ID NO: 17 - protein, 10 aa, Homo sapiens
ETEYHADGGK

SEQ ID NO: 18 - protein, 10 aa, Homo sapiens
TISNLFSNFA

SEQ ID NO: 19 - protein, 10 aa, Homo sapiens
LMCPQGAGKA

SEQ ID NO: 20 - protein, 10 aa, Homo sapiens
EILSGGVYIE

SEQ ID NO: 21 - protein, 10 aa, Homo sapiens
IVVKDNGRSC

SEQ ID NO: 22 - protein, 10 aa, Homo sapiens
GQFPMVPSGL

SEQ ID NO: 23 - protein, 10 aa, Homo sapiens
SKVPVTLAAV

SEQ ID NO: 24 - protein, 23 aa, Homo sapiens
NGPTSHDCIYYPWTGHSTLPQHA

SEQ ID NO: 25 - protein, 13 aa, Homo sapiens
IGSSIEDCIGLMD

SEQ ID NO: 26 - protein, 23 aa, Homo sapiens
NGPTSHDCIYYPWTGHSTLPQHA

SEQ ID NO: 27 - protein, 13 aa, Homo sapiens
IGSSIEDCIGLMD

(19) United States
(12) Patent Application Publication
(10) Pub. No.: US 2021/0346383 A1
(43) Pub. Date: Nov. 11, 2021

(54) COMPOUNDS, COMPOSITIONS AND METHODS FOR TREATING OR PREVENTING HER-DRIVEN CANCERS

(71) Applicant: Rain Therapeutics Inc., Newark, CA (US)

(72) Inventors: Avanish VELLANKI, Newark, CA (US); Vijaya TIRUNAGARU, Newark, CA (US)

(21) Appl. No.: 17/273,434

(22) PCT Filed: Sep. 4, 2019
(86) PCT No.: PCT/US2019/049507

§ 371 Date: Mar. 4, 2021

Related U.S. Application Data
(60) Provisional application No. 62/826,075, filed on Mar. 29, 2019.
(60) Provisional application No. 62/726,946, filed on Sep. 4, 2018.

(51) Int. Cl. A61K 31/519; A61P 35/04; A61P 35/00

(52) U.S. Cl. CPC A61K 31/519; A61P 35/00; A61P 35/04

(57) ABSTRACT

Disclosed herein are methods of treating or preventing HER-driven cancers. In some embodiments, the cancer comprises lung cancer or brain metastases.

### CROSS-REFERENCE TO RELATED APPLICATIONS

This application claims the benefit of U.S. Provisional Application No. 62/726,946, filed Sep. 4, 2018, and U.S. Provisional Application No. 62/826,075, filed Mar. 29, 2019, each of which is incorporated by reference herein in its entirety.

### SEQUENCE LISTING

The instant application contains a Sequence Listing which has been submitted in ASCII format via EFS-Web and is hereby incorporated by reference in its entirety. Said ASCII copy, created on Aug. 22, 2019, is named “RATH-006_SeqList.txt” and is 298 KB in size.

### BACKGROUND

The ErbB family of receptors is a subfamily of four closely related receptor tyrosine kinases: epidermal growth factor receptor or EGFR (ErbB-1; or HER1 in humans), HER2/c-neu (ErbB-2), HER3 (ErbB-3) and HER4 (ErbB-4).

EGFR is the cell-surface receptor for members of the epidermal growth factor family (EGF family) of extracellular protein ligands. Mutations that lead to EGFR overexpression (upregulation) or overactivity have been associated with a number of cancers, including squamous-cell carcinoma of the lung (80% of cases), anal cancers, glioblastoma (50% of cases), and epithelial tumors of the head and neck (80-100% of cases). These somatic mutations involving EGFR lead to its constant activation, which produces uncontrolled cell division. In a non-limiting example, in glioblastoma, a more or less specific mutation of EGFR, called EGFRvIII, is often observed. Mutations, amplifications, or misregulations of EGFR or family members are implicated in about 30% of all epithelial cancers.

The identification of EGFR as an oncogene has led to the development of anti cancer therapeutics directed against EGFR (called “EGFR tyrosine kinase inhibitors”), including gefitinib, erlotinib, afatinib, osimertinib, and icotinib for lung cancer. Cetuximab, panitumumab, necitumumab, zalutumumab, nimotuzumab and matuzumab are examples of monoclonal antibody EGFR inhibitors. Gefitinib, erlotinib, afatinib, dacomitinib, osimertinib, and lapatinib (mixed EGFR and ErbB-2 inhibitor) are examples of small molecule EGFR kinase inhibitors.

Unfortunately, many patients develop resistance of the existent EGFR inhibitors. Non-limiting sources of resistance are the EGFR T790M Mutation, HER2 and MET oncogenes, transformation to small cell lung cancer (SCLC), epithelial to mesenchymal transition (EMT), and fusions including those involving BRAF, NTRK1, RET, ALK, and/or ROS1. Options to combat resistance are limited, with only osimertinib being approved to treat EGFR T790M. While in frame deletions in exon 19 of EGFR and the L858R substitution in exon 21 of EGFR are sensitive to EGFR inhibitors (such as erlotinib, gefitinib, and afatinib), other mutations, such as in frame insertions in EGFR exon 20 and various other point mutations demonstrate intrinsic resistance to these inhibitors. Other rare mutations in EGFR, such as G719X (exon 18) and L861Q (exon 21), have variable response to EGFR inhibitors. A particular EGFR mutation, C797S, is resistant to all currently approved TKI inhibitors.

Similarly, somatic mutations of ErbB-2 (HER2) are found in a wide range of cancers, such as, for example, lung adenocarcinoma, and gastric, colorectal, and breast carcinomas. There is thus a need in the art to identify compounds and methods that can be used to treat or prevent HER-driven cancers and HER-driven drug-resistant cancers. In some embodiments, the compounds and methods can be used to treat or prevent EGFR-driven and/or HER2-driven cancers. The present application addresses and meets these needs.

### SUMMARY

In one aspect, provided herein is a method of treating or preventing a HER-driven cancer in a subject in need thereof, the method comprising:

(a) providing tumor cells of the subject;

(b) detecting presence or absence of an EGFR mutation in the provided tumor cells;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutation and the ErbB-2 mutation is detected; and

(d) administering a therapeutically effective amount of Compound A, or a salt or a solvate thereof.

In one aspect, provided herein is a method of treating or preventing a HER-driven cancer in a subject in need thereof, the method comprising:

(a) providing tumor cells of the subject;

(b) detecting presence or absence of an EGFR mutation in the provided tumor cells;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A) or its active metabolite (RN-4000E; also known as “(E)-N-(4-((3-bromo-4-chlorophenyl)amino)pyrido[3,4-d]pyrimidin-6-yl)-4-(dimethylamino)but-2-enamide”; also referred to herein as “(2E)-N-[4-(3-bromo-4-chloroanilino)pyrido[3,4-d]pyrimidin-6-yl]-4-(dimethylamino)-2-butenamide”); also referred to herein as Compound B), or a combination thereof, if at least one of the EGFR mutation and the ErbB-2 mutation is detected; and

(d) administering a therapeutically effective amount of Compound A or its active metabolite (RN-4000E; also known as “(E)-N-(4-((3-bromo-4-chlorophenyl)amino)pyrido[3,4-d]pyrimidin-6-yl)-4-(dimethylamino)but-2-enamide”; also referred to herein as “(2E)-N-[4-(3-bromo-4-chloroanilino)pyrido[3,4-d]pyrimidin-6-yl]-4-(dimethylamino)-2-butenamide”); also referred to herein as Compound B), or a combination thereof, or a salt or a solvate thereof.

In one aspect, provided herein is a method of treating or preventing a HER-driven cancer in a subject in need thereof, the method comprising:

(a) providing tumor cells of the subject;

(b) detecting presence or absence of a mutation in the provided tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutation and the ErbB-2 mutation is detected; and

(d) administering a therapeutically effective amount of Compound A, or a salt or a solvate thereof.

In another aspect, provided herein is a method of treating a HER-driven cancer in a subject with cancer, where at least one of an EGFR mutation or a ErbB-2 mutation is detected in tumor cells of the subject, wherein the method comprises administering a therapeutically effective amount of Compound A, or a salt or a solvate thereof; and wherein the EGFR mutation is selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; and the ErbB-2 mutation is selected from the group consisting of: D769H, D769Y, R896C, V777L, and V777_G778insCG.

In yet another aspect, provided herein is a method of predicting the responsiveness of a subject with a HER-driven cancer to treatment with Compound A, wherein the method comprises:

(a) providing tumor cells of the subject;

(b) detecting presence or absence of an EGFR mutation in the provided tumor cells of the subject;

(c) predicting the subject as being likely to be responsive to a treatment with Compound A if at least one of the EGFR mutation and the ErbB-2 mutation is detected in the tumor cell of the subject.

In yet another aspect, provided herein is a method of predicting the responsiveness of a subject with a HER-driven cancer to treatment with Compound A, wherein the method comprises:

(a) providing tumor cells of the subject;

(b) detecting presence or absence of a mutation in the provided tumor cells of the subject, wherein the mutation comprises an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation comprises an ErbB-2 mutation selected from the group consisting of: D769H, D769Y, R896C, V777L, and V777_G778insCG;

(c) predicting the subject as being likely to be responsive to a treatment with Compound A if at least one of the EGFR mutation and the ErbB-2 mutation is detected in the tumor cell of the subject.

In still another aspect, provided herein is a method of predicting the responsiveness of a subject with a HER-driven to treatment with Compound A, wherein the method comprises detecting presence or absence of a mutation in a sample of tumor cells from the subject; wherein the subject is likely to be responsive to the treatment with Compound A if the mutation is detected in the sample of tumor cells from the subject; and wherein the mutation comprises an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation comprises an ErbB-2 mutation selected from the group consisting of: D769H, D769Y, R896C, V777L, and V777_G778insCG.

In a further aspect, provided herein is a method of identifying a subject with cancer who is likely to be responsive to treatment with Compound A, wherein the method comprises:

(a) providing tumor cells of the subject;

(b) detecting presence or absence of an EGFR mutation in the provided tumor cells of the subject;

(c) identifying the subject as being likely to be responsive to treatment with Compound A if at least one of the EGFR mutation and the ErbB-2 mutation is detected in the provided tumor cell.

In a further aspect, provided herein is a method of identifying a subject with cancer who is likely to be responsive to treatment with Compound A, wherein the method comprises:

(a) providing tumor cells of the subject;

(b) detecting presence or absence of a mutation in the provided tumor cells of the subject, wherein the mutation comprises an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation comprises an ErbB-2 mutation selected from the group consisting of: D769H, D769Y, R896C, V777L, and V777_G778insCG;

(c) identifying the subject as being likely to be responsive to treatment with Compound A if at least one of the EGFR mutation and the ErbB-2 mutation is detected in the provided tumor cell.

In yet a further aspect, provided herein is a use of Compound A in the manufacture of a medicament for the treatment of a HER-driven cancer, wherein the HER-driven cancer comprises an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation comprises an ErbB-2 mutation selected from the group consisting of: D769H, D769Y, R896C, V777L, and V777_G778insCG.

In one aspect, provided herein is a method of treating or preventing a HER-driven cancer in a subject in need thereof, the method comprising

a) providing tumor cells of the subject;

(b) detecting presence or absence of a mutation in the provided tumor cells, wherein the mutation comprises an EGFR C797S mutation;

(c) predicting the subject as being likely to be responsive to treatment by Compound A. if the EGFR C797S mutation is detected; and

(d) administering a therapeutically effective amount of Compound A, or a salt or a solvate thereof.

In another aspect, provided herein is a method of treating a HER-driven cancer in a subject with cancer, where an EGFR C797S mutation is detected in tumor cells of the subject, wherein the method comprises administering a therapeutically effective amount of Compound A, or a salt or a solvate thereof.

In yet another aspect, provided herein is a method of predicting the responsiveness of a subject with a HER-driven cancer to treatment with Compound A, wherein the method comprises:

(a) providing tumor cells of the subject;

(b) detecting presence or absence of a mutation in the provided tumor cells of the subject, wherein the mutation comprises an EGFR C797S mutation;

(c) predicting the subject as being likely to be responsive to a treatment with Compound A if the EGFR C797S mutation is detected in the tumor cell of the subject.

In still a further aspect, provided herein ia a method of predicting the responsiveness of a subject with a HER-driven cancer to treatment with Compound A, wherein the method comprises detecting presence or absence of a mutation in a sample of tumor cells from the subject; wherein the subject is likely to be responsive to the treatment with Compound A if the mutation is detected in the sample of tumor cells from the subject; and wherein the mutation comprises an EGFR C797S mutation.

In another aspect, provided herein ia a method of identifying a subject with cancer who is likely to be responsive to treatment with Compound A, wherein the method comprises:

(a) providing tumor cells of the subject;

(b) detecting presence or absence of a mutation in the provided tumor cells of the subject, wherein the mutation comprises an EGFR C797S mutation;

(c) identifying the subject as being likely to be responsive to treatment with Compound A the EGFR C797S mutation is detected in the provided tumor cells.

In yet a further aspect, provided herein is a use of Compound A in the manufacture of a medicament for the treatment of a HER-driven cancer, wherein the HER-driven cancer comprises an EGFR C797S mutation.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 is a schematic illustration of the currently accepted mechanism of activation of Compound A (tarloxotinib or TRLX, indicated as RN-4000) to Compound B (tarloxotinib-TKI or TRLX-TKI, indicated as RN-4000E), and subsequent inhibition of EGFR by Compound B.

FIGS. 2A and 2B show % activity vs. concentration curves and calculated IC₅₀ values for (a) Tarlox-TKI and (b) Saturosporine for wild-type EGFR.

FIGS. 3A and 3B show % activity vs. concentration curves and calculated IC₅₀ values for (a) Tarlox-TKI and (b) Saturosporine for EGFR mutation A763_Y764insFHEA.

FIGS. 4A and 4B show % activity vs. concentration curves and calculated IC₅₀ values for (a) Tarlox-TKI and (b) Saturosporine for EGFR mutation C797S/L858R.

FIGS. 5A and 5B show % activity vs. concentration curves and calculated IC₅₀ values for (a) Tarlox-TKI and (b) Saturosporine for EGFR mutation d746-750.

FIGS. 6A and 6B show % % activity vs. concentration curves and calculated IC₅₀ values for (a) Tarlox-TKI and (b) Saturosporine for EGFR mutation d746-750/C797A.

FIGS. 7A and 7B show % activity vs. concentration curves and calculated IC₅₀ values for (a) Tarlox-TKI and (b) Saturosporine for EGFR mutation d746-750/C797S.

FIGS. 8A and 8B show % activity vs. concentration curves and calculated IC₅₀ values for (a) Tarlox-TKI and (b) Saturosporine for EGFR mutation d746-750/T790M/C797S.

FIGS. 9A and 9B show % activity vs. concentration curves and calculated IC₅₀ values for (a) Tarlox-TKI and (b) Saturosporine for EGFR mutation D770GY.

FIGS. 10A and 10B show % activity vs. concentration curves and calculated IC₅₀ values for (a) Tarlox-TKI and (b) Saturosporine for EGFR insertion mutation D770_N771 insNPG.

FIGS. 11A and 11B show % activity vs. concentration curves and calculated IC₅₀ values for (a) Tarlox-TKI and (b) Saturosporine for EGFR mutation G719C.

FIGS. 12A and 12B show % activity vs. concentration curves and calculated IC₅₀ values for (a) Tarlox-TKI and (b) Saturosporine for EGFR mutation G719S.

FIGS. 13A and 13B show % activity vs. concentration curves and calculated IC₅₀ values for (a) Tarlox-TKI and (b) Saturosporine for EGFR mutation L747S.

FIGS. 14A and 14B show % activity vs. concentration curves and calculated IC₅₀ values for (a) Tarlox-TKI and (b) Saturosporine for EGFR mutation L858R.

FIGS. 15A and 15B show % activity vs. concentration curves and calculated IC₅₀ values for (a) Tarlox-TKI and (b) Saturosporine for double EGFR mutation L858R/T790M.

FIGS. 16A and 16B show % activity vs. concentration curves and calculated IC₅₀ values for (a) Tarlox-TKI and (b) Saturosporine for EGFR mutation L861Q.

FIGS. 17A and 17B show % activity vs. concentration curves and calculated IC₅₀ values for (a) Tarlox-TKI and (b) Saturosporine for triple EGFR mutation T790M/C797S/L858R.

FIGS. 18A and 18B show % activity vs. concentration curves and calculated IC₅₀ values for (a) Tarlox-TKI and (b) Saturosporine for ErbB-2 mutation D769H.

FIGS. 19A and 19B show % activity vs. concentration curves and calculated IC₅₀ values for (a) Tarlox-TKI and (b) Saturosporine for ErbB-2 mutation D769Y.

FIGS. 20A and 20B show % activity vs. concentration curves and calculated IC₅₀ values for (a) Tarlox-TKI and (b) Saturosporine for ErbB-2 mutation R896C.

FIGS. 21A and 21B show % activity vs. concentration curves and calculated IC₅₀ values for (a) Tarlox-TKI and (b) Saturosporine for ErbB-2 mutation V777L.

FIGS. 22A and 22B show % activity vs. concentration curves and calculated IC₅₀ values for (a) Tarlox-TKI and (b) Saturosporine for ErbB-2 mutation V777_G778insCG.

FIGS. 23A and 23B show % activity vs. concentration curves and calculated IC₅₀ values for (a) Tarlox-TKI and (b) Saturosporine for wild-type ErbB-2 (HER2).

FIGS. 24A and 24B show % activity vs. concentration curves and calculated IC₅₀ values for (a) Tarlox-TKI and (b) Saturosporine for wild-type ErbB-4 (HER4).

FIGS. 25A and 25B show (a) plasma concentration vs. time and (b) brain concertation vs. time curves for Tarloxotinib in mice.

FIGS. 26A and 26B show (a) plasma concentration vs. time and (b) brain concertation vs. time curves for Tarlox-TKI in mice.

FIGS. 27A and 27B show the biochemical IC50s of tarloxotinib-E for various (A) EGFR and (B) HER2/ERBB2 mutation kinases in radiometric kinase assays at respective ATP Km.

FIGS. 28A, 28B, 28C, 28D, and 28E show the growth inhibition of Ba/F3 cell lines expressing various EGFR exon 20 insertion mutations, including (A) H773insNPH, (B) D770insSVD, (C) V769insASV, (D) A763insFQEA, and (E) H773insH.

FIGS. 29A, 29B, 29C, and 29D show the growth inhibition of Ba/F3 cell lines expressing EGFR C797S double and triple mutations with dell9 and L585R±T790M, including (A) dell9/C797S, (B) dell9/C797S/T790M, (C) L858R/C797S, and (D) L858R/C797S/T790M.

FIGS. 30A, 30B, and 30C show the growth inhibition of Ba/F3 cell lines expressing various HER2 mutations, including (A) A775_G776insYVMA, (B) G776delinsVC, and (C) P780_Y781insGSP.

FIGS. 31A and 31B show the growth inhibition of Ba/F3 cell lines expressing HER2 mutations with C805S, including (A) A775_G776insYVMA C805S and (B) G776delinsVC C805S.

FIGS. 32A and 32B show the growth inhibition of Ba/F3 cell lines expressing various tertiary osimertinib resistance mutations, including Tarloxotinib-E with (A) dell9+L718V, del 19+G724S, dell9+L792F, dell9+C797S, and dell9+L792H, and (B) L858R+L718Q, L858R+L718V, L858R+L792F, L858R+C797G, and L858R+L792H.

FIG. 33 shows intracranial tumor growth when treated with YH25448 or osimertinib from 13-day post-implantation.

FIGS. 34A and 34B show Tarloxotinib in comparison to erlotinib in HCC827 (Del 19 EGFR, FIG. 34A) and PC9 (Del 19/WT EGFR, FIG. 34B).

FIG. 35 shows Tarloxotinib activity against the HER2-positive NCI-N87 gastric tumor xenograft.

FIG. 36 shows Tarloxotinib activity in EGFR exon 20 insertion (A767_V769dupASV) patient-derived cell line CUTO-14 xenograft model.

FIG. 37 shows Tarloxotinib synergistic activity with VEGFR2 inhibitor in H1781 (HER2 G776Ins V G/C) xenograft model.

### DETAILED DESCRIPTION OF THE INVENTION

The present disclosure relates in part to the discovery that certain nitromethylaryl quaternary ammonium salts (also referred to as NMQ prodrugs) can be used as small molecule EGFR inhibitors to treat or prevent certain HER-driven cancers. In some embodiments, the cancer is a HER-driven drug-resistant cancer. In some embodiments, a small molecule EGFR inhibitor (RN-4000; also known as “(E)-4-((4-((3-bromo-4-chlorophenyl)amino)pyrido[3,4-d]pyrimidin-6-yl)amino)-N,N-dimethyl-N-((l-methyl-4-nitro-1H-imidazol-5-yl)methyl)-4-oxobut-2-en-1-aminium salt (bromide)”; also referred to herein as “(2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide”; also referred to herein as Compound A, RN-4000, TRLX, or tarloxotinib) and/or its active metabolite (RN-4000E; also known as “(E)-N-(4-((3-bromo-4-chlorophenyl)amino)pyrido[3,4-d]pyrimidin-6-yl)-4-(dimethylamino)but-2-enamide”; also referred to herein as “(2E)-N-[4-(3-bromo-4-chloroanilino)pyrido[3,4-d]pyrimidin-6-yl]-4-(dimethylamino)-2-butenamide”); also referred to herein as Compound B) are used to treat or prevent certain HER-driven cancers.

In some embodiments, the cancer is EGFR-driven.

It will be understood by one of ordinary skill in the art that Compound A may exist as a cation or salt, for example, a bromide salt, as depicted below.

Structures of Compound A and Compound B are provided below:

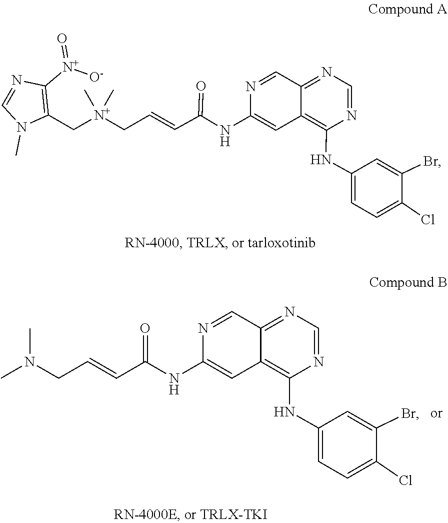 [SMILES: Cn1cnc([N+](=O)[O-])c1C[N+](C)(C)C/C=C/C(=O)Nc1cc2c(Nc3ccc(Cl)c(Br)c3)ncnc2cn1 ; CN(C)C/C=C/C(=O)Nc1cc2c(Nc3ccc(Cl)c(Br)c3)ncnc2cn1]

The disclosure further contemplates the use of other NMQ prodrugs and/or small molecule EGFR inhibitors, including any other small molecule analogues of Compound A and/or Compound B, to treat or prevent certain HER-driven cancers. In some embodiments, the cancer is a HER-driven drug-resistant cancer. Such NMQ prodrugs and/or small molecule EGFR inhibitors include, but are not limited to those disclosed in WO2010104406, WO2011028135, US20120077811, and US20120202832, each of which is incorporated herein by reference in its entirety. For example, the disclosure contemplates NMQ prodrugs of quaternary nitrogen salt compounds of Formula F

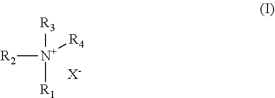

where:

X is any negatively charged counterion;

R₁ is a group of the formula —(CH₂)ₙTr, where Tr is an aromatic nitroheterocycle or an aromatic nitrocarbocycle and —(CH₂)ₙTr acts as a reductively-activated fragmenting trigger (“reductive trigger”); and

n is an integer from 0 to 6;

R₂, R₃ and R₄ are each independently an aliphatic or an aromatic group of a tertiary amine kinase inhibitor (R₂)(R3)(R₄)N, or two of R₂, R₃, and R₄ may form an aliphatic or aromatic heterocyclic amine ring of a kinase inhibitor, or one of R₂, R₃ and R₄ may be absent and two of R₂, R₃ and R₄ form an aromatic heterocyclic amine ring of a kinase inhibitor.

In some embodiments, the compounds are of Formula II:

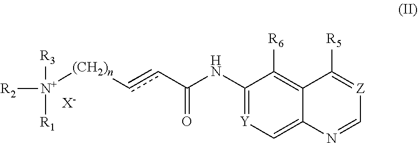

where:

X is any negatively charged counterion;

Y is N or C—R₇, where R₇ is selected from H, C₁-C₆ alkyl, C₁-C₆ alkoxy, and a group of the one of the following Formulas IIIa, IIIb, and IIIc:

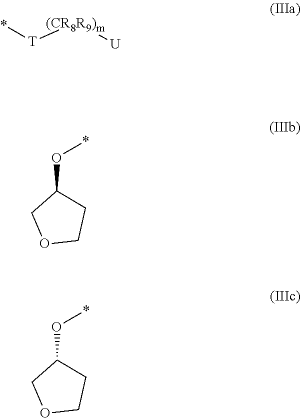

where * is the point of attachment;

T is selected from O, NH, N(C₁-C₆ alkyl), and a direct link;

m is an integer from 0 to 6;

U is selected from OR₁₀, CF₃, OCF₃, CN, NR₁₁R₁₂, pyrrolidinyl, piperidinyl, piperazinyl, N1-methylpiperazinyl, morpholinyl, CON(R₁₃)(R₁₄), SO₂N(R₁₅)(R₁₆), N(R₁₇)COR₁₈, N(R₁₉)SO₂R₂₀, COR₂₁, SOR₂₂, SO₂R₂₃, and COOR₂₄; and

R₈, R₉, R₁₀, R₁₁, R₁₂, R₁₃, R₁₄ R₁₅, R₁₆, R₁₇, R₁₈, R₁₉, R₂₀, R₂₁, R₂₂, R₂₃, R₂₄ are independently selected from H and C₁-C₆ alkyl;

Z is N or C—CN;

n is an integer from 0 to 6;

R₁ is a group of the formula (CH₂)ₙTr where Tr is an aromatic nitroheterocycle or aromatic nitrocarbocycle and —(CH₂)ₙTr acts as a reductive trigger;

and n is an integer from 0 to 6;

R₂ and R₃ are independently selected from C₁-C₆ alkyl, cyclopropyl, cyclobutyl, cyclopentyl, cyclohexyl, CH₂CH₂OH, and CH₂CH₂O(C₁-C₆ alkyl); or R₂ and R₃ may together form a non-aromatic carbocyclic ring or non-aromatic heterocyclic ring containing at least one heteroatom;

R₅ is selected from an aniline, an indole, an indoline, an amine, an aminoindole, and an aminoindazole, each of which may be optionally substituted with one or more substituents selected from H, C₁-C₆ alkyl, C₂-C₆ alkenyl, C2-G5 alkynyl, C₁-C₆ alkoxy, F, Cl, Br, I, CN, CH₂F, CHF₂, CF₃, OH, NH₂, NO₂, NH(C₁-C₆ alkyl), N(C₁-C₆ alkyl)₂, CONH₂, CO(C₁-C₆ alkyl), SO₂NH₂, and SO₂(C₁-C₆ alkyl); and

R₆ is selected from H, C₁-C₆ alkyl, C₁-C₆ alkoxy, NH(C₁-C₆ alkyl), N(C₁-C₆ alkyl)₂, and a group of the following Formula IV:

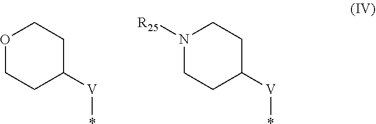

where

* is the point of attachment;

V is selected from (CH₂)ₖ, O, NH, and N(C₁-C₆ alkyl);

k is an integer from 0 to 6, and

R₂₅ is selected from H and C₁-C₆ alkyl.

In some embodiments, X is selected from halide (e.g., fluoride, chloride, bromide, iodide), methanesulfonate, trifluoromethanesulfonate, acetate, trifluoroacetate, tosylate, lactate, citrate, and formate. In some embodiments, X is a halide. In some embodiments, X is selected from fluoride, chloride, bromide, and iodide.

In some embodiments, R₁ is a group of one of the following Formulas Va-Vq:

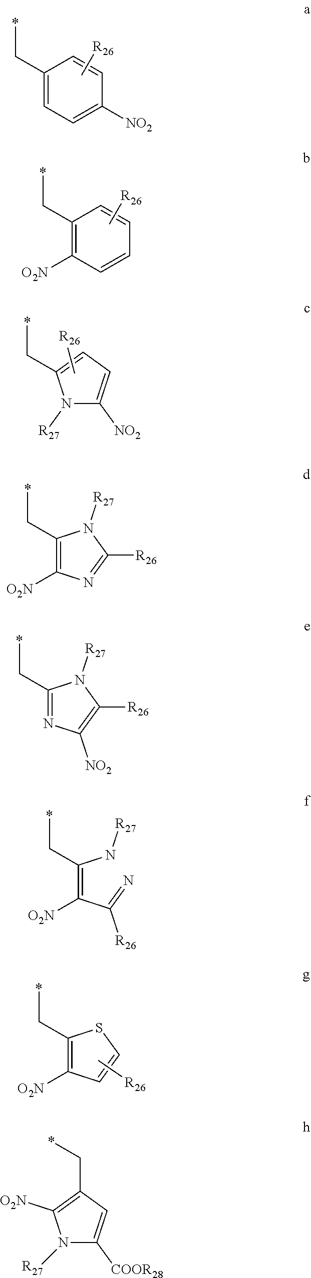

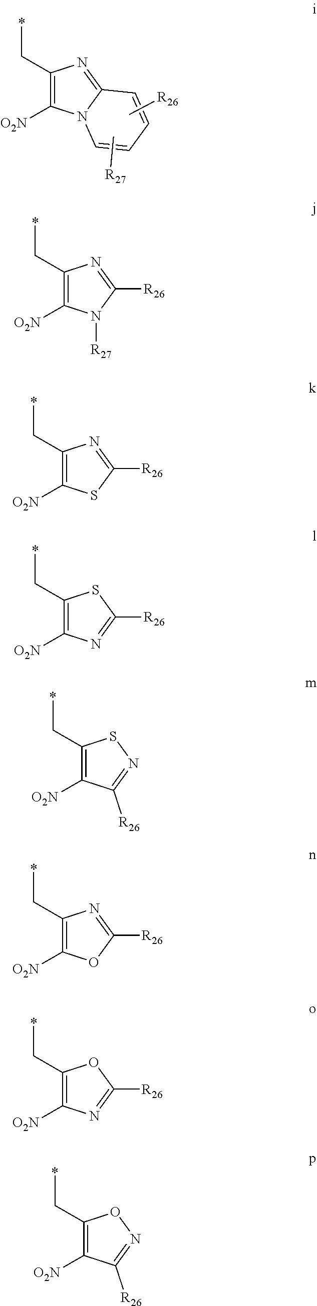

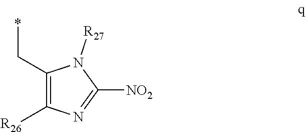

where:

* is the point of attachment to the quaternary nitrogen of a compound of Formula II;

R₂₆ is selected from H, C₁-C₆ alkyl, C₁-C₆ alkoxy, C₂-C₆ alkenyl, C₂-C₆ alkynyl, CF₃, CF₃, F, Cl, Br, I, NO₂, CN, COOH, COO(C₁-C₆ alkyl), CONH₂, CONH(C₁-C₆ alkyl), CON(C₁-C₆ alkyl)₂, CO(C₁-C₆ alkyl), SO₂NH₂, SO₂NH(C₁-C₆ alkyl), SO₂N(C₁-C₆ alkyl)₂, SO₂(C₁-C₆ alkyl), and a group of Formula IIIa, as defined above, where * is the point of attachment to a group of Formula V;

R₂₇ is selected from H, C₁-C₆ alkyl, and a group of Formula IIIa, as defined above, where * is the point of attachment to a group of Formula V; and

R₂₈ is selected from H and C₁-C₆ alkyl.

In some embodiments, R₁ is a group of one of the following Formulas Vr-Vae:

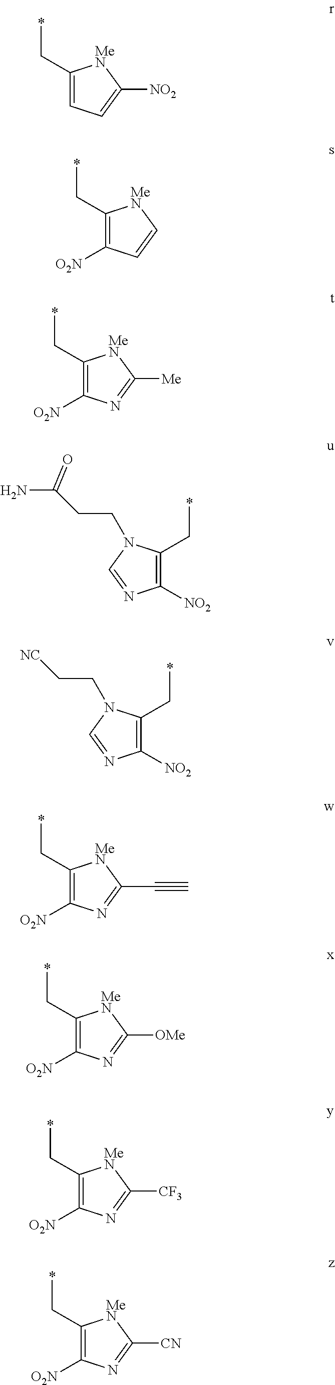

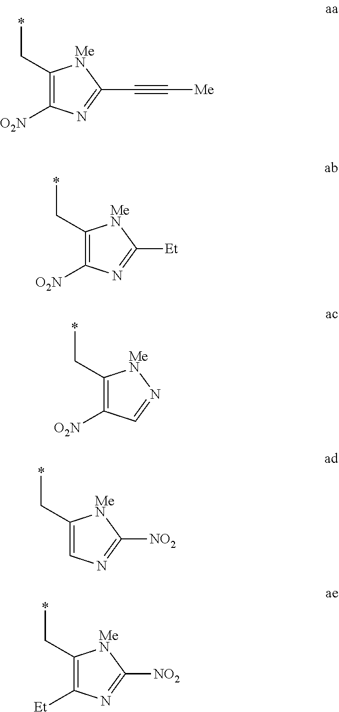 [SMILES: *Cc1c([N+](=O)[O-])nc(C#CC)n1C ; *Cc1c([N+](=O)[O-])nc(CC)n1C ; *Cc1c([N+](=O)[O-])cnn1C ; *Cc1cnc([N+](=O)[O-])n1C ; *Cc1c(CC)nc([N+](=O)[O-])n1C]

In some embodiments, R₁ is a group of Formula Vc, where R₂₆ is H and R₂₇ is CH₃.

In some embodiments, R₁ is a group of Formula Vd, where R₂₆ is selected from H, C₁-C₆ alkyl (e.g., methyl), C₁-C₆ alkoxy (e.g., OCH₃), C₂-C₆ alkynyl (e.g., ethynyl), CONH₂, CONHMe, CF₃, OCF₃, Br, NO₂, and CN, and R₂₇ is selected from CH₃, CH₂CH₂CONH₂, and CH₂CH₂CN.

In some embodiments, R₁ is a group of Formula Vd,

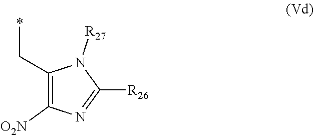

where * is a point of attachment, R₂₆ is selected from H and C₁-C₃ alkyl, and R₂₇ is selected from H and C₁-C₆ alkyl. In some embodiments, R₂₆ is selected from H, methyl, ethyl, trifluoromethyl, —CN, —CONH₂, and propyn-1-yl, and R₂₇ is C₁-C₆ alkyl.

In some embodiments, R₂₆ is H and R₂₇ is C₁-C₃ alkyl (e.g., methyl).

In some embodiments, R₁ is a group of Formula Vd, where R₂₆ is 1-propynyl and R₂₇ is CH₃.

In some embodiments, R₁ is a group of Formula Vq, where R₂₆ is selected from H, C₁-C₆ alkyl (e.g., methyl or ethyl) and C₁-C₆ alkoxy (e.g., OCH₃), and R₂₇ is CH₃.

In some embodiments. R₁ is a group of any one of Formulas Vd⁽¹⁾-Vd⁽⁷⁾:

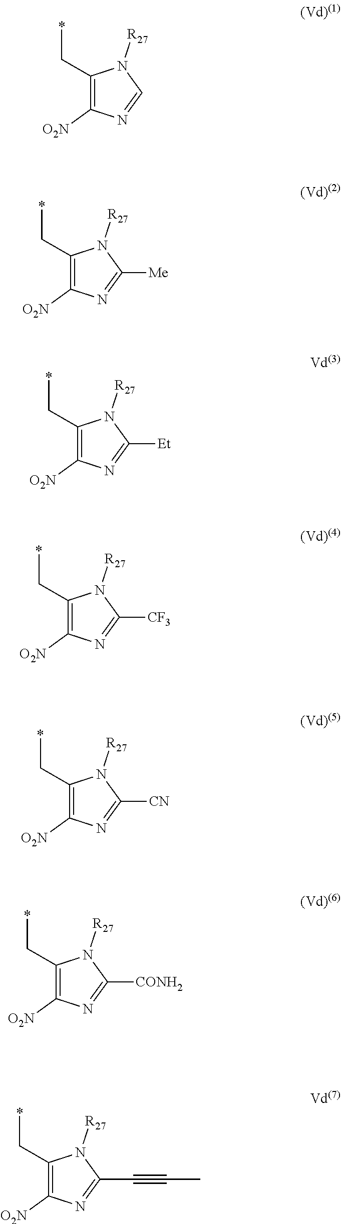

In some embodiments, R₂₇ is selected from methyl, ethyl and propyl. In some embodiments R₂₇ is methyl.

In some embodiments, R₂ and R₃ form a ring selected from pyrrolidinium, piperidinium, piperazinium, N1-methylpiperazinium, and morpholinium.

In some embodiments, R₅ is a group of one of the following Formulas VIa-VIg:

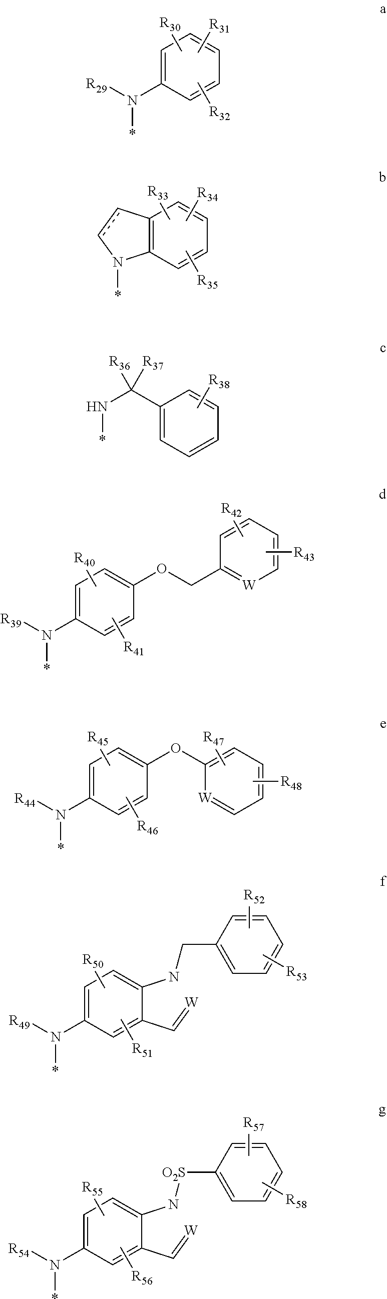

where:

* is the point of attachment;

R₂₉, R₃₆, R₃₇, R₃₉, R₄₄, R₄₉ and R₅₄, are independently selected from H and C₁-C₆ alkyl;

R₃₀, R₃₁, R₃₂, R₃₃, R₃₄, R₃₅, R₃₈, R₄₀, R₄₁, R₄₂, R₄₃, R₄₅, R₄₆, R₄₇, R₄₈, R₅₀, R₅₁, R₅₂, R₅₃, R₅₅, R₅₆, R₅₇ and R₅₈ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), N(C₁-C₆ alkyl)₂, CONH₂, CO(C₁-C₆ alkyl), SO₂NH₂, and SO₂(C₁-C₆ alkyl); and

W is N or C—H.

In some embodiments, Y is N, Z is N or C—CN; and

R₁ is selected from the following:

(a) a group of Formula Vc, where R₂₆ is H and R₂₇ is CH₃;

(b) a group of Formula Vd, where (i) R₂₆ is selected from H, C₁-C₆ alkyl (e.g., methyl), C₁-C₆ alkoxy (e.g., OCH₃), C₂-C₆ alkynyl (e.g., ethynyl), CF₃, OCF₃, Br, NO₂, and CN, and R₂₇ is selected from CH₃, CH₂CH₂CONH₂, and CH₂CH₂CN; or (ii) R₂₆ is 1-propynyl and R₂₇ is CH₃;

(c) a group of Formula Vf, where R₂₆ is H and R₂₇ is CH₃; and

(d) a group of Formula Vq, where R₂₆ is selected from H, C₁-C₆ alkyl (e.g., methyl and ethyl) and C₁-C₆ alkoxy (e.g., OCH₃), and R₂₇ is CH₃; where

R₂ and R₃ are independently selected from C₁-C₆ alkyl, or together form a ring selected from pyrrolidinium, piperidinium, piperazinium, N1-methylpiperazinium, and morpholinium; and

R₅ is selected from the following:

(a) a group of Formula Via, where * is the point of attachment, R₂₉ is H, and R₃₀, R₃₁, R₃₂ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂;

(b) a group of Formula VId, where * is the point of attachment, R₃₉ is H, and R₄₀ and R₄₁ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂; R₄₂ and R₄₃ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂; and W is N or C—H; and

(c) a group of Formula VIf, where * is the point of attachment, R₄₉ is H, and R₅₀ and R₅₁ are independently selected from H and F; R₅₂ and R₅₃ are independently selected from H, C₁-C₆ alkyl, F, Cl, Br, I, CH₂F, CHF₂, and CF₃; W is N or C—H; R₆ is H; X is any negatively charged counterion; and n=1 or 2.

In some embodiments, Y is C—H or C—(C₁-C₆ alkoxy), Z is N or C—CN; and

R₁ is selected from the following:

(a) a group of Formula Vc, where R₂₆ is H, and R₂₇ is CH₃;

(b) a group of Formula Vd, where R₂₆ is selected from H, C₁-C₆ alkyl, C₁-C₆ alkoxy, C₂-C₆ alkynyl, CF₃, OCF₃, Br, NO₂, and CN, and R₂₇ is selected from CH₃, CH₂CH₂CONH₂, and CH₂CH₂CN; or R₂₆ is 1-propynyl and R₂₇ is CH₃;

(c) a group of Formula Vf, where R₂₆ is H and R₂₇ is CH₃; and

(d) a group of Formula Vq, where R₂₆ is selected from H, C₁-C₆ alkyl (e.g., methyl and ethyl), and C₁-C₆ alkoxy (e.g., OCH₃), and R₂₇ is CH₃;

R₂ and R₃ are independently selected from C₁-C₆ alkyl, or together form a ring selected from pyrrolidinium, piperidinium, piperazinium, N1-methylpiperazinium, and morpholinium;

R₅ is selected from the following:

(a) a group of Formula Via, where * is the point of attachment; R₂₉ is H; and R₃₀, R₃₁, R₃₂ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂;

(b) a group of Formula VId, where * is the point of attachment; R₃₉ is H; and R₄₀ and R₄₁ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂; R₄₂ and R₄₃ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂; and W is N or C—H; and

(c) a group of Formula VIf, where * is the point of attachment; R₄₉ is H; and R₅₀ and R₅₁ are independently selected from H and F; R₅₂ and R₅₃ are independently selected from H, C₁-C₆ alkyl, F, Cl, Br, I, CH₂F, CHF₂, and CF₃; and W is N or C—H;

R₆ is H;

X is any negatively charged counterion; and

n=1 or 2.

In some embodiments, Y is C—R₇, where R₇ is a group of Formula IIIb; Z is N or C—CN;

R₁ is selected from the following:

(a) a group of Formula Vc, where R₂₆ is H, and R₂₇ is CH₃;

(b) a group of Formula Vd, where R₂₆ is selected from H, C₁-C₆ alkyl, C₁-C₆ alkoxy, C₂-C₆ alkynyl, CF₃, OCF₃, Br, NO₂, and CN, and R₂₇ is selected from CH₃, CH₂CH₂CONH₂, and CH₂CH₂CN; or R₂₆ is 1-propynyl; and R₂₇ is CH₃;

(c) a group of Formula Vf, where R₂₆ is H and R₂₇ is CH₃; and

(d) a group of Formula Vq, where R₂₆ is selected from H, C₁-C₆ alkyl (e.g., methyl and ethyl) and C₁-C₆ alkoxy (e.g., OCH₃); and R₂₇ is CH₃;

R₂ and R₃ are independently selected from C₁-C₆ alkyl, or together form a ring selected from pyrrolidinium, piperidinium, piperazinium, N1-methylpiperazinium, and morpholinium;

R₅ is selected from the following:

(a) a group of Formula Via, where * is the point of attachment to a compound of Formula II; R₂₉ is H; and R₃₀, R₃₁, R₃₂ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂;

(b) a group of Formula VId, where * is the point of attachment to a compound of Formula II; R₃₉ is H; and R₄₀ and R₄₁ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂; R₄₂ and R₄₃ are independently selected from H, C₁-C₆ alkyl, C₂-C₆ alkenyl, C₂-C₆ alkynyl, C₁-C₆ alkoxy, F, Cl, Br, I, CN, CH₂F, CHF₂, CF₃, OH, NH₂, NO₂, NH(C₁-C₆ alkyl), and N(C₁-C₆ alkyl)₂; and W is N or C—H; and

(c) a group of Formula VIf, where * is the point of attachment to a compound of Formula II; R₄₉ is H; and R₅₀ and R₅₁ are independently selected from H or F; R₅₂ and R₅₃ are independently selected from H, C₁-C₆ alkyl, F, Cl, Br, I, CH₂F, CHF₂, and CF₃; and W is N or C—H;

R₆ is H;

X is any negatively charged counterion; and

n=1 or 2.

In some embodiments, the compounds are of Formula VII:

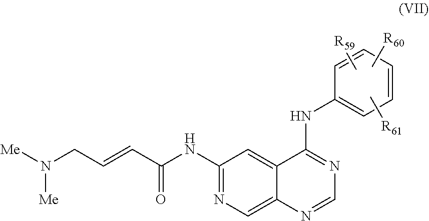

wherein either:
(1) R₅₉ is H, and
   (a) R₆₀ is (3-chlorobenzyl)oxy- and R₆₁ is chloro;
   (b) R₆₀ and R₆₁, together with the carbon atoms to which they are attached, form 1-(3-fluorobenzyl)-1H-pyrazole;
   (c) R₆₀ is 2-pyridinylmethoxy and R₆₁ is chloro;
   (d) R₆₀ and R₆₁ are both chloro;
   (e) R₆₀ is chloro and R₆₁ is bromo;
   (f) R₆₀ and R₆₁ are both bromo;
   (g) R₆₀ is fluoro and R₆₁ is ethynyl;
   (h) R₆₀ is chloro and R₆₁ is ethynyl;
   (i) R₆₀ is bromo and R₆₁ is ethynyl;
   (j) other than when R₆₀ is in the 3-position in combination with R₆₁ in the 4-position, R₆₀ is bromo and R₆₁ is fluoro;
   (k) R₆₀ is 2-pyridinylmethoxy and R₆₁ is fluoro; or
   (l) R₆₀ is 2-pyridinylmethoxy and R₆₁ is bromo;
   or
(2) at least one of R₅₉, R₆₀ and R₆₁ is selected from benzyloxy, 3-chlorobenzyloxy, and 2-pyridinylmethoxy, and when at least one of R₅₉, R₆₀ and R₆₁ is not benzyloxy, 3-chlorobenzyloxy or 2-pyridinylmethoxy, each of the others is independently selected from H, halogen, and C₂-C₄ alkynyl, with the proviso that when one of R₅₉, R₆₀ and R₆₁ is benzyloxy or 2-pyridinylmethoxy, the other two of R₅₉, R₆₀ and R₆₁ are not H;
or
(3) two of R₅₉, R₆₀ and R₆₁, together with the carbon atoms to which they are attached, form 1-(3-fluorobenzyl)-1H-pyrazole, and the other is selected from H, halogen, and C₂-C₄ alkynyl.

In some embodiments, the compound of Formula VII is a compound according to Formula VIII:

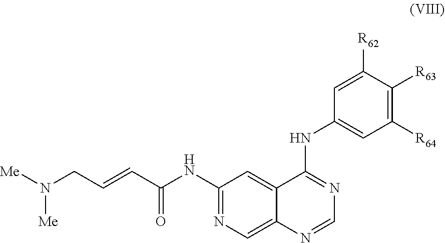

wherein R₆₂ is H, and either
(a) R₆₃ is (3-chlorobenzyl)oxy- and R₆₄ is chloro;
(b) R₆₃ and R₆₄, together with the carbon atoms to which they are attached, form 1-(3-fluorobenzyl)-1H-pyrazole;
(c) R₆₃ is 2-pyridinylmethoxy and R₆₄ is chloro;
(d) R₆₃ and R₆₄ are both chloro;
(e) R₆₃ is chloro and R₆₄ is bromo;
(f) R₆₃ is bromo and R₆₄ is chloro
(g) R₆₃ and R₆₄ are both bromo;
(h) R₆₃ is fluoro and R₆₄ is ethynyl;
(i) R₆₃ is chloro and R₆₄ is ethynyl;
(j) R₆₃ is bromo and R₆₄ is ethynyl;
(k) R₆₃ is bromo and R₆₄ is fluoro;
(l) R₆₃ is 2-pyridinylmethoxy and R₆₄ is fluoro; or
(m) R₆₃ is 2-pyridinylmethoxy and R₆₄ is bromo.

In some embodiments, the compound of Formula VII is selected from the group consisting of:
(2E)-N-(4-{3-chloro-4-[(3-chlorobenzyl)oxy]anilino}pyrido[3,4-d]pyrimidin-6-yl)-4-(dimethylamino)-2-butenamide (1),
(2E)-4-(dimethylamino)-N-(4-{[1-(3-fluorobenzyl)-1H-indazol-5-yl]amino}pyrido[3,4-d]pyrimidin-6-yl)-2-butenamide (2),
(2E)-N-{4-[3-chloro-4-(2-pyridinylmethoxy)anilino]pyrido[3,4-d]pyrimidin-6-yl}-4-(dimethylamino)-2-butenamide (3),
(2E)-N-4-(3,4-dichloroanilino)pyrido[3,4-d]pyrimidin-6-yl-4-(dimethylamino)-2-butenamide (4),
(2E)-N-4-(3-bromo-4-chloroanilino)pyrido[3,4-d]pyrimidin-6-yl-4-(dimethylamino)-2-butenamide (5),
(2E)-N-4-(4-bromo-3-chloroanilino)pyrido[3,4-d]pyrimidin-6-yl-4-(dimethylamino)-2-butenamide (6),
(2E)-N-4-(3,4-dibromoanilino)pyrido[3,4-d]pyrimidin-6-yl-4-(dimethylamino)-2-butenamide (7),
(2E)-4-(dimethylamino)-N-[4-(3-ethynyl-4-fluoroanilino)pyrido[3,4-(A]pyrimidin-6-yl]-2-butenamide (8),
(2E)-N-4-(4-chloro-3-ethynylanilino)pyrido[3,4-d]pyrimidin-6-yl-4-(dimethylamino)-2-butenamide (9),
(2E)-N-4-(4-bromo-3-ethynylanilino)pyrido[3,4-d]pyrimidin-6-yl-4-(dimethylamino)-2-butenamide (10),
(2E)-N-4-(4-bromo-3-fluoroanilino)pyrido[3,4-d]pyrimidin-6-yl-4-(dimethylamino)-2-butenamide (11),
(2E)-4-(dimethylamino)-N-{4-[3-fluoro-4-(2-pyridinylmethoxy)anilino]pyrido[3,4-d]pyrimidin-6-yl}-2-butenamide (89) and
(2E)-N-{4-[3-bromo-4-(2-pyridinylmethoxy)anilino]pyrido[3,4-d]pyrimidin-6-yl}-4-(dimethylamino)-2-butenamide (90).

The structures of the compounds provided in the list above are depicted below:

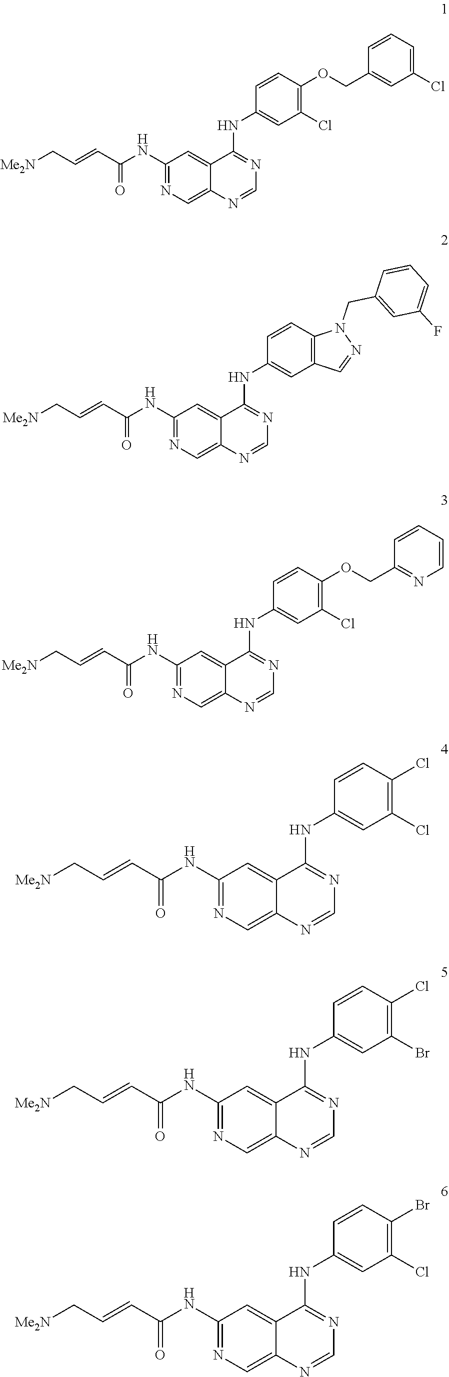 [SMILES: CN(C)C/C=C/C(=O)Nc1cc2c(Nc3ccc(OCc4cccc(Cl)c4)c(Cl)c3)ncnc2cn1 ; CN(C)C/C=C/C(=O)Nc1cc2c(Nc3ccc4c(cnn4Cc4cccc(F)c4)c3)ncnc2cn1 ; CN(C)C/C=C/C(=O)Nc1cc2c(Nc3ccc(OCc4ccccn4)c(Cl)c3)ncnc2cn1 ; CN(C)C/C=C/C(=O)Nc1cc2c(Nc3ccc(Cl)c(Cl)c3)ncnc2cn1 ; CN(C)C/C=C/C(=O)Nc1cc2c(Nc3ccc(Cl)c(Br)c3)ncnc2cn1 ; CN(C)C/C=C/C(=O)Nc1cc2c(Nc3ccc(Br)c(Cl)c3)ncnc2cn1]

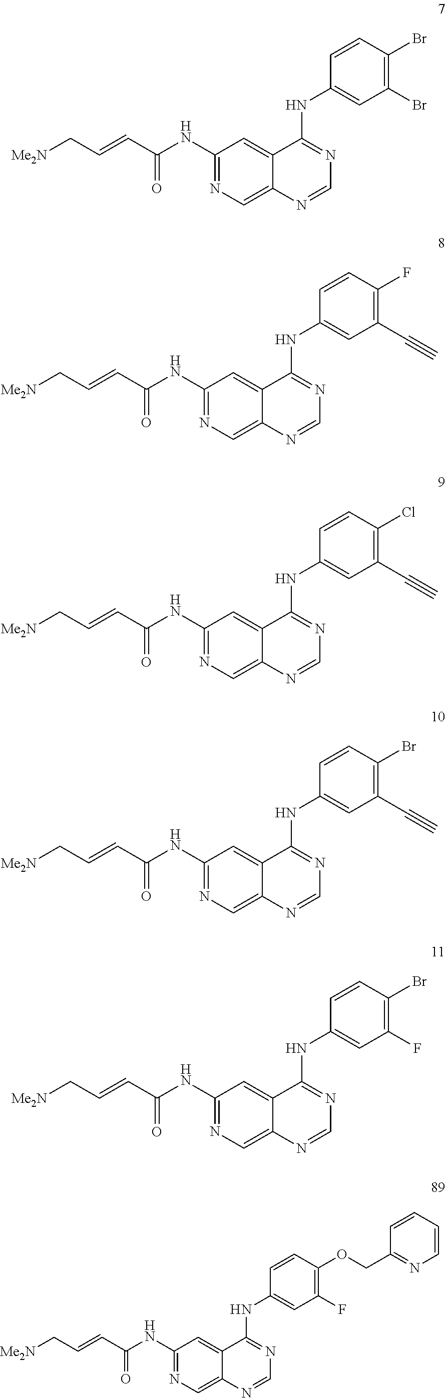 [SMILES: CN(C)C/C=C/C(=O)Nc1cc2c(Nc3ccc(Br)c(Br)c3)ncnc2cn1 ; C#Cc1cc(Nc2ncnc3cnc(NC(=O)/C=C/CN(C)C)cc23)ccc1F ; C#Cc1cc(Nc2ncnc3cnc(NC(=O)/C=C/CN(C)C)cc23)ccc1Cl ; C#Cc1cc(Nc2ncnc3cnc(NC(=O)/C=C/CN(C)C)cc23)ccc1Br ; CN(C)C/C=C/C(=O)Nc1cc2c(Nc3ccc(Br)c(F)c3)ncnc2cn1 ; CN(C)C/C=C/C(=O)Nc1cc2c(Nc3ccc(OCc4ccccn4)c(F)c3)ncnc2cn1]

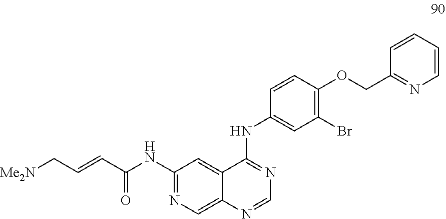 [SMILES: CN(C)C/C=C/C(=O)Nc1cc2c(Nc3ccc(OCc4ccccn4)c(Br)c3)ncnc2cn1]

In some embodiments, the compounds are of Formula IX:

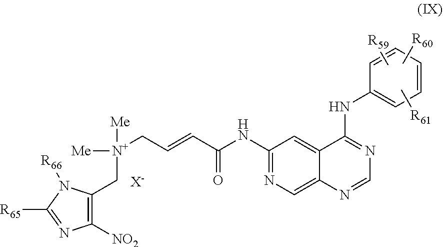

wherein X is any negatively charged counterion, R₅₉, R₆₀ and R₆₁ are as defined for Formula VII, R₆₅ is selected from H, methyl, ethyl, trifluoromethyl, —CN, —CONH₂, and propyn-1-yl, and R₆₆ is C₁-C₆ alkyl.

In some embodiments, the compounds are of Formula X:

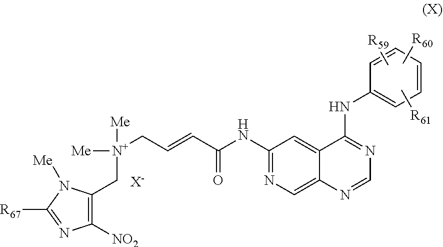

wherein X is any negatively charged counterion, R₅₉, R₆₀ and R₆₁ are as defined for Formula VII and R₆₇ is selected from H, methyl, ethyl, trifluoromethyl, —CN, —CONH₂, and propyn-1-yl.

In some embodiments, the compounds are of Formula XI:

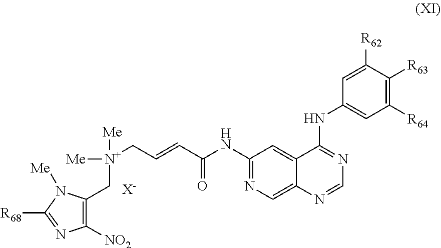

wherein X is any negatively charged counterion, R₆₂, R₆₃ and R₆₄ are as defined for Formula VIII and R₆₈ is selected from H, methyl, ethyl, trifluoromethyl, —CN, —CONH₂, and propyn-1-yl.

In some embodiments, X is selected from halide (e.g., fluoride, chloride, bromide, iodide), methanesulfonate, trifluoromethanesulfonate, acetate, trifluoroacetate, tosylate, lactate, citrate and formate.

In some embodiments, the compounds are selected from the group consisting of:
(2E)-4-[(4-{3-chloro-4-[(3-chlorobenzyl)oxy]anilino}pyrido[3,4-d]pyrimidin-6-yl)amino]-N,N-dimethyl-N-[(1-methyl-4-nitro-1H-imidazol-5-yl)methyl]-4-oxo-2-buten-1-ammonium bromide (12),
(2E)-4-[(4-{[1-(3-fluorobenzyl)-1H-indazol-5-yl]amino}pyrido[3,4-d]pyrimidin-6-yl)amino]-N,N-dimethyl-N-[(1-methyl-4-nitro-1H-imidazol-5-yl)methyl]-4-oxo-2-buten-1-ammonium bromide (13),
(2E)-N-[(1,2-dimethyl-4-nitro-1H-imidazol-5-yl)methyl]-4-[(4-{[1-(3-fluorobenzyl)-1H-indazol-5-yl]amino}pyrido[3,4-d]pyrimidin-6-yl)amino]-N,N-dimethyl-4-oxo-2-buten-1-ammonium bromide (14),
(2E)-4-({4-[3-chloro-4-(2-pyridinylmethoxy)anilino]pyrido[3,4-d]pyrimidin-6-yl}amino)-N,N-dimethyl-N-[(1-methyl-4-nitro-1H-imidazol-5-yl)methyl]-4-oxo-2-buten-1-ammonium bromide (15),
(2E)-4-{[4-(3,4-dichloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (16),
(2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (17),
(2E)-4-{[4-(4-bromo-3-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (18),
(2E)-4-{[4-(3,4-dibromoanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (19),
(2E)-4-{[4-(3-ethynyl-4-fluoroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (20),
(2E)-4-{[4-(4-chloro-3-ethynylanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (21),
(2E)-4-[(4-{3-chloro-4-[(3-chlorobenzyl)oxy]anilino}pyrido[3,4-d]pyrimidin-6-yl)amino-N-(1,2-dimethyl-4-nitro-1H-imidazol-5-yl)methyl]-N,N-dimethyl-4-oxo-2-buten-1-ammonium bromide (22),
(2E)-4-[(4-{3-chloro-4-[(3-chlorobenzyl)oxy]anilino}pyrido[3,4-d]pyrimidin-6-yl)amino]-N-[(2-ethyl-1-methyl-4-nitro-1H-imidazol-5-yl)methyl]-N,N-dimethyl-4-oxo-2-buten-1-ammonium bromide (23),
(2E)-4-[(4-{3-chloro-4-[(3-chlorobenzyl)oxy]anilino}pyrido[3,4-d]pyrimidin-6-yl)amino]-N,N-dimethyl-N-{[1-methyl-4-nitro-2-(trifluoromethyl)-1H-imidazol-5-yl]methyl}-4-oxo-2-buten-1-ammonium bromide (24),
(2E)-4-[(4-{3-chloro-4-(3-chlorobenzyl)oxy]anilino}pyrido[3,4-d]pyrimidin-6-yl)amino-N-[(2-cyano-1-methyl-4-nitro-1H-imidazol-5-yl)methyl]-N,N-dimethyl-4-oxo-2-buten-1-ammonium bromide (25),
(2E)-N-{[2-(aminocarbonyl)-1-methyl-4-nitro-1H-imidazol-5-yl]methyl}-4-[(4-{3-chloro-4-[(3-chlorobenzyl)oxy]anilino}pyrido[3,4-d]pyrimidin-6-yl)amino]-N,N-dimethyl-4-oxo-2-buten-1-ammonium bromide (26),
(2E)-4-[(4-{3-chloro-4-[(3-chlorobenzyl)oxy]anilino}pyrido[3,4-d]pyrimidin-6-yl)amino]-N,N-dimethyl-N-{[1-methyl-4-nitro-2-(1-propynyl)-1H-imidazol-5-yl]methyl}-4-oxo-2-buten-1-ammonium bromide (27),
(2E)-N-[(2-ethyl-1-methyl-4-nitro-1H-imidazol-5-yl)methyl]-4-[(4-{[1-(3-fluorobenzyl)-1H-indazol-5-yl]amino}pyrido[3,4-d]pyrimidin-6-yl)amino]-N,N-dimethyl-4-oxo-2-buten-1-ammonium bromide (28),
(2E)-4-[(4-{[1-(3-fluorobenzyl)-1H-indazol-5-yl]amino}pyrido[3,4-d]pyrimidin-6-yl)amino]-N,N-dimethyl-N-{[1-methyl-4-nitro-2-(trifluoromethyl)-1H-imidazol-5-yl]methyl}-4-oxo-2-buten-1-ammonium bromide (29),
(2E)-N-[(2-cyano-1-methyl-4-nitro-1H-imidazol-5-yl)methyl]-4-[(4-{[1-(3-fluorobenzyl)-1H-indazol-5-yl]amino}pyrido[3,4-d]pyrimidin-6-yl)amino]-N,N-dimethyl-4-oxo-2-buten-1-ammonium bromide (30),
(2E)-N-{[2-(aminocarbonyl)-1-methyl-4-nitro-1H-imidazol-5-yl]methyl}-4-(4-{[1-(3-fluorobenzyl)-1H-indazol-5-yl]amino}pyrido[3,4-d]pyrimidin-6-yl)amino-N,N-dimethyl-4-oxo-2-buten-1-ammonium bromide (31),
(2E)-4-[(4-{[1-(3-fluorobenzyl)-1H-indazol-5-yl]amino}pyrido[3,4-d]pyrimidin-6-yl)amino]-N,N-dimethyl-N-{[1-methyl-4-nitro-2-(1-propynyl)-1H-imidazol-5-yl]methyl}-4-oxo-2-buten-1-ammonium bromide (32),
(2E)-4-({4-[3-chloro-4-(2-pyridinylmethoxy)anilino]pyrido[3,4-d]pyrimidin-6-yl}amino)-N-[(1,2-dimethyl-4-nitro-1H-imidazol-5-yl)methyl]-N,N-dimethyl-4-oxo-2-buten-1-ammonium bromide (33),
(2E)-4-({4-[3-chloro-4-(2-pyridinylmethoxy)anilino]pyrido[3,4-d]pyrimidin-6-yl}amino)-N-[(2-ethyl-1-methyl-4-nitro-1H-imidazol-5-yl)methyl]-N,N-dimethyl-4-oxo-2-buten-1-ammonium bromide (34),
(2E)-4-({4-[3-chloro-4-(2-pyridinylmethoxy)anilino]pyrido[3,4-d]pyrimidin-6-yl}amino)-N,N-dimethyl-N-{[1-methyl-4-nitro-2-(trifluoromethyl)-1H-imidazol-5-yl]methyl}-4-oxo-2-buten-1-ammonium bromide (35),
(2E)-4-({4-[3-chloro-4-(2-pyridinylmethoxy)anilino]pyrido[3,4-d]pyrimidin-6-yl}amino)-N-[(2-cyano-1-methyl-4-nitro-1H-imidazol-5-yl)methyl]-N,N-dimethyl-4-oxo-2-buten-1-ammonium bromide (36),
(2E)-N-{[2-(aminocarbonyl)-1-methyl-4-nitro-1H-imidazol-5-yl]methyl}-4-({4-[3-chloro-4-(2-pyridinylmethoxy)anilino]pyrido[3,4-d]pyrimidin-6-yl}amino)-N,N-dimethyl-4-oxo-2-buten-1-ammonium bromide (37),
(2E)-4-({4-[3-chloro-4-(2-pyridinylmethoxy)anilino]pyrido[3,4-d]pyrimidin-6-yl}amino)-N,N-dimethyl-N-{[1-methyl-4-nitro-2-(1-propynyl)-1H-imidazol-5-yl]methyl}-4-oxo-2-buten-1-ammonium bromide (38),
(2E)-4-{[4-(3,4-dichloroanilino)pyrido[3,4-d]pyrimidin-6-yl]amino}-N-[(1,2-dimethyl-4-nitro-1H-imidazol-5-yl)methyl]-N,N-dimethyl-4-oxo-2-buten-1-ammonium bromide (39),
(2E)-4-{[4-(3,4-dichloroanilino)pyrido[3,4-d]pyrimidin-6-yl]amino}-N-[(2-ethyl-1-methyl-4-nitro-1H-imidazol-5-yl)methyl]-N,N-dimethyl-4-oxo-2-buten-1-ammonium bromide (40),
(2E)-4-{[4-(3,4-dichloroanilino)pyrido[3,4-d]pyrimidin-6-yl]amino}-N,N-dimethyl-N-{[1-methyl-4-nitro-2-(trifluoromethyl)-1H-imidazol-5-yl]methyl}-4-oxo-2-buten-1-ammonium bromide (41),
(2E)-N-(2-cyano-1-methyl-4-nitro-1H-imidazol-5-yl)methyl-4-{[4-(3,4-dichloroanilino)pyrido[3,4-d]pyrimidin-6-yl]amino}-N,N-dimethyl-4-oxo-2-buten-1-ammonium bromide (42),
(2E)-N-{[2-(aminocarbonyl)-1-methyl-4-nitro-1H-imidazol-5-yl]methyl}-4-{[4-(3,4-dichloroanilino)pyrido[3,4-d]pyrimidin-6-yl]amino}-N,N-dimethyl-4-oxo-2-buten-1-ammonium bromide (43),
(2E)-4-{[4-(3,4-dichloroanilino)pyrido[3,4-d]pyrimidin-6-yl]amino}-N,N-dimethyl-N-{[1-methyl-4-nitro-2-(1-propynyl)-1H-imidazol-5-yl]methyl}-4-oxo-2-buten-1-ammonium bromide (44),
(2E)-4-{[4-(3-bromo-4-chloroanilino)pyrido[3,4-d]pyrimidin-6-yl]amino}-N-[(1,2-dimethyl-4-nitro-1H-imidazol-5-yl)methyl]-N,N-dimethyl-4-oxo-2-buten-1-ammonium bromide (45),
(2E)-4-{[4-(3-bromo-4-chloroanilino)pyrido[3,4-d]pyrimidin-6-yl]amino}-N-[(2-ethyl-1-methyl-4-nitro-1H-imidazol-5-yl)methyl]-N,N-dimethyl-4-oxo-2-buten-1-ammonium bromide (46),
(2E)-4-{[4-(3-bromo-4-chloroanilino)pyrido[3,4-d]pyrimidin-6-yl]amino}-N,N-dimethyl-N-{[1-methyl-4-nitro-2-(trifluoromethyl)-1H-imidazol-5-yl]methyl}-4-oxo-2-buten-1-ammonium bromide (47),
(2E)-4-{[4-(3-bromo-4-chloroanilino)pyrido[3,4-d]pyrimidin-6-yl]amino}-N-[(2-cyano-1-methyl-4-nitro-1H-imidazol-5-yl)methyl]-N,N-dimethyl-4-oxo-2-buten-1-ammonium bromide (48),
(2E)-N-{[2-(aminocarbonyl)-1-methyl-4-nitro-1H-imidazol-5-yl]methyl}-4-{[4-(3-bromo-4-chloroanilino)pyrido[3,4-d]pyrimidin-6-yl]amino}-N,N-dimethyl-4-oxo-2-buten-1-ammonium bromide (49),
(2E)-4-{[4-(3-bromo-4-chloroanilino)pyrido[3,4-d]pyrimidin-6-yl]amino}-N,N-dimethyl-N-{[1-methyl-4-nitro-2-(1-propynyl)-1H-imidazol-5-yl]methyl}-4-oxo-2-buten-1-ammonium bromide (50),
(2E)-4-{[4-(4-bromo-3-chloroanilino)pyrido[3,4-d]pyrimidin-6-yl]amino}-N-[(1,2-dimethyl-4-nitro-1H-imidazol-5-yl)methyl]-N,N-dimethyl-4-oxo-2-buten-1-ammonium bromide (51),
(2E)-4-{[4-(4-bromo-3-chloroanilino)pyrido[3,4-d]pyrimidin-6-yl]amino}-N-[(2-ethyl-1-methyl-4-nitro-1H-imidazol-5-yl)methyl]-N,N-dimethyl-4-oxo-2-buten-1-ammonium bromide (52),
(2E)-4-{[4-(4-bromo-3-chloroanilino)pyrido[3,4-d]pyrimidin-6-yl]amino}-N,N-dimethyl-N-{[1-methyl-4-nitro-2-(trifluoromethyl)-1H-imidazol-5-yl]methyl}-4-oxo-2-buten-1-ammonium bromide (53),
(2E)-4-{[4-(4-bromo-3-chloroanilino)pyrido[3,4-d]pyrimidin-6-yl]amino}-N-[(2-cyano-1-methyl-4-nitro-1H-imidazol-5-yl)methyl]-N,N-dimethyl-4-oxo-2-buten-1-ammonium bromide (54),
(2E)-N-{[2-(aminocarbonyl)-1-methyl-4-nitro-1H-imidazol-5-yl]methyl}-4-{[4-(4-bromo-3-chloroanilino)pyrido[3,4-d]pyrimidin-6-yl]amino}-N,N-dimethyl-4-oxo-2-buten-1-ammonium bromide (55),
(2E)-4-{[4-(4-bromo-3-chloroanilino)pyrido[3,4-d]pyrimidin-6-yl]amino}-N,N-dimethyl-N-{[1-methyl-4-nitro-2-(1-propynyl)-1H-imidazol-5-yl]methyl}-4-oxo-2-buten-1-ammonium bromide (56),
(2E)-4-{[4-(3,4-dibromoanilino)pyrido[3,4-d]pyrimidin-6-yl]amino}-N-[(1,2-dimethyl-4-nitro-1H-imidazol-5-yl)methyl]-N,N-dimethyl-4-oxo-2-buten-1-ammonium bromide (57),
(2E)-4-{[4-(3,4-dibromoanilino)pyrido[3,4-d]pyrimidin-6-yl]amino}-N-[(2-ethyl-1-methyl-4-nitro-1H-imidazol-5-yl)methyl]-N,N-dimethyl-4-oxo-2-buten-1-ammonium bromide (58),
(2E)-4-{[4-(3,4-dibromoanilino)pyrido[3,4-d]pyrimidin-6-yl]amino}-N,N-dimethyl-N-{[1-methyl-4-nitro-2-(trifluoromethyl)-1H-imidazol-5-yl]methyl}-4-oxo-2-buten-1-ammonium bromide (59),
(2E)-N-[(2-cyano-1-methyl-4-nitro-1H-imidazol-5-yl)methyl]-4-{[4-(3,4-dibromoanilino)pyrido[3,4-d]pyrimidin-6-yl]amino}-N,N-dimethyl-4-oxo-2-buten-1-ammonium bromide (60),
(2E)-N-{[2-(aminocarbonyl)-1-methyl-4-nitro-1H-imidazol-5-yl]methyl}-4-{[4-(3,4-dibromoanilino)pyrido[3,4-d]pyrimidin-6-yl]amino}-N,N-dimethyl-4-oxo-2-buten-1-ammonium bromide (61),
(2E)-4-{[4-(3,4-dibromoanilino)pyrido[3,4-d]pyrimidin-6-yl]amino}-N,N-dimethyl-N-{[1-methyl-4-nitro-2-(1-propynyl)-1H-imidazol-5-yl]methyl}-4-oxo-2-buten-1-ammonium bromide (62),
(2E)-N-[2-dimethyl-4-nitro-1H-imidazol-5-yl)methyl]-4-{[4-(3-ethynyl-4-fluoroanilino)pyrido[3,4-d]pyrimidin-6-yl]amino}-N,N-dimethyl-4-oxo-2-buten-1-ammonium bromide (63),
(2E)-N-(2-ethyl-1-methyl-4-nitro-1H-imidazol-5-yl)methyl-4-{[4-(3-ethynyl-4-fluoroanilino)pyrido[3,4-d]pyrimidin-6-yl]amino}-N,N-dimethyl-4-oxo-2-buten-1-ammonium bromide (64),
(2E)-4-{[4-(3-ethynyl-4-fluoroanilino)pyrido[3,4-d]pyrimidin-6-yl]amino}-N,N-dimethyl-N-{[1-methyl-4-nitro-2-(trifluoromethyl)-1H-imidazol-5-yl]methyl}-4-oxo-2-buten-1-ammonium bromide (65),
(2E)-N-[(2-cyano-1-methyl-4-nitro-1H-imidazol-5-yl)methyl]-4-{[4-(3-ethynyl-4-fluoroanilino)pyrido[3,4-d]pyrimidin-6-yl]amino}-N,N-dimethyl-4-oxo-2-buten-1-ammonium bromide (66),
(2E)-N-{[2-(aminocarbonyl)-1-methyl-4-nitro-1H-imidazol-5-yl]methyl}-4-{[4-(3-ethynyl-4-fluoroanilino)pyrido[3,4-d]pyrimidin-6-yl]amino}-N,N-dimethyl-4-oxo-2-buten-1-ammonium bromide (67),
(2E)-4-{[4-(3-ethynyl-4-fluoroanilino)pyrido[3,4-d]pyrimidin-6-yl]amino}-N,N-dimethyl-N-{[1-methyl-4-nitro-2-(1-propynyl)-1H-imidazol-5-yl]methyl}-4-oxo-2-buten-1-ammonium bromide (68),
(2E)-4-{[4-(4-chloro-3-ethynylanilino)pyrido[3,4-d]pyrimidin-6-yl]amino}-N-[(1,2-dimethyl-4-nitro-1H-imidazol-5-yl)methyl]-N,N-dimethyl-4-oxo-2-buten-1-ammonium bromide (69),
(2E)-4-{[4-(4-chloro-3-ethynylanilino)pyrido[3,4-d]pyrimidin-6-yl]amino}-N-[(2-ethyl-1-methyl-4-nitro-1H-imidazol-5-yl)methyl]-N,N-dimethyl-4-oxo-2-buten-1-ammonium bromide (70),
(2E)-4-{[4-(4-chloro-3-ethynylanilino)pyrido[3,4-d]pyrimidin-6-yl]amino}-N,N-di methyl-N-{[1-methyl-4-nitro-2-(trifluoromethyl)-1H-imidazol-5-yl]methyl}-4-oxo-2-buten-1-ammonium bromide (71),
(2E)-4-{[4-(4-chloro-3-ethynylanilino)pyrido[3,4-d]pyrimidin-6-yl]amino}-N-[(2-cyano-1-methyl-4-nitro-1H-imidazol-5-yl)methyl]-N,N-dimethyl-4-oxo-2-buten-1-ammonium bromide (72),
(2E)-N-{[2-(aminocarbonyl)-1-methyl-4-nitro-1H-imidazol-5-yl]methyl}-4-{[4-(4-chloro-3-ethynylanilino)pyrido[3,4-d]pyrimidin-6-yl]amino}-N,N-dimethyl-4-oxo-2-buten-1-ammonium bromide (73),
(2E)-4-{[4-(4-chloro-3-ethynylanilino)pyrido[3,4-d]pyrimidin-6-yl]amino}-N,N-dimethyl-N-{[1-methyl-4-nitro-2-(1-propynyl)-1H-imidazol-5-yl]methyl}-4-oxo-2-buten-1-ammonium bromide (74),
(2E)-4-{[4-(4-bromo-3-ethynylanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (75),
(2E)-4-{[4-(4-bromo-3-ethynylanilino)pyrido[3,4-d]pyrimidin-6-yl]amino}-N-[(1,2-dimethyl-4-nitro-1H-imidazol-5-yl)methyl]-N,N-dimethyl-4-oxo-2-buten-1-ammonium bromide (76),
(2E)-4-{[4-(4-bromo-3-ethynylanilino)pyrido[3,4-d]pyrimidin-6-yl]amino}-N-[(2-ethyl-1-methyl-4-nitro-1H-imidazol-5-yl)methyl]-N,N-dimethyl-4-oxo-2-buten-1-ammonium bromide (77),
(2E)-4-{[4-(4-bromo-3-ethynylanilino)pyrido[3,4-d]pyrimidin-6-yl]amino}-N,N-dimethyl-N-{[1-methyl-4-nitro-2-(trifluoromethyl)-1H-imidazol-5-yl]methyl}-4-oxo-2-buten-1-ammonium bromide (78),
(2E)-4-{[4-(4-bromo-3-ethynylanilino)pyrido[3,4-d]pyrimidin-6-yl]amino}-N-[(2-cyano-1-methyl-4-nitro-1H-imidazol-5-yl)methyl]-N,N-dimethyl-4-oxo-2-buten-1-ammonium bromide (79),
(2E)-N-{[2-(aminocarbonyl)-1-methyl-4-nitro-1H-imidazol-5-yl]methyl}-4-{[4-(4-bromo-3-ethynylanilino)pyrido[3,4-d]pyrimidin-6-yl]amino}-N,N-dimethyl-4-oxo-2-buten-1-ammonium bromide (80),
(2E)-4-{[4-(4-bromo-3-ethynylanilino)pyrido[3,4-d]pyrimidin-6-yl]amino}-N,N-dimethyl-N-{[1-methyl-4-nitro-2-(1-propynyl)-1H-imidazol-5-yl]methyl}-4-oxo-2-buten-1-ammonium bromide (81),
(2E)-4-{[4-(4-bromo-3-fluoroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (82),
(2E)-4-{[4-(4-bromo-3-fluoroanilino)pyrido[3,4-d]pyrimidin-6-yl]amino}-N-[(1,2-dimethyl-4-nitro-1H-imidazol-5-yl)methyl]-N,N-dimethyl-4-oxo-2-buten-1-ammonium bromide (83),
(2E)-4-{[4-(4-bromo-3-fluoroanilino)pyrido[3,4-d]pyrimidin-6-yl]amino}-N-[(1,2-ethyl-1-methyl-4-nitro-1H-imidazol-5-yl)methyl]-N,N-dimethyl-4-oxo-2-buten-1-ammonium bromide (84),
(2E)-4-{[4-(4-bromo-3-fluoroanilino)pyrido[3,4-d]pyrimidin-6-yl]amino}-N,N-dimethyl-N-{[1-methyl-4-nitro-2-(trifluoromethyl)-1H-imidazol-5-yl]methyl}-4-oxo-2-buten-1-ammonium bromide (85),
(2E)-4-{[4-(4-bromo-3-fluoroanilino)pyrido[3,4-d]pyrimidin-6-yl]amino}-N-[(2-cyano-1-methyl-4-nitro-1H-imidazol-5-yl)methyl]-N,N-dimethyl-4-oxo-2-buten-1-ammonium bromide (86),
(2E)-N-{[2-(aminocarbonyl)-1-methyl-4-nitro-1H-imidazol-5-yl]methyl}-4-{[4-(4-bromo-3-fluoroanilino)pyrido[3,4-d]pyrimidin-6-yl]amino}-N,N-dimethyl-4-oxo-2-buten-1-ammonium bromide (87),
(2E)-4-{[4-(4-bromo-3-fluoroanilino)pyrido[3,4-d]pyrimidin-6-yl]amino}-N,N-dimethyl-N-{[1-methyl-4-nitro-2-(1-propynyl)-1H-imidazol-5-yl]methyl}-4-oxo-2-buten-1-ammonium bromide (88),
(2E)-4-({4-[3-fluoro-4-(2-pyridinylmethoxy)anilino]pyrido[3,4-d]pyrimidin-6-yl}amino)-N,N-dimethyl-N-[(1-methyl-4-nitro-1H-imidazol-5-yl)methyl]-4-oxo-2-buten-1-ammonium bromide (91),
(2E)-N-[(1,2-dimethyl-4-nitro-1H-imidazol-5-yl)methyl]-4-({4-[3-fluoro-4-(2-pyridinylmethoxy)anilino]pyrido[3,4-d]pyrimidin-6-yl}amino)-N,N-dimethyl-4-oxo-2-buten-1-ammonium bromide (92),
(2E)-N-(2-ethyl-1-methyl-4-nitro-1H-imidazol-5-yl)methyl-4-({4-[3-fluoro-4-(2-pyridinylmethoxy)anilino]pyrido[3,4-d]pyrimidin-6-yl}amino)-N,N-dimethyl-4-oxo-2-buten-1-ammonium bromide (93),
(2E)-4-({4-[3-fluoro-4-(2-pyridinylmethoxy)anilino]pyrido[3,4-d]pyrimidin-6-yl}amino)-N,N-dimethyl-N-{[1-methyl-4-nitro-2-(trifluoromethyl)-1H-imidazol-5-yl]methyl}-4-oxo-2-buten-1-ammonium bromide (94),
(2E)-N-[(2-cyano-1-methyl-4-nitro-1H-imidazol-5-yl)methyl]-4-({4-[3-fluoro-4-(2-pyridinylmethoxy)anilino]pyrido[3,4-d]pyrimidin-6-yl}amino)-N,N-dimethyl-4-oxo-2-buten-1-ammonium bromide (95),
(2E)-N-{[2-(aminocarbonyl)-1-methyl-4-nitro-1H-imidazol-5-yl]methyl}-4-({4-[3-fluoro-4-(2-pyridinylmethoxy)anilino]pyrido[3,4-d]pyrimidin-6-yl}amino)-N,N-dimethyl-4-oxo-2-buten-1-ammonium bromide (96),
(2E)-4-({4-[3-fluoro-4-(2-pyridinylmethoxy)anilino]pyrido[3,4-d]pyrimidin-6-yl}amino)-N,N-dimethyl-N-{[1-methyl-4-nitro-2-(1-propynyl)-1H-imidazol-5-yl]methyl}-4-oxo-2-buten-1-ammonium bromide (97),
(2E)-4-({4-[3-bromo-4-(2-pyridinylmethoxy)anilino]pyrido[3,4-d]pyrimidin-6-yl}amino)-N,N-dimethyl-N-[(1-methyl-4-nitro-1H-imidazol-5-yl)methyl]-4-oxo-2-buten-1-ammonium bromide (98),
(2E)-4-({4-[3-bromo-4-(2-pyridinylmethoxy)anilino]pyrido[3,4-d]pyrimidin-6-yl}amino)-N-[(1,2-dimethyl-4-nitro-1H-imidazol-5-yl)methyl]-N,N-dimethyl-4-oxo-2-buten-1-ammonium bromide (99),
(2E)-4-({4-[3-bromo-4-(2-pyridinylmethoxy)anilino]pyrido[3,4-d]pyrimidin-6-yl}amino)-N-[(2-ethyl-1-methyl-4-nitro-1H-imidazol-5-yl)methyl]-N,N-dimethyl-4-oxo-2-buten-1-ammonium bromide (100),
(2E)-4-({4-[3-bromo-4-(2-pyridinylmethoxy)anilino]pyrido[3,4-d]pyrimidin-6-yl}amino)-N,N-dimethyl-N-{[1-methyl-4-nitro-2-(trifluoromethyl)-1H-imidazol-5-yl]methyl}-4-oxo-2-buten-1-ammonium bromide (101),
(2E)-4-({4-[3-bromo-4-(2-pyridinylmethoxy)anilino]pyrido[3,4-d]pyrimidin-6-yl}amino)-N-[(2-cyano-1-methyl-4-nitro-1H-imidazol-5-yl)methyl]-N,N-dimethyl-4-oxo-2-buten-1-ammonium bromide (102),
(2E)-N-{[2-(aminocarbonyl)-1-methyl-4-nitro-1H-imidazol-5-yl]methyl}-4-({4-[3-bromo-4-(2-pyridinylmethoxy)anilino]pyrido[3,4-d]pyrimidin-6-yl}amino)-N,N-dimethyl-4-oxo-2-buten-1-ammonium bromide (103) and
(2E)-4-({4-[3-bromo-4-(2-pyridinylmethoxy)anilino]pyrido[3,4-d]pyrimidin-6-yl}amino)-N,N-dimethyl-N-{[1-methyl-4-nitro-2-(1-propynyl)-1H-imidazol-5-yl]methyl}-4-oxo-2-buten-1-ammonium bromide (104); or any other salt thereof (e.g., the listed counterion is exchanged for any other counterion, which In some embodiments is a pharmaceutically acceptable counterion, of same polarity—negative or positive), or any solvate thereof.

The structures of the compounds provided in the list above are depicted below:

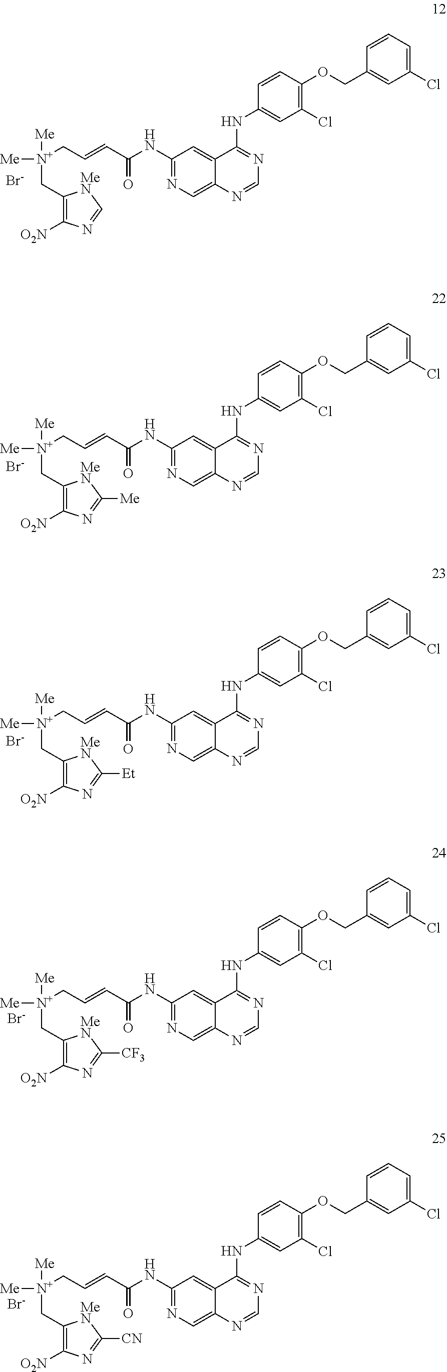

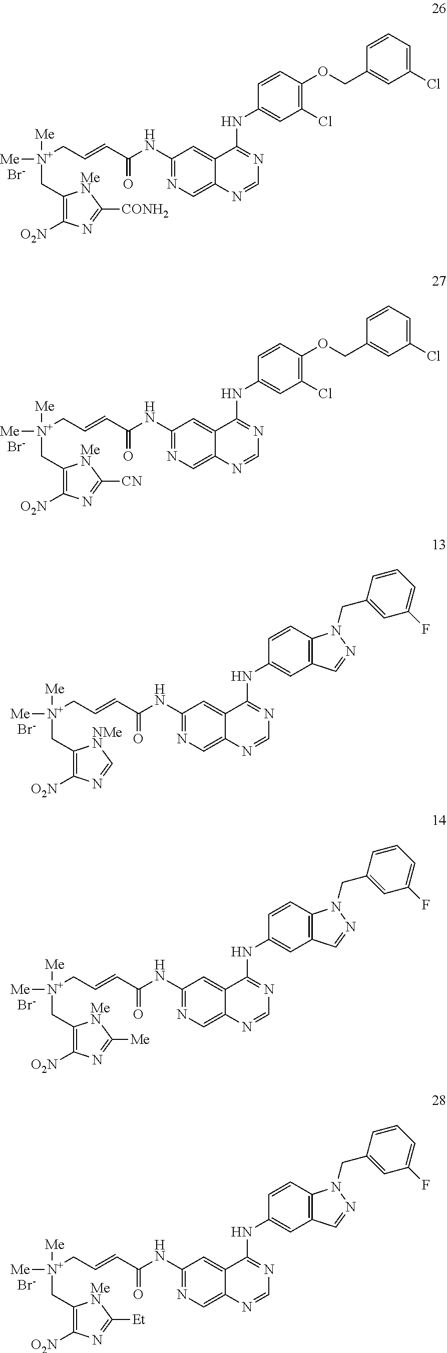

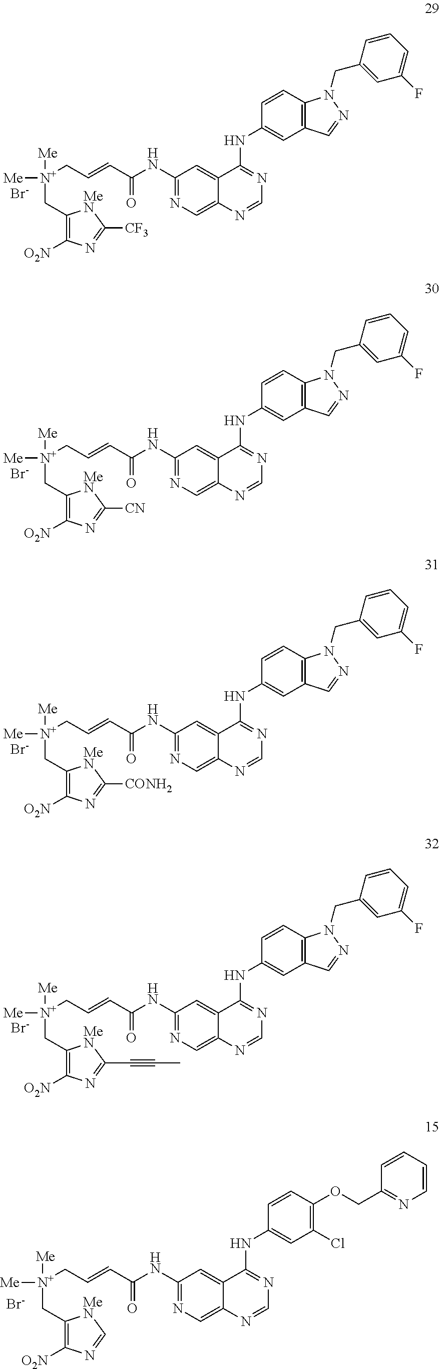

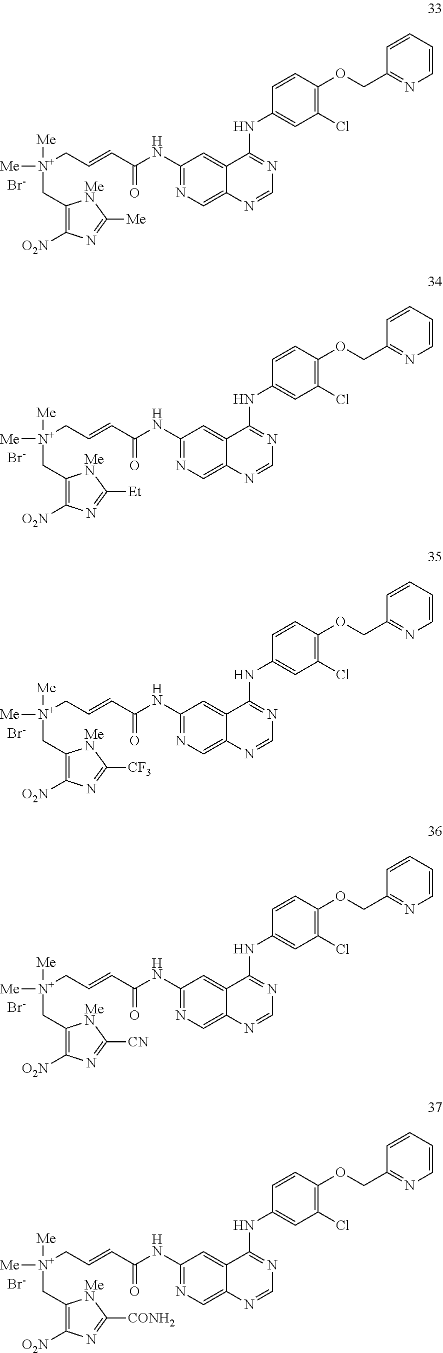

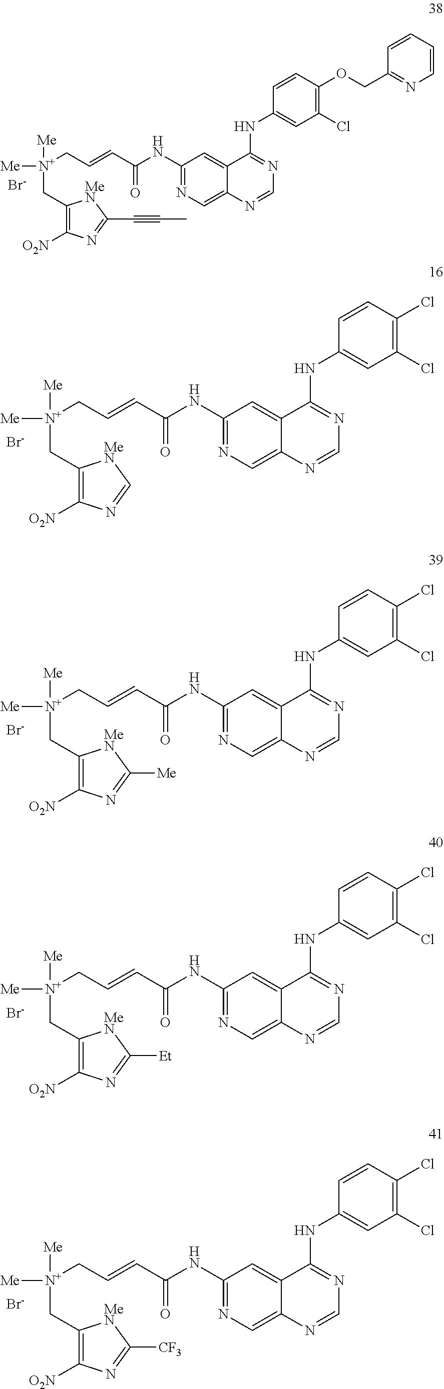

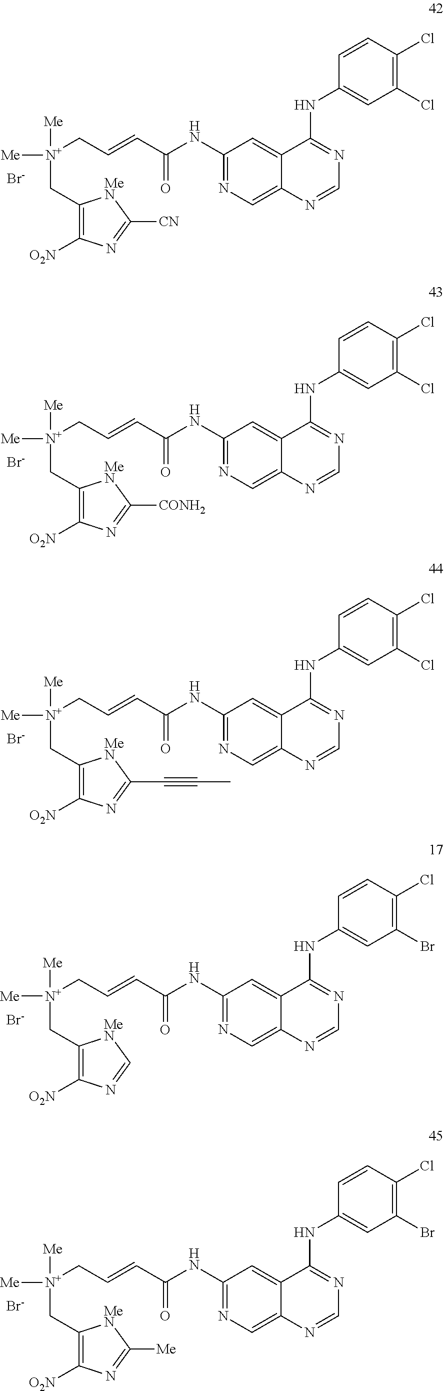

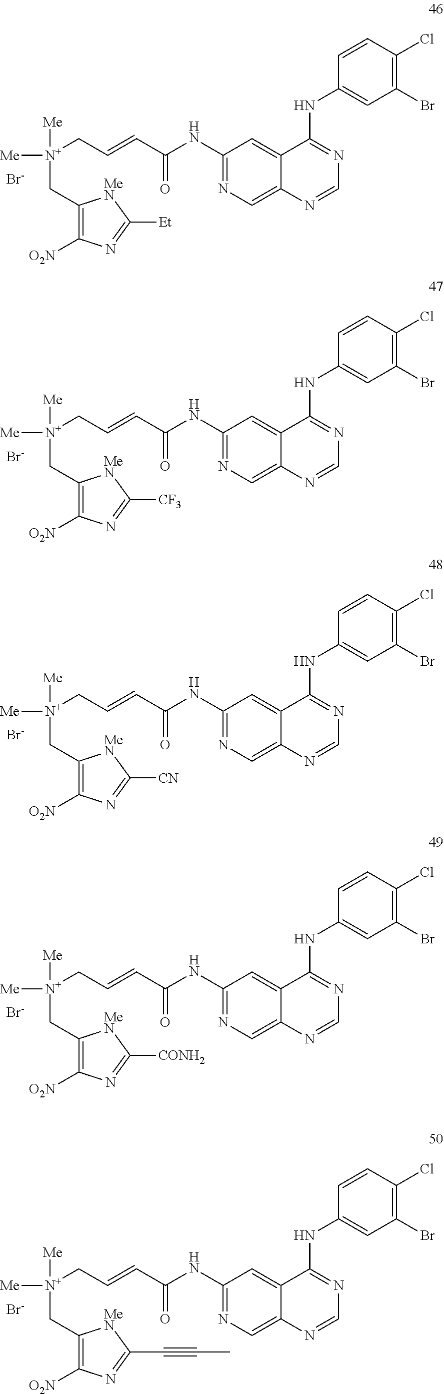

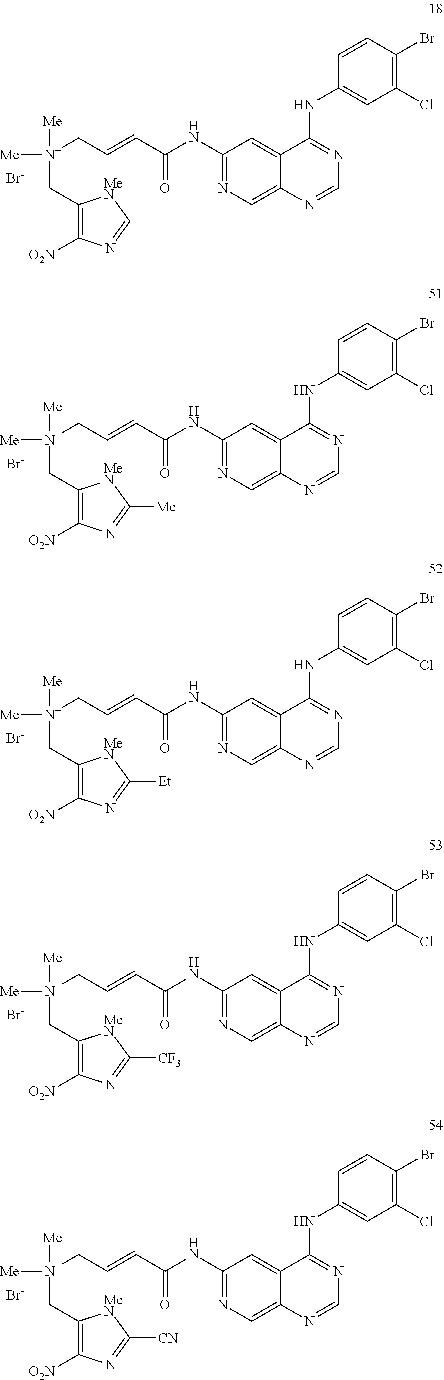

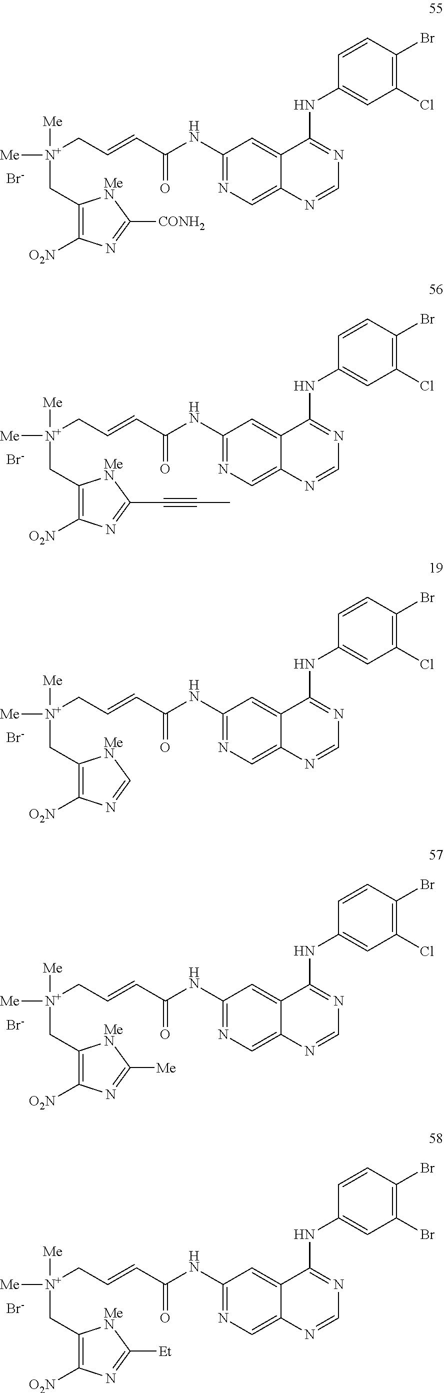

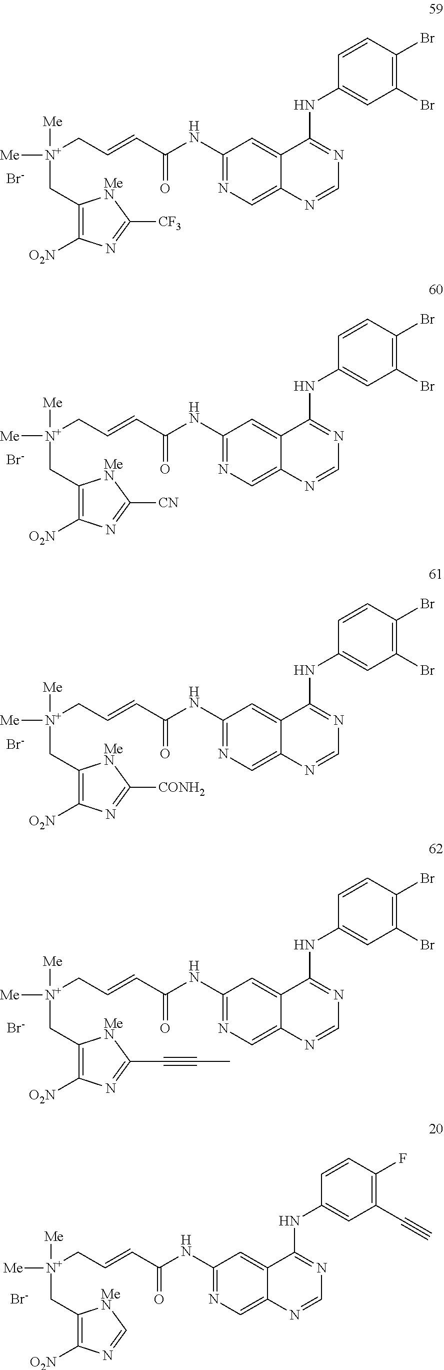

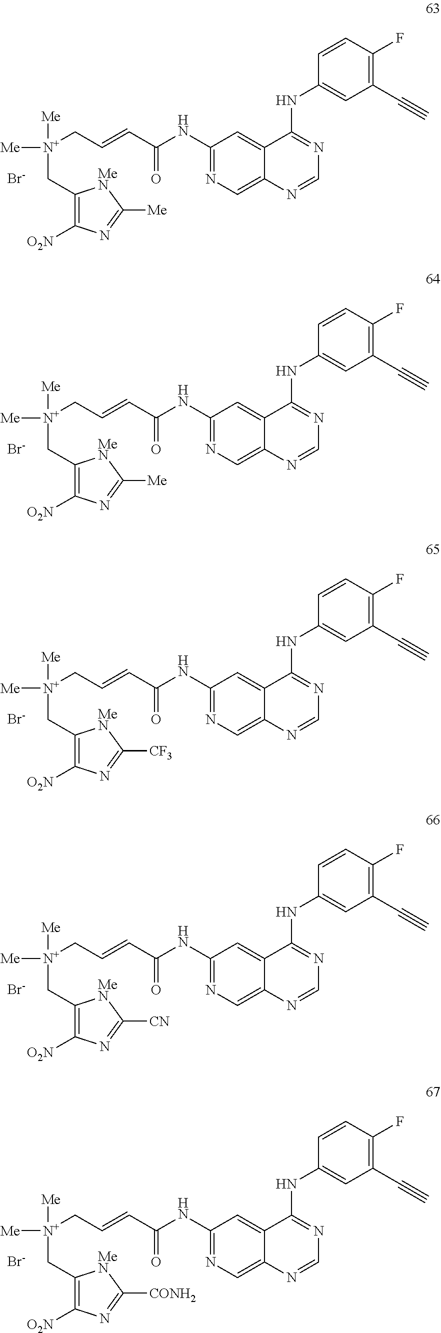

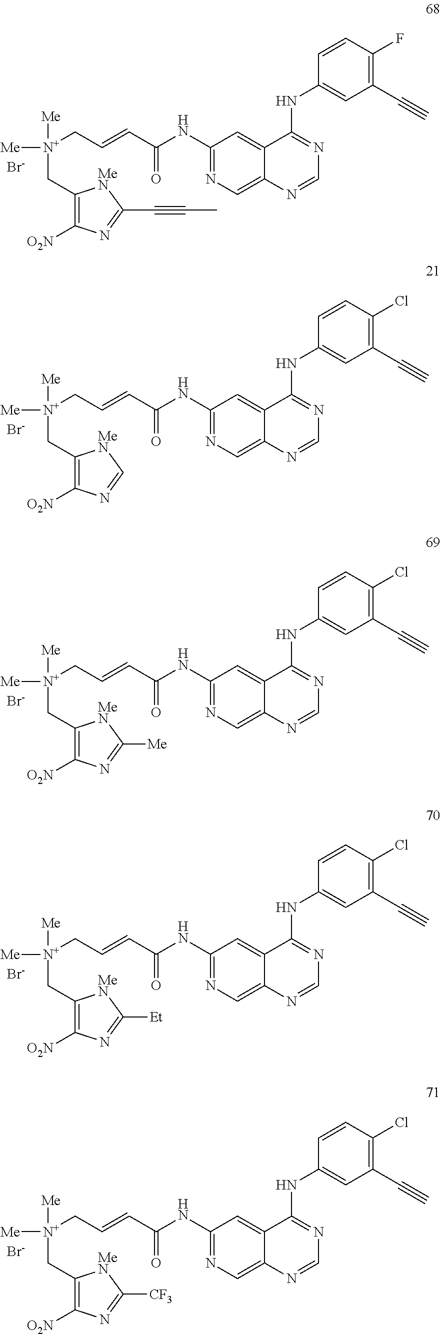

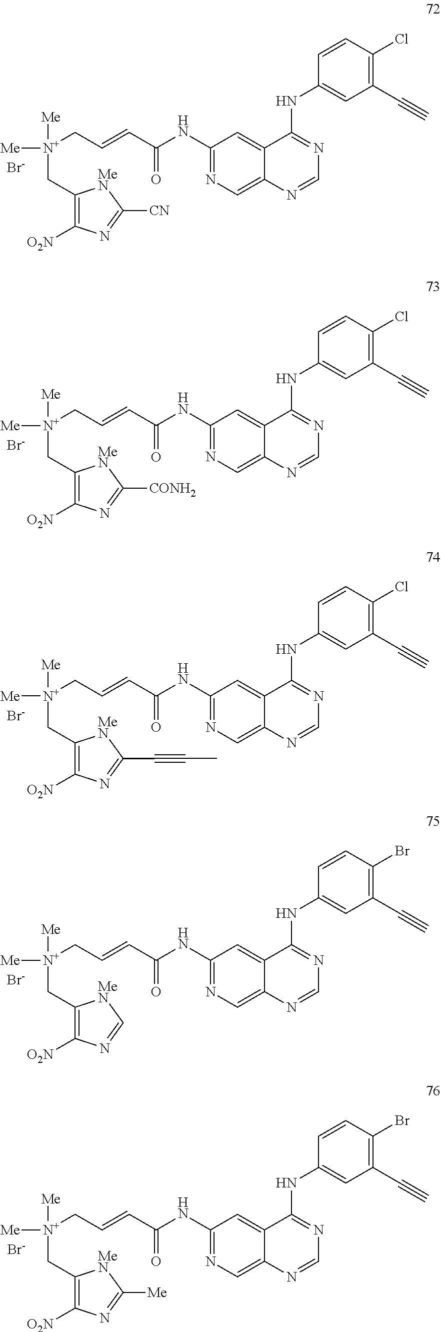

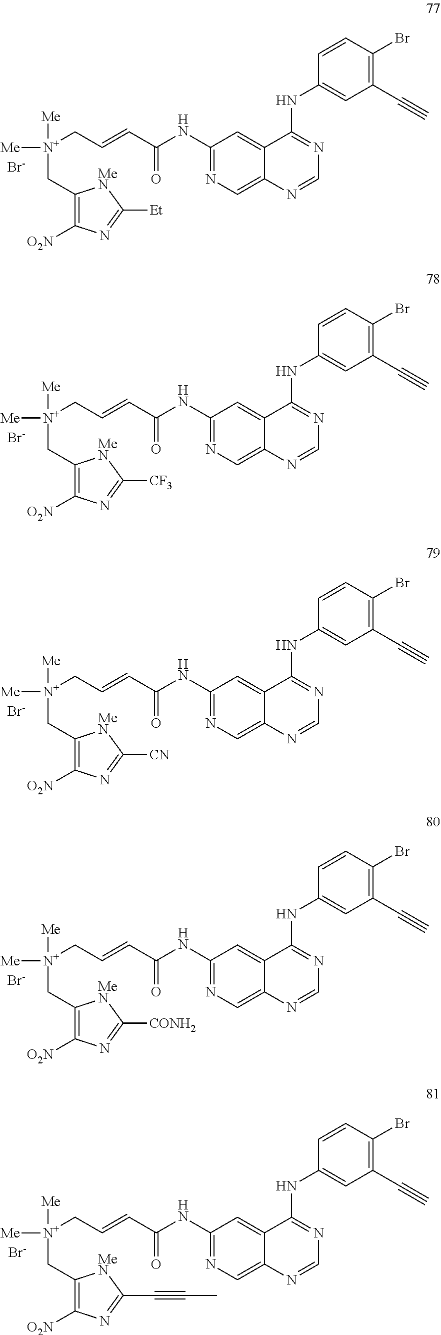

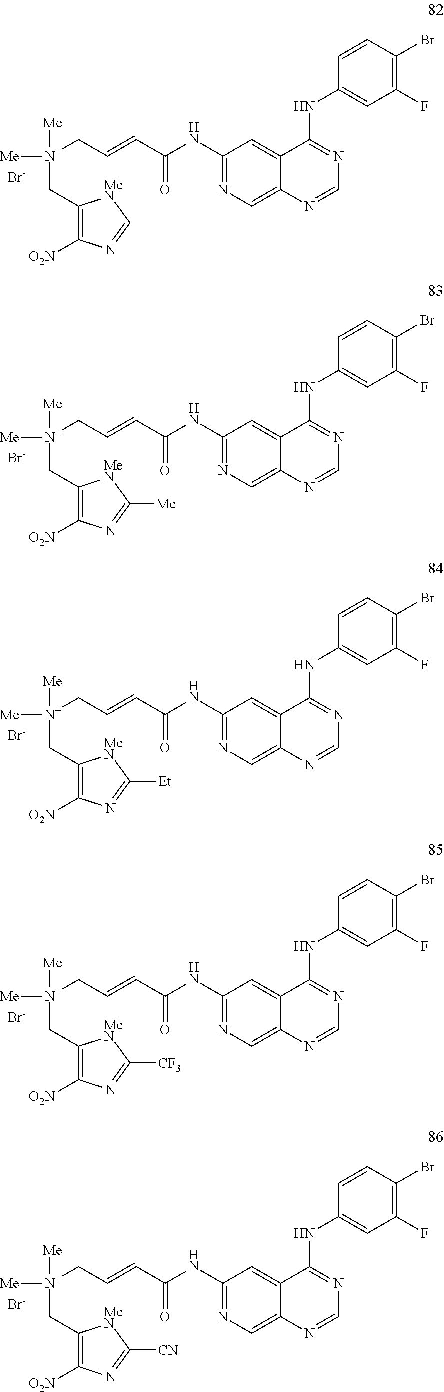

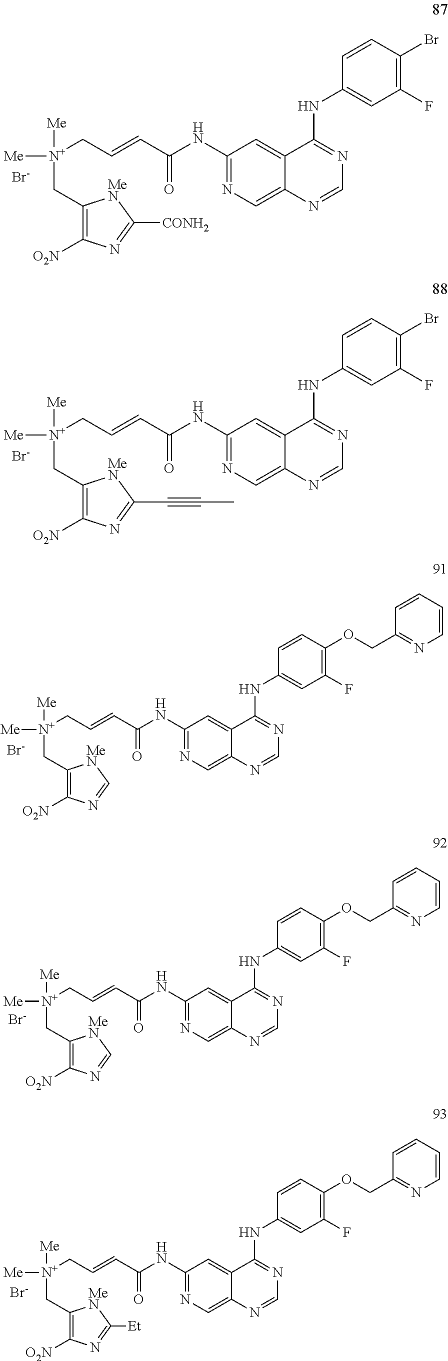

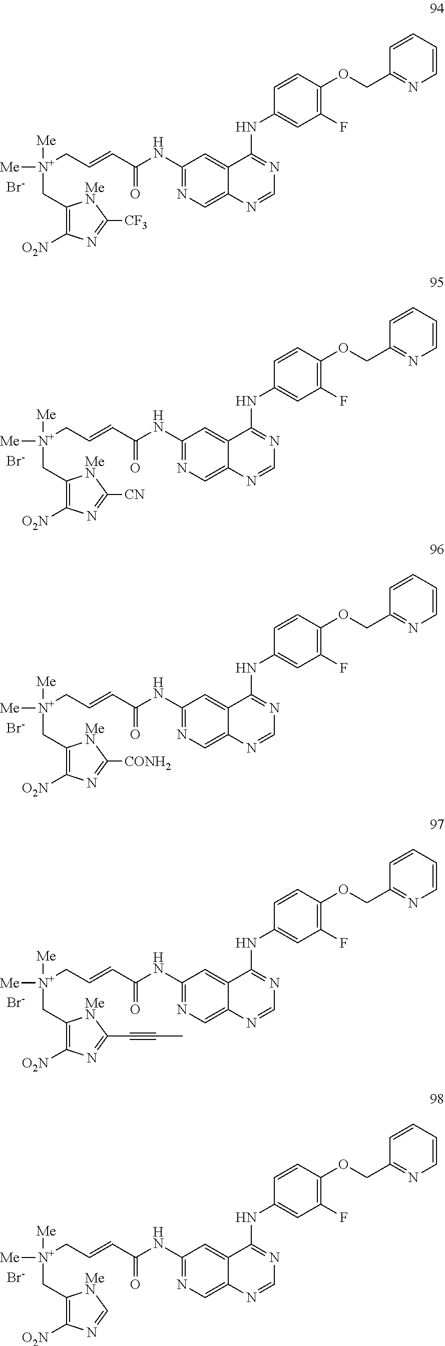

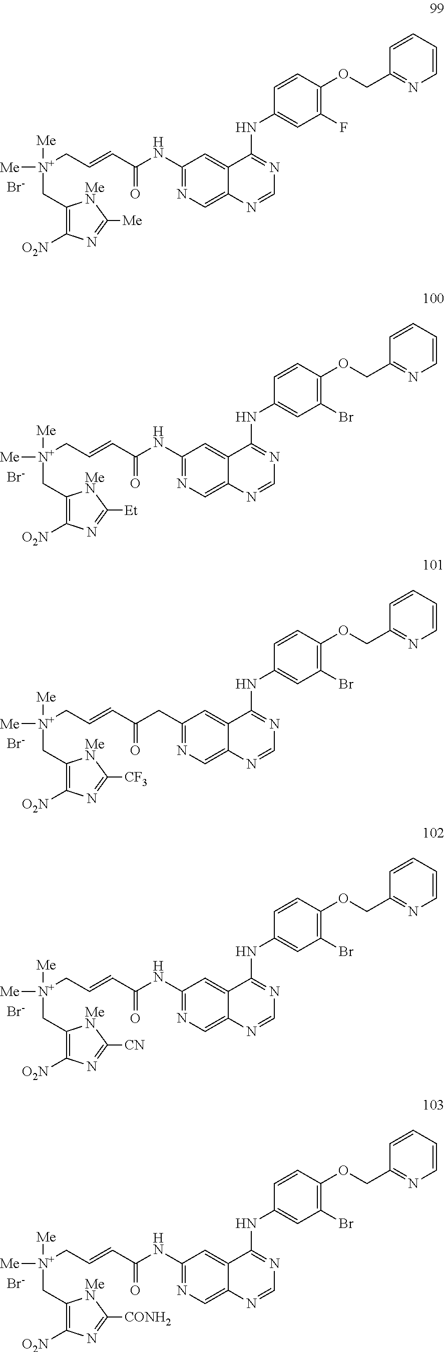

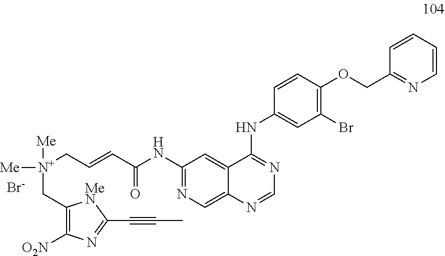 [SMILES: [Br-] ; CC#Cc1nc([N+](=O)[O-])c(C[N+](C)(C)C/C=C/C(=O)Nc2cc3c(Nc4ccc(OCc5ccccn5)c(Br)c4)ncnc3cn2)n1C]

In some embodiments, the compounds are selected from the group consisting of:
(2E)-4-{[4-(3-bromoanilino)-6-quinazolinyl]amino}-N,N-dimethyl-N-(4-nitro-benzyl)-4-oxo-2-buten-1-ammonium bromide;
(2E)-4-{[4-(3-bromoanilino)-6-quinazolinyl]amino}-N,N-dimethyl-N-(2-nitro-benzyl)-4-oxo-2-buten-1-ammonium bromide;
(2E)-4-{[4-(3-bromoanilino)-6-quinazolinyl]amino}-N,N-dimethyl-N-[(1-methyl-5-nitro-1H-pyrrol-2-yl)methyl]-4-oxo-2-buten-1-ammonium bromide;
(2E)-4-{[4-(3-bromoanilino)-6-quinazolinyl]amino}-N,N-dimethyl-N-[(1-methyl-4-nitro-1H-imidazol-5-yl)methyl]-4-oxo-2-buten-1-ammonium bromide;
(2E)-4-{[4-(3-bromoanilino)-6-quinazolinyl]amino}-N,N-dimethyl-N-[(1-methyl-4-nitro-1H-imidazol-2-yl)methyl]-4-oxo-2-buten-1-ammonium bromide;
(2E)-4-{[4-(3-bromoanilino)-6-quinazolinyl]amino}-N,N-dimethyl-N-[(1-methyl-4-nitro-1H-pyrazol-5-yl)methyl]-4-oxo-2-buten-1-ammonium bromide;
(2E)-4-{[4-(3-bromoanilino)-6-quinazolinyl]amino}-N,N-dimethyl-N-[(3-nitroimidazo[1,2-a]pyridin-2-yl)methyl]-4-oxo-2-buten-1-ammonium bromide;
1-((2E)-4-{[4-(3-bromoanilino)-6-quinazolinyl]amino}-4-oxo-2-butenyl)-1-[-(1-methyl-4-nitro-1H-imidazol-5-yl)methyl]piperidinium bromide;
4-((2E)-4-{[4-(3-bromoanilino)-6-quinazolinyl]amino}-4-oxo-2-butenyl)-4-[-(1-methyl-4-nitro-1H-imidazol-5-yl)methyl]morpholin-4-ium formate
(2E)-4-{[4-(3-chloro-4-fluoroanilino)-7-methoxy-6-quinazolinyl]amino}-N,N-dimethyl-N-[(1-methyl-4-nitro-1H-imidazol-5-yl)methyl]-4-oxo-2-buten-1-ammonium bromide;
(2E)-4-{[4-(3-bromo-4-fluoroanilino)-6-quinazolinyl]amino}-N,N-dimethyl-N-[(1-methyl-4-nitro-1H-imidazol-5-yl)methyl]-4-oxo-2-buten-1-ammonium bromide;
(2E)-4-{[4-(4-fluoro-3-methoxyanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide;
(2E)-4-{[4-(3-bromo-4-fluoroanilino)pyrido[3,4-d]pyrimidin-6-yl]amino}-N,—N-dimethyl-N-[(1-methyl-4-nitro-1H-imidazol-5-yl)methyl]-4-oxo-2-buten-1-ammonium bromide;
(2E)-4-{[4-(3-bromo-4-fluoroanilino)pyrido[3,4-d]pyrimidin-6-yl]amino}-N-[(1,2-dimethyl-4-nitro-1H-imidazol-5-yl)methyl]N,N-dimethyl-4-oxo-2-buten-1-ammonium bromide;
(2E)-4-{[4-(3-bromo-4-fluoroanilino)pyrido[3,4-d]pyrimidin-6-yl]amino}-N-[(2-methoxy-1-methyl-4-nitro-1H-imidazol-5-yl)methyl]-N,N-dimethyl-4-oxo-2-buten-1-ammonium bromide;
(2E)-4-{[4-(3-bromo-4-fluoroanilino)pyrido[3,4-d]pyrimidin-6-yl]amino}-N-[(2-ethynyl-1-methyl-4-nitro-1H-imidazol-5-yl)methyl]-N,N-dimethyl-4-oxo-2-buten-1-ammonium bromide;
(2E)-4-{[4-(3-bromo-4-fluoroanilino)pyrido[3,4-d]pyrimidin-6-yl]amino}-N,—N-dimethyl-N-{[1-methyl-4-nitro-2-(trifluoromethyl)-1H-imidazol-5-yl]methy-l}-4-oxo-2-buten-1-ammonium bromide;
(2E)-N-{[1-(3-amino-3-oxopropyl)-4-nitro-1H-imidazol-5-yl]methyl}-4-{[4-(−3-bromo-4-fluoroanilino)pyrido[3,4-d]pyrimidin-6-yl]amino}-N,N-dimethyl-4-oxo-2-buten-1-ammonium bromide;
(2E)-4-{[4-(3-bromo-4-fluoroanilino)pyrido[3,4-d]pyrimidin-6-yl]amino}-N-[(2-cyano-1-methyl-4-nitro-1H-imidazol-5-yl)methyl]-N,N-dimethyl-4-oxo-2-buten-1-ammonium bromide;
(2E)-4-{[4-(3-bromo-4-fluoroanilino)pyrido[3,4-d]pyrimidin-6-yl]amino}-N-[(2-cyano-1-methyl-4-nitro-1H-imidazol-5-yl)methyl]-N,N-dimethyl-4-oxo-2-buten-1-ammonium trifluoroacetate;
(2E)-4-{[4-(3-bromo-4-fluoroanilino)pyrido[3,4-d]pyrimidin-6-yl]amino}-N-{[1-(2-cyanoethyl)-4-nitro-1H-imidazol-5-yl]methyl}-N,N-dimethyl-4-oxo-2-buten-1-ammonium bromide;
(2E)-4-({4-[4-fluoro-3-(trifluoromethyl)anilino]pyrido[3,4-d]pyrimidin-6-yl}amino)-N,N-dimethyl-N-[(1-methyl-4-nitro-1H-imidazol-5-yl)methyl]-4-oxo-2-buten-1-ammonium bromide;
(2E)-N-[(1,2-dimethyl-4-nitro-1H-imidazol-5-yl)methyl]-4-({4-[4-fluoro-3-(trifluoromethyl)anilino]pyrido[3,4-d]pyrimidin-6-yl}amino)-N,N-dimethyl-4-oxo-2-buten-1-ammonium bromide;
(2E)-4-({4-[4-fluoro-3-(trifluoromethyl)anilino]pyrido[3,4-d]pyrimidin-6-yl}amino)-N-[(2-methoxy-1-methyl-4-nitro-1H-imidazol-5-yl)methyl]-N,N-dimethyl-4-oxo-2-buten-1-ammonium bromide;
(2E)-N-[(2-ethynyl-1-methyl-4-nitro-1H-imidazol-5-yl)methyl]-4-({4-[4-fluoro-3-(trifluoromethyl)anilino]pyrido[3,4-d]pyrimidin-6-yl}amino)-N,N-dimethyl-4-oxo-2-buten-1-ammonium bromide;
(2E)-4-({4-[4-fluoro-3-(trifluoromethyl)anilino]pyrido[3,4-d]pyrimidin-6-yl}amino)-N,N-dimethyl-N-{[1-methyl-4-nitro-2-(trifluoromethyl)-1H-imidazo-l-5-yl]methyl}-4-oxo-2-buten-1-ammonium bromide;
(2E)-N-{[1-(3-amino-3-oxopropyl)-4-nitro-1H-imidazol-5-yl]methyl}-4-({4-[-4-fluoro-3-(trifluoromethyl)anilino]pyrido[3,4-d]pyrimidin-6-yl}amino)-N,N-dimethyl-4-oxo-2-buten-1-ammonium bromide;
(2E)-N-[(2-cyano-1-methyl-4-nitro-1H-imidazol-5-yl)methyl]-4-({4-[4-fluoro-3-(trifluoromethyl)anilino]pyrido[3,4-d]pyrimidin-6-yl}amino)-N,N-dimethyl-4-oxo-2-buten-1-ammonium bromide;
(2E)-N-{[1-(2-cyanoethyl)-4-nitro-1H-imidazol-5-yl]methyl}-4-({4-[4-fluoro-3-(trifluoromethyl)anilino]pyrido[3,4-d]pyrimidin-6-yl}amino)-N,N-dimethyl-4-oxo-2-buten-1-ammonium bromide;
(2E)-4-{[4-(3-ethynylanilino)pyrido[3,4-d]pyrimidin-6-yl]amino}-N,N-dimet-hyl-N-[(l-methyl-4-nitro-1H-imidazol-5-yl)methyl]-4-oxo-2-buten-1-ammonium bromide;
(2E)-N-[(1,2-dimethyl-4-nitro-1H-imidazol-5-yl)methyl]-4-{[4-(3-ethynylanilino)pyrido[3,4-d]pyrimidin-6-yl]amino}-N,N-dimethyl-4-oxo-2-buten-1-ammonium bromide;
(2E)-4-{[4-(3-ethynylanilino)pyrido[3,4-d]pyrimidin-6-yl]amino}-N-[(2-methoxy-1-methyl-4-nitro-1H-imidazol-5-yl)methyl]-N,N-dimethyl-4-oxo-2-buten-1-ammonium bromide;
(2E)-4-{[4-(3-ethynylanilino)pyrido[3,4-d]pyrimidin-6-yl]amino}-N-[(2-ethynyl-1-methyl-4-nitro-1H-imidazol-5-yl)methyl]-N,N-dimethyl-4-oxo-2-buten-1-ammonium bromide;
(2E)-4-{[4-(3-ethynylanilino)pyrido[3,4-d]pyrimidin-6-yl]amino}-N,N-dimethyl-N-{[l-methyl-4-nitro-2-(trifluoromethyl)-1H-imidazol-5-yl]methyl}-4-oxo-2-buten-1-ammonium bromide;
(2E)-N-{[1-(3-amino-3-oxopropyl)-4-nitro-1H-imidazol-5-yl]methyl}-4-{[4-(−3-ethynylanilino)pyrido[3,4-d]pyrimidin-6-yl]amino}-N,N-dimethyl-4-oxo-2-buten-1-ammonium bromide;
(2E)-N-[(2-cyano-1-methyl-4-nitro-1H-imidazol-5-yl)methyl]-4-{[4-(3-ethynylanilino)pyrido[3,4-d]pyrimidin-6-yl]amino}-N,N-dimethyl-4-oxo-2-buten-1-ammonium bromide;
(2E)-N-{[1-(2-cyanoethyl)-4-nitro-1H-imidazol-5-yl]methyl}-4-{[4-(3-ethynylanilino)pyrido[3,4-d]pyrimidin-6-yl]amino}-N,N-dimethyl-4-oxo-2-buten-1-ammonium bromide;
(2E)-4-({4-(3-chloro-4-fluoroanilino)-7-[(3S)-tetrahydro-3-furanyloxy]-6-quinazolinyl}amino)-N,N-dimethyl-N-[(1-methyl-4-nitro-1H-imidazol-5-yl)methyl]-4-oxo-2-buten-1-ammonium trifluoroacetate;
(2E)-4-({4-[3-chloro-4-(2-pyridinylmethoxy)anilino]-3-cyano-7-ethoxy-6-quinolinyl}amino)-N,N-dimethyl-N-[(1-methyl-4-nitro-1H-imidazol-5-yl)methyl]-4-oxo-2-buten-1-ammonium trifluoroacetate;
(2E)-4-{[4-(3-chloro-4-fluoroanilino)-3-cyano-7-ethoxy-6-quinolinyl]amino-}-N,N-dimethyl-N-[(1-methyl-4-nitro-1H-imidazol-5-yl)methyl]-4-oxo-2-buten-1-ammonium bromide;
2-(4-{[6-(2,6-dichlorophenyl)-8-methyl-7-oxo-7,8-dihydropyrido[2,3-d]pyrimidin-2-yl]amino}phenoxy)-N,N-diethyl-N-[(1-methyl-4-nitro-1H-imidazol-5-yl)methyl]ethanammonium bromide;
2-(4-{[6-(2,6-dichlorophenyl)-8-methyl-7-oxo-7,8-dihydropyrido[2,3-d]pyrimidin-2-yl]amino}phenoxy)-N-[(1,2-dimethyl-4-nitro-1H-imidazol-5-yl)methyl-]N,N-diethylethanammonium bromide;
4-{[6-(2,6-dichlorophenyl)-8-methyl-7-oxo-7,8-dihydropyrido[2,3-d]pyrimidin-2-yl]amino}-1-[(1-methyl-4-nitro-1H-imidazol-5-yl)methyl]pyridinium bromide;
1-[2-(4-{[6-(2,6-dichlorophenyl)-8-methyl-7-oxo-7,8-dihydropyrido[2,3-d]pyrimidin-2-yl]amino}phenoxy)ethyl]-1-[(1-methyl-4-nitro-1H-imidazol-5-yl)methyl]piperidinium bromide;
N,N-diethyl-2-[({5-[(Z)-(5-fluoro-2-oxo-1,2-dihydro-3H-indol-3-ylidene)methyl]-2,4-dimethyl-1H-pyrrol-3-yl}carbonyl)amino]-N-[(1-methyl-4-nitro-1H-imidazol-5-yl)methyl]ethanammonium trifluoroacetate;
N-[(1,2-dimethyl-4-nitro-1H-imidazol-5-yl)methyl]-N,N-diethyl-2-[({5-[(Z)-(5-fluoro-2-oxo-1,2-dihydro-3H-indol-3-ylidene)methyl]2,4-dimethyl-1H-pyrrol-3-yl}carbonyl)amino]ethanammonium bromide;
4-({[4-(4-bromo-2-fluoroanilino)-6-methoxy-7-quinazolinyl]oxy}methyl)-1-m-ethyl-1-[(1-methyl-4-nitro-1H-imidazol-5-yl)methyl]piperidinium trifluoroacetate;
(2E)-4-{[4-(3-bromo-4-fluoroanilino)pyrido[3,4-d]pyrimidin-6-yl]amino}-N-[(2-ethyl-1-methyl-4-nitro-1H-imidazol-5-yl)methyl]-N,N-dimethyl-4-oxo-2-buten-1-ammonium bromide;
(2E)-4-{[4-(3-bromo-4-fluoroanilino)pyrido[3,4-d]pyrimidin-6-yl]amino}-N,—N-dimethyl-N-{[1-methyl-4-nitro-2-(1-propynyl)-1H-imidazol-5-yl]methyl}-4-oxo-2-buten-1-ammonium bromide;
(2E)-4-{[4-(3-bromo-4-fluoroanilino)pyrido[3,4-d]pyrimidin-6-yl]amino}-N,—N-dimethyl-N-[(1-methyl-2-nitro-1H-imidazol-5-yl)methyl]-4-oxo-2-buten-1-ammonium bromide;
(2E)-4-{[4-(3-bromo-4-fluoroanilino)pyrido[3,4-d]pyrimidin-6-yl]amino}-N-[(4-ethyl-1-methyl-2-nitro-1H-imidazol-5-yl)methyl]-N,N-dimethyl-4-oxo-2-buten-1-ammonium bromide; and
(2E)-N-[(2-ethyl-1-methyl-4-nitro-1H-imidazol-5-yl)methyl]-4-{[4-(3-ethynylanilino)pyrido[3,4-d]pyrimidin-6-yl]amino}-N,N-dimethyl-4-oxo-2-buten-1 ammonium bromide.

In some embodiments, the compounds disclosed herein are administered to treat cancer, and specifically, HER-driven cancer. In some embodiments, the cancer to be treated comprises lung cancer. In other embodiments, the lung cancer comprises non-small cell lung cancer. In yet other embodiments, the cancer comprises gastric cancer. In yet other embodiments, the cancer comprises breast cancer. In yet other embodiments, the cancer comprises head and neck squamous cell carcinoma (HNSCC). In yet other embodiments, the cancer comprises gastric/gastroesophageal (GE) junction cancer. In yet other embodiments, the cancer comprises esophageal cancer. In yet other embodiments, the cancer comprises salivary cancer. In yet other embodiments, the cancer comprises ovarian cancer. In yet other embodiments, the cancer comprises endometrial cancer. In yet other embodiments, the cancer comprises uterine cancer. In yet other embodiments, the cancer comprises pancreatic cancer. In some embodiments, the cancer comprises biliary tract cancer. In some embodiments, the cancer comprises bladder cancer. In some embodiments, the cancer comprises colorectal cancer. In some embodiments, the cancer comprises renal cancer. In some embodiments, the cancer comprises brain and/or spinal cord cancer (glioblastoma). In some embodiments, the cancer comprises lymphoma, e.g., primary central nervous system lymphoma. In some embodiments, the cancer comprises leukemia, e.g., acute lymphoblastic leukemia.

In some embodiments, the cancer is selected from the group of lung cancer, gastric cancer, breast cancer, HNSCC, GE junction cancer, esophageal cancer, salivary cancer, ovarian cancer, endometrial cancer, uterine cancer, prostate cancer, pancreatic cancer, colon cancer, biliary tract cancer, bladder cancer, coloreactal, renal, glioblastoma, mesothelioma, adenocarcinoma, lymphoma, and leukemia.

In some embodiments, the cancer is non-small cell lung cancer.

In some embodiments, the cancer is breast cancer.

In some embodiments, the cancer is brain cancer.

In some embodiments, the cancer is a brain metastasis. For example, the brain metastasis can originate from a cancer that it outside of the central nervous system, and then cross the blood-brain barrier to metastasize to the brain. Accordingly, certain brain tumors can be associated with (e.g., derived from) cancers that originate elsewhere in the body.

In some embodiments, the brain metastasis is associated with non-small cell lung cancer.

In some embodiments, the cancer is urothelial carcinoma.

In some embodiments, the cancer is lung adenocarcinoma.

In some embodiments, the cancer is gastric cancer.

In some embodiments, the cancer is spinal cord cancer.

In some embodiments, the cancer is Lynch and Lynch-like colorectal cancer.

In some embodiments, the cancer is non-small cell lung cancer associated with (e.g., characterized by) an EGFR mutation.

In some embodiments, the cancer is breast cancer associated with (e.g., characterized by) an EGFR mutation.

In some embodiments, the cancer is brain cancer associated with (e.g., characterized by) an EGFR mutation.

In some embodiments, the cancer is a brain metastasis associated with (e.g., characterized by) an EGFR mutation.

In some embodiments, the brain metastasis is associated with non-small cell lung cancer associated with (e.g., characterized by) an EGFR mutation.

In some embodiments, the cancer is urothelial carcinoma associated with (e.g., characterized by) an EGFR mutation.

In some embodiments, the cancer is lung adenocarcinoma associated with (e.g., characterized by) an EGFR mutation.

In some embodiments, the cancer is gastric cancer associated with (e.g., characterized by) an EGFR mutation.

In some embodiments, the cancer is spinal cord cancer associated with (e.g., characterized by) an EGFR mutation.

In some embodiments, the cancer is Lynch and Lynch-like colorectal cancer associated with (e.g., characterized by) an EGFR mutation.

In some embodiments, the compounds disclosed herein can cross the blood-brain barrier. Accordingly, In some embodiments, the cancer is brain cancer or spinal cord cancer.

In some embodiments, the cancer is selected from glioblastoma, glioma, brain stem and hypophtalmic glioma, cerebellar and cerebral astrocytoma, medulloblastoma, ependymoma, neuroectodermal tumors, pineal tumors, primary central nervous system lymphoma, and acute lymphoblastic leukemia that has crossed the blood-brain barrier. In some embodiments, the cancer is secondary brain metastases (e.g., cancers that originate elsewhere in the body but cross the blood-brain barrier and metastasize in the brain). In some embodiments, the cancer comprises NSCLC with brain metastases. In some embodiments, the concentration of the compounds disclosed herein in the brain is surprisingly sufficient to treat a HER-driven brain or spinal cord cancer.

In some embodiments, Compound A passes the blood-brain barrier. In some embodiments, Compound A passes the blood-brain barrier and convert to its active metabolite, Compound B. In some embodiments, Compound A converts to its active metabolite, Compound B, and Compound B then passes the blood-brain barrier. In some embodiments, Compound B passes the blood-brain barrier.

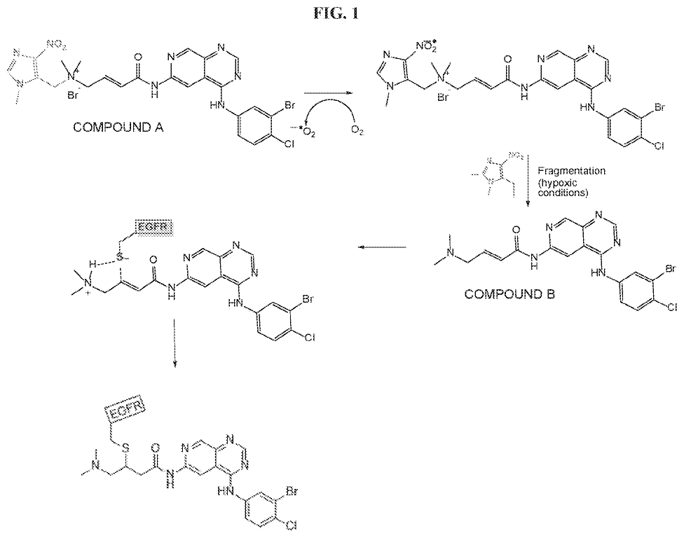

While not wishing to be bound by any theory, it is believed that the compounds disclosed herein bind to certain HER proteins, e.g., EGFR, via several mechanisms. In some embodiments, the compounds disclosed herein bind to EGFR via a covalent bond with the cysteine (EGFR C797) residue as shown in FIG. 1. In some embodiments, the covalent bond is an irreversible interaction. In some embodiments, the compounds disclosed herein bind to HER proteins, e.g., EGFR, via an alternative mechanism, e.g., a reversible, non-covalent interaction with the active site. In some embodiments, the compounds disclosed herein bind to HER proteins, e.g., EGFR, via multiple interactions, e.g, via covalent interactions and non-covalent interactions. In some embodiments, specific mutations in the HER protein, e.g., EGFR, can affect the binding of the compounds disclosed herein with the protein.

In some embodiments, the cancer comprises at least one of an EGFR mutation and an ErbB-2 mutation. In some embodiments, the cancer comprises an EGFR mutation. In some embodiments, the cancer comprises an ErbB-2 mutation. In some embodiments, the cancer comprises an ErbB-3 mutation. In some embodiments, the cancer comprises an ErbB-4 mutation.

In some embodiments, the EGFR mutation comprises A763_Y764insFHEA. In some embodiments, the EGFR mutation comprises C797S/L858R. In some embodiments, the EGFR mutation comprises d746-750. In some embodiments, the EGFR mutation comprises d746-750/C797A. In some embodiments, the EGFR mutation comprises d746-750/C797S. In some embodiments, the EGFR mutation comprises d746-750/T790M/C797S. In some embodiments, the EGFR mutation comprises D770GY. In some embodiments, the EGFR mutation comprises D770_N771insNPG. In some embodiments, the EGFR mutation comprises G719C. In some embodiments, the EGFR mutation comprises G719S. In some embodiments, the EGFR mutation comprises L747S. In some embodiments, the EGFR mutation comprises L858R. In some embodiments, the EGFR mutation comprises L858R/T790M. In some embodiments, the EGFR mutation comprises L861Q. In some embodiments, the EGFR mutation comprises T790M/C797S/L858R.

In some embodiments, the EGFR mutation comprises C797S. In some embodiments, the EGFR mutation is C797S. In some embodiments, the EGFR mutation is selected from a single, double, or triple mutation. In some embodiments, the EGFR mutation is selected from a double or triple EGFR mutation. In some embodiments, at least one of the EGFR mutations in the double or triple EGFR mutation is C797S.

In some embodiments, the EGFR mutation comprises at least one of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R. In some embodiments, the EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R.

In some embodiments, the ErbB-2 mutation comprises D769H. In some embodiments, the ErbB-2 mutation comprises D769Y. In some embodiments, the ErbB-2 mutation comprises R896C. In some embodiments, the ErbB-2 mutation comprises V777L. In some embodiments, the ErbB-2 mutation comprises V777_G778insCG.

In some embodiments, the ErbB-2 mutation comprises at least one of D769H, D769Y, R896C, V777L, and V777_G778insCG. In some embodiments, the ErbB-2 mutation selected from the group consisting of: D769H, D769Y, R896C, V777L, and V777_G778insCG. In some embodiments, the ErbB-2 mutation is selected from a single, double, or triple mutation. In some embodiments, the EGFR mutation is selected from a double or triple ErbB-2 mutation.

In some embodiments, the EGFR exon mutation is an EGFR exon mutation selected from a HER2, HER3, and HER4 mutation.

In some embodiments, the EGFR exon mutation is an EGFR exon mutation selected from the group consisting of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R, EGFR exon 20 insertion mutations (Reiss et al J Thorac Oncol. 2018 October; 13(10):1560-1568, Arcila et al Mol Cancer Ther. 2013 February; 12(2): 220-229), EGFR rare and compound mutations (Beau-Faller et al Annals of Oncology 25: 126-131, 2014, Martin et al Clin Lung Cancer. 2019 May 11. pii: S1525-7304(19)30103-2, Mishra et al Oncotarget, 2017, Vol. 8, (No. 69), pp: 114371-114392), tertiary Osimertinib resistance mutations L718Q/V, G724S, L792F/H, G796S, C797S/G (Thress et al., Nat. Med. 21 (6) (2015) 560-562; Nishino et al, Lung Cancer, Volume 126, 149-155; Le et al, Clin Cancer Res. 2018 Dec. 15; 24(24):6195-6203), EGFR gene fusions EGFR-SEPT14, EGFR-RAD51 (Konduri et al, Cancer Discov. 2016 June; 6(6):601-11, Shah et al BMC Genomics. 2013; 14:818-832; Frattini et al, Nat Genet. 2013; 45:1141-1149), EGFR-PSPH or additional C-terminal partners, and EGFR kinase domain duplication (EGFR-KDD) (Gallant et al, Cancer Discov. 2015 November; 5(11): 1155-63), D769H, D769Y, R896C, V777L, V777_G778insCG, G309A/E, S310F/Y, V659E/D, G660D, K753E, L755P/S, Del755-759, L768S, D769H/Y, V773L, A775_G776insYVMA, G776V/L, Cins, V777L, P780Ins, P780_Y781insGSP, V842I, L866M, R896C (Bose, et al, 2013, Cancer Discov 3:224-37; Greulich, et al., 2012, Proc Natl Acad Sci USA 109:14476-81; Kavuri, et al., 2015, Cancer Discov 5:832-41; Ou, et al., 2017, J Thorac Oncol 12:446-457; Zuo, et al., 2016, Clin Cancer Res 22:4859-4869; Verma, et al., 2018, PLoS One 13:e0190942, 2018; Bellmunt, et al., 2015, Cancer Med 4:844-52; Kloth, et al., 2016, Gut 65:1296-305; Xu, et al., 2017, Clin Cancer Res 23:5123-5134; Stephens, et al., 2004, Nature 431:525-6; Shigematsu, et al., 2005, Cancer Res 65:1642-6; Wang, et al., 2006, Cancer Cell 10:25-38), juxtamembrane and transmembrane domain mutations (Pahuja et al Cancer Cell 34, 792-806, Nov. 12, 2018) S653C, S656C, V659E, G660D, and G660R, and JMD mutants R677L, R678W, T686A, E693K, S649T, P650S, L651V, V659G, G660D, G660R, L663P, L674V, R677L, R678Q, R683Q, E693K, Q709L, and A710V and ErbB2 gene fusions (HER2 fusion Refs: Yu, et al., 2015, J Transl Med 13:116, 2015; Ross, et al., 2013, Clin Cancer Res 19:2668-76; Ross, et al., 2014, Clin Cancer Res 20:68-75; Chmielecki, et al., 2015, Oncologist 20:7-12; Gao, et al., 2018, Cell Rep 23:227-238 e3; Mishra et al Oncotarget, 2017, Vol. 8, (No. 69), pp: 114371-114392) ZNF207-HER2, MDK-HER2, NOS2-HER2, ERBB2-GRB7, ERBB2-CTTN, ERBB2-PPP1R1B, ERBB2-PSMB3 or additional N-terminal partners, N181S, T244R, Y285C, R306S, V348L, D595V, H618P, D931Y, K935I (Kurppa et al, Oncogene (2015), 1-9), E317K, E452K, E542K, R544W, E563K, E836K, E872K (Prickett et al, Nat Genet. 2009 October; 41(10): 1127-32) and HER4 gene fusions EZR-ERBB4, IKZF2-ERBB4, BGALT-ERBB4 or additional N-terminal partners, T355I, F94L, G284R, D297Y, T355I, E1261A; V104M, A232V, P262H, G284R, T389K, V714M, Q809R, S846I and E928G; any activating mutation, TKI resistance mutations, gene fusion, kinase domain duplication, and gene amplification of ErbB receptors.

In some embodiments, the EGFR exon mutation is an EGFR exon mutation selected from the group consisting of A763_Y764insFHEA, C797S/L858R, d746-750, d746-750/C797A, d746-750/C797S, d746-750/T790M/C797S, D770GY, D770_N771insNPG, G719C, G719S, G719X, L747S, S768I, L858R, L858R/T790M, L861Q, and T790M/C797S/L858R, EGFR exon 20 insertion mutations (Reiss et al J Thorac Oncol. 2018 October; 13(10): 1560-1568, Arcila et al Mol Cancer Ther. 2013 February; 12(2): 220-229), EGFR rare and compound mutations (Beau-Faller et al Annals of Oncology 25: 126-131, 2014, Martin et al Clin Lung Cancer. 2019 May 11. pii: S1525-7304(19)30103-2, Mishra et al Oncotarget, 2017, Vol. 8, (No. 69), pp: 114371-114392), tertiary Osimertinib resistance mutations L718Q/V, G724S, L792F/H, G796S, C797S/G, EGFR gene fusions EGFR-SEPT14, EGFR-RAD51, EGFR-PSPH or additional C-terminal partners, and EGFR kinase domain duplication (EGFR-KDD).

In some embodiments, the EGFR mutation is an L858R mutation.

In some embodiments, the L858R mutation is selected from the group consisting of L858R and T790M, L858R/C797S, and L858R/C797S/T790M.

In some embodiments, the EGFR mutation is an ErbB-2 (HER2) mutation.

In some embodiments, the ErbB-2 (HER2) mutation is selected from the group consisting of D769H, D769Y, R896C, V777L, V777_G778insCG, G309A/E, S310F/Y, V659E/D, G660D, K753E, L755P/S, Del755-759, L768S, D769H/Y, V773L, A775_G776insYVMA, G776V/L, Cins, V777L, P780Ins, P780_Y781insGSP, V842I, L866M, R896C, juxtamembrane and transmembrane domain mutations S653C, S656C, V659E, G660D, and G660R, and JMD mutants R677L, R678W, T686A, E693K, S649T, P650S, L651V, V659G, G660D, G660R, L663P, L674V, R677L, R678Q, R683Q, E693K, Q709L, and A710V and ErbB2 gene fusions ZNF207-HER2, MDK-HER2, NOS2-HER2, ERBB2-GRB7, ERBB2-CTTN, ERBB2-PPP1R1B, ERBB2-PSMB3 or additional N-terminal partners.

In some embodiments, the HER2 mutation is selection from the group consisting of A289D/I/N/TN, A466T, A775_G776insSVMA, A775_G776insV, A775_G776insYVMA, C311R, C334S, C797S/Y, D227G/H/V/Y, D769H, D769Y, del.755-759, E321G, E790A/K/Q, G309A, G309E, G598V, G660D/R, G719A, G719A/C/D/S, G776>VC, G776_V777>AVCV, G776_V777>AVGCV, G776_V777>AVGSGV, G776_V777>VCV, G776C, G776S, G776V, G778S, I263T, I675M, I767M, L755S, L755S/W, L858R, L861Q/R, L866M, L869Q, L869R, N1219S, N319D, P780_Y781insGSP, P780ins, R103Q, R108G/K, R222C, R252C, R678Q, R678Q+L755W, R868W, R896C, S310F, S310Y, S768G/I/T, T733I, T790M, T798I/M, T862A, V659_660VE, V659E/D, V659E/G660R, V659E/V660R, V664E, V664F, V665M, V769L, V777_G778insC, V777A, V777L, V777M, V842I, and Y772_V773insLMAY.

In some embodiments, the EGFR exon mutation is an ErbB-3 (HER3) mutation.

In some embodiments, the ErbB-3 (HER3) (Mishra et al Oncotarget. 2018 Jun. 12; 9(45): 27773-27788; Jaiswal, et al., 2013, Cancer Cell 23:603-17) mutation is selected from the group consisting of: T355I, F94L, G284R, D297Y, T355I, E1261A; V104M, A232V, P262H, G284R, T389K, V714M, Q809R, S846I and E928G; any activating mutation, TKI resistance mutations, gene fusion, kinase domain duplication, and gene amplification of ErbB receptors.

In some embodiments, the HER3 mutation is selected from the group consisting of A232A, A232V, D297Y, E332K, E928G, G284R, K329E, M91I, P262H, Q809R, R475W, R667C/H, S846I, T355I, T389K, V104L/M, and V855A.

In some embodiments, the EGFR exon mutation is an ErbB-4 (HER4) mutation.

In some embodiments, the ErbB-4 (HER4) mutation is selected from the group consisting of N181S, T244R, Y285C, R306S, V348L, D595V, H618P, D931Y, K935I, E317K, E452K, E542K, R544W, E563K, E836K, E872K and HER4 gene fusions EZR-ERBB4, IKZF2-ERBB4, BGALT-ERBB4 or additional N-terminal partners.

In some embodiments, the HER4 mutation is selected from the group consisting of A657V, A705V, A710V, D595V, D609N, D931Y, E317D, E317K, E452K, E542K, E563K, E693G, E693K, E695K, E836K, E872K, G660D, G660R, G668E/R, G672R, G704E/R, G936R, H618P, I654L/M/T, I655M, I673F/M/V, I675L/M/T, I682M/T, K935I, L39F, L662V, L674I/V, L798R, M313I, M712L. N181S, P1033S, P409L, P650L/S, P699S/del, P700S, P702L, P702S, pG776insV_G/C, Q679E/H, Q709K, Q709L, Q711H, R106C/H, R1174Q, R306S, R393W, R491K, R544W, R677L, R677Q, R678P, R678Q, R678W, R683Q, R683W, R688L/Q/W, R689I/K, R713L/Q/W, R771C, R847C/H, R992C/S, S1246N, S1289A, S303F/Y, S341L, S653C, S653P, T244R, T652M, T652R, T686A/M/R, V348L, V659D, V659D/ins, V659E, V664F/I, V665M, V665M/del, V669A/L, V697L, V697L/M/del, V840I, Y111H, Y285C, and Y685H.

In some embodiments, the EGFR mutation is an exon 19 deletion.

In some embodiments, the exon 19 deletion is selected from the group consisting of d746-750, d746-750/C797A, d746-750/C797S, and d746-750/T790M/C797S.

In some embodiments, the EGFR mutation is selected from the group consisting of A702T, A743V, A767_V769dup, A840T, A840V, C620W, C797G, C797S, D770_N771delinsP, D855Y, E709_T710delinsD, E734V, E749Q, G724C, G724S, G735D, G779F, G796S, G857E, G863S, G874D, H773_V774delinsLM, H773dup, H835fs*35, H870R, K713T, K753E, K755S, L718Q, L718V, L730R, L747P, L768S, L792F/H, L792K, L844V, L858R, L858R with C797G, L858R with C797S, L858R with L718Q, L858R with L718V, L858R with L792F/H, L858R with T790M, N771_H773dup, N772delinsGY, P596L, P699L, P741S, P848L, Q791H, R831C, S768 D770 dup, S768_V769delinsIL, T725M, T790M, T847I, V765M, V773L, V834L, V843I, and V843L.

In some embodiments, the EGFR mutation is an EGFR exon 20 insertion mutation.

In some embodiments, the EGFR exon 20 insertion mutation is selected from D770_N771>ASVDN, N771_P772>SVDNP, N771_H773dupNPH, N771_P772insHH, N771_P772insH, N771_P772insNN, N771_P772insG, N771_P772>GYP, N771_P772insGTDN, N771_P772insY, N771_P772insV, N771_P772insT, N771_P772>SVDSP, N771_P772>SPHP, N771_P772>SHP, N771_P772>SEDNS, N771_P772>RDP, N771_P772>KGP, N771_P772>KFP, N771dupN, N771>GY, V774_C775>AHVC, V774_C775>GNPHVC, V774_C775>GTNPHVC, V774_C775insHNPHV, V774_C775insHV, A769_D770insASV, A763_Y764insFQEA, A763_Y764insLQEA, A767_V769dupASV, S768_D779dupSVD, S768_V769>PL, S768_V769>TLASV, V769_D770insSAVS, V769_D770insSGSV, V769_D770insSLRD, V769_H773>LDNPNPH, V769_D770insE, V769_D770insGE, V769_D770insGTV, V769_D770insGVM, V769_N771dupVDN, D770_N771insG, D770_N771>GYN, D770_N771>GSVDN, D770_N771>GVVDN, D770_N771insH, D770_P772dupDNP, D770_N771>QVH, D770_N771insAVD, D770_N771insGT, D770_N771insGV, D770_N771>EGN, M793_P794>ITQLMP, H773_V774dupHV, H773_V774insY, H773_V774insNPY, H773_V774insTH, H773_V774insSH, H773_V774insPY, H773_V774insHPH, H773_V774>NPNPYV, H773_V774>PNPYV, H773dupH, H773>YNPY, P772_H773dupPH, P772_H773insGNP, P772_H773>RHPH, Y764_V765insHH, A767_S768insTLA, D770ins_N771insSVD, V774_C775insHV, P772_H773insGDP, I744_K745insKIPVAI, K745_E746insIPVAIK, K745_E746insVPVAIK, A763_Y764insFHEA, A775_G776insYVMA, G776>VC, V777_G778insCG, P780_Y781insGSP, D770>GY, D770_N771insY, D770_N771insGF, D770_N771insSVD, N771_P772insN, P772_H773insNP, P772_H773insNPH, H773_V774insAH, H773_V774insPH, H773_V774insH, H773_V774insNPH, and V774_C775insHV.

In some embodiments, the EGFR exon 20 insertion mutation is selected from A763_Y764insFHEA, A763insFQEA, D770_N771insNPG, D770GY, D770insSVD, H773insH, H773insNPH, V769insASV and V777_G778insCG.

In some embodiments, the EGFR exon 20 insertion mutation is selected from D770_N771>ASVDN, N771_P772>SVDNP, N771_H773dupNPH, and A763_Y764insFQEA.

In some embodiments, the EGFR exon mutation is an EGFR exon mutation of at least one of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R. In some embodiments, the ErbB-2 mutation is at least one of D769H, D769Y, R896C, V777L, and V777_G778insCG.

In some embodiments, the EGFR mutation is a compound mutation.

In some embodiments, the compound mutation is selected from the group consisting of p.E709G p.L858R, p.E746_A750del p.T751P, p.E746_R748del p.A750P, p.G719A P.L861Q, p.G719A p.L861R, p.G719C p.L833_V834delinsFL, p.G719C p.L861Q, p.G719C p.S768I, p.G721D p.E746_A750del, p.G724S p.S768I, p.L833_V834delinsFL p.L858R, p.L833V p.H835L, p.L858R p.A871E, p.N700S p.S784F, p.N700S p.T783A, p.S720F p.L861Q, p.T725M p.K728E, p.T751_I759del p.L798F, p.V738F p.L858R, and p.V834L p.L858R.

In some embodiments, the EGFR mutation is a fusion mutation.

In some embodiments, the fusion mutation is selected from the group consisting of MDK-HER2, NOS2-HER2, and ZNF207-HER2.

In some embodiments, the fusion mutation is selected from the group consisting of MDK_ex4/HER2_ex11, NOS2_ex2/HER2_ex2, and ZNF207_ex2/HER2_ex18.

In some embodiments, the fusion mutation is selected from the group consisting of EGFR-KDD (kinase domain duplication), EGFR-NTRK1, EGFR-PPM1H, EGFR-PSPH, EGFR-PSPHP1, EGFR-RP11, EGFR-RP11-745C, EGFR-SEPT14, and EGFR-SEPT14 fusions.

In some embodiments, the fusion mutation is selected from the group consisting of EGFR-SEPT14 and ERBB2-PSMB3.

In some embodiments, the EGFR mutation is EGFR atypical.

In some embodiments the atypical mutation is selected from the group consisting of G719C, G719S, L747S, and L861Q.

In some embodiments, the EGFR mutation is a rare mutation.

In some embodiments, the rare EGFR mutation is selected from the group consisting of complex mutations, exon 18 del/ins, exon 18 G719X mutations, exon 18 other substitutions, exon 20 insertions, exon 20 other substitutions, and L858R complex mutations.

In some embodiments, the rare EGFR mutation is selection from the group consisting of A767V769dupASV, A769D770insASV, E709D, E709X, G709A+G719S, G719A, G719A+S768I, G719C, G719S, G719S+L861Q, G719S+S768I, G719X, L858R+S768D770dupSVD, L858R+S768I, L858R+T790M, S768I, and T790M.

In some embodiments, the rare EGFR mutation is an exon 18 mutation.

In some embodiments, the rare EGFR mutation is an exon 19 mutation.

In some embodiments, the rare EGFR mutation is an exon 18 or exon 19 mutation selected from the group consisting of E697G, E709 T710delinsD, E709A+G719S, E709D, E709H T710del, E711K, Exon 19, G719A, G719C, G719S, G719S+L861Q, L692V, p.A702T, p.A743V, p.E709_T710delinsD, p.E709G, p.E734V, p.E749Q, p.G724C/S, p.G735D, p.K713T, p.K728E, p.L730R, p.L747P, p.P699L, p.P741S, p.S720F, p.T725M, p.V738F, P699L, T725M, and Y693I.

In some embodiments, the rare EGFR mutation is an exon 20 mutation.

In some embodiments, the rare EGFR mutation is an exon 20 mutation selected from the group consisting of A763 Y764insFQEA, A767 V769dupASV, A767S768insSVR, D761 E762insEAFQ, D770H773dupTTP, D770 N771insSVD, G796S, H773_V774dupH, H773_V774insPH, H773L+V774M, Ins 2AA, Ins 3AA, M766 V769insWPA, N771 delinsKPP, N771dupN, p.A767_V769dup, p.D770_N771delinsP, p.G779F, p.H773_V774delinsLM, p.H773dup, p.N771_H773dup, p.N771delinsGY, p.Q791H, p.S768_D770dup, p.S768_V769delinsIL, p.V765M, P772 C775dupPHVC, P772H773dupH, P772H773insDNP, P772H773insLGNP, P772H773insT, R776H, S768 D770dupSVD, S768D770dupSVD, associated with L858R, S768D770dupSVD associated with L858R in association with other mutations as T725M, V769M and R776H, S768I+V769L, S768I, associated with L858R, S768I, associated with L858R in association with other mutations as T725M, V769M and R776H, T790M, T790M associated with L858R, T790M associated with L858R in association with other mutations as T725M, V769M and R776H, V769 D770delinsGI, V769 D770insL, V769M, and V774M.

In some embodiments, the rare EGFR mutation is an exon 21 mutation.

In some embodiments, the rare EGFR mutation is an exon 21 mutation selected from the group consisting of p.A840T, p.A840V, p.A871E, p.D855Y, p.G857E, p.G863S, p.G874D, p.H835fs*55, p.H835L, p.L833_V834delinsFL, p.L833V, p.L861Q/R, p.P848L, P.R831C, p.T847I, p.V834L, p.V843I and p.V843L.

In some embodiments, the rare EGFR mutation is a complex mutation.

In some embodiments, the rare EGFR mutation is a complex mutation selected from the group consisting of G719A+P772H773dup, G719A+S768I, G719A+V769M, G719C+S768I, G719S+del 19 (E746_A750del), G719S+L861Q, G719S+S768I, P. G724S+p.S768I, p.E709G+p.L858R, p.E746_A750del+p.T751P, p.E746_R748del+p.A750P, p.G719A+p.L861Q, p.G719A+p.L861R, p.G719C+p.L833_V834delinsFL, p.G719C+P.L861Q, p.G719C+p.S768I, p.G721D+p.E746_A750del, p.L833_V834delinsFL+p.L858R, p.L833V+p.H835L, p.L858R+p.A871E, p.N700S+p.S784F, p.N700S+p.T783A, p.S720F+p.L861Q, p.T725M+p.K728E, p.T751_I759del+p.L798F, p.V738F+p.L858R, p.V834L+p.L858R, P772H773dup, S768 D770dupSVD+L858R, S768I+L858R, and T790M+L858R.

In some embodiments, the EGFR mutation is HER2^{YVMA} mutant.

In some embodiments, the EGFR mutation is an osimertinib resistant mutation.

In some embodiments, the osimertinib resistant mutation is selected from the group consisting of Dell9+C797S, Dell9+G724S, Dell9+L718V, Dell9+L792F, Dell9+L792H, L858R+C797G, L858R+C797S, L858R+L718Q, L858R+L718V, L858R+L792F, and L858R+L792H.

In some embodiments, the EGFR mutation is selected from the group consisting of del 746_L750, del 747_L751/T790M, A767_V769dupASV, G13C, L858R, L858R/T790M, N771_H773dupNPH, Q61H, Q61K, and S768_D770dupSVD.

In some embodiments, the EGFR mutation is an EGFR mutation associated with lung adenocarcinoma.

In some embodiments, the EGFR mutation associated with lung adenocarcinoma is selected from the group consisting of A763_Y764insFQEA, A767_S768insTLA, A767_V769dupASV, D770_N771insGT, D770N concurrently with an H773_V774insNPH, G776V/L, Cins, GSP 781-783 ins, M766_A767insAI, S768_D770dupAVD, S768I, S768I in conjunction with G719A, S768I in conjunction with V769L, V765insHH, V769_D770insASV, V774_C775insHV, and YVMA 776-779 ins.

In some embodiments, the EGFR mutation is an EGFR mutation associated with gastric cancer.

In some embodiments, the EGFR mutation associated with gastric cancer is selected from the group consisting of MDK_ex4/HER2_ex11, NOS2_ex2/HER2_ex2, and ZNF207_ex2/HER2_ex18.

In some embodiments, the EGFR mutation is an EGFR mutation associated with lung cancer.

In some embodiments, the EGFR mutation associated with lung cancer is L858R.

In some embodiments, the EGFR mutation is an EGFR mutation associated with non-small cell lung adenocarcinoma.

In some embodiments, the EGFR mutation associated with non-small cell lung adenocarcinoma is selected from the group consisting of L858R, L858R/T790M, del 19, dell9/T790M, dell9/T790M/C797S, L861Q, and G719C/S768I.

In some embodiments, the EGFR mutation is an EGFR mutation associated with urothelial carcinoma.

In some embodiments, the EGFR mutation associated with urothelial carcinoma is selected from the group consisting of R157W, S310F/Y, and V777L/A/M.

In some embodiments, the EGFR mutation is an EGFR mutation associated with breast cancer.

In some embodiments, the EGFR mutation associated with breast cancer is selected from the group consisting of I655V, K676R, K753E, L755S, L768S, Q680R, R647K, and V773L.

In some embodiments, the EGFR mutation is an EGFR mutation associated with Lynch and Lynch-like colorectal cancer.

In some embodiments, the EGFR mutation associated with Lynch and Lynch-like colorectal cancer is selected from the group consisting of A848T, G865R, L726F, L755S/F, L755S with A848T, L755S with V842I, and V842I.

In some embodiments, the cancer is resistant to at least one selected from the group consisting of osimertinib, gefitinib, afatinib, and erlotinib. In some embodiments, the cancer is resistant to osimertinib. In yet other embodiments, the cancer is resistant to gefitinib. In yet certain embodiments, the cancer is resistant to afatinib. In some embodiments, the cancer is resistant to erlotinib.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present application, illustrative methods and materials are described.

As used herein, each of the following terms has the meaning associated with it in this section.

As used herein, the articles “a” and “an” are used to refer to one or to more than one (i.e., to at least one) of the grammatical object of the article. By way of example, “an element” means one element or more than one element.

As used herein, “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “afatinib” refers to N-[4-[(3-Chloro-4-fluorophenyl)amino]-7-[[(3S)-tetrahydro-3-furanyl]oxy]-6-quinazolinyl]-4(dimethylamino)-2-butenamide, or a salt or solvate thereof.

A disease or disorder is “alleviated” if the severity of a symptom of the disease or disorder, the frequency with which such a symptom is experienced by a patient, or both, is reduced.

As used herein, the terms “alkyl”, “alkenyl”, “alkynyl” and “alkoxy” include both straight chain and branched chain groups, and unsubstituted and substituted groups. The optional substituents may include, without limitation, halogen, C₁-C₆ alkoxy, CN, OH, NH₂, NO₂, NH(C₁-C₆ alkyl), N(C₁-C₆ alkyl)₂, CONH₂, CO(C₁-C₆ alkyl), SO₂NH₂ and SO₂(C₁-C₆ alkyl).

As used herein, the term “aromatic nitroheterocycle” means an aromatic heterocyclic moiety substituted at any ring position by one or more nitro (NO₂) groups. The aromatic heterocyclic moiety may be a monocyclic or bicyclic ring containing 4 to 12 atoms of which at least one atom is chosen from nitrogen, sulphur or oxygen. The aromatic heterocyclic moiety may be carbon or nitrogen linked. The aromatic heterocyclic moiety may additionally be substituted by one or more additional substituents at any available ring carbon or heteroatom. The substituents may include, but are not limited to the groups as defined for R₂₆ in Formula V.

As used herein, the term “aromatic nitrocarbocycle” means a benzene moiety substituted at any position by one or more nitro (NO₂) groups. In addition, two adjacent ring carbon atoms may optionally be linked to form a fused carbocyclic or heterocyclic ring. The benzene moiety (and optional fused ring) may additionally be substituted by one or more additional substituents at any available carbon or heteroatom. The substituents may include, but are not limited to, the groups as defined for R₂₆ in Formula V.

As used herein, the terms “co-administered” and “co-administration” refer to administering to the subject a compound contemplated herein or salt thereof along with a compound that may also treat the disorders or diseases contemplated herein. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, the term “composition” or “pharmaceutical composition” refers to a mixture of at least one compound contemplated herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

The term “container” includes any receptacle for holding the pharmaceutical composition or to add protection to manage stability and or water-uptake. For example, in one embodiment, the container is the packaging that contains the pharmaceutical composition such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a breathing disorder in a patient.

The term “determining” as used herein generally refers to any form of measurement, and includes detecting the presence of a mutation, including, for example, an EGFR exon 20 insertion mutation in the tumor cells, as disclosed herein. The term “determining” includes both quantitative and/or qualitative determination. The mutation (e.g., EGFR exon 20 insertion mutation) may be determined by any suitable method known to those skilled in the art, including those as further disclosed herein.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of a compound or agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation. A therapeutic benefit or improvement need not be complete ablation of any one, most or all symptoms, complications, consequences or underlying causes associated with the disorder or disease. Thus, In some embodiments, a satisfactory endpoint is achieved when there is a transient, medium or long term, incremental improvement in a subject's condition, or a partial reduction in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of the disorder or disease, over a duration of time (hours, days, weeks, months, and so forth).

As used herein, the term “erlotinib” refers to N-(3-ethynylphenyl)-6,7-bis(2-methoxyethoxy)quinazolin-4-amine, or a salt or solvate thereof.

As used herein, the term “HER-driven cancer” refers to a cancer that is caused or promoted in any way by a mutation in one of the HER proteins, such as EGER gene fusion, a EGER kinase domain duplication, a ErbB-2 gene fusion, a ErbB-2 mutation, a NRG1 gene fusion, a ErbB-3 mutation, and/or a ErbB-4 fusion and the like.

The HER-driven cancer may be indicated by the presence of a EGER gene fusion, a EGER kinase domain duplication, a ErbB-2 gene fusion, a ErbB-2 mutation, a NRG1 gene fusion, a ErbB-3 mutation, and/or a ErbB-4 fusion. The HER-driven cancer may be resistant to osimertinib, gefitinib, afatinib, and/or erlotinib, as described herein. In some embodiments, the HER-driven cancer has an EGFR mutation, or an ErbB-2 mutation, where the EGFR and/or ErbB-2 mutation is indicated phenotypically, for example, by histopathology, imaging, tumor growth, DNA analysis, RNA analysis or other diagnostic means, as described herein. In some embodiments, a mutation can be identified from general biological samples, e.g., from blood, tissue, urine, and the like, by detecting, e.g., downstream biochemical markers, metabolism markers, circulating RNA, or circulating DNA, and the like, that are indicative of the specific mutations. In some embodiments, the mutations can be tested using, e.g., direct tumor biopsy or liquid biopsy using ctDNA or CTCs.

“Instructional material,” as that term is used herein, includes a publication, a recording, a diagram, or any other medium of expression that can be used to communicate the usefulness of a composition and/or compound contemplated herein in a kit. The instructional material of the kit may, for example, be affixed to a container that contains the compound and/or composition contemplated herein or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, “likelihood”, “likely to”, and similar generally refers to an increase in the probability of an event. Thus, “likelihood”, “likely to”, and similar, when used in reference to responsiveness to cancer therapy, generally contemplates an increased probability that the individual will exhibit a reduction in the severity of cancer or the symptoms of cancer or the retardation or slowing of the cancer progression. The term “likelihood”, “likely to”, and similar, when used in reference to responsiveness to cancer therapy, can also generally mean the increase of indicators that may evidence an increase in cancer treatment.

As used herein, the term “osimertinib” refers to N-(2-{2-dimethylaminoethyl-methylamino}-4-methoxy-5-{[4-(1-methylindol-3-yl)pyrimidin-2-yl]amino}phenyl)prop-2-enamide, or a salt or solvate thereof.

The terms “patient,” “subject” or “individual” are used interchangeably herein, and refer to any animal, or cells thereof whether in vitro or in situ, amenable to the methods described herein. In non-limiting embodiments, the patient is a human. In some embodiments, the subject is a subject in need of treatment thereof. In other embodiments, the subject has a cancer comprising at least one of an EGFR mutation and an ErbB-2 mutation. In some embodiments, the subject as a cancer comprising a single, double, or triple EGFR mutation. In some embodiments, the subject as a cancer comprising a single, double, or triple ErbB-2 mutation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound disclosed herein or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including compounds disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of compounds disclosed herein, and are physiologically acceptable to the patient. Supplementary active compounds may also be incorporated into the compositions.

The “pharmaceutically acceptable carrier” may further include a pharmaceutically acceptable salt of the compounds disclosed herein. Other additional ingredients that may be included in the pharmaceutical compositions disclosed herein are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “predict” can mean to determine or tell in advance. When used to “predict” the responsiveness to a treatment for example, the term “predict” can mean that the likelihood of the outcome of the cancer treatment can be determined at the outset, before the treatment has begun, or before the treatment period has progressed substantially. A predictive method may also be described as a prognostic method.

The term “prevent,” “preventing” or “prevention,” as used herein, means avoiding or delaying the onset of symptoms associated with a disease or condition in a subject that has not developed such symptoms at the time the administering of an agent or compound commences.

As used herein, the term “prodrug” refers to a compound that, after administration, is metabolized or otherwise converted to a biologically active or more active compound (or drug) with respect to at least one property. A prodrug, relative to the drug, is modified chemically in a manner that renders it, relative to the drug, less active or inactive, but the chemical modification is such that the corresponding drug is generated by metabolic or other biological processes after the prodrug is administered. A prodrug may have, relative to the active drug, altered metabolic stability or transport characteristics, fewer side effects or lower toxicity, or improved flavour (for example, see the reference Nogrady, 1985, Medicinal Chemistry A Biochemical Approach, Oxford University Press, New York, pages 388-392, incorporated herein by reference). A prodrug may be synthesized using reactants other than the corresponding drug.

As used herein, the phrase “providing tumor cells” refers to the step of obtaining cells of the individual (e.g. by way of biopsy or otherwise), and/or refers to the step of receiving a sample of tumor cells that has previously been obtained from the individual.

A “therapeutic” treatment is a treatment administered to a subject who exhibits signs of pathology, for the purpose of diminishing or eliminating those signs.

The term “responsiveness” or “responsive,” when used in reference to a treatment, refers to the degree of effectiveness of the treatment in lessening or decreasing the symptoms of a disease, disorder, or condition being treated. For example, the term “increased responsiveness,” when used in reference to a treatment of a cell or a subject, refers to an increase in the effectiveness in lessening or decreasing the symptoms of the disease when measured using any methods known in the art. In some embodiments, the increase in the effectiveness is at least about 5%, at least about 10%, at least about 20%, at least about 30%, at least about 40%, or at least about 50%.

In some embodiments, tumor cells comprise a “sample.” In other embodiments, the sample comprises a biological sample and can be, for instance, a cell, a cell culture, a tissue, and/or a biological fluid. Suitably, the biological sample can comprise a tumor cell biopsy, a plurality of samples from a clinical trial, or the like. The sample can be a crude sample, or can be purified to various degrees prior to storage, processing, or measurement.

As used herein, the term “treatment” or “treating” is defined as the application or administration of a therapeutic agent, i.e., a compound disclosed herein (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

In some embodiments, the term “treatment” or “treating” refers to an action that occurs while an individual is suffering from the specified cancer, which reduces the severity of the cancer or the symptoms of the cancer, and/or retards or slows the progression of the cancer. For instance, In some embodiments, “treatment” or “treat” refers to a 5%, 10%, 25%, 50%, or 100% decrease in the rate of progress of a tumor. In other embodiments, “treatment” refers to a 5%, 10%, 25%, 50%, or 100% decrease in determined tumor burden (i.e., number of cancerous cells present in the individual, and/or the size of the tumor). In yet other embodiments, “treatment” refers to a 5%, 10%, 25%, 50%, or 100% decrease in any physical symptom(s) of a cancer. In yet other embodiments, “treatment” refers to a 5%, 10%, 25%, 50%, or 100% increase in the general health of the individual, as determined by any suitable means, such as cell counts, assay results, or other suitable means. As used herein, the cancer can be any cancer, including those contemplated herein, including, for example, a HER-driven cancer. In some embodiments, the cancer is a HER-driven drug-resistant cancer.

As used herein, the term “EGFR” or “ErbB1” or “ErbB-1” or “HER1” refers to epidermal growth factor receptor. The amino acid sequence for the human EGFR (isoform 1; canonical—UniProt ID P00533-1) is recited in SEQ ID NOT.

The following non-limiting alternative isoforms of EGFR (as relating to the canonical isoform) are also contemplated:

Isoform 2 (UniProt ID P00533-2):
404-405: FL → LS
406-1210: Missing
Isoform 3 (UniProt ID P00533-3):
628-705: CTGPGLEGCP (SEQ ID NO: 10) ...
GEAPNQALLR (SEQ ID NO: 11) → PGNESLKAML
(SEQ ID NO: 12) ... SVIITASSCH (SEQ ID NO: 13)
706-1210: Missing
Isoform 4 (UniProt ID P00533-4):
628-628: C → S
629-1210: Missing

The nucleotide sequence for the EGFR gene, complete cds, alternatively spliced, is recited in SEQ ID NO:2.

The nucleotide sequence for the EGFR exon 18 is recited in SEQ ID NO:3.

The nucleotide sequence for the EGFR exon 19 is recited in SEQ ID NO:4.

The nucleotide sequence for the EGFR exon 20 is recited in SEQ ID NO:5.

The nucleotide sequence for the EGFR exon 21 is recited in SEQ ID NO:6.

As used herein, the term “ErbB2” or “ErbB-2” or “HER2” or “HER-2” refers to receptor tyrosine-protein kinase erbB-2. The amino acid sequence for the human ErbB2 (isoform 1; canonical—UniProt ID P04626-1) is recited in SEQ ID NO:7.

The following non-limiting alternative isoforms of ErbB2 (as relating to the canonical isoform) are also contemplated:

Isoform 2 (UniProt ID P04626-2):
1-610: Missing
Isoform 3 (UniProt ID P04626-3):
1-686: Missing
Isoform 4 (UniProt ID P04626-4):
1-23: MELAALCRWGLLLALLPPGAAST (SEQ ID NO:
14) → MPRGSWKP (SEQ ID NO: 15)
Isoform 5 (UniProt ID P04626-5):
1-30: Missing
Isoform 6 (UniProt ID P04626-6):
633-648: Missing
771-883: AYVMAGVGSP (SEQ ID NO: 16) ...
ETEYHADGGK (SEQ ID NO: 17) → TISNLFSNFA
(SEQ ID NO: 18) ... LMCPQGAGKA (SEQ ID
NO: 19)
884-1255: Missing

As used herein, the term “ErbB-3” or “ErbB-3” or “HER3” or “HER-3” refers to receptor tyrosine-protein kinase ErbB-3. The amino acid sequence for the human ErbB-3 (isoform 1; canonical—(UniProt ID P21860-1) is recited in SEQ ID NO:8.

The following non-limiting alternative isoforms of ErbB-3 (as relating to the canonical isoform) are also contemplated:

Isoform 2 (UniProt ID P21860-2):
141-183: EILSGGVYIE (SEQ ID NO: 20) ...
IVVKDNGRSC (SEQ ID NO: 21) → GQFPMVPSGL
(SEQ ID NO: 22) ... SKVPVTLAAV (SEQ
ID NO: 23)
184-1342: Missing
Isoform 3 (UniProt ID P21860-3):
331-331 C → F
332-1342: Missing
Isoform 4 UniProt ID P21860-4):
1-59: Missing
Isoform 5 (UniProt ID P21860-5):
1-643: Missing

As used herein, the term “ErbB-4” or “ErbB-4” or “HER4” or “HER-4” refers to receptor tyrosine-protein kinase ErbB-4. The amino acid sequence for the human ErbB-4 (isoform JM-A CYT-1; canonical—UniProt ID Q15303-1) is recited in SEQ ID NO:9.

The following non-limiting alternative isoforms of ErbB-4 (as relating to the canonical isoform) are also contemplated:

Isoform JM-B CYT-1 (UniProt ID Q15303-2):
626-648: NGPTSHDCIYYPWTGHSTLPQHA (SEQ ID
NO: 24) → IGSSIEDCIGLMD (SEQ ID NO: 25)
Isoform JM-A CYT-2 (UniProt ID Q15303-3):
1046-1061: Missing
Isoform JM-B CYT-2 (UniProt ID Q15303-4):
626-648: NGPTSHDCIYYPWTGHSTLPQHA (SEQ ID
NO: 26) → IGSSIEDCIGLMD (SEQ ID NO: 27)
1046-1061: Missing.

Throughout this disclosure, various aspects of the disclosure can be presented in a range format. It should be understood that the description in range format is merely for convenience and brevity and should not be construed as an inflexible limitation on the disclosure herein. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 96%, 97%, etc., as well as 91.1%, 91.2%, 91.3%, 91.4%, 91.5%, etc., 92.1%, 92.2%, 92.3%, 92.4%, 92.5%, and so forth. A series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth. This applies regardless of the breadth of the range.

### Compositions and Methods

Provided herein are compositions and methods using compound of Formula I, II, VII, VIII, IX, X and/or XI, as disclosed herein.

In some embodiments, the compound is RN-4000 [“(E)-4-((4-((3-bromo-4-chlorophenyl)amino)pyrido[3,4-d]pyrimidin-6-yl)amino)-N,N-dimethyl-N-((l-methyl-4-nitro-1H-imidazol-5-yl)methyl)-4-oxobut-2-en-1-aminium salt (bromide)”; also referred to herein as “(2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide” Compound A], and/or RN-4000E [“(2E)-N-[4-(3-bromo-4-chloroanilino)pyrido[3,4-d]pyrimidin-6-yl]-4-(dimethylamino)-2-butenamide”: also referred to herein “(E)-N-(4-((3-bromo-4-chlorophenyl)amino)pyrido[3,4-d]pyrimidin-6-yl)-4-(dimethylamino)but-2-enamide”; Compound B].

In some embodiments, the compounds of Formula I, II, VII, VIII, IX, X and/or XI, as disclosed herein, are part of a pharmaceutical composition, which optionally further comprises at least one additional agent that treats or prevents a HER-driven (such as, in a non-limiting example, an EGFR-driven) drug-resistant cancer.

In some embodiments, Compound A and/or Compound B is part of a pharmaceutical composition, which optionally further comprises at least one additional agent that treats or prevents a HER-driven (such as, in a non-limiting example, an EGFR-driven) drug-resistant cancer.

Also provided herein is a method of treating or preventing HER-driven cancer in a subject. Further provided herein is a method of treating or preventing EGFR-driven drug-resistant cancer in a subject. In some embodiments, the method comprises administering to the subject in need thereof a therapeutically effective amount of at least one compound contemplated herein, for example, a compound of Formula I, II, VII, VIII, IX, X and/or XI, as disclosed herein, including Compound A and/or Compound B, or a salt or solvate thereof. HER-driven cancers include, but are not limited to, cancers caused by a EGER gene fusion, a EGER kinase domain duplication, a ErbB-2 gene fusion, a ErbB-2 mutation, a NRG1 gene fusion, a ErbB-3 mutation, and/or a ErbB-4 fusion.

The oncogenic alterations involving a. EGER proto-oncogene are illustrated in Table 1, and each of those alterations is contemplated herein.

TABLE 1

| Exemplary cancer | |
|---|---|
| types with EGFR gene | |
| amplification and/or | Exemplary EGFR |
| mutation | mutations |
| Lung¹ | A763_Y764insFHEA^{1, 2} |
| Breast¹⁰ | C797S/L858R³ |
| Head and Neck | d746-750^{1, 3} |
| Squamous Cell | |
| Carcinoma¹⁰ | |
| Esophageal¹⁰ | d746-750/C797A |
| Glioma⁶ | d746-750/C797S³ |
| Colorectal¹⁰ | d746-750/T790M/C797S³ |
| Renal⁵ | D770GY |
| Glioblastoma⁶ | D770_N771insNPG¹ |
| Mesothelioma⁷ | G719C⁸ |
| | G719S⁸ |
| | L747S⁹ |
| | L858R¹ |
| | L858R/T790M^{1, 4} |
| | L861Q¹ |
| | T790M/C797S/L858R^{3, 4} |

References relating to Table 1:
¹Hirano et al., 2015 Oncotarget 6(36): 38798-38803.
²Yasuda et al., 2013 Sci Transl Med 6: 225.
³Grabe et al., 2017 ACS Med. Chem. Lett. 139: 674-697.
⁴Lu et al., 2019, Med. Res. Rev. 38(5): 1550-1581.
⁵Minner et al., 2012 Cancer 118(5): 1268-1275.
⁶Manfred et al., 2017 CNS Drugs 31(9): 723-735.
⁷Salvi et al., 2016 J. Thor. Onc. 11(6): e78-e80.
⁸Mishra et al. 2017 Oncotarget, 8(69), 114371-114392.
⁹Tu et al. 2017 Cancer 114: 96-102.
¹⁰Wang, Z. 2017 Methods Mol. Biol. 1652: 3-30.

The oncogenic alterations involving a ErbB-2 (HER2) proto-oncogene are illustrated in Table 2, and each of those alterations is contemplated herein.

TABLE 2

| ErbB-2 gene amplification | ErbB-2 mutations |
|---|---|
| Breast¹ | V777L^{2, 5, 15} |
| Lung^{3, 4} | R896C² |
| Gastric/GE Junction^{6, 7} | D769H¹⁶ |
| Esophageal⁸ | V777_G778insCG¹⁷ |
| Salivary^{9, 10} | D769Y¹⁸ |
| Ovarian¹¹ | R896C² |
| Endometrial¹² | |
| Uterine¹³ | |
| Pancreatic¹⁴ | |

References relating to Table 2:
¹Lebeau, et al., 2001, J Clin Oncol 19: 354-63.
²Bose, et al., 2013, Cancer Discov 3: 224-37.
³Li, et al., 2016, J Thorac Oncol 11: 414-9.
⁴Cancer Genome Atlas Research N: Comprehensive molecular profiling of lung adenocarcinoma, 2014, Nature 511: 543-50.
⁵Kavuri, et al., 2015, Cancer Discov 5: 832-41.
⁶Gordon, et al., 2013, Ann Oncol 24: 1754-61.
⁷Das, et al., 2014, Cancer Lett 353: 167-75.
⁸Gonzaga, et al., 2012, BMC Cancer 12: 569.
⁹Nardi, et al., 2013, Clin Cancer Res 19: 480-90.
¹⁰Williams, et al., 2010, Clin Cancer Res 16: 2266-74.
¹¹Tuefferd, et al., 2007, PLoS One 2: e1138.
¹²Morrison, et al., 2006, J Clin Oncol 24: 2376-85.
¹³Slomovitz, et al., 2004, J Clin Oncol 22: 3126-32.
¹⁴Chou, et al., 2013, Genome Med 5: 78.
¹⁵Bellmunt, et al., 2015, Cancer Med 4: 844-52.
¹⁶Lee, et al., 2006, Clin Cancer Res 12(1): 57-61.
¹⁷Oh et al. 2018, (19)5: e775-e781.
¹⁸Hyman et al., 2018, Nature 554: 189-194.

The present application contemplates methods of treating a subject with cancer with the compounds contemplated herein, where an EGFR and/or an ErbB-2 mutation is present in the tumor cells of the subject. The present application also contemplates related uses of such methods.

Cancers with such EGFR mutations and/or ErbB-2 mutations exhibit certain characteristics which indicate the presence of the mutation(s). For example, cancers with certain EGFR mutations or ErbB-2 mutations exhibit resistance and/or poor response to EGFR-TKIs such as osimertinib, gefitinib, afatinib, and erlotinib (see, e.g., Takeda et al., 2018, Oncotarget 9(30): 21132, Hyman et al., 2018, Nature 554: 189-194, incorporated herein by reference in their entireties). Accordingly, the present application further contemplates methods of treating a subject with cancer with the compounds contemplated herein, where the presence of an EGFR mutation and/or ErbB-2 mutation in the tumor cells of a subject is indicated by resistance and/or poor response to an oncology agent, such an EGFR-TKI.

An EGFR mutation and/or ErbB-2 mutation may also be indicated from a particular phenotype characteristic of the cancer, for example, histopathology, imaging, tumor growth, DNA analysis, RNA analysis or other diagnostic means, and/or survival rate of the patient (see, e.g., Naidoo et al., 2015, Cancer 121(18): 3212, incorporated herein by reference in its entirety). In some embodiments, the mutation can be identified from general samples, e.g, from blood, tissue, urine, and the like, by detecting downstream biochemical markers, metabolism markers, circulating RNA, or circulating DNA that are indicative of the specific mutations. In some embodiments, the mutations can be tested using, e.g., direct tumor biopsy or liquid biopsy using ctDNA or CTCs.

The present application further contemplates methods of treating a subject with cancer with the compounds contemplated herein, wherein the treatment is part of a maintenance therapy for subjects with recurring or refractory cancer. For example, the present application contemplates a method of treating a resistant or refractory cancer in a subject with the compounds disclosed herein. In some embodiments, the treatment leads to a full response, remission, and/or complete cure in the subject with recurring or refractory cancer. In some embodiments, the treatment maintains a stable disease, leads to a partial response (e.g., some tumor regression), or prevents the return of tumors which have fully regressed. In some embodiments, the cancer has an EGFR mutation. In some embodiments, the EGFR insertion mutation is at least one of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R. In some embodiments, the cancer has an ErbB-2 mutation. In some embodiments, the ErbB-2 mutation is at least one of D769H, D769Y, R896C, V777L, and V777_G778insCG.

In some embodiments, the compound of the methods and related uses disclosed herein is of a compound of Formula I, II, VII, VIII, IX, X and/or XI. In some embodiments, the compound is of Formula I, II, IX, X and/or XI. In some embodiments, the compound is of Formula VII and/or VIII. In some embodiments, the compound is one of compounds 12-88 and 91-104. In some embodiments, the compound is one of compounds 1-11, 89 and 90. In some embodiments, the compound is compound 17. In some embodiments, the compound is compound 5. In some embodiments, the compound is Compound A. In some embodiments, the compound is Compound B.

In one aspect, the present application provides a method of treating or preventing a HER-driven cancer in a subject, the method comprising administering to the subject in need thereof a therapeutically effective amount of at least one compound selected from the group consisting of “(2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide” (Compound A), and (2E)-N-[4-(3-bromo-4-chloroanilino)pyrido[3,4-d]pyrimidin-6-yl]-4-(dimethylamino)-2-butenamide (Compound B), or a salt or solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an EGFR (HER1) mutation;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an HER2 mutation;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an HER3 mutation;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an HER4 mutation;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an EGFR (HER1) mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an HER2 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an HER3 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an HER4 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven lung cancer in a subject in need thereof, the method comprising:

(a) providing tumor cells of the subject;

(b) detecting presence or absence of a mutation or gene fusion or gene amplification in the provided tumor cells, wherein the mutation comprises an EGFR (HER1) mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven lung cancer in a subject in need thereof, the method comprising:

(a) providing tumor cells of the subject;

(b) detecting presence or absence of a mutation or gene fusion or gene amplification in the provided tumor cells, wherein the mutation comprises an HER2 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven lung cancer in a subject in need thereof, the method comprising:

(a) providing tumor cells of the subject;

(b) detecting presence or absence of a mutation or gene fusion or gene amplification in the provided tumor cells, wherein the mutation comprises an HER3 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven lung cancer in a subject in need thereof, the method comprising:

(a) providing tumor cells of the subject;

(b) detecting presence or absence of a mutation or gene fusion or gene amplification in the provided tumor cells, wherein the mutation comprises an HER4 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven brain metastasis in a subject in need thereof, the method comprising:

(a) providing tumor cells of the subject;

(b) detecting presence or absence of a mutation or gene fusion or gene amplification in the provided tumor cells, wherein the mutation comprises an EGFR (HER1) mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven brain metastasis in a subject in need thereof, the method comprising:

(a) providing tumor cells of the subject;

(b) detecting presence or absence of a mutation or gene fusion or gene amplification in the provided tumor cells, wherein the mutation comprises an HER2 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven brain metastasis in a subject in need thereof, the method comprising:

(a) providing tumor cells of the subject;

(b) detecting presence or absence of a mutation or gene fusion or gene amplification in the provided tumor cells, wherein the mutation comprises an HER3 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven brain metastasis in a subject in need thereof, the method comprising:

(a) providing tumor cells of the subject;

(b) detecting presence or absence of a mutation or gene fusion or gene amplification in the provided tumor cells, wherein the mutation comprises an HER4 mutation;

(c) predicting the subject as being likely to be responsive to treatment by Compound A, Compound B, or a combination thereof, if at least one of the EGFR mutation and the ErbB-2 mutation is detected; and

(d) administering a therapeutically effective amount of Compound A, or a salt or a solvate thereof.

In another aspect, the present disclosure provides a method of treating or preventing a HER-driven cancer in a subject in need thereof, the method comprising:

(a) providing tumor cells of the subject;

(b) detecting presence or absence of a mutation or gene fusion or gene amplification in the provided tumor cells, wherein the mutation comprises an EGFR (HER1) mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R, EGFR exon 20 insertion mutations (Reiss et al J Thorac Oncol. 2018 October; 13(10):1560-1568, Arcila et al Mol Cancer Ther. 2013 February; 12(2): 220-229), EGFR rare and compound mutations (Beau-Faller et al Annals of Oncology 25: 126-131, 2014, Martin et al Clin Lung Cancer. 2019 May 11. pii: S1525-7304(19)30103-2, Mishra et al Oncotarget, 2017, Vol. 8, (No. 69), pp: 114371-114392), tertiary Osimertinib resistance mutations L718Q/V, G724S, L792F/H, G796S, C797S/G, EGFR gene fusions EGFR-SEPT14, EGFR-RAD51, EGFR-PSPH or additional C-terminal partners, EGFR kinase domain duplication (EGFR-KDD); or wherein the mutation comprises an ErbB-2 (HER2) mutation selected from the group consisting of: D769H, D769Y, R896C, V777L, V777_G778insCG, G309A/E, S310F/Y, V659E/D, G660D, K753E, L755P/S, Del755-759, L768S, D769H/Y, V773L, A775_G776insYVMA, G776V/L, Cins, V777L, P780Ins, P780_Y781insGSP, V842I, L866M, R896C, juxtamembrane and transmembrane domain mutations S653C, S656C, V659E, G660D, and G660R, and JMD mutants R677L, R678W, T686A, E693K, S649T, P650S, L651V, V659G, G660D, G660R, L663P, L674V, R677L, R678Q, R683Q, E693K, Q709L, and A710V and ErbB2 gene fusions ZNF207-HER2, MDK-HER2, NOS2-HER2, ERBB2-GRB7, ERBB2-CTTN, ERBB2-PPP1R1B, ERBB2-PSMB3 or additional N-terminal partners; or wherein the mutation comprises an ErbB-4 (HER4) mutation selected from the group consisting of: N181S, T244R, Y285C, R306S, V348L, D595V, H618P, D931Y, K935I, E317K, E452K, E542K, R544W, E563K, E836K, E872K and HER4 gene fusions EZR-ERBB4, IKZF2-ERBB4, BGALT-ERBB4 or additional N-terminal partners; or wherein the mutation comprises an ErbB-3 (HER3) mutation selected from the group consisting of: T355I, F94L, G284R, D297Y, T355I, E1261A; V104M, A232V, P262H, G284R, T389K, V714M, Q809R, S846I and E928G; any activating mutation, TKI resistance mutations, gene fusion, kinase domain duplication, and gene amplification of ErbB receptors;

(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutation and the ErbB-2 mutation is detected; and

(d) administering a therapeutically effective amount of Compound A, or a salt or a solvate thereof.

In another aspect, the present application provides a method of treating a HER-driven cancer in a subject with cancer, where a mutation is detected in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the method comprises administering a therapeutically effective amount of at 10 least one compound selected from the group consisting of Compound A and Compound B, or a salt or a solvate thereof.

In another aspect, the present application provides a method of treating cancer in a subject with cancer. In other embodiments, the method comprises:

(a) providing tumor cells of the subject;

(b) detecting presence or absence of a mutation in the provided tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG in provided tumor cells of the subject;

(c) predicting the subject as being likely to be responsive to treatment by a compound contemplated herein if the mutation is detected;

(d) administering a therapeutically effective amount of a compound contemplated herein to the subject.

In some embodiments, the cancer is a HER-driven cancer.

In some embodiments, the cancer is a HER-driven drug-resistant cancer.

In some embodiments, the compound is Compound A. In some embodiments, the compound is Compound B.

In another aspect, the present application provides a method of treating cancer in a subject with cancer. In other embodiments, the method comprises:

(a) detecting presence or absence of a mutation in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(b) predicting the subject as being likely to be responsive to treatment by a compound contemplated herein if the mutation is detected;

(c) administering a therapeutically effective amount of a compound contemplated herein to the subject.

In some embodiments, the cancer is a HER-driven cancer.

In some embodiments, the cancer is a HER-driven drug-resistant cancer.

In some embodiments, the compound is Compound A. In some embodiments, the compound is Compound B.

In yet another aspect, the present application provides a method of treating cancer in a subject with cancer, where a mutation is detected in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG.

In other embodiments, the method comprises administering a therapeutically effective amount of a compound contemplated herein to the subject. In yet other embodiments, the cancer is a HER-driven cancer. In yet other embodiments, the cancer is a HER-driven drug-resistant cancer. In yet other embodiments, the compound is Compound A or Compound B. In yet another aspect, the present application provides the use of a compound contemplated herein in the manufacture of a composition for the treatment of cancer in a subject with cancer, where a mutation is detected in the tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG.

In other embodiments, the present application provides the use of a compound contemplated herein in the manufacture of a composition for the treatment of cancer in a subject with cancer, where a mutation is detected in a sample of tumor cells from the subject wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG. In yet other embodiments, the cancer is a HER-driven cancer. In yet other embodiments, the cancer is a HER-driven drug-resistant cancer. In yet other embodiments, the compound is Compound A or Compound B.

In yet another aspect, the present application provides the use of a compound contemplated herein in the treatment of cancer in a subject with cancer, where a mutation is detected in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG.

In other embodiments, the present application provides the use of a compound contemplated herein in the treatment of cancer in a subject with cancer, where a mutation is detected in a sample of tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG. In yet other embodiments, the cancer is a HER-driven cancer. In yet other embodiments, the cancer is a HER-driven drug-resistant cancer. In yet other embodiments, the compound is Compound A or Compound B.

In other embodiments, the present application provides use of a compound contemplated herein in the manufacture of a medicament for the treatment of a HER-driven cancer, wherein the mutation comprises an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation comprises an ErbB-2 mutation selected from the group consisting of: D769H, D769Y, R896C, V777L, and V777_G778insCG. In some embodiments, the cancer is a HER-driven drug-resistant cancer.

In yet another aspect, the present application provides a compound contemplated herein for use in the treatment of cancer in a subject with cancer, where a mutation is detected in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG. In other embodiments, the present application provides a compound contemplated herein for use in the treatment of cancer in a subject with cancer, where a mutation is detected in a sample of tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG. In yet other embodiments, the cancer is a HER-driven drug-resistant cancer. In yet other embodiments, the compound is Compound A or Compound B.

In yet another aspect, the present application provides a compound contemplated herein for use in the treatment of cancer in a subject with cancer, comprising:

(a) providing tumor cells of the subject;

(b) detecting presence or absence of a mutation in the provided tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG; and

(c) administering a therapeutically effective amount of a compound contemplated herein to the subject if the mutation is detected.

In some embodiments, the cancer is a HER-driven cancer.

In some embodiments, the cancer is a HER-driven drug-resistant cancer.

In some embodiments, the compound is Compound A. In some embodiments, the compound is Compound B.

In yet another aspect, the present application provides a compound contemplated herein for use in the treatment of cancer in a subject with cancer. In some embodiments, the method comprises:

(a) detecting presence or absence of a mutation in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG; and

(b) administering a therapeutically effective amount of a compound contemplated herein to the subject if the mutation is detected in the provided tumor cells of the subject.

In some embodiments, the cancer is a HER-driven cancer.

In some embodiments, the cancer is a HER-driven drug-resistant cancer.

In some embodiments, the compound is Compound A. In some embodiments, the compound is Compound B.

In some embodiments, the subject is further administered at least one additional agent, or a salt or solvate thereof, that treats or prevents the drug-resistant cancer. Non-limiting examples of additional anti-proliferative agents contemplated include, but are not limited to, compounds listed on the cancer chemotherapy drug regimens in the 14^{th} Edition of the Merck Index (2006), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine. Additional anti-proliferative agents include other molecular targeted agents that modulate parallel pathways such as MEK 1/2 inhibitors, AKT inhibitors and mTOR inhibitors, monoclonal antibodies (such as Cetuximab), oxaliplatin, gemcitabine, gefinitib, taxotere, ara A, ara C, herceptin, BCNU, CCNU, DTIC, and actinomycin D. Still further anti-proliferative agents include but are not limited to those compounds acknowledged to be used in the treatment of neoplastic diseases in Goodman and Gilman's The Pharmacological Basis of Therapeutics (Eleventh Edition), editor Molinoff et al., publ. by McGraw-Hill, pages 1225-1287 (2006), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, tenipdside, testosterone propionate, thiotepa, trimethylmelamine, uridine, and vinorelbine.

In some embodiments, a compound of any one of Formulas I, II, VII, VIII, IX, X and/or XI, as disclosed herein, and at least one additional agent, are co-administered to the subject. In other embodiments, a compound of any one of Formulas I, II, VII, VIII, IX, X and/or XI, as disclosed herein, and at least one additional agent, are co-formulated.

In some embodiments, Compound A or Compound B, and at least one additional agent, are co-administered to the subject. In other embodiments, Compound A or Compound B, and at least one additional agent, are co-formulated.

In some embodiments, a compound of any one of Formulas I, II, VII, VIII, IX, X and/or XI, as disclosed herein, is administered by at least one route selected from the group consisting of inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical. In other embodiments, the subject is a mammal. In yet other embodiments, the mammal is a human. In some embodiments, the subject is a human in need of treatment thereof.

In some embodiments, Compound A or Compound B is administered by at least one route selected from the group consisting of inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical. In other embodiments, the subject is a mammal. In yet other embodiments, the mammal is a human.

In some embodiments, the subject is a human in need of treatment thereof.

Also provided herein is a kit comprising a compound of any one of Formulas I, II, VII, VIII, IX, X and/or XI, as disclosed herein, an applicator and instructional material for use thereof, wherein the instructional material comprises instructions for preventing or treating HER-driven cancers. In some embodiments, the cancer is a HER-driven drug-resistant cancer.

Further provided herein is a kit comprising Compound A or Compound B, an applicator and instructional material for use thereof, wherein the instructional material comprises instructions for preventing or treating HER-driven cancers. In some embodiments, the cancer is a HER-driven drug-resistant cancer.

### Salts

The compounds described herein may form salts with acids, and such salts are included in the present application. In one embodiment, the salts are pharmaceutically acceptable salts. The term “salts” embraces addition salts of free acids that are useful within the methods disclosed herein. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present application, such as for example utility in process of synthesis, purification or formulation of compounds useful within the methods disclosed herein.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g. saccharinate, saccharate). Salts may be comprised of a fraction of one, one or more than one molar equivalent of acid or base with respect to any compound contemplated herein.

Suitable pharmaceutically acceptable base addition salts of compounds contemplated herein include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

### Predictive/Identification Methods

The present application also contemplates methods of predicting responsiveness of a subject with cancer to treatment with a compound contemplated herein.

In some embodiments, the compound is of a compound of Formula I, II, VII, VIII, IX, X and/or XI. In some embodiments, the compound is of Formula I, II, IX, X and/or XI. In some embodiments, the compound is of Formula VII and/or VIII. In some embodiments, the compound is one of compounds 12-88 and 91-104. In some embodiments, the compound is one of compounds 1-11, 89 and 90. In some embodiments, the compound is compound 17. In some embodiments, the compound is compound 5. In some embodiments, the compound is Compound A. In some embodiments, the compound is Compound B.

In one aspect, the present application provides a method of predicting the responsiveness of a subject with cancer to treatment with a compound contemplated herein.

In some embodiments, the method comprises:

(a) providing tumor cells of the subject;

(b) detecting presence or absence of a mutation in the provided tumor cells of the subject, wherein the mutation is an EGFR mutation;

(c) predicting the subject as being likely to be responsive to a treatment with a compound contemplated herein if the mutation is detected in the provided tumor cells of the subject.

In one aspect, the present application provides a method of predicting the responsiveness of a subject with cancer to treatment with a compound contemplated herein.

In some embodiments, the method comprises:

(a) providing tumor cells of the subject;

(b) detecting presence or absence of a mutation in the provided tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(c) predicting the subject as being likely to be responsive to a treatment with a compound contemplated herein if the mutation is detected in the provided tumor cells of the subject.

In some embodiments, the compound is Compound A. In some embodiments, the compound is Compound B.

In another aspect, the present application provides a method of predicting the responsiveness of a subject with cancer to treatment with a compound contemplated herein.

In some embodiments, the method comprises:

(a) detecting presence or absence of a mutation in a sample from the subject, wherein the sample comprises tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(b) predicting the subject as being likely to be responsive to a treatment with a compound contemplated herein if the mutation is detected in the sample from the subject.

In some embodiments, the compound is Compound A. In some embodiments, the compound is Compound B.

In yet another aspect, the present application provides a method of predicting the responsiveness of a subject with cancer to treatment with a compound contemplated herein.

In some embodiments, the method comprises:

(a) detecting presence or absence of a mutation in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG; and

(b) predicting the subject as being likely to be responsive to treatment with a compound contemplated herein if the mutation is detected in the tumor cells of the subject.

In some embodiments, the compound is Compound A. In some embodiments, the compound is Compound B.

In yet another aspect, the present application provides a method of predicting the responsiveness of a subject with cancer to treatment with a compound contemplated herein.

In some embodiments, the method comprises:

(a) detecting presence or absence of a mutation in a sample of tumor cells from the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG; and

(b) predicting the subject as being likely to be responsive to treatment with a compound contemplated herein if the mutation is detected in the sample of tumor cells from the subject.

In some embodiments, the compound is Compound A. In some embodiments, the compound is Compound B.

In yet another aspect, the present application provides a method of predicting the responsiveness of a subject with cancer to treatment with a compound contemplated herein.

In some embodiments, the method comprises:

(a) providing tumor cells of the subject;

(b) detecting presence or absence of a mutation in the provided tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is likely to be responsive to the treatment with a compound contemplated herein if the mutation is detected in the provided tumor cells of the subject.

In some embodiments, the compound is Compound A. In some embodiments, the compound is Compound B.

In yet another aspect, the present application provides a method of predicting the responsiveness of a subject with cancer to treatment with a compound contemplated herein. In some embodiments, the method comprises detecting presence or absence of a mutation in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is likely to be responsive to the treatment with a compound contemplated herein if the mutation is detected in the tumor cells of the subject.

In some embodiments, the compound is Compound A. In some embodiments, the compound is Compound B.

In yet another aspect, the present application provides a method of predicting the responsiveness of a subject with cancer to treatment with a compound contemplated herein.

In some embodiments, the method comprises detecting presence or absence of a insertion mutation in a sample of tumor cells from the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is likely to be responsive to the treatment with a compound contemplated herein if the mutation is detected in the sample of tumor cells from the subject.

In some embodiments, the compound is Compound A. In some embodiments, the compound is Compound B.

In yet another aspect, the present application provides a method of predicting the responsiveness of a subject with cancer to treatment with a compound contemplated herein.

In some embodiments, the method comprises:

(a) providing tumor cells of the subject; and

(b) detecting presence or absence of a mutation in the provided tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the presence of the mutation in the provided tumor cells of the subject correlates with an increased likelihood of responsiveness or an increased responsiveness of the subject to the treatment.

In some embodiments, the compound is Compound A. In some embodiments, the compound is Compound B.

In some embodiments of the various methods provided herein, the prediction of the responsiveness of the subject with cancer to treatment by a compound contemplated herein is made by detecting the presence of a mutation in the tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG. In other embodiments, the method further comprises administration of a compound contemplated herein to the subject. In yet other embodiments, the method further comprises administration of a compound contemplated herein to the subject if the subject is predicted to be likely to be responsive to the treatment. In yet other embodiments, a compound contemplated herein is administered in an therapeutically effective amount.

The present application also contemplates methods of predicting whether a subject with cancer is likely to be responsive to treatment with a compound contemplated herein.

In one aspect, the present application provides a method of predicting whether a subject with cancer is likely to be responsive to treatment with a compound contemplated herein. In some embodiments, the method comprises:

(a) providing tumor cells of the subject;

(b) detecting presence or absence of a mutation in the provided tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(c) predicting the subject as being likely to be responsive to a treatment with a compound contemplated herein if the mutation is detected in the provided tumor cells of the subject.

In some embodiments, the compound is Compound A. In some embodiments, the compound is Compound B.

In another aspect, the present application provides a method of predicting whether a subject with cancer is likely to be responsive to treatment with a compound contemplated herein. In some embodiments, the method comprises:

(a) detecting presence or absence of a mutation in a sample from the subject, wherein the sample comprises tumor cells, and wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(b) predicting the subject as being likely to be responsive to a treatment with a compound contemplated herein if the mutation is detected in the sample from the subject.

In some embodiments, the compound is Compound A. In some embodiments, the compound is Compound B.

In yet another aspect, the present application provides a method of predicting whether a subject with cancer is likely to be responsive to treatment with a compound contemplated herein. In some embodiments, the method comprises:

(a) detecting presence or absence of a mutation in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG; and

(b) predicting the subject as being likely to be responsive to treatment with a compound contemplated herein if the mutation is detected in the tumor cells of the subject.

In some embodiments, the compound is Compound A. In some embodiments, the compound is Compound B.

In yet another aspect, the present application provides a method of predicting whether a subject with cancer is likely to be responsive to treatment with a compound contemplated herein. In some embodiments, the method comprises:

(a) detecting presence or absence of a mutation in a sample of tumor cells from the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG; and

(b) predicting the subject as being likely to be responsive to treatment with a compound contemplated herein if the mutation is detected in the sample of tumor cells from the subject.

In some embodiments, the compound is Compound A. In some embodiments, the compound is Compound B.

In yet another aspect, the present application provides a method of predicting whether a subject with cancer is likely to be responsive to treatment with a compound contemplated herein. In some embodiments, the method comprises:

(a) providing tumor cells of the subject;

(b) detecting presence or absence of a mutation in the provided tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is likely to be responsive to the treatment with a compound contemplated herein if the mutation is detected in the provided cells.

In some embodiments, the compound is Compound A. In some embodiments, the compound is Compound B.

In yet another aspect, the present application provides a method of predicting whether a subject with cancer is likely to be responsive to treatment with a compound contemplated herein. In some embodiments, the method comprises detecting presence or absence of a mutation in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is likely to be responsive to the treatment with a compound contemplated herein if the mutation is detected.

In some embodiments, the compound is Compound A. In some embodiments, the compound is Compound B.

In another aspect, the present application provides a method of predicting whether a subject with cancer is likely to be responsive to treatment with a compound contemplated herein. In some embodiments, the method comprises detecting presence or absence of a insertion mutation in a sample of tumor cells from the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is likely to be responsive to the treatment with a compound contemplated herein if the mutation is detected.

In some embodiments, the compound is Compound A. In some embodiments, the compound is Compound B.

In another aspect, the present application provides a method of predicting whether a subject with cancer is likely to be responsive to treatment with a compound contemplated herein. In some embodiments, the method comprises:

(a) providing tumor cells of the subject; and

(b) detecting presence or absence of a mutation in the tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the presence of the mutation correlates with an increased likelihood of responsiveness or an increased responsiveness of the subject to the treatment.

In some embodiments, the compound is Compound A. In some embodiments, the compound is Compound B.

In some embodiments of the various methods provided herein, the prediction of whether a subject with cancer is likely to be responsive to treatment by a compound contemplated herein is made by detecting the presence of a mutation in the tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG. In other embodiments, the method further comprises administration of a compound contemplated herein to the subject. In yet other embodiments, the method further comprises administration of a compound contemplated herein to the subject if the subject is predicted to be likely to be responsive to the treatment. In yet other embodiments, a compound contemplated herein is administered in a therapeutically effective amount. The present application also contemplates methods of identifying a subject with cancer who is likely to be responsive to treatment with a compound contemplated herein.

In one aspect, the present application provides a method of identifying a subject with cancer who is likely to be responsive to treatment with a compound contemplated herein. In some embodiments, the method comprises:

(a) providing tumor cells of the subject;

(b) detecting presence or absence of a mutation in the provided tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(c) identifying the subject as being likely to be responsive to treatment with a compound contemplated herein if the mutation is detected in the provided tumor cells.

In some embodiments, the compound is Compound A. In some embodiments, the compound is Compound B.

In another aspect, the present application provides a method of identifying a subject with cancer who is likely to be responsive to treatment with a compound contemplated herein. In some embodiments, the method comprises:

(a) detecting presence or absence of a mutation in a sample from the subject, wherein the sample comprises tumor cells, and wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(b) identifying the subject as being likely to be responsive to a treatment with a compound contemplated herein if the mutation is detected in the sample from the subject.

In some embodiments, the compound is Compound A. In some embodiments, the compound is Compound B.

In yet another aspect, the present application provides a method of identifying a subject with cancer who is likely to be responsive to treatment with a compound contemplated herein. In some embodiments, the method comprises:

(a) detecting presence or absence of a mutation in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG; and

(b) identifying the subject as being likely to be responsive to a treatment with a compound contemplated herein if the mutation is detected in the tumor cells of the subject.

In some embodiments, the compound is Compound A. In some embodiments, the compound is Compound B.

In another aspect, the present application provides a method of identifying a subject with cancer who is likely to be responsive to treatment with a compound contemplated herein. In some embodiments, the method comprises:

(a) detecting presence or absence of a mutation in a sample of tumor cells from the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG; and

(b) identifying the subject as being likely to be responsive to a treatment with a compound contemplated herein if the mutation is detected in the sample.

In some embodiments, the compound is Compound A. In some embodiments, the compound is Compound B.

In another aspect, the present application provides a method of identifying a subject with cancer who is likely to be responsive to treatment with a compound contemplated herein. In some embodiments, the method comprises:

(a) providing tumor cells of the subject;

(b) detecting presence or absence of a in the provided tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is identified as likely to be responsive to the treatment with a compound contemplated herein if the mutation is detected in the provided tumor cells.

In some embodiments, the compound is Compound A. In some embodiments, the compound is Compound B.

In another aspect, the present application provides a method of identifying a subject with cancer who is likely to be responsive to treatment with a compound contemplated herein. In some embodiments, the method comprises detecting presence or absence of a mutation in tumor cells of the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is identified as likely to be responsive to the treatment with a compound contemplated herein if the mutation is detected in the tumor cells.

In some embodiments, the compound is Compound A. In some embodiments, the compound is Compound B.

In another aspect, the present application provides a method of identifying a subject with cancer who is likely to be responsive to treatment with a compound contemplated herein. In some embodiments, the method comprises detecting presence or absence of a mutation in a sample of tumor cells from the subject, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the subject is identified as likely to be responsive to the treatment with a compound contemplated herein if the mutation is detected in the sample.

In some embodiments, the compound is Compound A. In some embodiments, the compound is Compound B.

In another aspect, the present application provides a method of identifying a subject with cancer who is likely to be responsive to treatment with a compound contemplated herein. In some embodiments, the method comprises:

(a) providing tumor cells of the subject; and

(b) detecting presence or absence of a mutation in the provided tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

wherein the presence of the mutation identifies the subject as likely to be responsive to the treatment with a compound contemplated herein.

In some embodiments, the compound is Compound A. In some embodiments, the compound is Compound B.

In some embodiments of the various methods provided herein, the identification of a subject with cancer who is likely to be responsive to treatment by a compound contemplated herein is made by detecting the presence of a mutation in the tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG. In other embodiments, the method further comprises administration of a compound contemplated herein to the subject. In yet other embodiments, the method further comprises administration of a compound contemplated herein to the subject that is identified to be likely to be responsive to the treatment. In yet other embodiments, a compound contemplated herein is administered in a therapeutically effective amount.

In yet another aspect, provided herein is a method for determining whether a subject with cancer is sensitive to a treatment with a compound contemplated herein. In some embodiments, the method comprises:

(a) providing tumor cells of the subject;

(b) detecting presence or absence of a mutation in the provided tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(c) diagnosing the subject as being sensitive to the treatment with a compound contemplated herein if the mutation is detected in the provided tumor cells.

In some embodiments, the compound is Compound A. In some embodiments, the compound is Compound B.

In some embodiments, the method further comprises administration of a compound contemplated herein to the subject. In other embodiments, the method further comprises administration of a compound contemplated herein to the subject if the subject is determined to be sensitive to the treatment. In yet other embodiments, a compound contemplated herein is administered in a therapeutically effective amount.

In yet another aspect, the present application provides a method of determining whether a subject with cancer is sensitive to treatment with a compound contemplated herein.

In some embodiments, the method comprises:

(a) providing tumor cells of the subject;

(b) detecting presence or absence of a mutation in the provided tumor cells, wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(c) diagnosing the subject as being sensitive to treatment to the treatment with a compound contemplated herein if the mutation is detected in the provided tumor cells.

In some embodiments, the compound is Compound A. In some embodiments, the compound is Compound B.

In some embodiments, the method further comprises administration of a compound contemplated herein to the subject. In other embodiments, the method further comprises administration of a compound contemplated herein to the subject if the subject is determined to be sensitive to the treatment. In yet other embodiments, a compound contemplated herein is administered in a therapeutically effective amount.

In yet another aspect, the present application provides a method of determining whether a subject with cancer is sensitive to treatment with a compound contemplated herein.

In some embodiments, the method comprises:

(a) detecting presence or absence of a mutation in a sample from the subject, wherein the sample comprises tumor cells, and wherein the mutation is an EGFR mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or wherein the mutation is an ErbB-2 mutation selected from the group consisting of: D769H, D769Y, R896C, V777L, and V777_G778insCG;

(b) diagnosing the subject as being sensitive to treatment to the treatment with a compound contemplated herein if the mutation is detected in the sample.

In some embodiments, the compound is Compound A. In some embodiments, the compound is Compound B.

### Combination and Concurrent Therapies

In one embodiment, the compounds contemplated herein are useful in the methods of the present application when used concurrently with at least one additional compound useful for preventing and/or treating diseases and/or disorders contemplated herein.

In one embodiment, the compounds contemplated herein are useful in the methods of present application in combination with at least one additional compound useful for preventing and/or treating diseases and/or disorders contemplated herein.

These additional compounds may comprise compounds of the present application or other compounds, such as commercially available compounds, known to treat, prevent, or reduce the symptoms of diseases and/or disorders contemplated herein. In some embodiments, the combination of at least one compound contemplated herein or a salt thereof, and at least one additional compound useful for preventing and/or treating diseases and/or disorders contemplated herein, has additive, complementary or synergistic effects in the prevention and/or treatment of diseases and/or disorders contemplated herein.

In another non-limiting example, the compounds contemplated herein, or a salt or solvate thereof, can be used concurrently or in combination with one or more agents known to be useful in treating or preventing HER-driven (such as an EGFR-driven) drug-resistant cancer.

As used herein, combination of two or more compounds may refer to a composition wherein the individual compounds are physically mixed or wherein the individual compounds are physically separated. A combination therapy encompasses administering the components separately to produce the desired additive, complementary or synergistic effects.

In one embodiment, the compound and the agent are physically mixed in the composition. In another embodiment, the compound and the agent are physically separated in the composition.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326), the median-effect equation (Chou & Talalay, 1984, Adv. Enzyme Regul. 22: 27-55), and through the use of isobolograms (Tallarida & Raffa, 1996, Life Sci. 58: 23-28). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present application to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated herein. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions disclosed herein may be varied so as to obtain an amount of the active ingredient that is effective to achieve the desired therapeutic response for a particular patient, composition, and mode of administration, without being toxic to the patient.

The therapeutically effective amount or dose of a compound of the present application depends on the age, sex and weight of the patient, the current medical condition of the patient and the progression of a disease or disorder contemplated herein.

A medical doctor, e.g., physician or veterinarian, having ordinary skill in the art may readily determine and prescribe the effective amount of the pharmaceutical composition required. For example, the physician or veterinarian could start doses of the compounds contemplated herein employed in the pharmaceutical composition at levels lower than that required in order to achieve the desired therapeutic effect and gradually increase the dosage until the desired effect is achieved.

A suitable dose of a compound of the present application may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

Compounds contemplated herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In some embodiments, the dose of a compound contemplated herein is from about 1 mg and about 2,500 mg. In some embodiments, a dose of a compound contemplated herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the compositions contemplated herein are administered to the patient in dosages that range from one to five times per day or more. In another embodiment, the compositions contemplated herein are administered to the patient in range of dosages that include, but are not limited to, once every day, every two, days, every three days to once a week, and once every two weeks. It is readily apparent to one skilled in the art that the frequency of administration of the various combination compositions contemplated herein varies from individual to individual depending on many factors including, but not limited to, age, disease or disorder to be treated, gender, overall health, and other factors. Thus, the present disclosure should not be construed to be limited to any particular dosage regime and the precise dosage and composition to be administered to any patient is determined by the attending physical taking all other factors about the patient into account.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound contemplated herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms and/or infection.

The compounds for use in the method disclosed herein may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g. about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

In one embodiment, the compositions contemplated herein are formulated using one or more pharmaceutically acceptable excipients or carriers. In one embodiment, the pharmaceutical compositions contemplated herein comprise a therapeutically effective amount of a compound contemplated herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal and the like. In many cases, it is preferable to include isotonic agents, for example, sugars, sodium chloride, or polyalcohols such as mannitol and sorbitol, in the composition.

In one embodiment, the present application is directed to a packaged pharmaceutical composition comprising a container holding a therapeutically effective amount of a compound contemplated herein, alone or in combination with a second pharmaceutical agent; and instructions for using the compound to treat, prevent, or reduce one or more symptoms of a disease or disorder contemplated in the present disclosure.

Formulations may be employed in admixtures with conventional excipients, i.e., pharmaceutically acceptable organic or inorganic carrier substances suitable for any suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., analgesic agents.

Routes of administration of any of the compositions contemplated herein include inhalational, oral, nasal, rectal, parenteral, sublingual, transdermal, transmucosal (e.g, sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical administration.

### Additional Administration Forms

Additional dosage forms include dosage forms as described in U.S. Pat. Nos. 6,340,475; 6,488,962; 6,451,808; 5,972,389; 5,582,837; and 5,007,790. Additional dosage forms also include dosage forms as described in U.S. Patent Applications Nos. 20030147952; 20030104062; 20030104053; 20030044466; 20030039688; and 20020051820. Further dosage forms include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present application may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use in the methods disclosed herein may be administered in the form of microparticles, for example, by injection or in the form of wafers or discs by implantation.

In one embodiment, the compounds contemplated herein are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of the present disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present application. Moreover, all values that fall within these ranges, as well as the upper or lower limits of a range of values, are also contemplated by the present application.

The following examples further illustrate aspects of the present application. However, they are in no way a limitation of the teachings or disclosure of the present application as set forth herein.

### EXAMPLES

Certain aspects of the disclosure are now described with reference to the following Examples. These Examples are provided for the purpose of illustration only and the disclosure should in no way be construed as being limited to these Examples, but rather should be construed to encompass any and all variations which become evident as a result of the teaching provided herein.

### Example 1: TRLX-TKI (Compound B) Kinase Assay

Tarloxotinib (TRLX or Compound A) is a hypoxia-activated EGFR/HER2/HER4 TKI prodrug that releases an irreversible EGFR/HER2 tyrosine kinase inhibitor (TRLX-TKI; in this case, Compound B) under pathologically hypoxic conditions. While most EGFR (also known as HER1 or ErbB-1) mutations predict a response to several FDA-approved tyrosine kinase inhibitors (TKI), several mutations of EGFR, including C797S single, double, and triple mutants, are activating mutations in the tyrosine kinase domain that have significantly decreased sensitivity to EGFR inhibitors and currently have no approved targeted therapies.

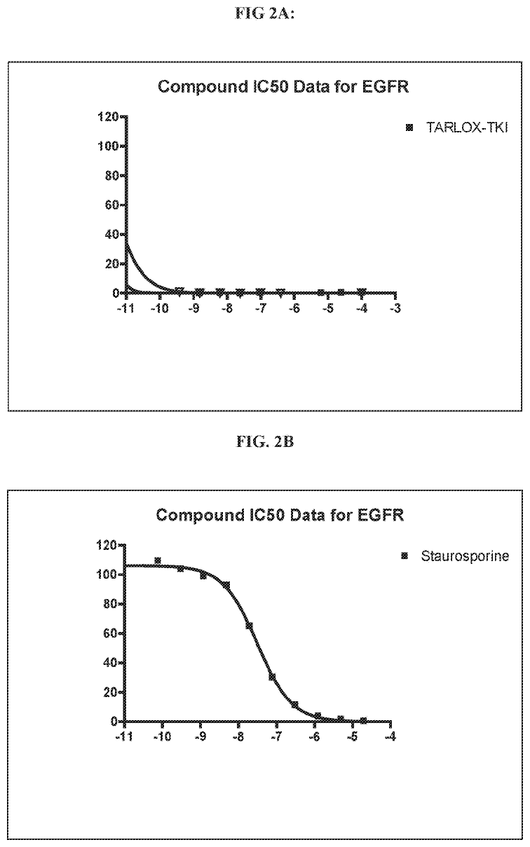

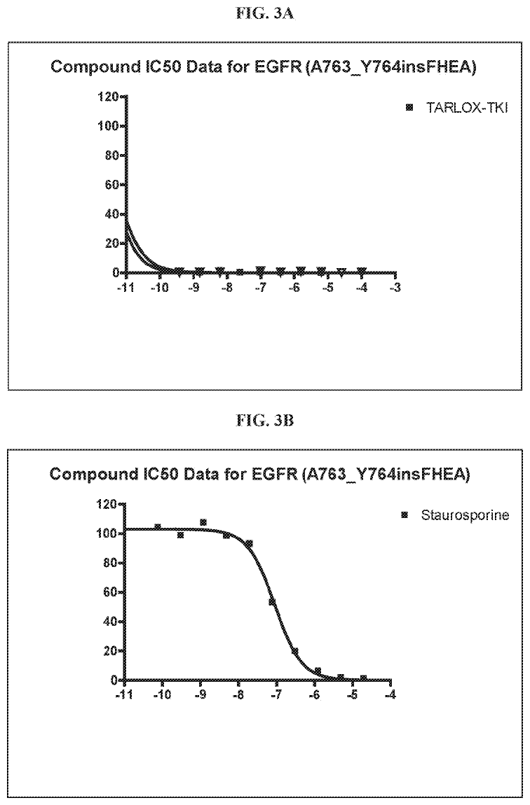

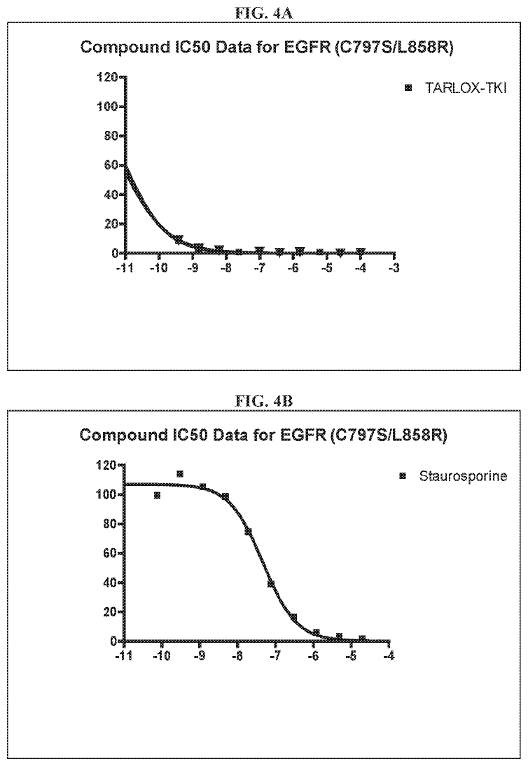

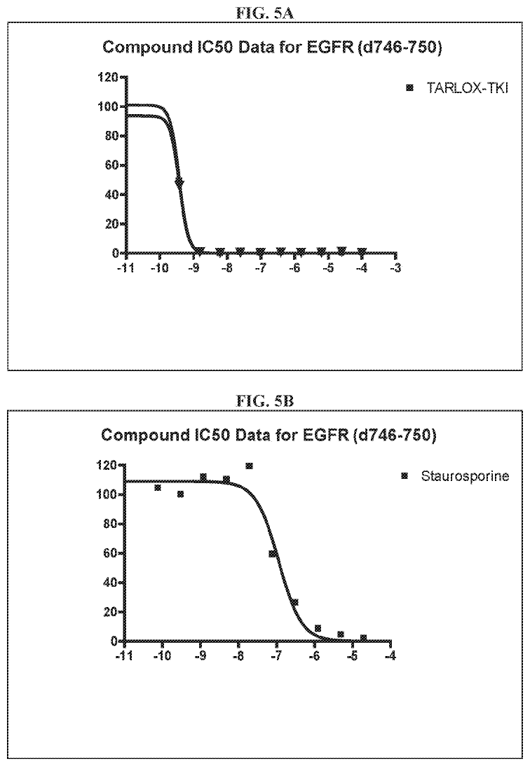

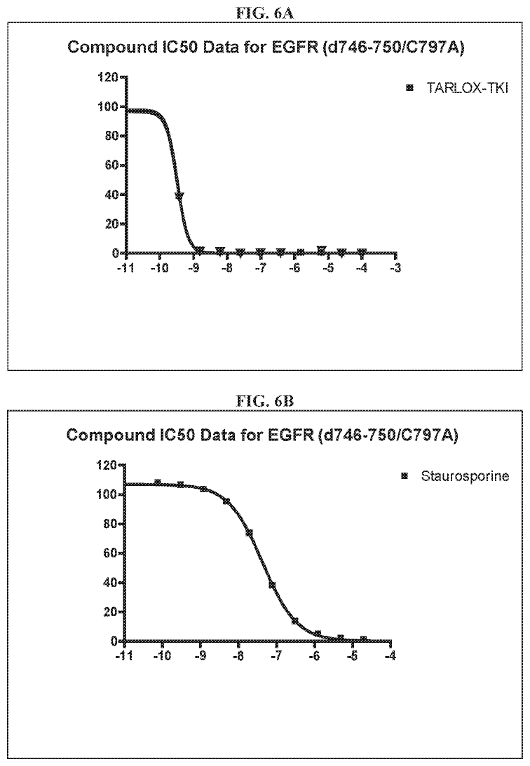

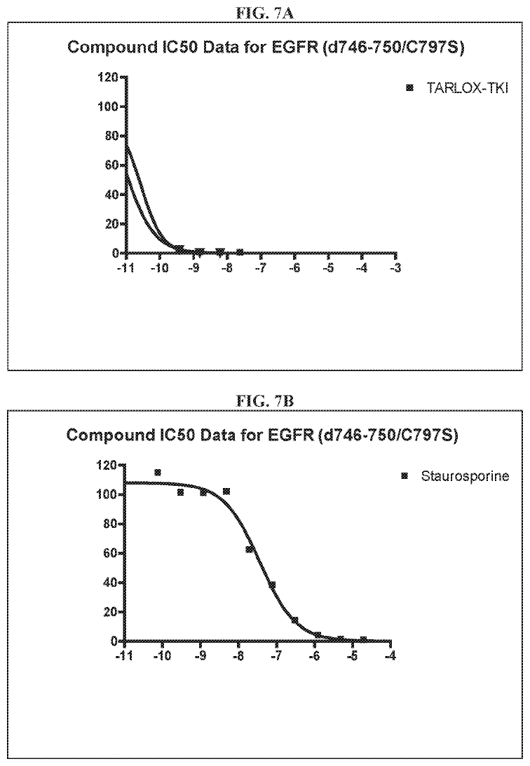

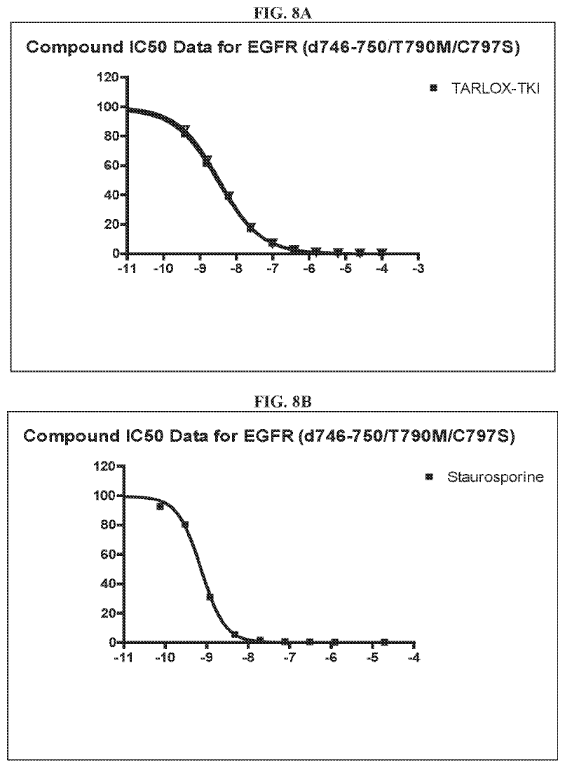

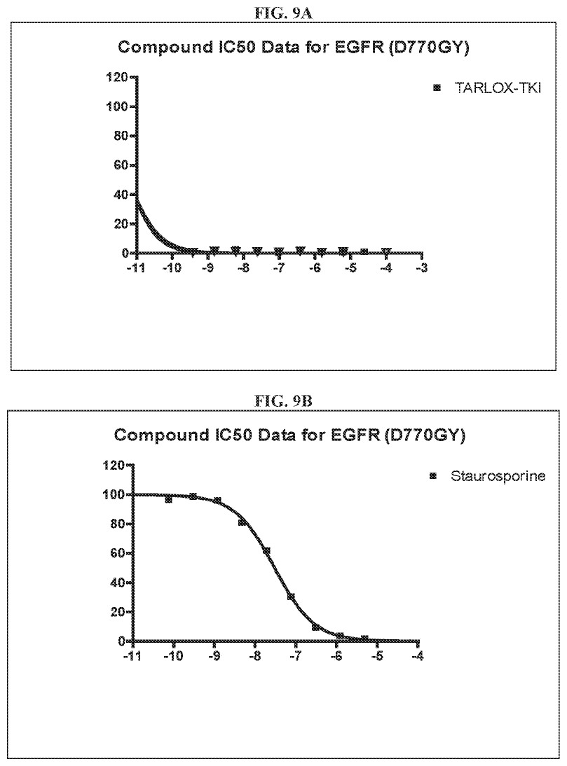

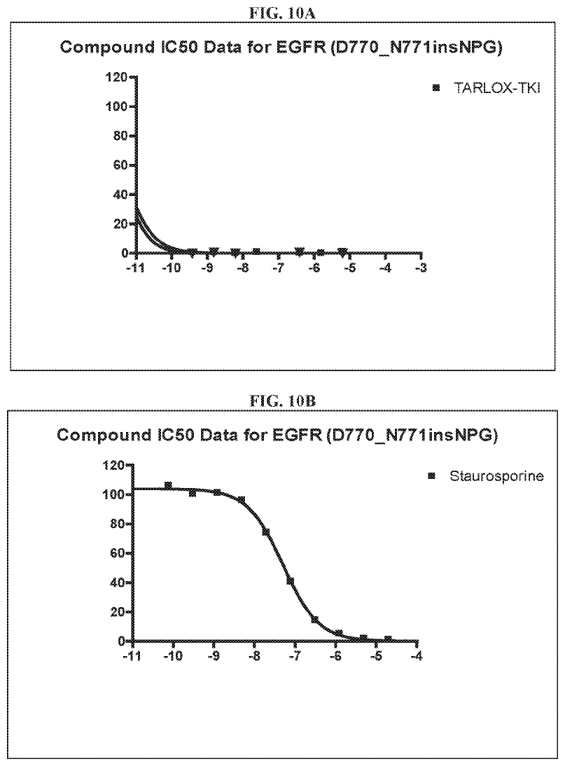

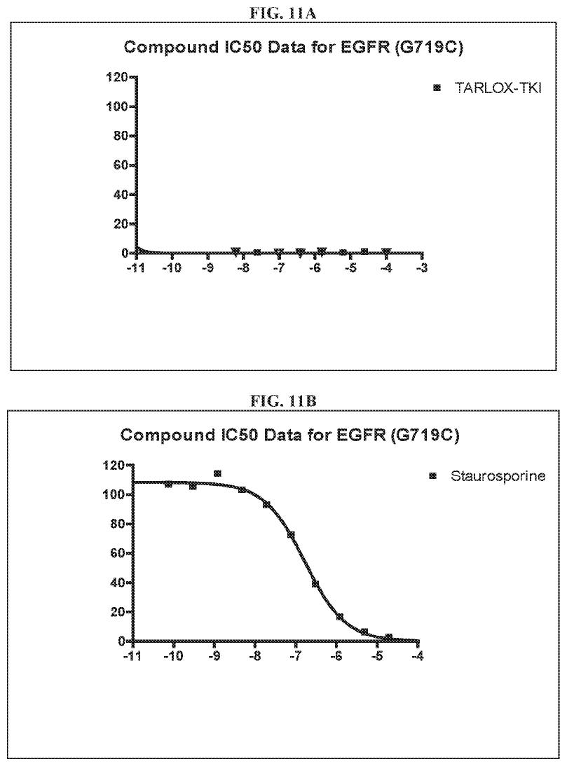

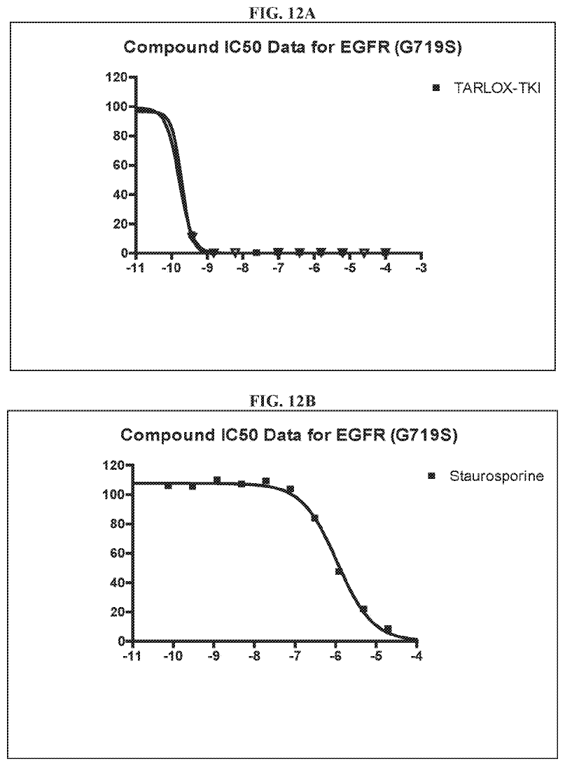

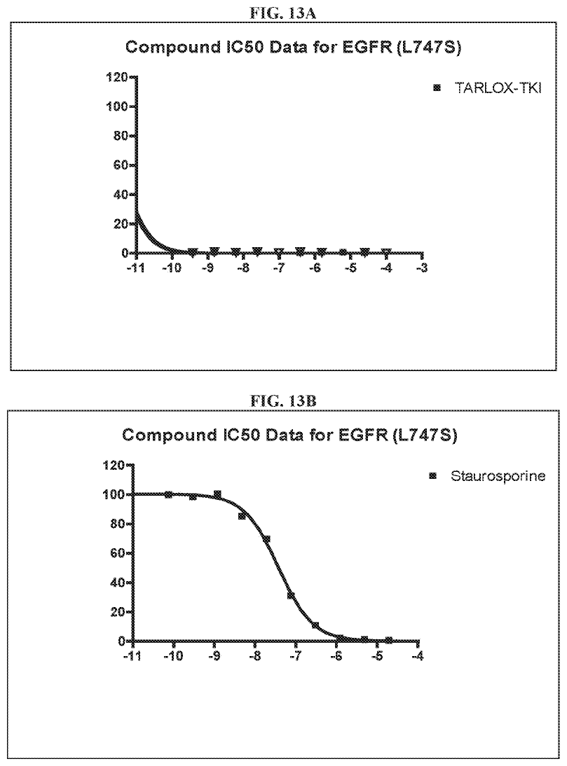

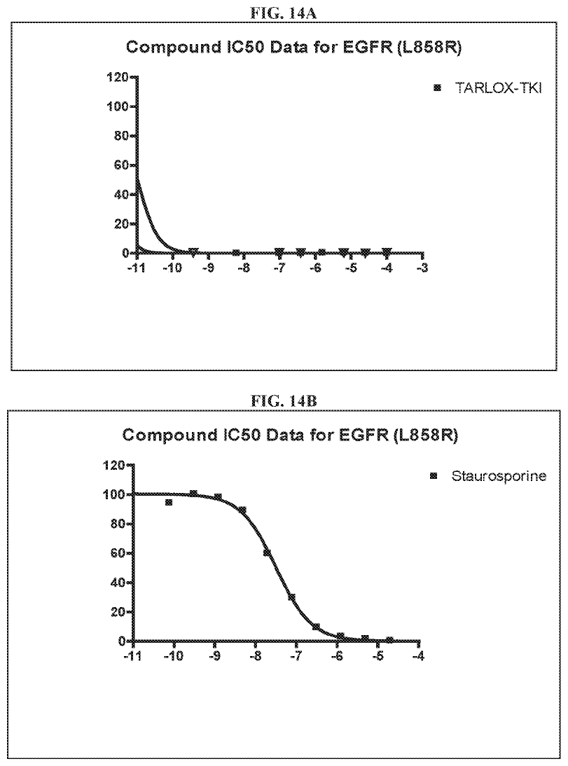

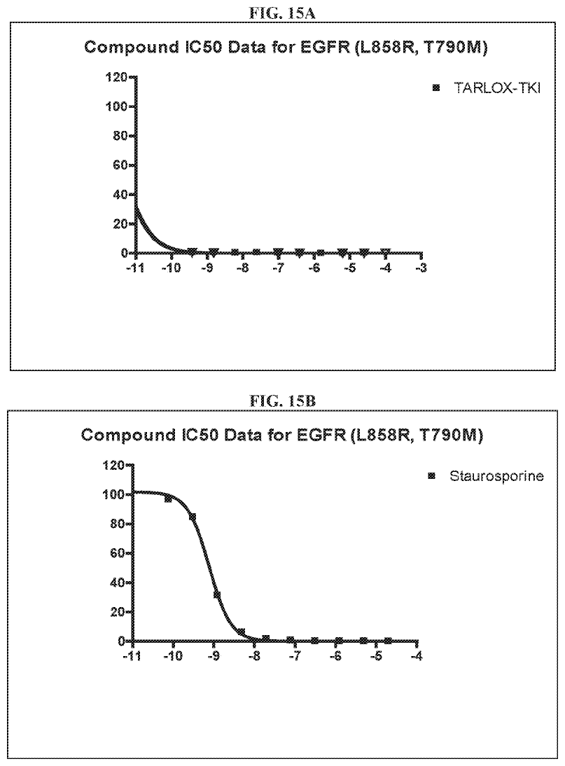

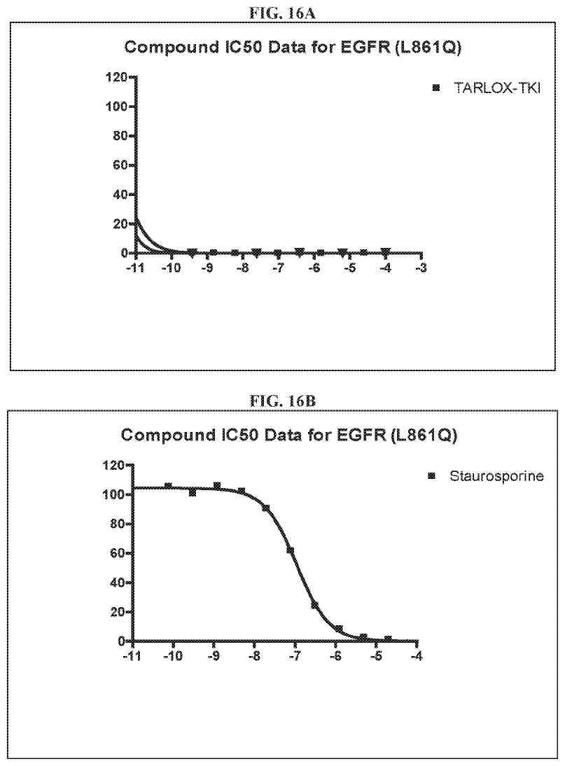

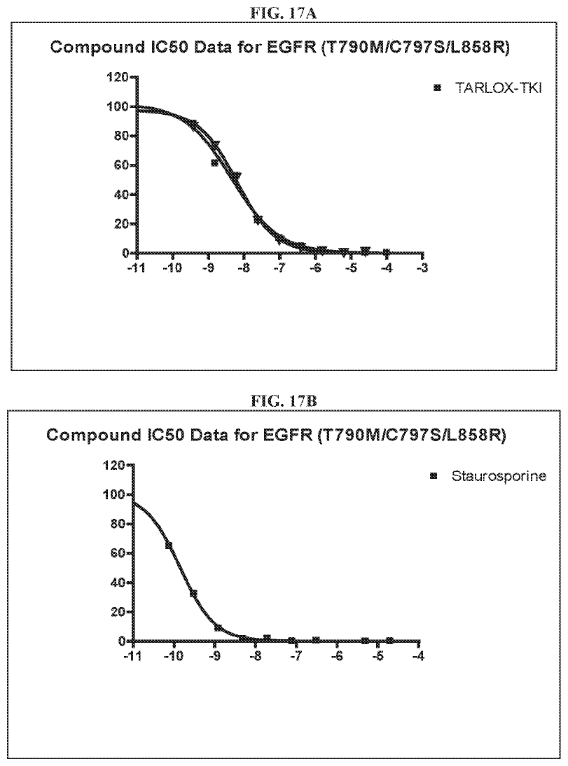

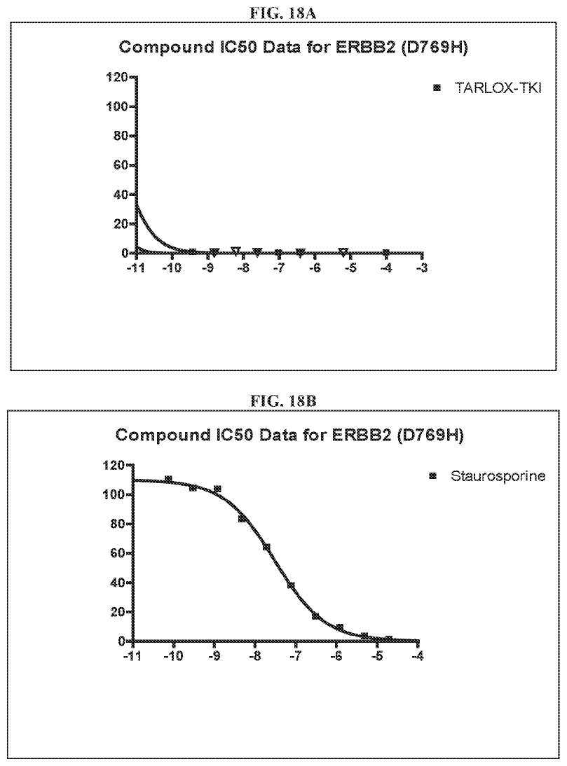

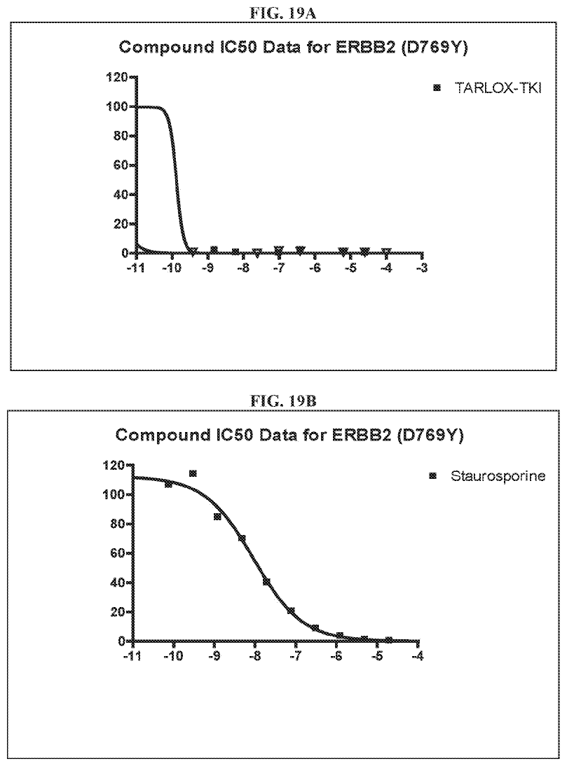

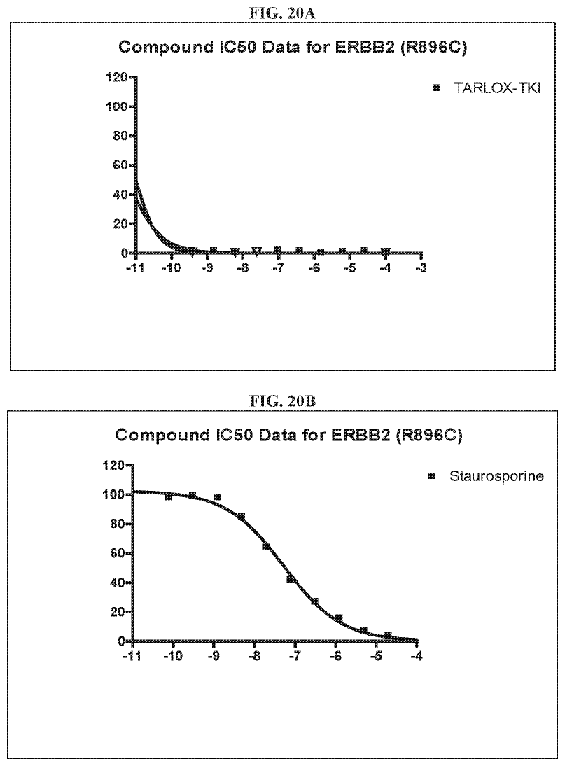

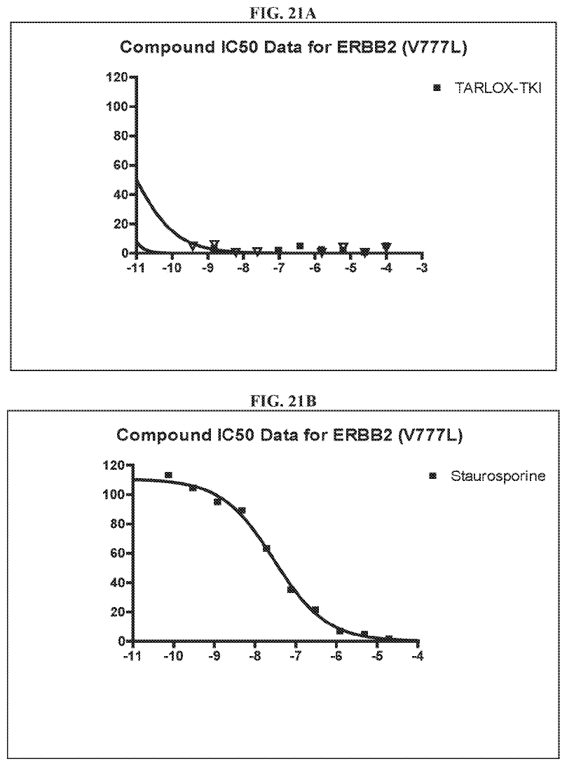

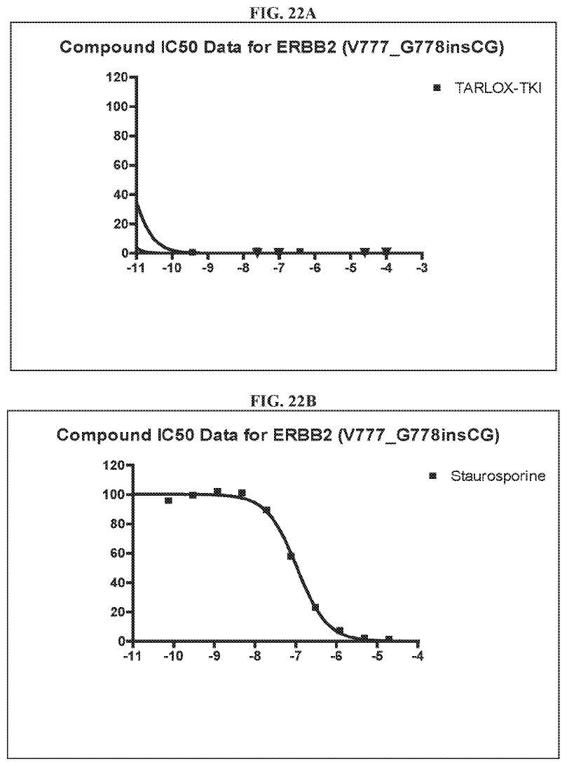

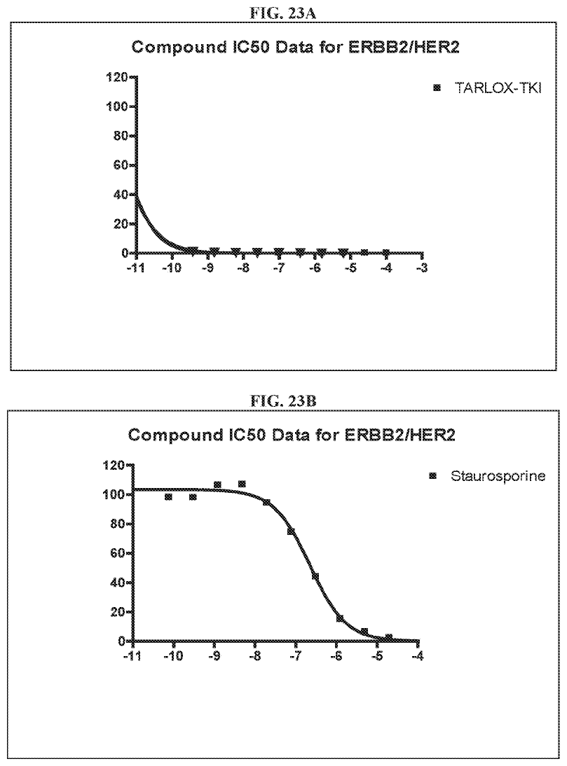

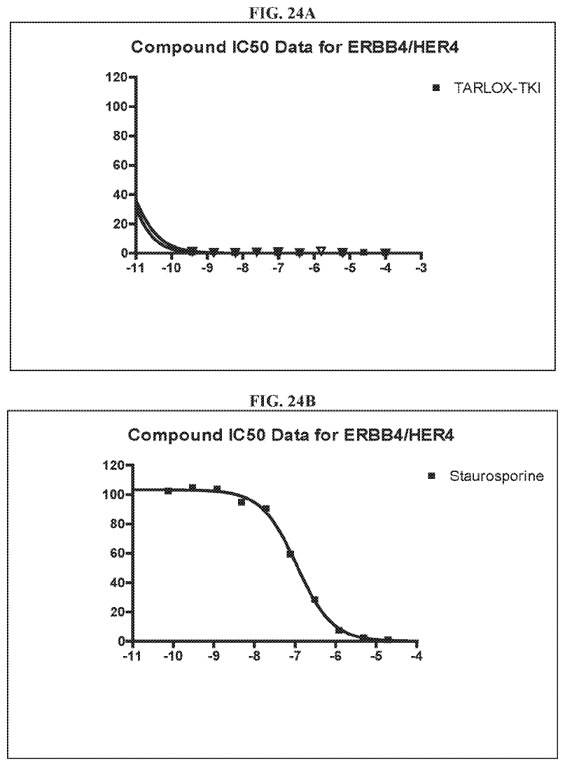

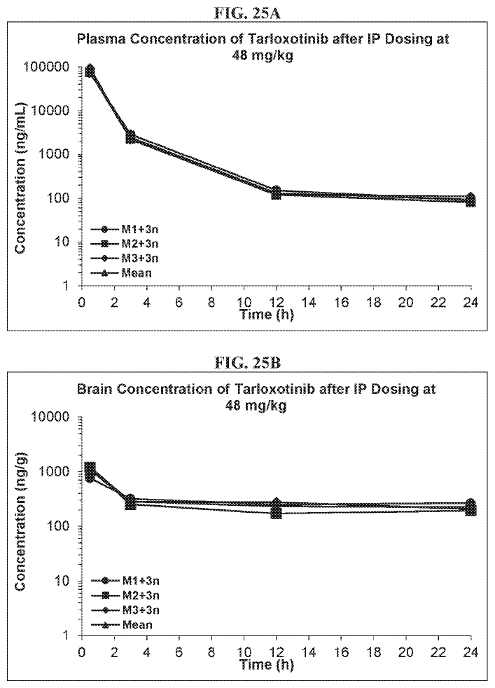

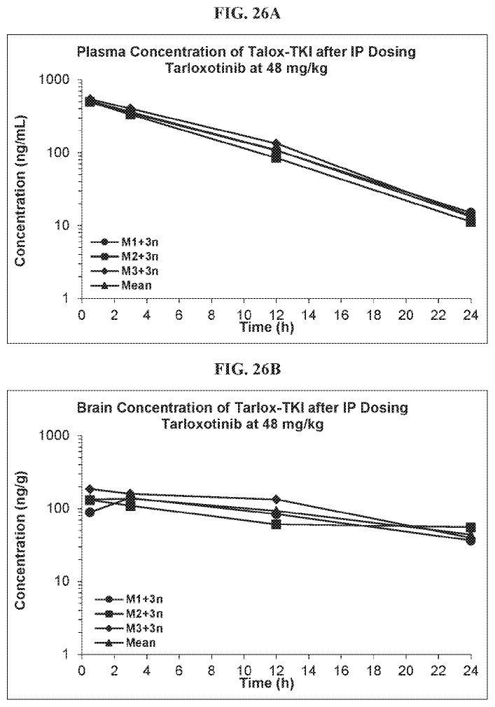

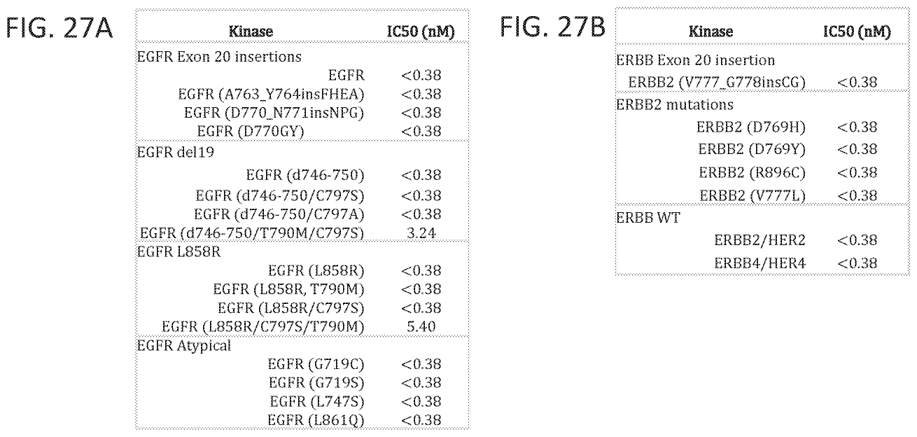

Compound B was tested against 23 kinases in 10-dose IC₅₀ duplicate mode with a 4-fold serial dilution starting at 100 μM. Control compound, Staurosporine, was tested in 10-dose IC₅₀ mode with 4-fold serial dilution starting at 20 μM. Reactions were carried out at Kₘ ATP according to the RBC Km binding structure. The resulting IC₅₀ data is shown in Table 3. Activity curves for both Compound B and Staurosporine are shown in FIGS. 2-24. Compound B shows potent activity across a spectrum of in vitro kinase assays for various ErbB kinases.

TABLE 3
IC₅₀ for Compound B and various ErbB kinases compared to Staurosporine control

| | | Tarlox-TKI | | Staurosporine |
|---|---|---|---|---|
| | | (Compound B) IC₅₀(nM): | | (control |
| | [ATP] | Data | Data | compound) |
| Kinase | (μM) | Set 1 | Set 2 | IC₅₀ (nM) |
| EGFR (wt) | 1 | <0.381 | <0.381 | 31.4 |
| EGFR (A763_Y764insFHEA) | 5 | <0.381 | <0.381 | 89.6 |
| EGFR (C797S/L858R) | 20 | <0.381 | <0.381 | 46.8 |
| EGFR (d746-750) | 50 | <0.381 | <0.381 | 114 |
| EGFR (d746-750/C797A) | 30 | <0.381 | <0.381 | 43.2 |
| EGFR (d746-750/C797S) | 20 | <0.381 | <0.381 | 36.4 |
| EGFR (d746-750/T790M/C797S) | 5 | 3.10 | 3.38 | 0.733 |
| EGFR (D770GY) | 2.5 | <0.381 | <0.381 | 30.1 |
| EGFR (D770_N771insNPG) | 2.5 | <0.381 | <0.381 | 50.5 |
| EGFR (G719C) | 5 | <0.381 | <0.381 | 167 |
| EGFR (G719S) | 5 | <0.381 | <0.381 | 1100 |
| EGFR (L747S) | 2.5 | <0.381 | <0.381 | 37.7 |
| EGFR (L858R) | 10 | <0.381 | <0.381 | 31.8 |
| EGFR (L858R/T790M) | 1 | <0.381 | <0.381 | 0.766 |
| EGFR (L861Q) | 5 | <0.381 | <0.381 | 110 |
| EGFR (T790M/C797S/L858R) | 1 | 4.52 | 6.27 | 0.141 |
| ErbB-2 (D769H) | 5 | <0.381 | <0.381 | 30.4 |
| ErbB-2 (D769Y) | 10 | <0.381 | <0.381 | 9.21 |
| ErbB-2 (R896C) | 10 | <0.381 | <0.381 | 52.6 |
| ErbB-2 (V777L) | 5 | <0.381 | <0.381 | 29.3 |
| ErbB-2 (V777_G778insCG) | 10 | <0.381 | <0.381 | 107 |
| ErbB-2/HER2 (wt) | 30 | <0.381 | <0.381 | 223 |
| ErbB-4/HER4 (wt) | 2.5 | <0.381 | <0.381 | 111 |

The calculated IC₅₀ values for Compound B and the various kinases were compared to the IC₅₀ values for several kinases for known the EGFR inhibitors: osimer, afatinib, gefitinib, EGF816, and TTI-2341.

TABLE 4
IC₅₀ for Compound B and various kinases compared to known EGFR inhibitors
(osimertinib, afatinib, gefitinib, EGF816, and TTI-2341)

| | Tarlox-TKI | | | | | |
|---|---|---|---|---|---|---|
| | (Compound B) | Osimertinib^{1, 2, 4} | Afatinib^{1, 5} | Gefitinib^{1, 4} | EGF816² | TTI-2341³ |
| Mutation | IC₅₀(nM) | IC₅₀(nM) | IC₅₀(nM) | IC₅₀(nM) | IC₅₀(nM) | IC₅₀(nM |
| EGFR (wt) | <0.4 | 184 | 0.2 | 3 | | <0.5 |
| EGFR (L858R) | <0.4 | 12 | 0.2 | <1 | | |
| EGFR (d746-750) | <0.4 | | | | | |
| EGFR (d746- | <0.4 | | | | | <0.5 |
| 750/C797S) | | | | | | |
| EGFR (d746- | 3.2 | 107 | 39 | 103 | | 62 |
| 750/T790M/C797S) | | | | | | |
| EGFR (L858R/T790M) | <0.4 | <1 | <1 | 309 | <1 | 2.9 |
| EGFR | 5.4 | 77 | 58 | 153 | 196 | 389 |
| (L858R/T790M/C797S) | | | | | | |
| ErbB-2/HER2 (wt) | <0.4 | | 14 | | | <0.5 |
| ErbB-4/HER4 (wt) | <0.4 | | | | | |

References relating to Table 4:
¹Park et al., Angew Chem Int Ed Engl. 2017 Jun. 19; 56(26): 7634-7638
²Engel et al., Angew Chem Int Ed Engl. 2016 Aug. 26; 55(36): 10909-12
³Wang et al., Abstract # 4329, SNO 2017
⁴Cross et al., Cancer Discov; 4(9); 1046-61
⁵Li et al., Oncogene. 2008 Aug. 7; 27(34): 4702-4711

The data in Table 4 shows that Compound B has potent activity in the in vitro kinase assays for various EGFR (ErbB-1) and HER2 (ErbB-2) mutations, especially the C797S double and triple mutations d746-750/C797S, d746-750/T790M/C797S, and L858R/T790M/C797S as compared to the published data for other EGFR inhibitors. This is particularly important to the C797S population, as that population is resistant to currently approved covalent, irreversible EGFR inhibitors.

### Example 2: Evaluation of TRLX (Compound A) or TRLX-TKI (Compound B) on Various EGFR Mutant Non-Small Cell Lung Cancer Cell Lines

Non-small cell lung cancer (NSCLC) has been characterized as a hypoxic disease and approximately 15% of lung adenocarcinomas harbor EGFR mutations. Various human lung adenocarcinoma cell lines with the EGFR mutations set forth in Table 5 are derived and characterized, in order to accelerate development of targeted therapies for these mutations.

TABLE 5
Various EGFR mutations in derived NSCLC cell line

| | | | |
|---|---|---|---|
| EGFR(wt) | EGFR (d746- | EGFR (L858R) | EGFR |
| | 750/T790M/C797S) | | (d746-750/C797A) |
| EGFR | EGFR (D770GY) | EGFR (L858R, T790M) | EGFR |
| (A763_Y764insFHEA) | | | (d746-750/C797S) |
| EGFR | EGFR | EGFR (L861Q) | EGFR (G719S) |
| (C797S/L858R) | (D770_N771insNPG) | | |
| EGFR | EGFR (G719C) | EGFR | EGFR (L747S) |
| (d746-750) | | (T790M/C797S/L858R) | |

These cell lines are treated with TRLX-TKI or TRLX, and evaluated for tumor growth to confirm the applicability of TRLX-TKI and TRLX as therapeutic agents for tumors harboring these types of mutations. Evaluation is performed using, e.g., cell proliferation and growth assays, in vitro inhibitory enzyme kinetic assays, mass spectrometry, equilibrium binding assays, and the like. The results are compared to known EGFR inhibitors, including osimertinib, afatinib, gefitinib, EGF816, and TTI-2341.

### Example 3: Evaluation of Potency of TRLX (Compound A) and TRLX-TKI (Compound B) Against Cancer Cell Lines Under Normal Oxygenation (Aerobic) and Under Low Oxygenation (Hypoxic) Conditions

Tarloxotinib (TRLX) is a Hypoxia-Activated Prodrug (HAP) that is reduced to a nitro radical anion which acts as a direct ‘oxygen sensor’ releasing an irreversible pan-ErbB inhibitor (Tarloxotinib-E, TRLX-TKI)) under hypoxic conditions. TRLX undergoes hypoxia-selective cellular metabolism to release TRLX-TKI in various cell lines. TRLX-TKI antiproliferative activity is greater following exposure of cells to hypoxia. TRLX-TKI is active in mutant-positive EGFR-driven NSCLC, but also demonstrates nanomolar activity against WT EGFR and HER2-driven cell lines under hypoxia.

TABLE 6
Antiproliferative activity of TRLX in various cancer cell lines

| Human Cancer Cell Line | | | | | | |
|---|---|---|---|---|---|---|
| | | Driver | | TRLX IC₅₀ (μM) | | |
| ID | Origin | Kinase | Mutation Status¹ | Aerobic² | Hypoxic³ | HCR⁴ |
| HCC827 | NSCLC | EGFR | Del746_L750 | 0.12 | 0.006 | 34 |
| PC9 | NSCLC | EGFR | Del746_L750 | 0.17 | 0.006 | 45 |
| HCC4006 | NSCLC | EGFR | Del747_L749 | 0.064 | 0.01 | 14 |
| H3255 | NSCLC | EGFR | L858R | 0.022 | 0.001 | 47 |
| H2126 | NSCLC | EGFR | WT | 24 | 0.4 | 75 |
| H1648 | NSCLC | EGFR | WT | 3.8 | 0.15 | 61 |
| H2073 | NSCLC | EGFR | WT | 0.05 | 0.002 | 49 |
| H1838 | NSCLC | EGFR | WT | 29 | 0.81 | 63 |
| FaDu | SCCHN | EGFR | WT | 0.2 | 0.002 | 88 |
| A431 | Epidermoid | EGFR | WT | 0.11 | 0.013 | 16 |
| H1975 | NSCLC | EGFR | L858R/T790M | 9.7 | 0.17 | 71 |
| H820 | NSCLC | EGFR | Del747_L751/T790M | 1.3 | 0.02 | 59 |
| Calu3 | NSCLC | HER2 | WT | 3.3 | 0.11 | 49 |
| SKOV3 | Ovarian | HER2 | WT | 15.6 | 0.59 | 48 |
| H661 | NSCLC | HER4 | WT | 8.5 | 0.08 | 136 |
| H1734 | NSCLC | K-Ras | G13C | 77 | 5.9 | 18 |
| H460 | NSCLC | K-Ras | Q61H | 109 | 9.3 | 13 |
| H1299 | NSCLC | N-Ras | Q61K | 83 | 15 | 7 |

¹Determined by Sequenom massARRAY
²24 h aerobic exposure with 96 h drug-free recovery
³4 h hypoxic exposure (<1 ppm O2) followed by 20 h aerobic recovery and 96 h drag-free recovery
⁴HCR = hypoxic cytotoxicity ratio (average of intra-experimental ratio of IC₅₀ values under aerobic and hypoxic conditions)

The data in Table 6 shows that Compound A has potent activity in in vitro cancer cell lines. Further the potency is greater under hypoxic conditions in which Compound A, without wishing to be bound by theory, is expected to be converted to the active TRLX-TKI (Compound B). Compound A and Compound B have potent activity in the in vitro cell assays for various EGFR (ErbB-1) and HER2 (ErbB-2) mutations, i.e. L858R/T790M, Del747_L751/T790M, and WT EGFR and HER driven cancer lines. Without wishing to be bound by theory, this mutant population is resistant to currently approved covalent, irreversible EGFR inhibitors.

### Example 4. Potency of TRLX (Compound A) and TRLX-TKI (Compound B) Against Ba/F3 Cells Expressing Various EGFR and HER2 Mutations

### Establishment of Ba/F3 Cells Expressing Mutant Forms of EGFR or HER2

The murine pro-B cell line Ba/F3 (RCB0805) was obtained from RIKEN Bio Resource Center (Tsukuba, Japan). Ba/F3 cells which express a human EGFR activating mutation (either E746_A750 del (exon 19 deletion), L858R mutation, or one of exon 20 insertions (A763insFQEA, V769insASV, D770insSVD, or H773insNPH)) with/without a secondary EGFR mutation were established as previously described (Nishino M. and Suda K., et al. Lung Cancer 2018). Several Ba/F3 lines which were established in the previous study were also used in this study. All Ba/F3 cells which express a HER2 exon 20 insertion mutation (A775_G776insYVMA, G776delinsVC, P780_Y781insGSP) were previously established (Koga T. and Kobayashi Y., et al. Lung Cancer 2018). Ba/F3 cells with the secondary HER2 C805S mutation, which confers resistance to poziotinib, were also previously established.

### Cell Growth Inhibition Assay

Cell growth inhibition assay for one of the following TKIs, tarloxotinib-E (activated form), tarloxotinib (pro-drug before activation), afatinib, poziotinib, and osimertinib, were performed as previously described (Nishino M. and Suda K., et al. Lung Cancer 2018). Briefly, 2000 cells were seeded in each well of 96-well plates. Twenty-four hours later, DMSO or a TKI at indicated drug concentration were added, and the cells were cultured for additional 72 hours. A colorimetric assay was used to estimate the growth inhibition of each drug using the Cell Counting Kit-8 reagent (Dojindo Laboratories, Kumamoto, Japan). Each experiment was performed in triplicate.

Various EGFR and HER2 mutations were introduced into Ba/F3 cells using retroviral vector. Growth inhibitory assays for TRLX (Compound A) and TRLX-TKI (Compound B), poziotinib, afatinib, and osimertinib were performed in Ba/F3 cells with various EGFR exon 20 and HER2 mutations.

TABLE 7
Cellular potency in Ba/F3 cells expressing
various EGFR exon 20 insertion mutations

| IC₅₀ (nM) | Afatinib | Poziotinib | Osimertinib | Tarloxotinib | Tarloxotinib-TKI |
|---|---|---|---|---|---|
| A763insFQEA | 0.7 | 0.7 | 14.6 | 15.2 | <0.5 |
| V769insASV | 35.5 | 28 | 118.4 | 675.9 | 7.6 |
| D770insSVD | 86 | 27.9 | 184.7 | 990.1 | 7.3 |
| H773insNPH | 35.8 | 2.2 | 61.9 | 714 | 9.9 |
| H773insH | 325 | 22.8 | 77.7 | >1000 | 73.1 |

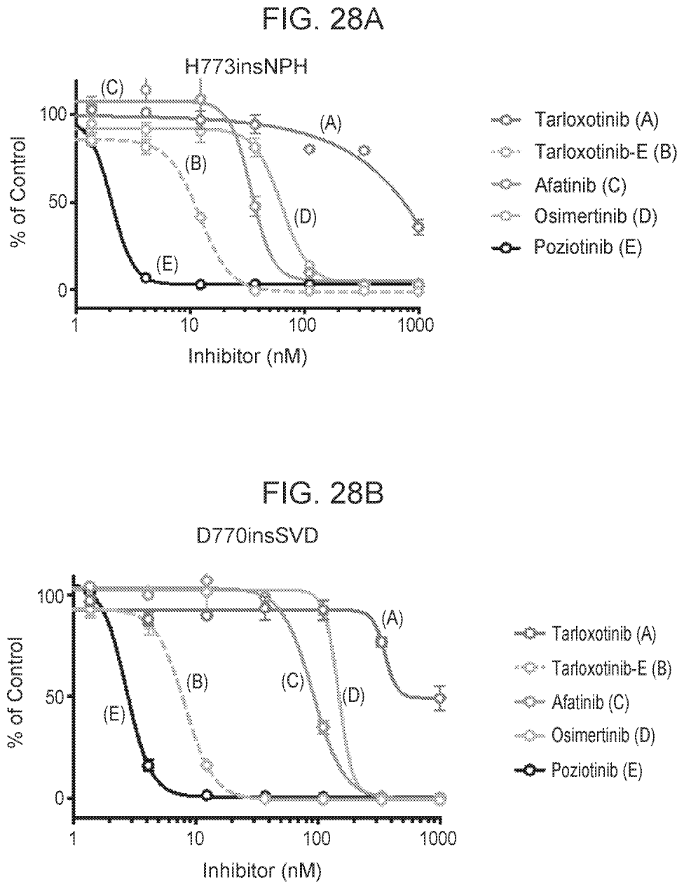

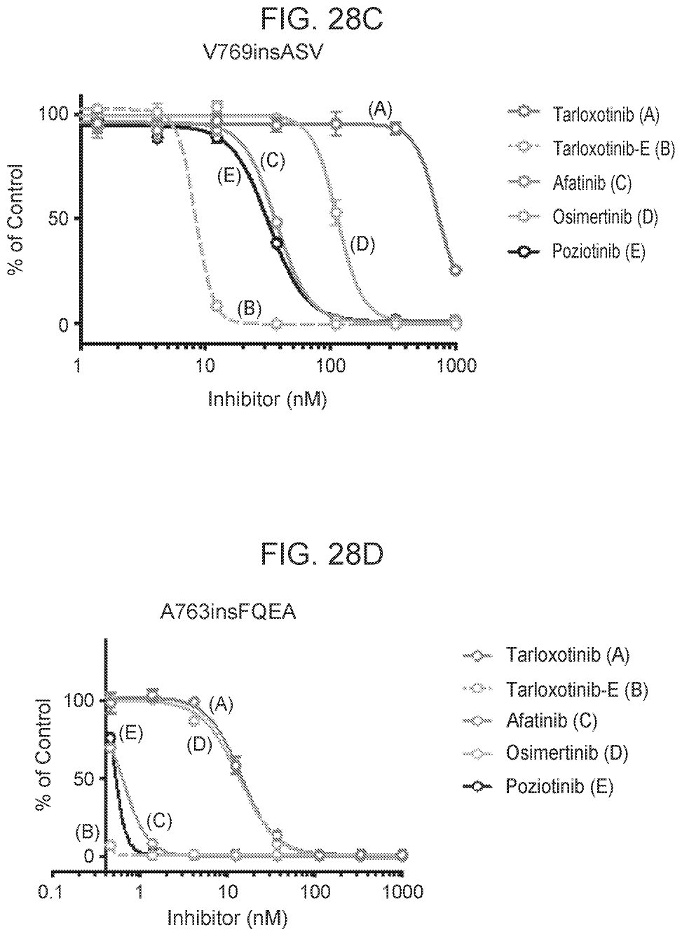

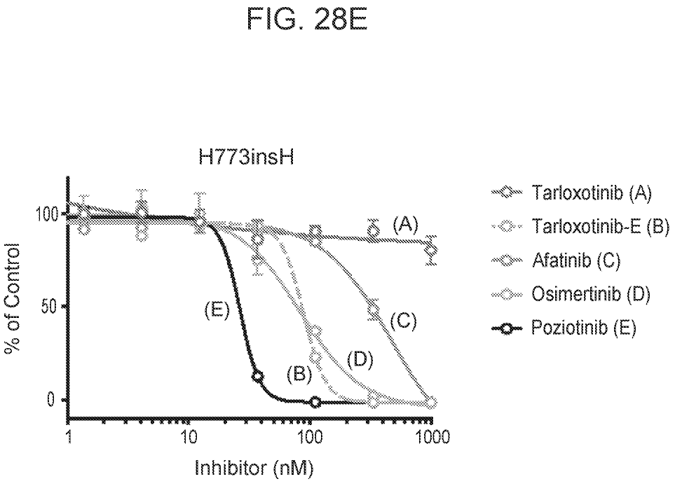

Table 7 shows Tarloxotinib-E cellular potency in Ba/F3 cells, wherein Tarloxotinib-TKI exhibits potent in vitro inhibition of EGFR exon20 insertion mutations (FIG. 28A-E).

TABLE 8
Cellular potency in Ba/F3 cells expressing EGFR C797S double
and triple mutations with del19 and L585R ± T790M

| IC₅₀ (nM) | Afatinib | Poziotinib | Osimertinib | Tarloxotinib | Tarloxotinib-TKI |
|---|---|---|---|---|---|
| Del 19 | <0.457 | <0.457 | 0.6 | 16.2 | <0.5 |
| Del 19 + C797S | 2.8 | 1.6 | 791.4 | 408.2 | 5.1 |
| Del 19 + T790M + | 821.7 | >1000 | 464.2 | >1000 | 197.9 |
| C797S | | | | | |
| L858R | <0.457 | <0.457 | 2.6 | 27.4 | <0.5 |
| L858R + C797S | 19 | 7.1 | >1000 | >1000 | 54.2 |
| L858R + T790M + | 845 | >1000 | >1000 | >1000 | 348.9 |
| C797S | | | | | |

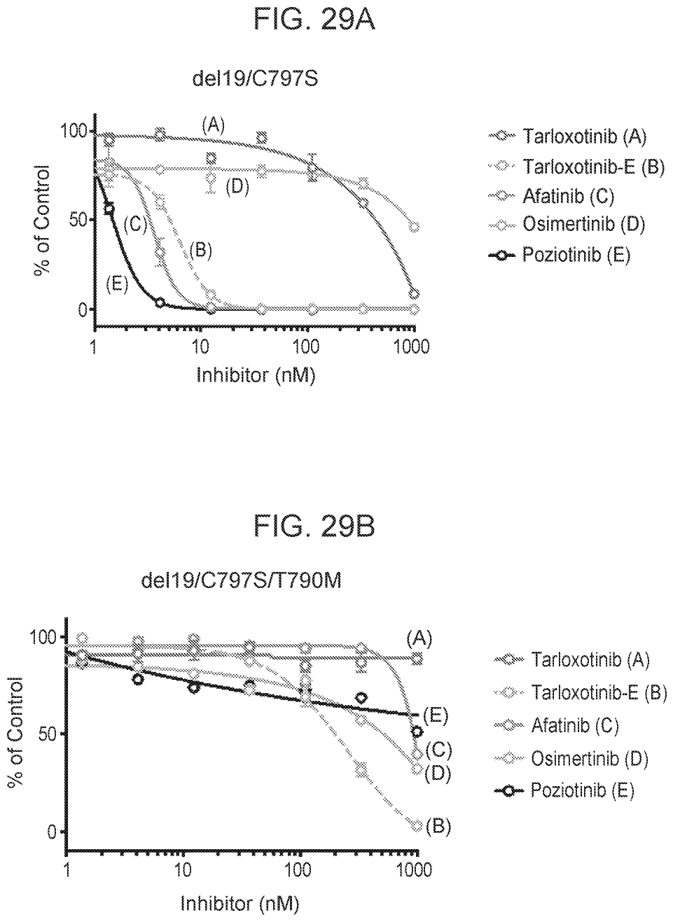

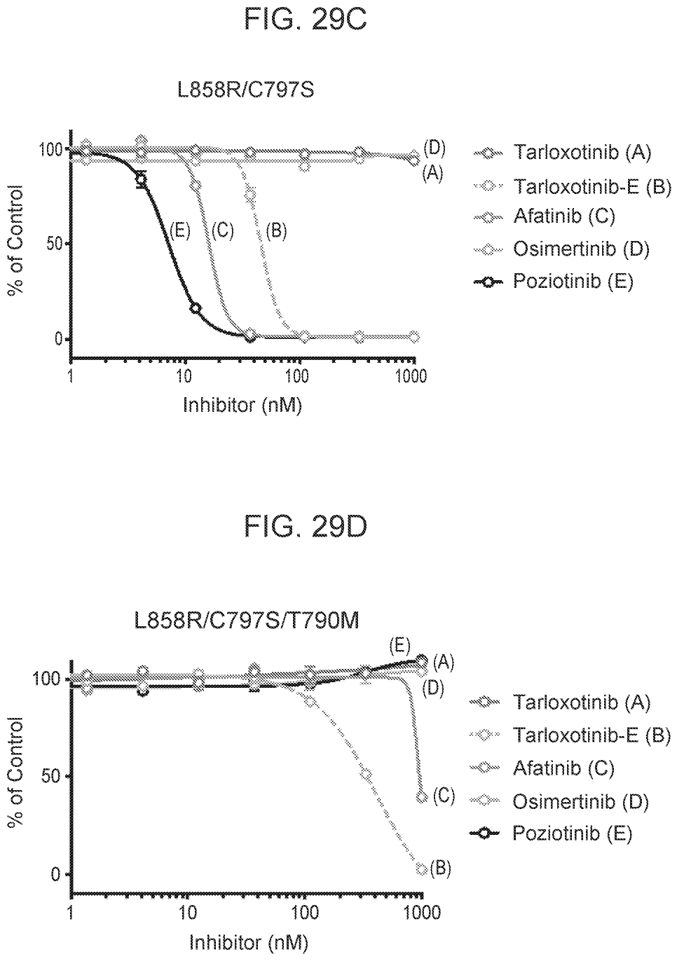

Table 8 shows cellular potency of mutated EGFR, wherein Tarloxotinib-E shows activity in EGFR C797S mutations in vivo (FIG. 29A-D)

TABLE 9
Antiproliferative activity of TRLX in Ba/F3 cell lines expressing
Osimertinib resistant tertiary EGFR mutations with del19 and L585R

| IC₅₀ (nM) | Afatinib⁸ | Osimertinib⁸ | Tarloxotinib | Tarloxotinib-E |
|---|---|---|---|---|
| Del19 + L718V | 0.5 | 10.6 | 46.7 | 0.68 |
| Del19 + G724S | 0.03 | 3.79 | 1.1 | <0.5 |
| Del19 + L792F | 0.3 | 4.01 | 16.9 | <0.5 |
| Del19 + L792H | 0.6 | 11.9 | 0.6 | <0.5 |
| Del19 + C797S | 2.8 | 791.4 | 408.2 | 5.1 |
| L858R + L718Q | 3.37 | 533 | 190.1 | 1.9 |
| L858R + L718V | 0.7 | 167.9 | 41.3 | 0.4 |
| L858R + L792F | 0.97 | 29.5 | 189.9 | <0.5 |
| L858R + L792H | 1.25 | 44.7 | 6.5 | 7.8 |
| L858R + C797G | 1.38 | 561.2 | 465.3 | <0.5 |
| L858R + C797S | 6.68 | 918 | >1000 | 54.2 |

TABLE 10
Antiproliferative activity of TRLX in patient-derived
cells with various EGFR exon 20 insertion mutations

| | | Cellular Anti-Proliferative Assay IC₅₀ (nM) | | | | |
|---|---|---|---|---|---|---|
| Cell Line | Kinase | Gefitinib | Afatinib | Osimertinib | Tarloxotinib | Tarloxotinib-E |
| CUTO-14 | EGFR | 3741 ± 1.4 | 111 ± 5 | 303 ± 1.9 | 4645 ± 5.5 | 72 ± 5.2 |
| | A767_V769dupASV | | | | | |
| CUTO-17 | EGFR | 4197 ± 1.4 | 220 ± 2.6 | 426 ± 3 | 3090 ± 1.6 | 48 ± 1.3 |
| | N771_H773dupNPH | | | | | |
| CUTO-18 | EGFR | >10000 | 841 ± 3 | 647 ± 3 | >10000 | 158 ± 1.3 |
| | S768_D770dupSVD | | | | | |

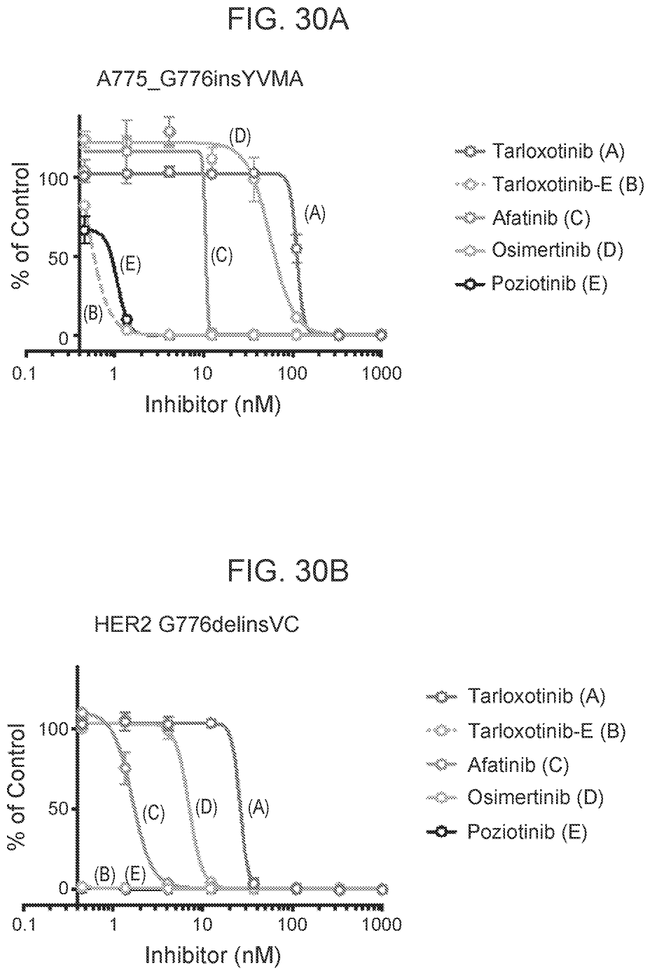

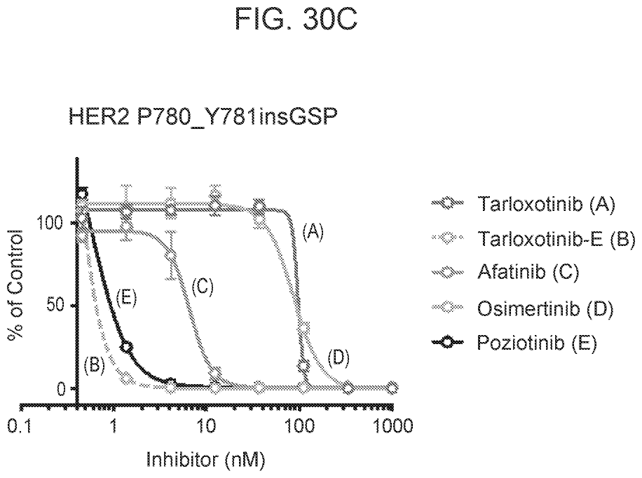

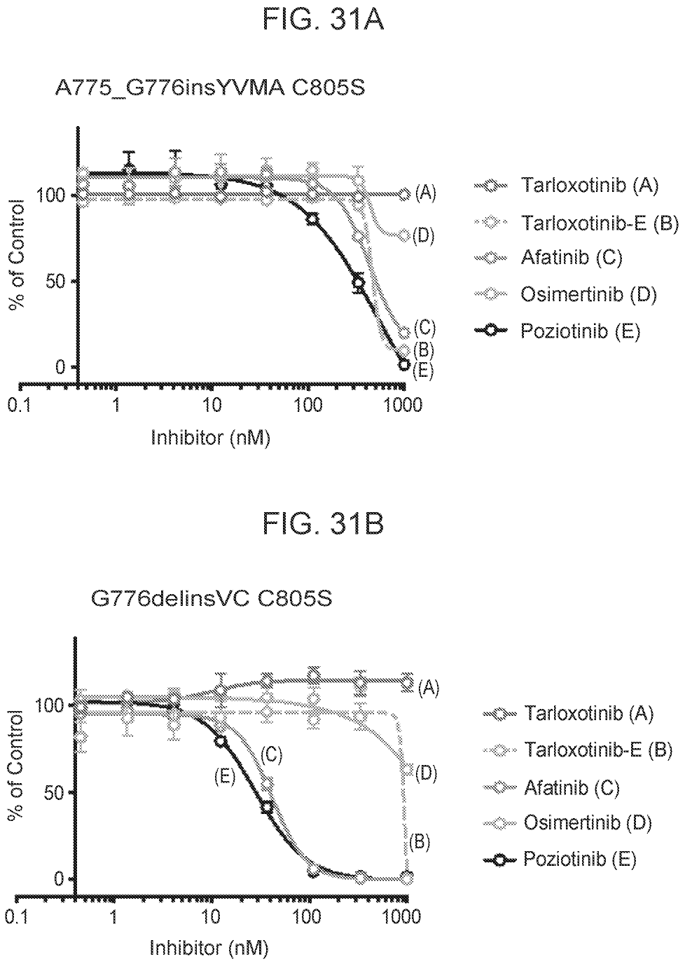

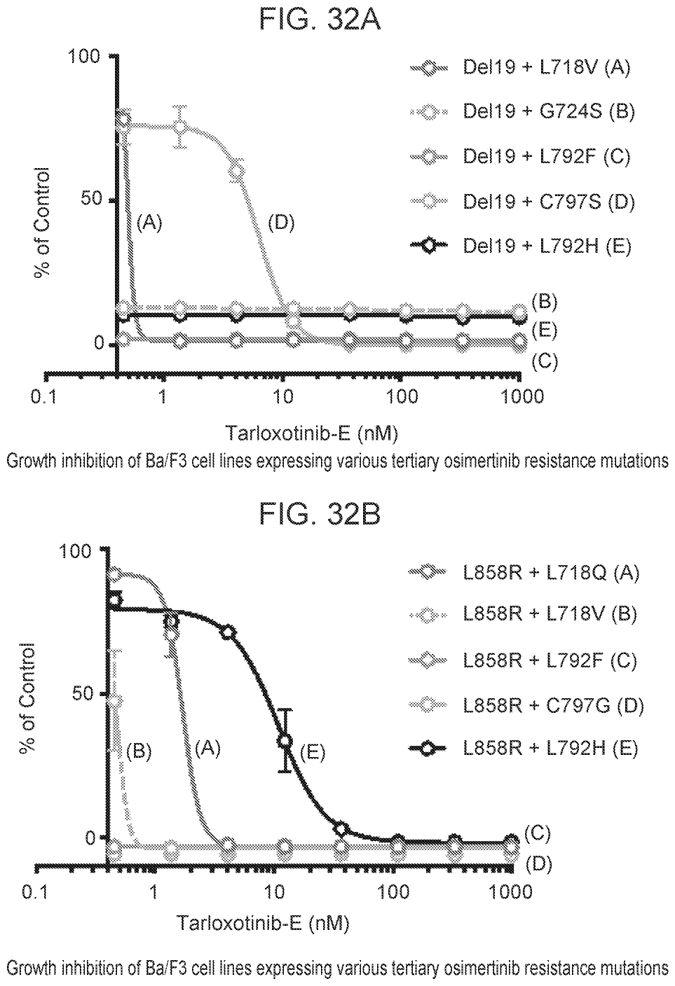

Tarloxotinib-E displayed potent activity compared to tarloxotinib, suggesting a wide therapeutic window. Tarloxotinib-E showed efficacy against all tested Ba/F3 cells with EGFR exon 20 insertion mutations (Table 10, FIGS. 30A-30C). The H773insH exon 20 insertion mutations were insensitive to all tested EGFR-TKIs. Tarloxotinib-E was potent for single mutations dell9 and L858R and double mutations dell9+C797S and L858R+C797S (Table 9, FIG. 32A-B). Some loss of potency was observed for the triple mutations Del 19+T790M+C797S and L858R+T790M+C797S suggesting, without wishing to be bound by theory, C797S and T790M as potential resistance mechanisms. Tarloxotinib-E displayed potent activity for various tertiary Osimertinib resistance mutations that are refractory to other TKIs. Tarloxotinib-E was most potent on the patient-derived cell lines with EGFR exon20 insertion mutations compared other TKIs.

### Example 5: Evaluation of Efficacy of TRLX (Compound A) or TRLX-TKI (Compound B) on Various HER2 (ErbB-2) Mutant Cancer Cell Lines

Many NSCLCs, breast, colorectal, lung, bladder, cervical, endometrial, ovarian, biliary tract, salivary duct and gastric cancers harbor ErbB-2 mutations. Various human cancer cell lines with the ErbB-2 mutations set forth in Table 11 are derived and characterized, in order to accelerate development of targeted therapies for these mutations. Additionally, various Ba/F3 cell lines are derived and characterized, in order to accelerate development of targeted therapies for these mutations.

TABLE 11
Various ErbB-2 mutations in derived various cancer cell lines.

| | | | |
|---|---|---|---|
| ErbB-2 (D769H) | ErbB-2 (R896C) | ErbB-2 (V777_G778insCG) | ErbB-2 (D769Y) |
| ErbB-2 (V777L) | ErbB-2/HER2 (wt) | ErbB-4/HER4 (wt) | — |

These cell lines are treated with TRLX-TKI or TRLX, and evaluated for tumor growth to confirm the applicability of TRLX-TKI and TRLX as therapeutic agents for tumors harboring these types of mutations. Evaluation is performed using, for example, cell proliferation and growth assays, in vitro inhibitory enzyme kinetic assays, mass spectrometry, peptide digestion and peptide analysis, and equilibrium binding assays. The results are compared to known small molecule ErbB-2 inhibitors, including such existing therapies as lapatinib and neratinib.

### Example 6. Potency of TRLX (Compound A) and TRLX-TKI (Compound B) Against Cancer Cell Lines Under Normal Oxygenation (Aerobic) and Under Low Oxygenation (Hypoxic) Conditions Showing Greater Potency Under Hypoxic Conditions

TRLX-E antiproliferative activity is greater following exposure of cells to hypoxia. TRLX-E shows potent activity in mutant-positive EGFR-driven NSCLC, but also demonstrates nanomolar activity against HER2/HER4 driven cell lines and moderate potency for Ras mutant cell lines under hypoxia (Table 12).

TABLE 12
Antiproliferative activity of TRLX in cell
lines with wild type HER and Ras mutations

| Human Cancer Cell Line | | | | | | |
|---|---|---|---|---|---|---|
| | | Driver | Mutation | TRLX IC50 (μM) | | |
| ID | Origin | Kinase | status¹ | Aerobic² | Hypoxic³ | HCR⁴ |
| Calu3 | NSCLC | HER2 | WT | 3.3 | 0.11 | 49 |
| | | amplification | | | | |
| SKOV3 | Ovarian | HER2 | WT | 15.6 | 0.59 | 48 |
| | | amplification | | | | |
| H661 | NSCLC | HER4 | WT | 8.5 | 0.08 | 136 |
| | | overexpression | | | | |
| H1734 | NSCLC | K-Ras | G13C | 77 | 5.9 | 18 |
| H460 | NSCLC | K-Ras | Q61H | 109 | 9.3 | 13 |
| H1299 | NSCLC | N-Ras | Q61K | 83 | 15 | 7 |

¹Determined by Sequenom massARRAY
²24 h aerobic exposure with 96 h drug-free recovery
³4 h hypoxic exposure (<1 ppm O2) followed by 20 h aerobic recovery and 96 h drug-free recovery
⁴HCR = hypoxic cytotoxicity ratio (average of intra-experimental ratio of IC50 values under aerobic and hypoxic conditions)

### Example 7: Potency of TRLX (Compound A) and TRLX-TKI (Compound B) Against HER2 Exon 20 Insertion Mutations in Ba/F3 Cells

HER2 A775_G776insYVMA, G776delinsVC, P780_Y781insGSP, YVMA+C805S (YVMACS) and VC+C805S (VCCS) mutations were introduced into Ba/F3 cells using retroviral vector. Growth inhibitory assays for TRLX (Compound A) and TRLX-TKI (Compound B), poziotinib, afatinib, and osimertinib were performed in Ba/F3 cells with each HER2 exon 20 mutation (Table 13).

TABLE 13
Cellular potency in Ba/F3 cells expressing HER2 exon20 insertion mutations

| IC₅₀ (nM) | Afatinib | Poziotinib | Osimertinib | Tarloxotinib | Tarloxotinib-E |
|---|---|---|---|---|---|
| A775_G776insYVMA | 8.08 | 0.64 | 68.29 | 122.93 | 0.72 |
| G776delinsVC | 2.05 | 0.02 | 7.23 | 22.21 | <0.5 |
| P780_Y781insGSP | 6.5 | 1.03 | 88.45 | 73.53 | 0.87 |
| YVMA + C805S | 544.9 | 315.8 | >1000 | >1000 | 592 |
| (YVMACS) | | | | | |
| VC + C805S (VCCS) | 41.3 | 27.9 | >1000 | >1000 | 608.9 |

Tarloxotinib had >84 times higher IC₅₀ value compared to tarloxotinib-E, demonstrating the potential to generate a wide therapeutic window with this prodrug strategy. Tarloxotinib-E showed efficacy against all tested Ba/F3 cells with HER2 exon 20 insertion mutations except for YVMA+C805S (YVMACS) and VC+C805S (VCCS) where C805S mutation was introduced along with HER2 exon20 insertion mutations, suggesting, without wishing to be bound by theory, that C805S could be a resistance mechanism to tarloxotinib and tarloxotinib-E.

### Example 8: Potency of TRLX (Compound A) and TRLX-TKI (Compound B) Against HER2 Amplified/Mutant Cell Lines

TABLE 14
Cellular potency in HER2 amplified/mutant cell lines

| Cell | | Cellular Anti-Proliferative Assay IC₅₀ (nM) | | | | |
|---|---|---|---|---|---|---|
| Line | Kinase | Gefitinib | Afatinib | Osimertinib | Tarloxotinib | Tarloxotinib-E |
| H-2170 | ERBB2 | 1156 | 11 | 113 | 588 | 4 |
| | amplification | | | | | |
| CALU-3 | ERBB2 | 1324 | 31 | 188 | 325 | 2 |
| | amplification | | | | | |
| H-1781 | ERBB2 | 4168 | 66 | 406 | 816 | 15 |
| | p.G776Ins | | | | | |
| | V_G/C | | | | | |

Tarloxotinib-E inhibits in vitro proliferation of HER2 amplified and HER2 mutant cell lines. In HER2 dependent cell lines, tarloxotinib-E has 60-150 fold higher activity compared to tarloxotinib in vitro under normoxic conditions (Table 14).

### Example 9: Pharmacokinetic (PK) Analysis of TRLX (Compound A) or TRLX-TKI (Compound B) in Mice

### Experimental Details

Unfasted female nude mice (n=3) were administered by injection TRLX (nominal dose of 48.0 mg/kg; administered dose 31.1 mg/kg in water containing 20% hydroxylpropyl-β-cyclodextrin). The dosing solution was prepared immediately prior to dosing. The administered dose was ˜35% lower than the nominal dose (acceptable range ±20%), of which ˜13% was attributable to the bromide salt in the molecule. For TRLX LC-MS/MS analysis, an aliquot of 20 μL sample was protein precipitated with 200 μL IS solution (100 ng/mL labetalol & 100 ng/mL tolbutamide in acetonitrile). The mixture was vortexed and centrifuged at 4000 rpm for 15 min at 4° C. An aliquot of 100 μL supernatant was transferred to a sample plate and mixed with 100 μL of water, then the plate was shaken at 800 rpm for 10 minutes. Subsequently, a 1-2 μL supernatant was injected for LC-MS/MS analysis of TRLX (Compound A). For TRLX-TKI analysis, an aliquot of 20 μL sample was protein precipitated with 200 μL IS solution (100 ng/mL labetalol & 100 ng/mL tolbutamide in acetonitrile). The mixture was vortexed and centrifuged at 4000 rpm for 15 minutes at 4° C. An aliquot of 160 μL supernatant was transferred to a sample plate, dried under N₂ gas, and resuspended in 120 μL pure acetonitrile. Subsequently, a 0.42 μL supernatant was injected for LC-MS/MS analysis of TRLX-TKI (Compound B).

### TRLX (Compound A) PK Analysis

The results of mouse plasma and brain pharmacokinetic studies of Compound A are shown in Tables 15-16 and FIGS. 25A and 25B. These data indicate that Compound A surprisingly crosses effectively the blood-brain barrier.

TABLE 15
TRLX (Compound A) concentration vs. time
data in mouse plasma and brain (n = 3)

| | Mean TRLX Plasma | Mean TRLX Brain |
|---|---|---|
| Time | Concentration | Concentration |
| (h) | (ng/mL); n = 3 | (ng/g); n = 3 |
| 0.500 | 81567 ± 11222 | 1062 ± 259 |
| 3.00 | 2447 ± 369 | 284 ± 34.5 |
| 12.0 | 131 ± 21.9 | 232 ± 17.3 |
| 24.0 | 93.0 ± 14.4 | 224 ± 38.0 |
| 72.0 | 39.4 ± 4.65 | 191 ± 49.7 |

TABLE 16
TRLX (Compound A) PK parameter in
mouse plasma and brain (n = 3)

| Parameter | Plasma Mean | Brain Mean |
|---|---|---|
| (units) | Value (n = 3) | Value (n = 3) |
| Cₘₐₓ (ng/mL) | 81567 | 1062 |
| Tₘₐₓ (h) | 0.500 | 0.500 |
| T_{1/2} (h) | 35.7 | 213 |
| AUC₀₋ₗₐₛₜ (ng · h/mL) | 88244 | 16714 |
| AUC_{0-inf} (ng · h/mL) | 90274 | 75256 |

### TRLX-TKI (Compound B) PK Analysis

The results of mouse plasma and brain pharmacokinetic studies of Compound B are shown in Tables 17-18 and FIGS. 26A and 26B. These data indicate that Compound B surprisingly crosses effectively the blood-brain barrier.

TABLE 17
TRLX-TKI (Compound B) concentration vs. time
data in mouse plasma and brain (n = 3)

| | Mean TRLX-TKI Plasma | Mean TRLX-TKI Brain |
|---|---|---|
| Time | Concentration | Concentration |
| (h) | (ng/mL); n = 3 | (ng/g); n = 3 |
| 0.500 | 512 ± 25.5 | 135 ± 48.1 |
| 3.00 | 362 ± 35.9 | 136 ± 25.4 |
| 12.0 | 109 ± 19.1 | 92.9 ± 35.1 |
| 24.0 | 13.3 ± 1.88 | 44.0 ± 10.1 |
| 72.0 | 4.12 ± 1.92 | 39.1 ± 11.7 |

TABLE 18
TRLX-TKI (Compound B) PK parameter

| Parameter | Plasma Mean | Brain Mean |
|---|---|---|
| (units) | Value (n = 3) | Value (n = 3) |
| Cₘₐₓ (ng/mL) | 512 | 136 |
| Tₘₐₓ (h) | 0.500 | 3.00 |
| T_{1/2} (h) | 11.8 | 63.0 |
| AUC₀₋ₗₐₛₜ (ng · h/mL) | 4023 | 4170 |
| AUC_{0-inf} (ng · h/mL) | 4093 | 7723 |

### Example 10: Osimertinib PC9 Brain Xenograft Data

EGFR TKIs that can cross the blood brain barrier show efficacy in PC9 brain metastasis model. Osimertinib A exhibits a dose dependent tumor regression with Osimertinib, which may be correlated with overall survival. The dose of osimertinib 25 mg/kg QD, roughly equating to the 80 mg QD clinical dose of osimertinib in terms of exposure, was well tolerated and induced sustained tumor regression until study end at day 60, with a little weight loss at the initial time point and no subsequent decrease throughout the dosing period.

The lower 5 mg/kg QD dose of osimertinib in the first three weeks caused tumor regression, which was more transient. In contrast, no tumor regression was achieved with rociletinib 100 mg/kg, approximately equivalent to a 500 mg twice daily human dose, and no survival benefit was observed (Ballard et al., Clin Cancer Res; 22(20); 5130-40, 2016).

Another EGFR TKI, YH25448 that penetrates into the brain showed intracranial tumor regression in H1975 xenograft model (Yun et al., Clin Cancer Res. 2019 Apr. 15; 25(8):2575-2587). YH25448 more effectively inhibited intracranial tumor growth than osimertinib at 10 mg/kg and 25 mg/kg once daily during a 49-day treatment (P=0.0125 and P=0.0274, respectively, by one-way ANOVA), with no obvious loss of the body weight. Consistent with brain tumor reduction detected by BLI in the YH25448-treated mice, histological analysis showed that the area of the brain tumor was reduced, and the number of proliferating cells also significantly decreased in the YH25448 (10 mg/kg)-treated mice compared with that in the vehicle-treated mice.

### H1975 Subcutaneous Implantation and Brain Metastasis (BM) Model

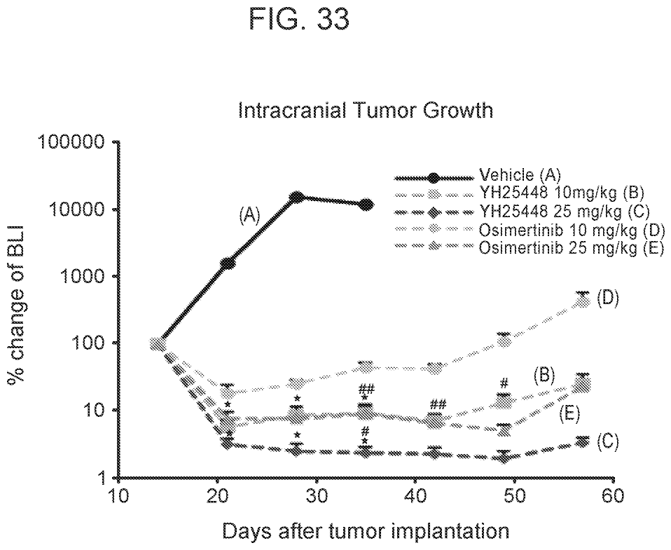

H1975-luc human NSCLC cells were implanted into the right flank and/or brain of female BALB/c nude mice. The tumor burden of intracranial lesions and the tumor size in the right flank were measured using a BLI technique with a real time in vivo imaging system (IVIS Spectrum, Caliper Life Sciences) and a digital calipers (Mitutoyo Corp.), respectively. Tumor-bearing mice were treated with a once-daily oral dose of YH25448 or osimertinib from 13-days post-implantation (FIG. 33).

Tarloxotinib crosses the blood-brain barrier, despite being a charged molecule. Without wishing to be bound by theory, as the brain levels are comparable to Osimertinib, tarloxotinib is anticipated to show comparable efficacy in the intracranial PC9 xenograft model. Based on the potent activity for Osimertinib resistance mutations and other EGFR mutations outlined above, it is anticipated to be efficacious in the brain metastasis models.

### Example 11: In Vivo Assays (Mouse Model) Examining Efficacy of TRLX (Compound A) or TRLX-TKI (Compound B) on Various Mutant Cell Lines

Generation of Mouse Cohorts and Treatment with TRLX-TKI or TRLX

Mice are generated with the desired mutations, e.g., the mutations set forth in Tables 11-14, above. The mice undergo MRI several weeks after mutation introduction to document the cancer burden before being assigned to various treatment study cohorts. Mice are then treated with vehicle alone or vehicle+TRLX-TKI or TRLX.

### MRI Scanning and Tumor Volume Measurement

Mice undergo MRI scanning to document their response to treatment.

### Immunohistochemical Analyses

Staining of tumor sections is performed. Immunohistochemistry is performed on certain tumor sections. Apoptosis is measure by counting nuclear bodies in stained tumor section and by a terminal deoxynucleotidyl-transferase mediated dUTP-biotin nick end labeling (TUNEL) assay.

### Pharmacokinetic Analyses

Healthy mice are provided. All mice are weighed before dose administration and subsequently randomized. For intravenous administration, freshly prepared solution of TRLX-TKI or TRLX is administered at a certain dose level via tail vein at a slow and steady rate. For oral administration, freshly prepared solution of a TRLX-TKI or TRLX is administered at a certain oral dose by stomach intubation using an oral feeding needle.

Blood samples and bioanalysis: Blood samples are collected from saphenous vein of each mouse at regular intervals. During each sampling point, blood samples are collected in labeled micro-tubes containing an anticoagulant. Bioanalysis is performed to determine compound concentration of TRLX-TKI or TRLX in mouse plasma using various methods, e.g., LC-MS/MS.

Pharmacokinetic analysis: The pharmacokinetic parameters of TRLX-TKI or TRLX such as Tₘₐₓ, Cₘₐₓ, AUC, CL, V_{d}, T_{1/2} A and bioavailability in mouse plasma are determined from concentration-time data.

Tarloxotinib was tested in subcutaneous xenograft models using cell lines harboring various EGFR mutations.

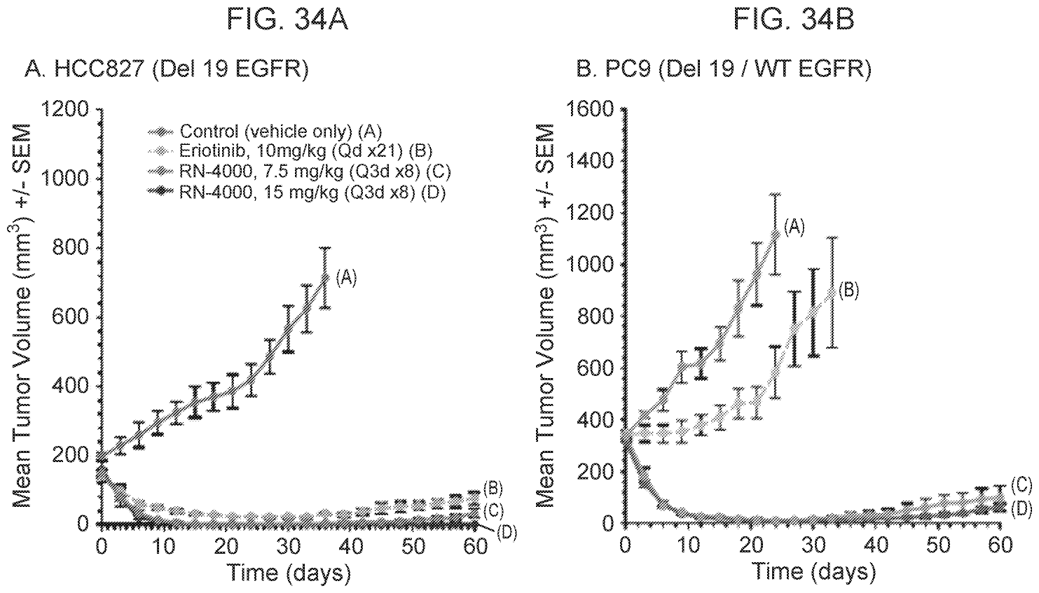

Tarloxotinib shows higher potency in comparison to erlotinib in HCC827 (Del 19 EGFR) and PC9 (Del 19/WT EGFR) xenograft models (FIG. 34A-B).

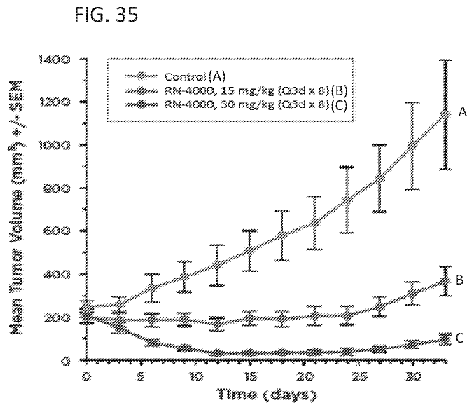

Tarloxotinib shows activity against the HER2-positive NCI-N87 gastric tumor xenograft, as seen in FIG. 35.

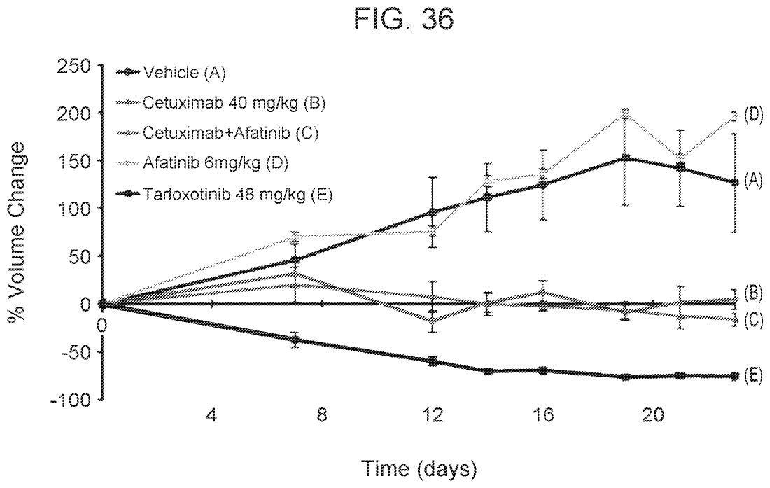

Potent Activity of tarloxotinib in EGFR exon 20 insertion (A767_V769dupASV) patient-derived cell line CUTO-14 xenograft model is seen in FIG. 36.

Tarloxotinib displayed potent single agent activity in HCC827 (EGFR Dell9), PC9 (EGFR dell9/ET EGFR), NCI-N87 (HER2 positive), CUTO-14 (EGFR exon 20 insertion mutation A767_V769dupASV patient-derived cell line) xenograft models consistent with the in vitro potency. Without wishing to be bound by theory, it is anticipated that in vitro activity at other mutations to display similar potent activity in xenograft models.

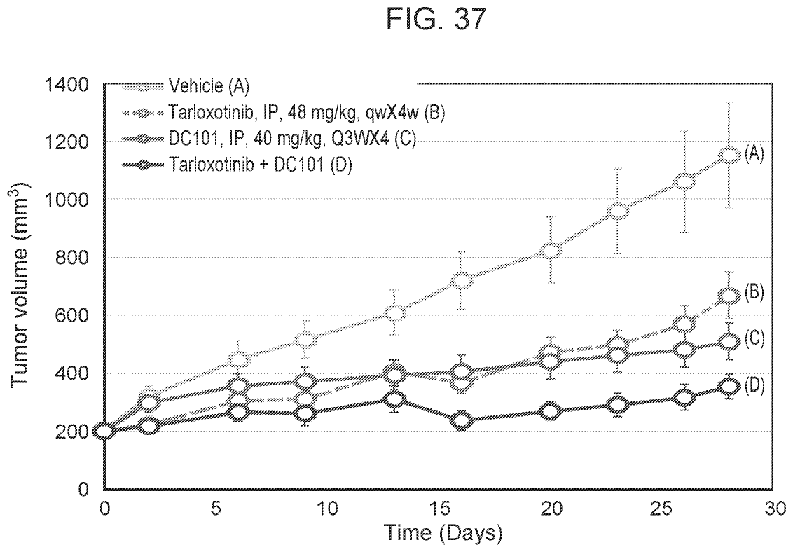

Tarloxotinib displayed synergistic activity with VEGFR2 inhibitor in H1781 (HER2 G776Ins VG/C) xenograft model, as shown in FIG. 37. Tarloxotinib showed significant single agent activity in H1781 (HER2 G776Ins V G/C) xenograft model. Tarloxotinib activity was enhanced by combining with DC101, a VEGFR2 mab. Without wishing to be bound by theory, this combination can be used clinically to enhance tarloxotinib activity.

### REFERENCES

1. Reiss et al J Thorac Oncol. 2018 October; 13(10):1560-1568.
2. Arcila et al Mol Cancer Ther. 2013 February; 12(2): 220-229.
3. Beau-Faller et al Annals of Oncology 25: 126-131, 2014.
4. Martin et al Clin Lung Cancer. 2019 May 11. pii: S1525-7304(19)30103-2.
5. Mishra et al Oncotarget, 2017, Vol. 8, (No. 69), pp: 114371-114392.
6. Thress et al., Nat. Med. 21 (6) (2015) 560-562
7. Nishino et al, Lung Cancer, Volume 126, 149-155
8. Le et al, Clin Cancer Res. 2018 Dec. 15; 24(24):6195-6203.
9. Konduri et al, Cancer Discov. 2016 June; 6(6):601-11
10. Shah et al BMC Genomics. 2013; 14:818-832
11. Frattini et al, Nat Genet. 2013; 45:1141-1149
12. Gallant et al, Cancer Discov. 2015 November; 5(11): 1155-63
13. Bose, et al., 2013, Cancer Discov 3:224-37.
14. Greulich, et al., 2012, Proc Natl Acad Sci USA 109:14476-81.
15. Kavuri, et al., 2015, Cancer Discov 5:832-41.
16. Ou, et al., 2017, J Thorac Oncol 12:446-457.
17. Zuo, et al., 2016, Clin Cancer Res 22:4859-4869.
18. Verma, et al., 2018, PLoS One 13:e0190942, 2018.
19. Bellmunt, et al., 2015, Cancer Med 4:844-52.
20. Kloth, et al., 2016, Gut 65:1296-305.
21. Xu, et al., 2017, Clin Cancer Res 23:5123-5134.
22. Stephens, et al., 2004, Nature 431:525-6.
23. Shigematsu, et al., 2005, Cancer Res 65:1642-6.
24. Wang, et al., 2006, Cancer Cell 10:25-38.
25. Pahuja et al Cancer Cell 34, 792-806, Nov. 12, 2018
26. Yu, et al., 2015, J Transl Med 13:116, 2015.
27. Ross, et al., 2013, Clin Cancer Res 19:2668-76.
28. Ross, et al., 2014, Clin Cancer Res 20:68-75.
29. Chmielecki, et al., 2015, Oncologist 20:7-12.
30. Gao, et al., 2018, Cell Rep 23:227-238 e3.
31. Mishra et al Oncotarget, 2017, Vol. 8, (No. 69), pp: 114371-114392
32. Kruppa et al Oncogene. 2016 Mar. 10; 35(10): 1283-91. (Link)
33. Kurppa et al, Oncogene (2015), 1-9.
34. Prickett et al, Nat Genet. 2009 October; 41(10): 1127-32.
35. Mishra et al Oncotarget. 2018 Jun. 12; 9(45): 27773-27788.
36. Jaiswal, et al., 2013, Cancer Cell 23:603-17.
37. Prickett et al, Nat Genet. 2009 October; 41(10): 1127-32 for use of lapatinib for HER4 mutations

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.
The foregoing description has been presented only for the purposes of illustration and is not intended to limit the disclosure to the precise form disclosed, but by the claims appended hereto.

What is claimed:

1. A method of treating or preventing a HER-driven cancer in a subject in need thereof, the method comprising:
(a) providing tumor cells of the subject;
(b) detecting presence or absence of a mutation or gene fusion or gene amplification in the provided tumor cells;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one mutation, fusion, or amplification is detected; and
(d) administering a therapeutically effective amount of Compound A, or a salt or a solvate thereof.

2. A method of treating or preventing a HER-driven cancer in a subject in need thereof, the method comprising:
(a) providing tumor cells of the subject;
(b) detecting presence or absence of an EGFR mutation in the provided tumor cells;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR mutation is detected; and
(d) administering a therapeutically effective amount of Compound A, or a salt or a solvate thereof.

3. The method of claim 2, wherein the EGFR mutation is selected from the group consisting of A702T, A743V, A767_V769dup, A840T, A840V, C620W, C797G, C797S, D770_N771delinsP, D855Y, E709_T710delinsD, E734V, E749Q, G724C, G724S, G735D, G779F, G796S, G857E, G863S, G874D, H773_V774delinsLM, H773dup, H835fs*35, H870R, K713T, K753E, K755S, L718Q, L718V, L730R, L747P, L768S, L792F/H, L792K, L844V, L858R, L858R with C797G, L858R with C797S, L858R with L718Q, L858R with L718V, L858R with L792F/H, L858R with T790M, N771_H773dup, N772delinsGY, P596L, P699L, P741S, P848L, Q791H, R831C, S768 D770 dup, S768_V769delinsIL, T725M, T790M, T847I, V765M, V773L, V834L, V843I, and V843L.

4. A method of treating or preventing a HER-driven cancer in a subject in need thereof, the method comprising:
(a) providing tumor cells of the subject;
(b) detecting presence or absence of an HER2 mutation in the provided tumor cells;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one HER2 mutation is detected; and
(d) administering a therapeutically effective amount of Compound A, or a salt or a solvate thereof.

5. The method of claim 4, wherein the HER2 mutation is selected from the group consisting of D769H, D769Y, R896C, V777L, V777_G778insCG, G309A/E, S310F/Y, V659E/D, G660D, K753E, L755P/S, Del755-759, L768S, D769H/Y, V773L, A775_G776insYVMA, G776V/L, Cins, V777L, P780Ins, P780_Y781insGSP, V842I, L866M, R896C, juxtamembrane and transmembrane domain mutations S653C, S656C, V659E, G660D, and G660R, and JMD mutants R677L, R678W, T686A, E693K, S649T, P650S, L651V, V659G, G660D, G660R, L663P, L674V, R677L, R678Q, R683Q, E693K, Q709L, and A710V and ErbB2 gene fusions ZNF207-HER2, MDK-HER2, NOS2-HER2, ERBB2-GRB7, ERBB2-CTTN, ERBB2-PPP1R1B, ERBB2-PSMB3 or additional N-terminal partners.

6. The method of claim 4, wherein the HER2 mutation is selected from the group consisting of A289D/I/N/T/V, A466T, A775_G776insSVMA, A775_G776insV, A775_G776insYVMA, C311R, C334S, C797S/Y, D227G/H/V/Y, D769H, D769Y, del.755-759, E321G, E790A/K/Q, G309A, G309E, G598V, G660D/R, G719A, G719A/C/D/S, G776>VC, G776_V777>AVCV, G776_V777>AVGCV, G776_V777>AVGSGV, G776_V777>VCV, G776C, G776S, G776V, G778S, I263T, I675M, I767M, L755S, L755S/W, L858R, L861Q/R, L866M, L869Q, L869R, N1219S, N319D, P780_Y781insGSP, P780ins, R103Q, R108G/K, R222C, R252C, R678Q, R678Q+L755W, R868W, R896C, S310F, S310Y, S768G/I/T, T733I, T790M, T798I/M, T862A, V659_660VE, V659E/D, V659E/G660R, V659E/V660R, V664E, V664F, V665M, V769L, V777_G778insC, V777A, V777L, V777M, V842I, and Y772_V773insLMAY.

7. A method of treating or preventing a HER-driven cancer in a subject in need thereof, the method comprising:
(a) providing tumor cells of the subject;
(b) detecting presence or absence of an HER3 mutation in the provided tumor cells;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one HER3 mutation is detected; and
(d) administering a therapeutically effective amount of Compound A, or a salt or a solvate thereof.

8. The method of claim 7, wherein the HER3 mutation is selected from the group consisting of: T355I, F94L, G284R, D297Y, T355I, E1261A, V104M, A232V, P262H, G284R, T389K, V714M, Q809R, S846I, E928G, any activating mutation, TKI resistance mutations, gene fusion, kinase domain duplication, and gene amplification of ErbB receptors.

9. The method of claim 7, wherein the HER3 mutation is selected from the group consisting of: A232A, A232V, D297Y, E332K, E928G, G284R, K329E, M91I, P262H, Q809R, R475W, R667C/H, S846I, T355I, T389K, V104L/M, and V855A.

10. A method of treating or preventing a HER-driven cancer in a subject in need thereof, the method comprising:
(a) providing tumor cells of the subject;
(b) detecting presence or absence of an HER4 mutation in the provided tumor cells;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one HER4 mutation is detected; and
(d) administering a therapeutically effective amount of Compound A, or a salt or a solvate thereof.

11. The method of claim 10, wherein the HER4 mutation is selected from the group consisting of: N181S, T244R, Y285C, R306S, V348L, D595V, H618P, D931Y, K935I, E317K, E452K, E542K, R544W, E563K, E836K, E872K and HER4 gene fusions EZR-ERBB4, IKZF2-ERBB4, BGALT-ERBB4 or additional N-terminal partners.

12. The method of claim 10, wherein the HER4 mutation is selected from the group consisting of: A657V, A705V, A710V, D595V, D609N, D931Y, E317D, E317K, E452K, E542K, E563K, E693G, E693K, E695K, E836K, E872K, G660D, G660R, G668E/R, G672R, G704E/R, G936R, H618P, I654L/M/T, I655M, I673F/M/V, I675L/M/T, I682M/T, K935I, L39F, L662V, L674I/V, L798R, M313I, M712L. N181S, P1033S, P409L, P650L/S, P699S/del, P700S, P702L, P702S, pG776insV_G/C, Q679E/H, Q709K, Q709L, Q711H, R106C/H, R1174Q, R306S, R393W, R491K, R544W, R677L, R677Q, R678P, R678Q, R678W, R683Q, R683W, R688L/Q/W, R689I/K, R713L/Q/W, R771C, R847C/H, R992C/S, S1246N, S1289A, S303F/Y, S341L, S653C, S653P, T244R, T652M, T652R, T686A/M/R, V348L, V659D, V659D/ins, V659E, V664F/I, V665M, V665M/del, V669A/L, V697L, V697L/M/del, V840I, Y111H, Y285C, and Y685H.

13. A method of treating or preventing a HER-driven cancer in a subject in need thereof, the method comprising:
(a) providing tumor cells of the subject;
(b) detecting presence or absence of a mutation in the provided tumor cells, wherein the mutation is an L858R mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one L858R mutation is detected; and
(d) administering a therapeutically effective amount of Compound A, or a salt or a solvate thereof.

14. The method of claim 13, wherein the L858R mutation is selected from the group consisting of L858R and T790M, L858R/C797S, and L858R/C797S/T790M.

15. A method of treating or preventing a HER-driven cancer in a subject in need thereof, the method comprising:
(a) providing tumor cells of the subject;
(b) detecting presence or absence of a mutation in the provided tumor cells, wherein the mutation is an exon 19 deletion;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one exon 19 deletion is detected; and
(d) administering a therapeutically effective amount of Compound A, or a salt or a solvate thereof.

16. The method of claim 15, wherein the exon 19 deletion is selected from the group consisting of d746-750, d746-750/C797A, d746-750/C797S, and d746-750/T790M/C797S.

17. A method of treating or preventing a HER-driven cancer in a subject in need thereof, the method comprising:
(a) providing tumor cells of the subject;
(b) detecting presence or absence of a mutation in the provided tumor cells, wherein the mutation is an exon 20 insertion;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one exon 20 insertion is detected; and
(d) administering a therapeutically effective amount of Compound A, or a salt or a solvate thereof.

18. The method of claim 17, wherein the exon 20 insertion is selected from the group consisting of D770_N771>ASVDN, N771_P772>SVDNP, N771_H773dupNPH, N771_P772insHH, N771_P772insH, N771_P772insNN, N771_P772insG, N771_P772>GYP, N771_P772insGTDN, N771_P772insY, N771_P772insV, N771_P772insT, N771_P772>SVDSP, N771_P772>SPHP, N771_P772>SHP, N771_P772>SEDNS, N771_P772>RDP, N771_P772>KGP, N771_P772>KFP, N771dupN, N771>GY, V774_C775>AHVC, V774_C775>GNPHVC, V774_C775>GTNPHVC, V774_C775insHNPHV, V774_C775insHV, A769_D770insASV, A763_Y764insFQEA, A763_Y764insLQEA, A767_V769dupASV, S768_D779dupSVD, S768_V769>PL, S768_V769>TLASV, V769_D770insSAVS, V769_D770insSGSV, V769_D770insSLRD, V769_H773>LDNPNPH, V769_D770insE, V769_D770insGE, V769_D770insGTV, V769_D770insGVM, V769_N771dupVDN, D770_N771insG, D770_N771>GYN, D770_N771>GSVDN, D770_N771>GVVDN, D770_N771insH, D770_P772dupDNP, D770_N771>QVH, D770_N771insAVD, D770_N771insGT, D770_N771insGV, D770_N771>EGN, M793_P794>ITQLMP, H773_V774dupHV, H773_V774insY, H773_V774insNPY, H773_V774insTH, H773_V774insSH, H773_V774insPY, H773_V774insHPH, H773_V774>NPNPYV, H773_V774>PNPYV, H773dupH, H773>YNPY, P772_H773dupPH, P772_H773insGNP, P772_H773>RHPH, Y764_V765insHH, A767_S768insTLA, D770ins_N771insSVD, V774_C775insHV, P772H773insGDP, I744_K745insKIPVAI, K745_E746insIPVAIK, K745_E746insVPVAIK, A763_Y764insFHEA, A775_G776insYVMA, G776>VC, V777_G778insCG, P780_Y781insGSP, D770>GY, D770_N771insY, D770_N771insGF, D770_N771insSVD, N771_P772insN, P772_H773insNP, P772_H773insNPH, H773_V774insAH, H773_V774insPH, H773_V774insH, H773_V774insNPH, and V774_C775insHV.

19. The method of claim 17, wherein the exon 20 insertion is selected from the group consisting of A763_Y764insFHEA, A763insFQEA, D770_N771insNPG, D770GY, D770insSVD, H773insH, H773insNPH, V769insASV and V777_G778insCG.

20. The method of claim 17, wherein the exon 20 insertion is selected from the group consisting of D770_N771>ASVDN, N771_P772>SVDNP, N771_H773dupNPH, and A763_Y764insFQEA.

21. A method of treating or preventing a HER-driven cancer in a subject in need thereof, the method comprising:
(a) providing tumor cells of the subject;
(b) detecting presence or absence of a mutation in the provided tumor cells, wherein the mutation is a compound mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one compound mutation is detected; and
(d) administering a therapeutically effective amount of Compound A, or a salt or a solvate thereof.

22. The method of claim 21, wherein the compound mutation is selected from the group consisting of p.E709G p.L858R, p.E746_A750del p.T751P, p.E746_R748del p.A750P, P.G719A p.L861Q, p.G719A p.L861R, p.G719C p.L833_V834delinsFL, p.G719C p.L861Q, P.G719C p.S768I, p.G721D p.E746_A750del, p.G724S p.S768I, p.L833_V834delinsFL p.L858R, p.L833V p.H835L, p.L858R p.A871E, p.N700S p.S784F, p.N700S p.T783A, p.S720F p.L861Q, p.T725M p.K728E, p.T751_I759del p.L798F, p.V738F p.L858R, and p.V834L p.L858R.

23. A method of treating or preventing a HER-driven cancer in a subject in need thereof, the method comprising:
(a) providing tumor cells of the subject;
(b) detecting presence or absence of a mutation in the provided tumor cells, wherein the mutation is a fusion mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one fusion mutation is detected; and
(d) administering a therapeutically effective amount of Compound A, or a salt or a solvate thereof.

24. The method of claim 23, wherein the fusion mutation is selected from the group consisting of MDK-HER2, NOS2-HER2, and ZNF207-HER2.

25. The method of claim 23, wherein the fusion mutation is selected from the group consisting of MDK_ex4/HER2_ex11, NOS2_ex2/HER2_ex2, and ZNF207_ex2/HER2_ex18.

26. The method of claim 23, wherein the fusion mutation is selected from the group consisting of EGFR-KDD (kinase domain duplication), EGFR-NTRK1, EGFR-PPM1H, EGFR-PSPH, EGFR-PSPHP1, EGFR-RP11, EGFR-RP11-745C, EGFR-SEPT14, and EGFR-SEPT14 fusions.

27. The method of claim 23, wherein the fusion mutation is selected from the group consisting of EGFR-SEPT14 and ERBB2-PSMB3.

28. A method of treating or preventing a HER-driven cancer in a subject in need thereof, the method comprising:
(a) providing tumor cells of the subject;
(b) detecting presence or absence of a mutation in the provided tumor cells, wherein the mutation is an atypical mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one atypical mutation is detected; and
(d) administering a therapeutically effective amount of Compound A, or a salt or a solvate thereof.

29. The method of claim 28, wherein the atypical mutation is selected from the group consisting of G719C, G719S, L747S, and L861Q.

30. A method of treating or preventing a HER-driven cancer in a subject in need thereof, the method comprising:
(a) providing tumor cells of the subject;
(b) detecting presence or absence of a mutation in the provided tumor cells, wherein the mutation is a rare mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one rare mutation is detected; and
(d) administering a therapeutically effective amount of Compound A, or a salt or a solvate thereof.

31. The method of claim 30, wherein the rare mutation is selected from the group consisting of a complex mutation, an exon 18 del/ins, an exon 18 G719X mutation, an exon 18 other substitution, an exon 20 insertion, an exon 20 other substitution, and an L858R complex mutation.

32. The method of claim 30, wherein the rare mutation is selected from the group consisting of A767V769dupASV, A769D770insASV, E709D, E709X, G709A+G719S, G719A, G719A+S768I, G719C, G719S, G719S+L861Q, G719S+S768I, G719X, L858R+S768D770dupSVD, L858R+S768I, L858R+T790M, S768I, and T790M.

33. A method of treating or preventing a HER-driven cancer in a subject in need thereof, the method comprising:
(a) providing tumor cells of the subject;
(b) detecting presence or absence of a mutation in the provided tumor cells, wherein the mutation is an exon 18 or exon 19 mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one exon 18 or exon 19 mutation is detected; and
(d) administering a therapeutically effective amount of Compound A, or a salt or a solvate thereof.

34. The method of claim 33, wherein the exon 18 or exon 19 mutation selected from the group consisting of E697G, E709 T710delinsD, E709A+G719S, E709D, E709H T710del, E711K, Exon 19, G719A, G719C, G719S, G719S+L861Q, L692V, p.A702T, p.A743V, p.E709_T710delinsD, p.E709G, p.E734V, p.E749Q, p.G724C/S, p.G735D, p.K713T, p.K728E, p.L730R, p.L747P, p.P699L, p.P741S, p.S720F, p.T725M, p.V738F, P699L, T725M, and Y693I.

35. A method of treating or preventing a HER-driven cancer in a subject in need thereof, the method comprising:
(a) providing tumor cells of the subject;
(b) detecting presence or absence of a mutation in the provided tumor cells, wherein the mutation is an exon 20 mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one exon 20 mutation is detected; and
(d) administering a therapeutically effective amount of Compound A, or a salt or a solvate thereof.

36. The method of claim 35, wherein the exon 20 mutation selected from the group consisting of A763 Y764insFQEA, A767 V769dupASV, A767S768insSVR, D761 E762insEAFQ, D770H773dupTTP, D770 N771insSVD, G796S, H773_V774dupH, H773_V774insPH, H773L+V774M, Ins 2AA, Ins 3 AA, M766 V769insWPA, N771 delinsKPP, N771dupN, p.A767_V769dup, p.D770_N771delinsP, p.G779F, p.H773_V774delinsLM, p.H773dup, p.N771_H773dup, p.N771delinsGY, p.Q791H, p.S768_D770dup, p.S768_V769delinsIL, p.V765M, P772 C775dupPHVC, P772H773dupH, P772H773insDNP, P772H773insLGNP, P772H773insT, R776H, S768 D770dupSVD, S768D770dupSVD, associated with L858R, S768D770dupSVD associated with L858R in association with other mutations as T725M, V769M and R776H, S768I+V769L, S768I, associated with L858R, S768I, associated with L858R in association with other mutations as T725M, V769M and R776H, T790M, T790M associated with L858R, T790M associated with L858R in association with other mutations as T725M, V769M and R776H, V769 D770delinsGI, V769 D770insL, V769M, and V774M.

37. A method of treating or preventing a HER-driven cancer in a subject in need thereof, the method comprising:
(a) providing tumor cells of the subject;
(b) detecting presence or absence of a mutation in the provided tumor cells, wherein the mutation is an exon 21 mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one exon 21 mutation is detected; and
(d) administering a therapeutically effective amount of Compound A, or a salt or a solvate thereof.

38. The method of claim 37, wherein the exon 21 mutation selected from the group consisting of p.A840T, p.A840V, p.A871E, p.D855Y, p.G857E, p.G863S, p.G874D, p.H835fs*55, p.H835L, p.L833_V834delinsFL, p.L833V, p.L861Q/R, p.P848L, p.R831C, P.T847I, p.V834L, p.V843I and p.V843L.

39. A method of treating or preventing a HER-driven cancer in a subject in need thereof, the method comprising:
(a) providing tumor cells of the subject;
(b) detecting presence or absence of a mutation in the provided tumor cells, wherein the mutation is a complex mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one complex mutation is detected; and
(d) administering a therapeutically effective amount of Compound A, or a salt or a solvate thereof.

40. The method of claim 39, wherein the complex mutation selected from the group consisting of G719A+P772H773dup, G719A+S768I, G719A+V769M, G719C+S768I, G719S+del 19 (E746_A750del), G719S+L861Q, G719S+S768I, P. G724S+p.S768I, p.E709G+p.L858R, p.E746_A750del+p.T751P, p.E746_R748del+p.A750P, p.G719A+p.L861Q, p.G719A+p.L861R, p.G719C+p.L833_V834delinsFL, p.G719C+p.L861Q, p.G719C+p.S768I, p.G721D+p.E746_A750del, p.L833_V834delinsFL+p.L858R, p.L833V+p.H835L, p.L858R+p.A871E, p.N700S+p.S784F, p.N700S+p.T783A, p.S720F+p.L861Q, p.T725M+p.K728E, p.T751_I759del+p.L798F, p.V738F+p.L858R, p.V834L+p.L858R, P772H773dup, S768 D770dupSVD+L858R, S768I+L858R, and T790M+L858R.

41. A method of treating or preventing a HER-driven cancer in a subject in need thereof, the method comprising:
(a) providing tumor cells of the subject;
(b) detecting presence or absence of a mutation in the provided tumor cells, wherein the mutation is a HER2^{YVMA} mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one HER2^{YVMA} mutation is detected; and
(d) administering a therapeutically effective amount of Compound A, or a salt or a solvate thereof.

42. A method of treating or preventing a HER-driven cancer in a subject in need thereof, the method comprising:
(a) providing tumor cells of the subject;
(b) detecting presence or absence of a mutation in the provided tumor cells, wherein the mutation is an osimertinib resistant mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one osimertinib resistant mutation is detected; and
(d) administering a therapeutically effective amount of Compound A, or a salt or a solvate thereof.

43. The method of claim 42, wherein the osimertinib resistant mutation is selected from the group consisting of Dell9+C797S, Dell9+G724S, Dell9+L718V, Dell9+L792F, Del 19+L792H, L858R+C797G, L858R+C797S, L858R+L718Q, L858R+L718V, L858R+L792F, and L858R+L792H.

44. A method of treating or preventing a HER-driven cancer in a subject in need thereof, the method comprising:
(a) providing tumor cells of the subject;
(b) detecting presence or absence of a mutation in the provided tumor cells, wherein the mutation is an EGFR mutation associated with lung adenocarcinoma;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR mutation associated with lung adenocarcinoma is detected; and
(d) administering a therapeutically effective amount of Compound A, or a salt or a solvate thereof.

45. The method of claim 44, wherein the EGFR mutation associated with lung adenocarcinoma is selected from the group consisting of A763_Y764insFQEA, A767_S768insTLA, A767_V769dupASV, D770_N771insGT, D770N concurrently with an H773_V774insNPH, G776V/L, Cins, GSP 781-783 ins, M766_A767insAI, S768_D770dupAVD, S768I, S768I in conjunction with G719A, S768I in conjunction with V769L, V765insHH, V769_D770insASV, V774_C775insHV, and YVMA 776-779 ins.

46. A method of treating or preventing a HER-driven cancer in a subject in need thereof, the method comprising:
(a) providing tumor cells of the subject;
(b) detecting presence or absence of a mutation in the provided tumor cells, wherein the mutation is an EGFR mutation associated with gastric cancer;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR mutation associated with gastric cancer is detected; and
(d) administering a therapeutically effective amount of Compound A, or a salt or a solvate thereof.

47. The method of claim 46, wherein the EGFR mutation associated with gastric cancer is selected from the group consisting of MDK_ex4/HER2_ex11, NOS2_ex2/HER2_ex2, and ZNF207_ex2/HER2_ex18.

48. A method of treating or preventing a HER-driven cancer in a subject in need thereof, the method comprising:
(a) providing tumor cells of the subject;
(b) detecting presence or absence of a mutation in the provided tumor cells, wherein the mutation is an EGFR mutation associated with lung cancer;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR mutation associated with lung cancer is detected; and
(d) administering a therapeutically effective amount of Compound A, or a salt or a solvate thereof.

49. The method of claim 48, wherein the EGFR mutation associated with lung cancer is L858R.

50. A method of treating or preventing a HER-driven cancer in a subject in need thereof, the method comprising:
(a) providing tumor cells of the subject;
(b) detecting presence or absence of a mutation in the provided tumor cells, wherein the mutation is an EGFR mutation associated with non-small cell lung adenocarcinoma;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR mutation associated with non-small cell lung adenocarcinoma is detected; and
(d) administering a therapeutically effective amount of Compound A, or a salt or a solvate thereof.

51. The method of claim 50, wherein the EGFR mutation associated with non-small cell lung adenocarcinoma is selected from the group consisting of L858R, L858R/T790M, dell9, dell9/T790M, dell9/T790M/C797S, L861Q, and G719C/S768I.

52. A method of treating or preventing a HER-driven cancer in a subject in need thereof, the method comprising:
(a) providing tumor cells of the subject;
(b) detecting presence or absence of a mutation in the provided tumor cells, wherein the mutation is an EGFR mutation associated with urothelial carcinoma;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR mutation associated with urothelial carcinoma is detected; and
(d) administering a therapeutically effective amount of Compound A, or a salt or a solvate thereof.

53. The method of claim 52, wherein the EGFR mutation associated with urothelial carcinoma is selected from the group consisting of R157W, S310F/Y, and V777L/A/M.

54. A method of treating or preventing a HER-driven cancer in a subject in need thereof, the method comprising:
(a) providing tumor cells of the subject;
(b) detecting presence or absence of a mutation in the provided tumor cells, wherein the mutation is an EGFR mutation associated with breast cancer;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR mutation associated with breast cancer is detected; and
(d) administering a therapeutically effective amount of Compound A, or a salt or a solvate thereof.

55. The method of claim 54, wherein the EGFR mutation associated with breast cancer is selected from the group consisting of I655V, K676R, K753E, L755S, L768S, Q680R, R647K, and V773L.

56. A method of treating or preventing a HER-driven cancer in a subject in need thereof, the method comprising:
(a) providing tumor cells of the subject;
(b) detecting presence or absence of a mutation in the provided tumor cells, wherein the mutation is an EGFR mutation associated with Lynch and Lynch-like colorectal cancer;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR mutation associated with Lynch and Lynch-like colorectal cancer is detected; and
(d) administering a therapeutically effective amount of Compound A, or a salt or a solvate thereof.

57. The method of claim 56, wherein the EGFR mutation associated with Lynch and Lynch-like colorectal cancer is selected from the group consisting of A848T, G865R, L726F, L755S/F, L755S with A848T, L755S with V842I, and V842I.

58. A method of treating or preventing a HER-driven cancer in a subject in need thereof, the method comprising:
(a) providing tumor cells of the subject;
(b) detecting presence or absence of a mutation or gene fusion or gene amplification in the provided tumor cells, wherein the mutation comprises an EGFR, HER2, HER3, or HER4 mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one EGFR, HER2, HER3, or HER4 mutation is detected; and
(d) administering a therapeutically effective amount of Compound A, or a salt or a solvate thereof.

59. A method of treating or preventing a HER-driven cancer in a subject in need thereof, the method comprising:
(a) providing tumor cells of the subject;
(b) detecting presence or absence of a mutation or gene fusion or gene amplification in the provided tumor cells, wherein the mutation comprises an EGFR (HER1) mutation selected from the group consisting of EGFR exon 20 insertion mutations, EGFR rare and compound mutations, tertiary Osimertinib resistance mutations, EGFR fusion mutations; or
wherein the mutation comprises an ErbB-2 (HER2) mutation selected from the group consisting of juxtamembrane and transmembrane domain mutations, JMD mutants, and ErbB2 gene fusions; or
wherein the mutation comprises an ErbB-4 (HER4) mutation selected from the group consisting of HER4 gene fusions EZR-ERBB4, IKZF2-ERBB4, BGALT-ERBB4 or additional N-terminal partners; or
wherein the mutation comprises an ErbB-3 (HER3) mutation; or
any activating mutation, TKI resistance mutations, gene fusion, kinase domain duplication, and gene amplification of ErbB receptors;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the mutations is detected; and
(d) administering a therapeutically effective amount of Compound A, or a salt or a solvate thereof.

60. A method of treating or preventing a HER-driven cancer in a subject in need thereof, the method comprising:
(a) providing tumor cells of the subject;
(b) detecting presence or absence of a mutation or gene fusion or gene amplification in the provided tumor cells, wherein the mutation comprises an EGFR (HER1) mutation selected from the group consisting of: A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R, D770_N771>ASVDN, N771_P772>SVDNP, N771_H773dupNPH, N771_P772insHH, N771_P772insH, N771_P772insNN, N771_P772insG, N771_P772>GYP, N771_P772insGTDN, N771_P772insY, N771_P772insV, N771_P772insT, N771_P772>SVDSP, N771_P772>SPHP, N771_P772>SHP, N771_P772>SEDNS, N771_P772>RDP, N771_P772>KGP, N771_P772>KFP, N771dupN, N771>GY, V774_C775>AHVC, V774_C775>GNPHVC, V774_C775>GTNPHVC, V774_C775insHNPHV, V774_C775insHV, A769_D770insASV, A763_Y764insFQEA, A763_Y764insLQEA, A767_V769dupASV, S768_D779dupSVD, S768_V769>PL, S768_V769>TLASV, V769_D770insSAVS, V769_D770insSGSV, V769_D770insSLRD, V769_H773>LDNPNPH, V769_D770insE, V769_D770insGE, V769_D770insGTV, V769_D770insGVM, V769_N771dupVDN, D770_N771insG, D770_N771>GYN, D770_N771>GSVDN, D770_N771>GVVDN, D770_N771insH, D770_P772dupDNP, D770_N771>QVH, D770_N771insAVD, D770_N771insGT, D770_N771insGV, D770_N771>EGN, M793_P794>ITQLMP, H773_V774dupHV, H773_V774insY, H773_V774insNPY, H773_V774insTH, H773_V774insSH, H773_V774insPY, H773_V774insHPH, H773_V774>NPNPYV, H773_V774>PNPYV, H773dupH, H773>YNPY, P772_H773dupPH, P772_H773insGNP, P772_H773>RHPH, Y764_V765insHH, A767_S768insTLA, D770ins_N771insSVD, V774_C775insHV, P772H773insGDP, I744_K745insKIPVAI, K745_E746insIPVAIK, K745_E746insVPVAIK, A763_Y764insFHEA, A775_G776insYVMA, G776>VC, V777_G778insCG, P780_Y781insGSP, D770>GY, D770_N771insY, D770_N771insGF, D770_N771insSVD, N771_P772insN, P772_H773insNP, P772_H773insNPH, H773_V774insAH, H773_V774insPH, H773_V774insH, H773_V774insNPH, and V774_C775insHV, EGFR rare and compound mutations, tertiary Osimertinib resistance mutations L718Q/V, G724S, L792F/H, G796S, C797S/G, EGFR gene fusions EGFR-SEPT14, EGFR-RAD51, EGFR-PSPH or additional C-terminal partners, EGFR kinase domain duplication (EGFR-KDD); or
wherein the mutation comprises an ErbB-2 (HER2) mutation selected from the group consisting of: D769H, D769Y, R896C, V777L, V777_G778insCG, G309A/E, S310F/Y, V659E/D, G660D, K753E, L755P/S, Del755-759, L768S, D769H/Y, V773L, A775_G776insYVMA, G776V/L, Cins, V777L, P780Ins, P780_Y781insGSP, V842I, L866M, R896C, juxtamembrane and transmembrane domain mutations S653C, S656C, V659E, G660D, and G660R, and JMD mutants R677L, R678W, T686A, E693K, S649T, P650S, L651V, V659G, G660D, G660R, L663P, L674V, R677L, R678Q, R683Q, E693K, Q709L, and A710V and ErbB2 gene fusions ZNF207-HER2, MDK-HER2, NOS2-HER2, ERBB2-GRB7, ERBB2-CTTN, ERBB2-PPP1R1B, ERBB2-PSMB3 or additional N-terminal partners; or
wherein the mutation comprises an ErbB-4 (HER4) mutation selected from the group consisting of: N181S, T244R, Y285C, R306S, V348L, D595V, H618P, D931Y, K935I, E317K, E452K, E542K, R544W, E563K, E836K, E872K and HER4 gene fusions EZR-ERBB4, IKZF2-ERBB4, BGALT-ERBB4 or additional N-terminal partners; or
wherein the mutation comprises an ErbB-3 (HER3) mutation selected from the group consisting of: T355I, F94L, G284R, D297Y, T355I, E1261A; V104M, A232V, P262H, G284R, T389K, V714M, Q809R, S846I and E928G; or
any activating mutation, TKI resistance mutations, gene fusion, kinase domain duplication, and gene amplification of ErbB receptors;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the mutations is detected; and
(d) administering a therapeutically effective amount of Compound A, or a salt or a solvate thereof.

61. A method of treating or preventing a HER-driven cancer in a subject in need thereof, the method comprising:
(a) providing tumor cells of the subject;
(b) detecting presence or absence of a mutation or gene fusion or gene amplification in the provided tumor cells, wherein the mutation comprises an EGFR (HER1) mutation selected from the group consisting of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R, EGFR exon 20 insertion mutations, EGFR rare and compound mutations, tertiary Osimertinib resistance mutations L718Q/V, G724S, L792F/H, G796S, C797S/G, EGFR gene fusions EGFR-SEPT14, EGFR-RAD51, EGFR-PSPH or additional C-terminal partners, EGFR kinase domain duplication (EGFR-KDD); or
wherein the mutation comprises an ErbB-2 (HER2) mutation selected from the group consisting of: D769H, D769Y, R896C, V777L, V777_G778insCG, G309A/E, S310F/Y, V659E/D, G660D, K753E, L755P/S, Del755-759, L768S, D769H/Y, V773L, A775_G776insYVMA, G776V/L, Cins, V777L, P780Ins, P780_Y781insGSP, V842I, L866M, R896C, juxtamembrane and transmembrane domain mutations S653C, S656C, V659E, G660D, and G660R, and JMD mutants R677L, R678W, T686A, E693K, S649T, P650S, L651V, V659G, G660D, G660R, L663P, L674V, R677L, R678Q, R683Q, E693K, Q709L, and A710V and ErbB2 gene fusions ZNF207-HER2, MDK-HER2, NOS2-HER2, ERBB2-GRB7, ERBB2-CTTN, ERBB2-PPP1R1B, ERBB2-PSMB3 or additional N-terminal partners; or
wherein the mutation comprises an ErbB-4 (HER4) mutation selected from the group consisting of: N181S, T244R, Y285C, R306S, V348L, D595V, H618P, D931Y, K935I, E317K, E452K, E542K, R544W, E563K, E836K, E872K and HER4 gene fusions EZR-ERBB4, IKZF2-ERBB4, BGALT-ERBB4 or additional N-terminal partners; or
wherein the mutation comprises an ErbB-3 (HER3) mutation selected from the group consisting of: T355I, F94L, G284R, D297Y, T355I, E1261A; V104M, A232V, P262H, G284R, T389K, V714M, Q809R, S846I and E928G; or
any activating mutation, TKI resistance mutations, gene fusion, kinase domain duplication, and gene amplification of ErbB receptors;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the mutations is detected; and
(d) administering a therapeutically effective amount of Compound A, or a salt or a solvate thereof.

62. A method of treating or preventing a HER-driven cancer in a subject in need thereof, the method comprising:
(a) providing tumor cells of the subject;
(b) detecting presence or absence of a TKI drug resistant mutation or gene fusion or gene amplification in the provided tumor cells, wherein the mutation is a pan-HER mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the mutations, fusions, or amplifications is detected; and
(d) administering a therapeutically effective amount of Compound A, or a salt or a solvate thereof.

63. A method of treating or preventing a HER-driven cancer in a subject in need thereof, the method comprising:
(a) providing tumor cells of the subject;
(b) detecting presence or absence of a TKI drug resistant mutation or gene fusion or gene amplification in the provided tumor cells, wherein the mutation comprises an EGFR (HER1) mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the EGFR mutations is detected; and
(d) administering a therapeutically effective amount of Compound A, or a salt or a solvate thereof.

64. A method of treating or preventing a HER-driven cancer in a subject in need thereof, the method comprising:
(a) providing tumor cells of the subject;
(b) detecting presence or absence of a TKI drug resistant mutation or gene fusion or gene amplification in the provided tumor cells, wherein the mutation comprises an HER2 (ErbB-2) mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the HER2 mutations is detected; and
(d) administering a therapeutically effective amount of Compound A, or a salt or a solvate thereof.

65. A method of treating or preventing a HER-driven cancer in a subject in need thereof, the method comprising:
(a) providing tumor cells of the subject;
(b) detecting presence or absence of a TKI drug resistant mutation or gene fusion or gene amplification in the provided tumor cells, wherein the mutation comprises an HER3 (ErbB-3) mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the HER3 mutations is detected; and
(d) administering a therapeutically effective amount of Compound A, or a salt or a solvate thereof.

66. A method of treating or preventing a HER-driven cancer in a subject in need thereof, the method comprising:
(a) providing tumor cells of the subject;
(b) detecting presence or absence of a TKI drug resistant mutation or gene fusion or gene amplification in the provided tumor cells, wherein the mutation comprises an HER4 (ErbB-4) mutation;
(c) predicting the subject as being likely to be responsive to treatment by (2E)-4-{[4-(3-bromo-4-chloroanilino)pyrido[3,4-d]pyrimidin-6-yl]amino}-N,N-dimethyl-N-[(1-methyl-4-nitro-1H-imidazol-5-yl)methyl]-4-oxo-2-buten-1-ammonium bromide (Compound A), if at least one of the HER4 mutations is detected; and
(d) administering a therapeutically effective amount of Compound A, or a salt or a solvate thereof.

67. A method of treating a HER-driven cancer in a subject with cancer, where at least one of an EGFR mutation or a ErbB-2 mutation is detected in tumor cells of the subject, wherein the method comprises administering a therapeutically effective amount of Compound A, or a salt or a solvate thereof; and
wherein the EGFR mutation is selected from the group consisting of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; and
the ErbB-2 mutation is selected from the group consisting of D769H, D769Y, R896C, V777L, and V777_G778insCG.

68. A method of predicting the responsiveness of a subject with a HER-driven cancer to treatment with Compound A, wherein the method comprises:
(a) providing tumor cells of the subject;
(b) detecting presence or absence of a mutation in the provided tumor cells of the subject, wherein the mutation comprises an EGFR mutation selected from the group consisting of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or
wherein the mutation comprises an ErbB-2 mutation selected from the group consisting of D769H, D769Y, R896C, V777L, and V777_G778insCG;
(c) predicting the subject as being likely to be responsive to a treatment with Compound A if at least one of the EGFR mutation and the ErbB-2 mutation is detected in the tumor cell of the subject.

69. A method of predicting the responsiveness of a subject with a HER-driven cancer to treatment with Compound A, wherein the method comprises detecting presence or absence of a mutation in a sample of tumor cells from the subject;
wherein the subject is likely to be responsive to the treatment with Compound A if the mutation is detected in the sample of tumor cells from the subject; and
wherein the mutation comprises an EGFR mutation selected from the group consisting of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or
wherein the mutation comprises an ErbB-2 mutation selected from the group consisting of: D769H, D769Y, R896C, V777L, and V777_G778insCG.

70. A method of identifying a subject with cancer who is likely to be responsive to treatment with Compound A, wherein the method comprises:
(a) providing tumor cells of the subject;
(b) detecting presence or absence of a mutation in the provided tumor cells of the subject, wherein the mutation comprises an EGFR mutation selected from the group consisting of:
A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or
wherein the mutation comprises an ErbB-2 mutation selected from the group consisting of: D769H, D769Y, R896C, V777L, and V777_G778insCG;
(c) identifying the subject as being likely to be responsive to treatment with Compound A if at least one of the EGFR mutation and the ErbB-2 mutation is detected in the provided tumor cells.

71. The method of any one of claims 58-70, wherein the HER-driven cancer comprises an EGFR-driven cancer.

72. The method of claim 71, wherein the EGFR-driven cancer comprises lung cancer.

73. The method of claim 72, wherein the lung cancer comprises non-small cell lung cancer (NSCLC).

74. The method of claim 71, wherein the EGFR-driven cancer comprises brain metastases.

75. The method of any one of claims 58-70, wherein the HER-driven cancer comprises a HER1 mutation.

76. The method of any one of claims 58-69, wherein the HER-driven cancer comprises a HER2 mutation.

77. The method of any one of claims 58-62, wherein the HER-driven cancer comprises a HER3 mutation.

78. The method of any one of claims 58-62, wherein the HER-driven cancer comprises a HER4 mutation.

79. The method of any one of claims 1-78, wherein at least one mutation is selected from the group consisting of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R.

80. The method of any one of claims 1-78, wherein at least one mutation is selected from the group consisting of D769H, D.769Y, R896C, V777L, and V777_G778insCG.

81. The method of any one of claims 1-78, wherein at least one mutation is selected from the group consisting of C797S/L858R, d746-750/C797S, d746-750/T790M/C797S, and T790M/C797S/L858R.

82. The method of any one of claims 1-81, wherein the cancer is resistant to at least one agent selected from the group consisting of osimertinib, gefitinib, afatinib, erlotinib, dacomitinib, lapatinib, neratinib, avitinib, olmutinib, pelitinib, poziotinib, trastuzumab, pertuzumab, cetuximab, panitumumab.

83. The method of any one of claims 1-82, further comprising administering at least one additional agent, or a salt or solvate thereof, in combination with Compound A.

84. The method of claim 83, wherein the cancer is lung cancer.

85. The method of claim 84, wherein the lung cancer is non-small cell lung cancer.

86. The method of claim 83, wherein the cancer is brain cancer.

87. The method of claim 83, wherein the cancer is a brain metastasis.

88. The method of claim 83, wherein Compound A and the at least one additional agent are co-administered to the subject.

89. The method of claim 88, wherein Compound A and the at least one additional agent are coformulated.

90. The method of any one of claims 1-89, wherein Compound A is administered by at least one route selected from the group consisting of inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical.

91. The method of any one of claims 1-90, wherein the subject is a mammal.

92. The method of claim 91, wherein the mammal is a human.

93. The method of claim 92, wherein the subject is a human in need of treatment thereof.

94. The method of any one of claims 1-93, wherein the cancer is a HER-driven drug-resistant cancer.

95. Use of Compound A in the manufacture of a medicament for the treatment of a HER-driven cancer, wherein the HER-driven cancer comprises an EGFR mutation.

96. Use of Compound A in the manufacture of a medicament for the treatment of a HER-driven cancer, wherein the HER-driven cancer comprises an EGFR mutation,
wherein the at least one mutation is selected from the group consisting of A763_Y764insFHEA, C797S/L858R, d746-750, d746-750/C797A, d746-750/C797S, d746-750/T790M/C797S, D770GY, D770_N771insNPG, G719C, G719S, L747S, L858R, L858R/T790M, L861Q, and T790M/C797S/L858R; or
an ErbB-2 mutation selected from the group consisting of D769H, D769Y, R896C, V777L, V777_G778insCG, and HER4.

97. A method of treating or preventing a HER-driven cancer in a subject in need thereof, the method comprising:
(a) providing tumor cells of the subject;
(b) detecting presence or absence of a mutation in the provided tumor cells, wherein the mutation comprises an EGFR C797S mutation;
(c) predicting the subject as being likely to be responsive to treatment by Compound A. if the EGFR C797S mutation is detected; and
(d) administering a therapeutically effective amount of Compound A, or a salt or a solvate thereof.

98. A method of treating a HER-driven cancer in a subject with cancer, where an EGFR C797S mutation is detected in tumor cells of the subject, wherein the method comprises administering a therapeutically effective amount of Compound A, or a salt or a solvate thereof.

99. A method of predicting the responsiveness of a subject with a HER-driven cancer to treatment with Compound A, wherein the method comprises:
(a) providing tumor cells of the subject;
(b) detecting presence or absence of a mutation in the provided tumor cells of the subject, wherein the mutation comprises an EGFR C797S mutation;
(c) predicting the subject as being likely to be responsive to a treatment with Compound A if the EGFR C797S mutation is detected in the tumor cell of the subject.

100. A method of predicting the responsiveness of a subject with a HER-driven cancer to treatment with Compound A, wherein the method comprises detecting presence or absence of a mutation in a sample of tumor cells from the subject;
wherein the subject is likely to be responsive to the treatment with Compound A if the mutation is detected in the sample of tumor cells from the subject; and
wherein the mutation comprises an EGFR C797S mutation.

101. A method of identifying a subject with cancer who is likely to be responsive to treatment with Compound A, wherein the method comprises:
(a) providing tumor cells of the subject;
(b) detecting presence or absence of a mutation in the provided tumor cells of the subject, wherein the mutation comprises an EGFR C797S mutation;
(c) identifying the subject as being likely to be responsive to treatment with Compound A the EGFR C797S mutation is detected in the provided tumor cells.

102. The method of any one of claims 97-101, wherein the HER-driven cancer comprises an EGFR-driven cancer.

103. The method of claim 102, wherein the EGFR-driven cancer comprises lung cancer.

104. The method of claim 103, wherein the lung cancer comprises non-small cell lung cancer (NSCLC).

105. The method of claim 102, wherein the cancer comprises brain cancer.

106. The method of claim 105, wherein the brain cancer is brain metastasis.

107. The method of any one of claims 97-106, wherein the mutation comprises a double or triple EGFR mutation.

108. The method of any one of claims 97-107, wherein the EGFR C797S mutation comprises at least one mutation selected from the group consisting of C797S/L858R, d746-750/C797S, d746-750/T790M/C797S, and T790M/C797S/L858R.

109. The method of any one of claims 97-108, wherein the cancer is resistant to at least one agent selected from the group consisting of osimertinib, gefitinib, afatinib, and erlotinib.

110. The method of any one of claims 97-109, further comprising administering at least one additional agent, or a salt or solvate thereof, that treats or prevents the cancer.

111. The method of claim 110, wherein Compound A and the at least one additional agent are co-administered to the subject.

112. The method of any one of claims 110-111, wherein Compound A and the at least one additional agent are coformulated.

113. The method of any one of claims 97-112 wherein Compound A is administered by at least one route selected from the group consisting of inhalational, oral, nasal, rectal, parenteral, sublingual, transdermal, transmucosal, intravesical, intrapulmonary, intraduodenal, intragastrical, intrathecal, epidural, intrapleural, intraperitoneal, intratracheal, otic, intraocular, subcutaneous, intramuscular, intradermal, intra-arterial, intravenous, intrabronchial, inhalation, and topical.

114. The method of any one of claims 97-113, wherein the subject is a mammal.

115. The method of claim 114, wherein the mammal is a human.

116. The method of claim 115, wherein the subject is a human in need of treatment thereof.

117. The method of any one of claims 97-116, wherein the cancer is a HER-driven drug-resistant cancer.

118. Use of Compound A in the manufacture of a medicament for the treatment of a HER-driven cancer, wherein the HER-driven cancer comprises an EGFR C797S mutation.

119. Use of Compound A in the manufacture of a medicament for the treatment of a HER-driven cancer, wherein the HER-driven cancer comprises an EGFR mutation.

120. Use of Compound A in the manufacture of a medicament for the treatment of a HER-driven cancer, wherein the HER-driven cancer comprises a HER2 mutation.

121. Use of Compound A in the manufacture of a medicament for the treatment of a HER-driven cancer, wherein the HER-driven cancer comprises a HER3 mutation.

122. Use of Compound A in the manufacture of a medicament for the treatment of a HER-driven cancer, wherein the HER-driven cancer comprises a HER4 mutation.

123. Use of Compound A in the manufacture of a medicament for the treatment of a HER-driven cancer, wherein the HER-driven cancer comprises HER mutation.

124. Use of Compound A in the manufacture of a medicament for the treatment of a HER-driven cancer, wherein the HER-driven cancer comprises a L858R mutation.

125. Use of Compound A in the manufacture of a medicament for the treatment of a HER-driven cancer, wherein the HER-driven cancer comprises an exon 19 deletion.

126. Use of Compound A in the manufacture of a medicament for the treatment of a HER-driven cancer, wherein the HER-driven cancer comprises an exon 20 insertion.

127. Use of Compound A in the manufacture of a medicament for the treatment of a HER-driven cancer, wherein the HER-driven cancer comprises a compound mutation.

128. Use of Compound A in the manufacture of a medicament for the treatment of a HER-driven cancer, wherein the HER-driven cancer comprises a fusion mutation.

129. Use of Compound A in the manufacture of a medicament for the treatment of a HER-driven cancer, wherein the HER-driven cancer comprises an atypical mutation.

130. Use of Compound A in the manufacture of a medicament for the treatment of a HER-driven cancer, wherein the HER-driven cancer comprises a rare mutation.

131. Use of Compound A in the manufacture of a medicament for the treatment of a HER-driven cancer, wherein the HER-driven cancer comprises an exon 18 or exon 19 mutation.

132. Use of Compound A in the manufacture of a medicament for the treatment of a HER-driven cancer, wherein the HER-driven cancer comprises an exon 20 mutation.

133. Use of Compound A in the manufacture of a medicament for the treatment of a HER-driven cancer, wherein the HER-driven cancer comprises an exon 21 mutation.

134. Use of Compound A in the manufacture of a medicament for the treatment of a HER-driven cancer, wherein the HER-driven cancer comprises a complex mutation.

135. Use of Compound A in the manufacture of a medicament for the treatment of a HER-driven cancer, wherein the HER-driven cancer comprises a HER2^{YVMA} mutation.

136. Use of Compound A in the manufacture of a medicament for the treatment of a HER-driven cancer, wherein the HER-driven cancer comprises an osimertinib resistant mutation.

137. Use of Compound A in the manufacture of a medicament for the treatment of a HER-driven cancer, wherein the HER-driven cancer comprises an EGFR mutation associated with lung adenocarcinoma.

138. Use of Compound A in the manufacture of a medicament for the treatment of a HER-driven cancer, wherein the HER-driven cancer comprises an EGFR mutation associated with gastric cancer.

139. Use of Compound A in the manufacture of a medicament for the treatment of a HER-driven cancer, wherein the HER-driven cancer comprises an EGFR mutation associated with lung cancer.

140. Use of Compound A in the manufacture of a medicament for the treatment of a HER-driven cancer, wherein the HER-driven cancer comprises an EGFR mutation associated with non-small cell lung adenocarcinoma.

141. Use of Compound A in the manufacture of a medicament for the treatment of a HER-driven cancer, wherein the HER-driven cancer comprises an EGFR mutation associated with urothelial carcinoma.

142. Use of Compound A in the manufacture of a medicament for the treatment of a HER-driven cancer, wherein the HER-driven cancer comprises an EGFR mutation associated with breast cancer.

143. Use of Compound A in the manufacture of a medicament for the treatment of a HER-driven cancer, wherein the HER-driven cancer comprises an EGFR mutation associated with Lynch and Lynch-like colorectal cancer.
